(19) United States
(12) Patent Application Publication
(10) Pub. No.: US 2026/0274859 A1
(43) Pub. Date: Sep. 17, 2026

(54) FUSED POLYCYCLIC DERIVATIVE AND USE THEREOF

(71) Applicants: ZHEJIANG HISUN PHARMACEUTICAL CO., LTD., Taizhou, Zhejiang (CN); HANGZHOU HEALZEN THERAPEUTICS CO., LTD., Hangzhou, Zhejiang (CN)

(72) Inventors: Houjiang ZHOU, Taizhou, Zhejiang (CN); Yizhe WU, Hangzhou, Zhejiang (CN); Xiaohe ZHENG, Taizhou, Zhejiang (CN); Yanling HE, Taizhou, Zhejiang (CN); Lifei MAO, Taizhou, Zhejiang (CN); Jinglai HUANG, Hangzhou, Zhejiang (CN); Penghao LUO, Taizhou, Zhejiang (CN); Jiangfeng XIE, Hangzhou, Zhejiang (CN); Haibo QIU, Shanghai (CN); Xingguo LIU, Hangzhou, Zhejiang (CN); Panpan ZHANG, Taizhou, Zhejiang (CN); Xinglu ZHOU, Hangzhou, Zhejiang (CN); Cheng YE, Shanghai (CN); Wenhua LUO, Hangzhou, Zhejiang (CN); Daiwang XU, Shanghai (CN); Xiaojie XU, Taizhou, Zhejiang (CN)

(73) Assignees: ZHEJIANG HISUN PHARMACEUTICAL CO., LTD., Taizhou, Zhejiang (CN); HANGZHOU HEALZEN THERAPEUTICS CO., LTD., Hangzhou, Zhejiang (CN)

(21) Appl. No.: 19/166,887

(22) PCT Filed: Mar. 28, 2024
(86) PCT No.: PCT/CN2024/084360

§ 371 Date: Sep. 19, 2025

(30) Foreign Application Priority Data
Mar. 30, 2023 (CN) 202310328028.7

(51) Int. Cl. C07D 495/04; A61K 31/4365; A61K 31/437; A61K 31/444; A61K 31/496; A61K 31/506; A61K 31/5377; A61P 35/02; C07D 519/00

(52) U.S. Cl. CPC C07D 495/04; A61K 31/4365; A61K 31/437; A61K 31/444; A61K 31/496; A61K 31/506; A61K 31/5377; A61P 35/02; C07D 519/00

(57) ABSTRACT

The present invention relates to a fused polycyclic Myc regulator and a preparation method therefor, a pharmaceutical composition containing the regulator, and a use of the regulator in medicine. Disclosed in the present invention are a fused polycyclic derivative represented by general formula (I), or a stereoisomer thereof, a tautomer thereof, a pharmaceutically acceptable salt thereof and a preparation method therefor, and a use thereof as a Myc regulator.

This application claims the priority to Chinese Patent Application No. 2023 10328028.7, titled “FUSED POLYCYCLIC DERIVATIVE AND USE THEREOF”, filed on Mar. 30, 2023 with the China National Intellectual Property Administration, which is incorporated herein by reference in its entirety.

### FIELD

The present disclosure relates to a fused polycyclic derivative, a production method thereof, a pharmaceutical composition containing the derivative and use thereof as a therapeutic agent, particularly as a Myc regulator.

### BACKGROUND

Myc, as an important transcription factor in cells, can regulate many biological functions in cells, such as cell proliferation, cell differentiation, cell cycle progress and metabolism. Myc gene family is mainly composed of three members: c-Myc, N-Myc and L-Myc. Myc and partner protein MAX form heterodimers, which target and bind to DNA sequences or E-boxes, thereby regulating gene transcription related to the growth and proliferation of cells.

Under normal physiological conditions, the expression of Myc is strictly regulated. A large number of studies have shown that it is very common in human cancer that the overexpression or dysregulation of c-Myc leads to tumor occurrence (The MYC oncogene—the grand orchestrator of cancer growth and immune evasion, Nat.Rev.Clin.Oncol., 2022, 19, 23-36). Myc can be activated abnormally through gene amplification, epigenetics, post-translation modification, etc. A variety of cancer-promoting programs such as cell proliferation, inhibition of cell apoptosis, genomic instability, metabolism and invasiveness, angiogenesis and immune evasion are stimulated to maintain and accelerate the malignant growth of tumors.

A large number of studies have shown that Myc is the key driver of cancer occurrence, and inactivation of Myc can inhibit the growth of tumors (such as lymphoma, leukemia, osteosarcoma, liver cancer, kidney cancer, lung cancer, pancreatic cancer and breast cancer) and lead to tumor regression (Modelling Myc inhibition as a cancer therapy, Nature, 2008, 455, 679-683; Myc Cooperates with Ras by Programming inflammation and immune suppression, Cell, 2017, 171, 1301-1315; MYC Instructs and Maintains Pancreatic Adenocarcinoma Phenotype, Cancer. Discov., 2020,10,588-607), which proves that targeting Myc has great clinical value for a variety of tumors.

Due to the high disorder of structure and the lack of specific binding sites, Myc is considered as a difficult-to-drug target. Small molecule inhibitors targeting Myc (such as inhibitor of MYC identified in a Kronhnke pyridine library, Proc. Natl. Acad. Sci., 2014, 111, 12556-12561; A selective high affinity MYC-binding compound inhibits, Sci. Rep., 2018,8,10064; CN114174265A) can inhibit the transcription function of Myc to a certain extent, showing a certain potential, but there is still a huge gap from small molecular drugs, and so far, no small molecular drugs with excellent druggabilities have entered the clinical application stage. Therefore, it is of great clinical significance to develop Myc regulators with high druggabilities.

### SUMMARY

An object of the present disclosure is to provide a fused polycyclic compound represented by formula (I) or a stereoisomer, a tautomer or a pharmaceutically acceptable salt thereof:

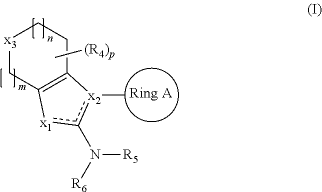

wherein:
X₁ is selected from the group consisting of N, NR₁, O and S;
X₂ is selected from the group consisting of C, CH and N;
X₃ is selected from the group consisting of C(O), C(═NR₃), CR₂R₂′, NR₃, O, S, S(O)₂ and S(═NH)O;
R₁ is selected from the group consisting of H, C₁-C₈ alkyl, C₁-C₈ haloalkyl, C₃-C₁₂ cycloalkyl, 3- to 12-membered heterocyclyl, C₆-C₁₀ aryl, 5- to 12-membered heteroaryl, amino, hydroxyl, nitro and cyano;
R₂ and R₂′ are each independently selected from the group consisting of H, deuterium, halogen, C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, amino, hydroxyl, nitro, cyano, C₁-C₈ alkylamino, C₁-C₈ haloalkylamino, C₁-C₈ alkyl-C(O)O—, and C₁-C₈ alkyl, —C(O)NH—; or R₂ and R₂′ form a 3- to 12-membered ring together with the carbon atom to which they are jointly attached, and the 3- to 12-membered ring contains 0, 1 or 2 heteroatoms selected from the group consisting of N, O, S and P; the alkyl, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, nitro, cyano, —C(O)—(C₁-C₈)alkyl, —C(O)O—(C₁-C₈)alkyl, —C(O)NH—(C₁-C₈)alkyl and —NHC(O)—(C₁-C₈)alkyl, and R₂ and R₂′ are not both H;
R₃ and R₄ are each independently selected from the group consisting of H, deuterium, halogen, C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, amino, hydroxyl, nitro, cyano, carboxyl, C₁-C₈alkylamino, C₁-C₈ haloalkylamino, —OR_{g}, —SR_{g}, —C₁-C₈ alkylene-R_{g}, —OC(O)R_{g}, —C(O)R_{g}, —C(O)OR_{g}, —C(O)N(Rₓ)R_{y}, —NRₓR_{y}, —N(CH₃)R_{g}, —N(Rₓ)C(O)R_{y}, —N(Rₓ)C(O)NRₓR_{y}, —N(Rₓ)C(O)OR_{g}, —N(Rₓ)S(O)NRₓR_{y}, —N(Rₓ)S(O)₂NRₓR_{y}, —N(Rₓ)S(O)₂R_{g}, —S(O)R_{g}, —S(O)₂R_{g}, —S(O)₂NRₓR_{y} and —P(O)RₓR_{y}; the alkyl, alkylene, alkenyl, alkynyl, cycloalkyl, heterocyclyl, alkoxy, aryl, heteroaryl, amino or hydroxyl is optionally further substituted by one or more Rₙ; or,
two R₄ form a 3- to 8-membered ring together with one or more atoms to which they are attached (provided that the valence is satisfied), the 3- to 8-membered ring contains 0, 1 or 2 heteroatoms selected from the group consisting of N, O, S and P, and the 3- to 8-membered ring is optionally further substituted by one or more substituents selected from the group consisting of halogen, C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano; or,
R₃ and R₄ form a 3- to 8-membered heterocyclic ring together with the atoms to which they are attached, and the 3- to 8-membered heterocyclic ring is optionally further substituted by one or more substituents selected from the group consisting of halogen, C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano; or,
R₂ and R₄ form a 3- to 8-membered ring together with the atom to which they are attached, the 3- to 8-membered ring contains 0, 1 or 2 heteroatoms selected from the group consisting of N, O, S and P, and the 3- to 8-membered ring is optionally further substituted by one or more substituents selected from the group consisting of halogen, C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano;
R₆ is selected from the group consisting of H, C₁-C₈ alkyl, C₃-C₁₂ cycloalkyl, 3- to 12-membered heterocyclyl and C₁-C₈ haloalkyl, and the alkyl, cycloalkyl, heterocyclyl or haloalkyl is optionally further substituted by one or more substituents selected from the group consisting of C₁-C₈ alkyl, C₃-C₁₂ cycloalkyl, 3- to 12-membered heterocyclyl, C₁-C₈ alkoxy, halogen, amino, hydroxyl, nitro and cyano; R₆ is selected from the group consisting of —C(RₐR_{b})R_{c}, —C(O)OR_{c}, —C(O)NRₕR_{c}, —C(S)C(RₐR_{b})R_{c}, —C(S)N(Rₕ)C(RₐR_{b})R_{c}, C₆-C₁₀ aryl, 5- to 12-membered heteroaryl-NR_{d}Rₑ, 3- to 12-membered heterocyclyl, C₃-C₁₂ cycloalkyl, —C(O)—C(O)—C(RₐR_{b})R_{c}, —C(O)—C(O)—N(Rₕ)—C(RₐR_{b})R_{c},

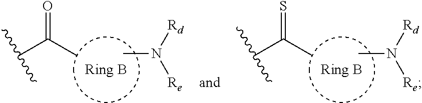

or,

R₅ and R₆ form a 3- to 14-membered heterocyclic ring substituted by one or more substituents together with the atom to which they are attached, the substituent is selected from the group consisting of C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, oxo, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —C₁-C₈ alkylene-O—(C₁-C₈)alkyl and —NR_{d}Rₑ; or, when two substituents are substituted on the same atom on the 3- to 14-membered heterocyclic ring, the substituents form a 3- to 12-membered ring together with the atom to which they are attached, and the 3- to 12-membered ring contains 0, 1 or 2 heteroatoms selected from the group consisting of N, O, S and P; the alkyl, alkylene, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈ alkyl, C₃-C₁₂ cycloalkyl, 3- to 12-membered heterocyclyl, C₁-C₈ alkoxy, C₁-C₈ haloalkoxy, halogen, amino, hydroxyl, nitro and cyano;
Rₐ and R_{b} are each independently selected from the group consisting of H, halogen, C₁-C₈ alkyl, C₃-C₁₂ cycloalkyl, C₁-C₈ haloalkyl, 3- to 12-membered heterocyclyl, C₆-C₁₀ aryl, 5- to 12-membered heteroaryl, C₂-C₈ alkynyl, C₂-C₈ alkenyl, —OH, —NH₂, C₁-C₈ alkoxy, C₁-C₈ alkylamino and —CN, the alkyl, cycloalkyl, haloalkyl, heterocyclyl, aryl, heteroaryl, alkynyl or alkenyl is optionally further substituted by one or more Rₙ;
R_{c} is selected from —(CH₂)s-NR_{d}Rₑ, wherein one or more CH₂ are optionally replaced by one or more groups selected from the group consisting of —N(Rₕ′)—, —C(RₐR_{b})—, —O—, —S—, —C(O)—, C₃-C₁₂ cycloalkyl, 3- to 12-membered heterocyclyl, phenyl, 5- to 12-membered heteroaryl, —C(Rₐ)═C(R_{b})—, alkyne bond, —S(O)—, —S(O)₂— and —P(O)Rₕ′;
R_{d} is selected from the group consisting of H, —(CH₂)s-Rₕ, wherein one or more CH₂ are optionally replaced by one or more groups selected from the group consisting of —N(Rₕ′)—, —C(RₐR_{b})—, —O—, —S—, —C(O)—, C₃-C₁₂ cycloalkyl, 3- to 12-membered heterocyclyl, phenyl, 5- to 12-membered heteroaryl, —C(Rₐ)—C(R_{b})—, alkyne bond, —S(O)—, —S(O)₂— and —P(O)Rₕ′, the —CH₂—, cycloalkyl, heterocyclyl, phenyl or heteroaryl is optionally further substituted by one or more Rₙ;
Rₑ is selected from the group consisting of H, C₁-C₈ alkyl, C₃-C₁₂ cycloalkyl, C₁-C₈ haloalkyl, 3- to 12-membered heterocyclyl, phenyl and 5- to 12-membered heteroaryl; the alkyl, cycloalkyl, heterocyclyl, phenyl or heteroaryl is optionally further substituted by one or more Rₘ;
Rₘ is selected from the group consisting of H, halogen, C₁-C₈ alkyl, C₃-C₁₂ cycloalkyl, C₁-C₈ alkoxy, C₁-C₈ alkylamino, amino, hydroxyl, cyano and nitro;
Rₕ and Rₕ′ are each independently selected from the group consisting of H, C₁-C₈ alkyl, C₁-C₈ haloalkyl, C₃-C₁₂ cycloalkyl, phenyl, 5- to 12-membered heteroaryl and 3- to 12-membered heterocyclyl; the alkyl, haloalkyl, cycloalkyl, phenyl, heteroaryl or heterocyclyl is optionally further substituted by one or more Rₙ;
Ring A is selected from the group consisting of 5- to 6-membered heterocyclic ring, 5- to 12-membered heteroaromatic ring, benzene ring, 9- to 10-membered bicyclic heterocyclyl and 9- to 10-membered fused ring; the heterocyclic ring, heteroaromatic ring, benzene ring or fused ring is optionally further substituted by one or more Rₙ, or, when two Rₙ are attached to the same atom, the two Rₙ form a 3- to 6-membered ring together with the atom to which they are attached; or, when two Rₙ are attached to adjacent atoms, the two Rₙ form a 3- to 12-membered ring together with the atoms to which they are attached;
Ring B is selected from the group consisting of C₃-C₁₂ cycloalkyl, 3- to 12-membered heterocyclyl, C₆-C₁₀ aryl and 5- to 12-membered heteroaryl, the cycloalkyl, heterocyclyl, aryl or heteroaryl is optionally further substituted by one or more Rₙ′;
when Ring B is selected from benzene ring, Rₐ is selected from H;
Rₙ and Rₙ′ are each independently selected from the group consisting of H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heterocyclyloxy, amino sulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —OR_{g}, —SR_{g}, —C₁-C₈ alkylene-R_{g}, —OC(O)R_{g}, —C(O)R_{g}, —C(O)OR_{g}, —C(O)N(Rₓ)R_{y}, —NRₓR_{y}, —N(CH₃)R_{g}, —N(Rₓ)C(O)R_{y}, —N(Rₓ)C(O)NRₓR_{y}, —N(Rₓ)C(O)OR_{g}, —N(Rₓ)S(O)NRₓR_{y}, —N(Rₓ)S(O)₂NRₓR_{y}, —N(Rₓ)S(O)₂R_{g}, —S(O)R_{g}, —S(O)₂R_{g}, —S(O)₂NRₓR_{y} and —P(O)RₓR_{y}; or,
two Rₙ form a 3- to 12-membered ring together with the atom to which they are jointly attached, and the 3- to 12-membered ring contains 0, 1 or 2 heteroatoms selected from the group consisting of N, O, S and P; the alkyl, alkylene, alkoxy, alkenyl, alkynyl, alkenyloxy, cycloalkyl, cycloalkoxy, heterocyclyl, heterocyclyloxy, aryl, heteroaryl, 3- to 12-membered ring, amino, hydroxyl or amide is optionally further substituted by one or more Rₒ; or,
two Rₙ′ form a 3- to 12-membered ring together with the atom to which they are jointly attached, and the 3- to 12-membered ring contains 0, 1 or 2 heteroatoms selected from the group consisting of N, O, S and P; the alkyl, alkylene, alkoxy, alkenyl, alkynyl, alkenyloxy, cycloalkyl, cycloalkoxy, heterocyclyl, heterocyclyloxy, aryl, heteroaryl, 3- to 12-membered ring, amino, hydroxyl or amide is optionally further substituted by one or more Rₒ; or, when two Rₒ are attached to the same atom, the two Rₒ form a 3- to 6-membered ring together with the atom to which they are attached; or, when two Rₒ are attached to adjacent atoms, the two Rₒ form a 3- to 12-membered ring together with the atom to which they are attached;
R_{g}, Rₓ, R_{y} and Rₒ are each independently selected from the group consisting of H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heterocyclyloxy, aminosulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —ORₛ, —SRₛ, —C₁-C₈ alkylene-Rₛ, —OC(O)Rₛ, —C(O)Rₛ, —C(O)ORₛ, —C(O)N(Rₛ)Rₜ, —NRₛRₜ, —N(CH₃)Rₛ, —N(Rₛ)C(O)Rₜ, —N(Rₛ)C(O)NRₛRₜ, —N(Rₛ)C(O)ORₜ, —N(Rₛ)S(O)NRₛRₜ, —N(Rₛ)S(O)₂NRₛRₜ, —N(Rₛ)S(O)₂Rₜ, —S(O)Rₛ, —S(O)₂Rₛ, —S(O)₂NRₛRₜ and —P(O)RₛRₜ, and the alkyl, alkylene, cycloalkyl, heterocyclyl, aryl or heteroaryl is optionally further substituted by one or more Rᵣ, or,
when two Rᵣ are attached to the same atom, the two Rᵣ form a 3- to 6-membered ring together with the atom to which they are attached, or when the two Rᵣ are attached to adjacent atoms, the two Rᵣ form a 3- to 12-membered ring together with the atom to which they are attached;
Rᵣ, Rₛ and Rₜ are each independently selected from the group consisting of H, deuterium, C₁-C₈ alkyl, C₂-C₈ alkenyl, C₂-C₈ alkynyl, halogen, cyano, amino, nitro, hydroxyl, oxo, C₁-C₈ alkoxy, C₁-C₈ haloalkyl, hydroxyalkyl, aminoalkyl, C₁-C₈ alkylamino, alkylcarbonyl, alkoxycarbonyl, halohydroxyalkyl, C₁-C₈ haloalkylamino, C₃-C₁₂ cycloalkyl, 3- to 12-membered heterocyclyl, carboxyl, amide, C₆-C₁₀ aryl and 5- to 12-membered heteroaryl;
[symbol] represents a chemical bond or is absent, and at most one chemical bond presents;
m is selected from the group consisting of 0, 1 and 2;
n is selected from the group consisting of 0, 1 and 2;
p is selected from the group consisting of 0, 1, 2, 3, 4, 5 and 6; and
s is selected from the group consisting of 0, 1, 2, 3, 4, 5, 6, 7, 8 and 9.

In a preferred embodiment of the present disclosure, provided is the compound represented by formula (I) or the stereoisomer, the tautomer or the pharmaceutically acceptable salt thereof, which is a compound represented by formula (IIa) or formula (IIb) or a stereoisomer, a tautomer or a pharmaceutically acceptable salt thereof:

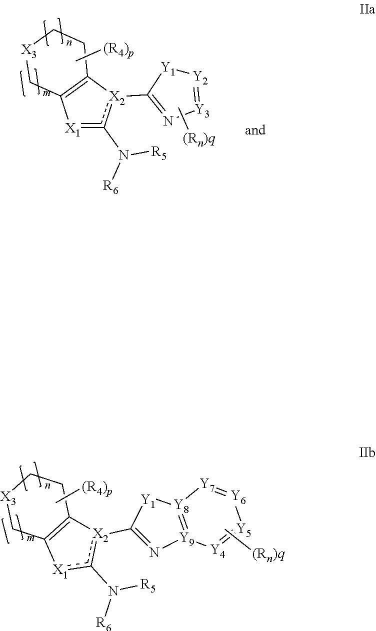

wherein:
Y₁ is selected from the group consisting of CH₂, NH, O and S;
Y₂ is selected from the group consisting of CH and N;
Y₃ is selected from the group consisting of CH and N;
Y₈ is selected from C;
Y₉ is selected from C;
Y₄, Y₅, Y₆ and Y₇ are each independently selected from the group consisting of CH and N;
q is selected from the group consisting of 0, 1, 2, 3, 4 and 5; and
Rₙ is as defined in formula (I).

In a preferred embodiment of the present disclosure, provided is the compound represented by formula (I) or the stereoisomer, the tautomer or the pharmaceutically acceptable salt thereof, which is a compound represented by formula (IIIa) or formula (IIIb) or a stereoisomer, a tautomer or a pharmaceutically acceptable salt thereof:

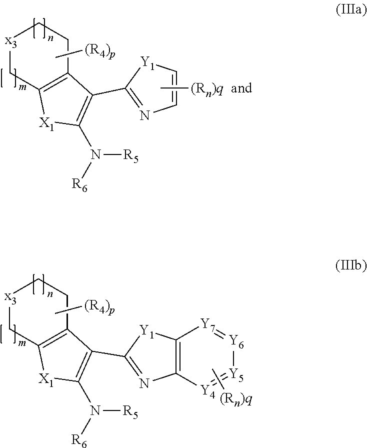

wherein:
X₁ is selected from the group consisting of NH, O and S;
Y₁ is selected from the group consisting of CH₂, NH, O and S;
Y₄, Y₅, Y₆ and Y₇ are each independently selected from the group consisting of CH and N; and
X₃, R₄, R₅, R₆, Rₙ, m, n, p and q are as defined in formula (I).

In a preferred embodiment of the present disclosure, provided is the compound represented by formula (I) or the stereoisomer, the tautomer or the pharmaceutically acceptable salt thereof, which is a compound represented by formula (IVa) or formula (IVb) or a stereoisomer, a tautomer or a pharmaceutically acceptable salt thereof:

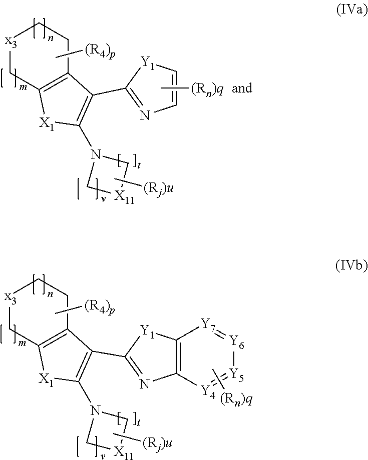

wherein:
X₁₁ is selected from the group consisting of CR₇R₇′, NR₇ and O;
R₇ and R₇′ are each independently selected from the group consisting of H, C₁-C₈ alkyl, C₂-C₅ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —(C₁-C₈)alkylene-O—(C₁-C₈)alkyl and —NR_{d}Rₑ; or, R₇ and R₇′ form a 3- to 12-membered ring together with the carbon atom to which they are jointly attached, and the 3- to 12-membered ring contains 0, 1 or 2 heteroatoms selected from the group consisting of N, O, S and P; and the alkyl, alkylene, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈ alkyl, C₃-C₁₂ cycloalkyl, 3- to 12-membered heterocyclyl, C₁-C₈ alkoxy, C₁-C₈ haloalkoxy, halogen, amino, hydroxyl, nitro and cyano;
Rⱼ is selected from the group consisting of C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, oxo, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —(C₁-C₈)alkylene-O—(C₁-C₈)alkyl and —NR_{d}Rₑ, or two Rⱼ form a 3- to 12-membered ring together with the atom to which they are jointly attached, and the 3- to 12-membered ring contains 0, 1 or 2 heteroatoms selected from the group consisting of N, O, S and P;
v is selected from the group consisting of 0, 1, 2, 3, 4, 5 and 6;
t is selected from the group consisting of 0, 1, 2, 3, 4, 5 and 6;
u is selected from the group consisting of 0, 1, 2, 3, 4, 5 and 6;
wherein, v and t are not both 0; and
X₁, X₃, Y₁, Y₄, Y₅, Y₆, Y₇, R₄, Rₙ, Rₐ, Rₑ, m, n, p and q are as defined in formula (I).

In a preferred embodiment of the present disclosure, provided is the compound represented by formula (I) or the stereoisomer, the tautomer or the pharmaceutically acceptable salt thereof, which is a compound represented by formula (V) or a stereoisomer, a tautomer or a pharmaceutically acceptable salt thereof:

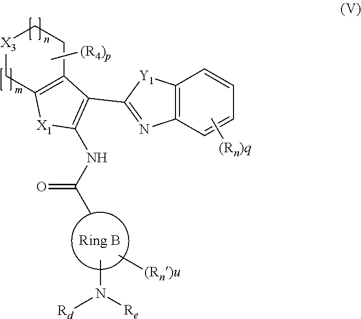

wherein:
Y₁ is selected from the group consisting of CH₂, NH, O and S;
X₁ is selected from the group consisting of NH, O and S;
Ring B is selected from the group consisting of C₃-C₁₂ cycloalkyl, 3- to 12-membered heterocyclyl, C₆-C₁₀ aryl and 5- to 12-membered heteroaryl;
u is selected from the group consisting of 0, 1, 2, 3, 4, 5 and 6; and
X₃, R₄, Rₐ, Rₑ, Rₙ, Rₙ′, m, n, p and q are as defined in formula (I).

In a preferred embodiment of the present disclosure, provided is a compound represented by formula (IIIa) or formula (IIIb) or a stereoisomer, a tautomer or a pharmaceutically acceptable salt thereof, wherein:
Rₛ is selected from H;
R₆ is selected from the group consisting of —C(RₐR_{b})R_{c}, —C(O)OR_{c}, —C(O)NRₕR_{c}, —C(O)—C(RₐR_{b})R_{c} and —C(O)—C(O)—N(Rₕ)—C(RₐR_{b})R_{c}; and
Rₐ, R_{b}, R_{c} and Rₕ are as defined in formula (I).

Further preferably, R_{c} is selected from the group consisting of:

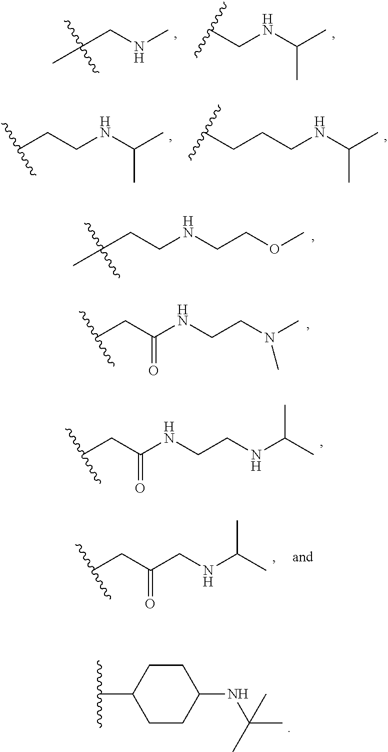

Further preferably, Rₕ is selected from the group consisting of H and methyl. In a preferred embodiment of the present disclosure, provided is a compound represented by formula (VIa), formula (VIb), formula (VIc) or formula (VId), or a stereoisomer, a tautomer or a pharmaceutically acceptable salt thereof, wherein:

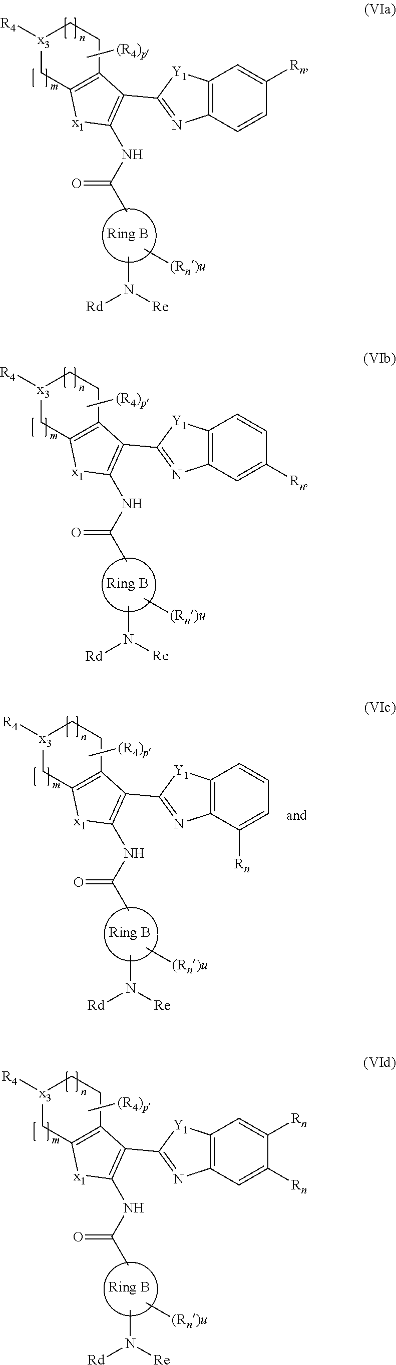

P′ is selected from the group consisting of 0, 1, 2, 3, 4 and 5; and
X₃, R₄, R_{d}, Rₑ, Rₙ, Rₙ′, m, n and q are as defined in formula (V).

Further preferably, Ring B is preferably selected from the group consisting of:

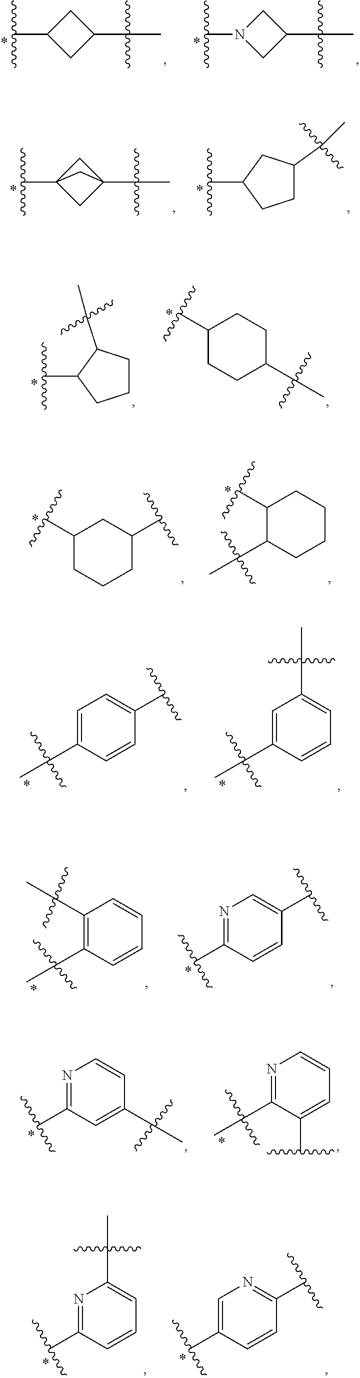

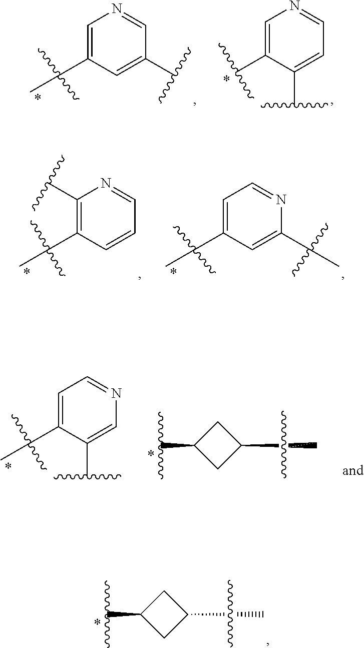

and
* end is attached to carbonyl or carbon-sulfur group.

Further preferably, Rₐ is selected from the group consisting of C₁-C₄ alkyl,

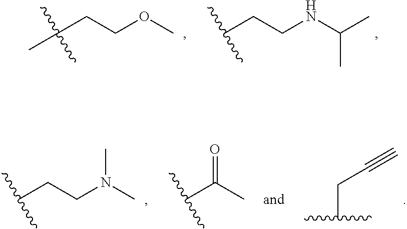

Further preferably, Rₑ is selected from the group consisting of H and methyl.

Further preferably, Rₙ is selected from the group consisting of H, methyl, halogen, trifluoromethyl, cyano, methoxy,

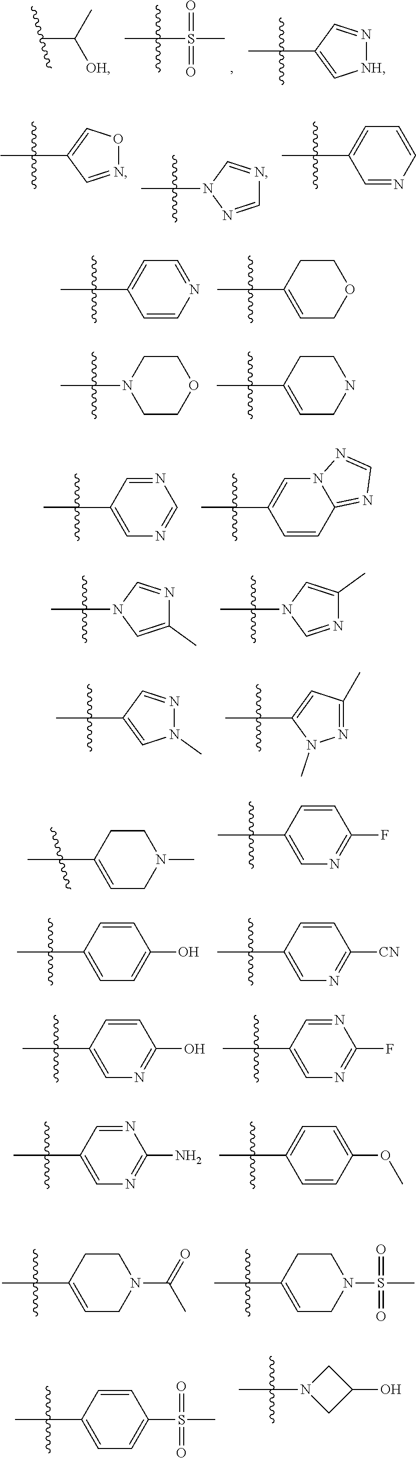

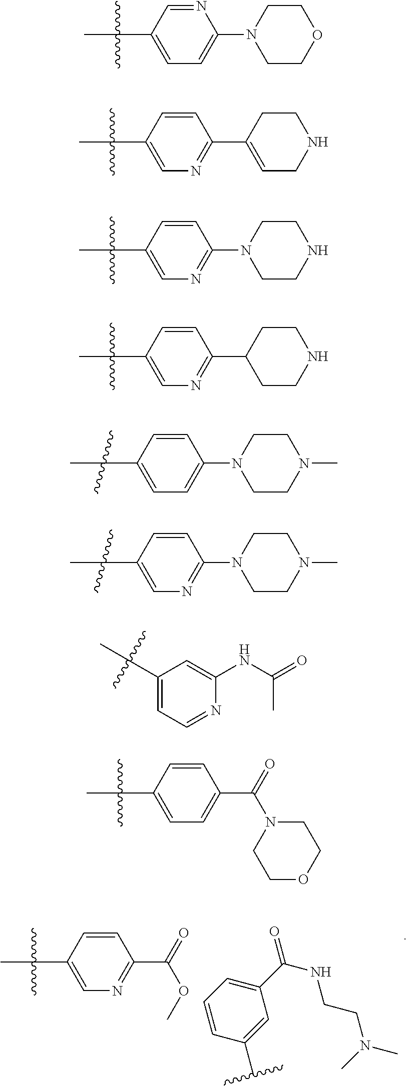

Further preferably, R₄ is selected from the group consisting of H, deuterium, halogen, C₁-C₃ alkyl, C₁-C₃ haloalkyl, C₃-C₆ cycloalkyl, 4- to 6-membered heterocyclyl and —C(O)R_{g}; and

R_{g} is selected from the group consisting of H and C₁-C₃ alkyl.

The specific structure of the compound represented by the formula (I) of the present disclosure includes, but is not limited to:

| Structure of the compound | Name |
|---|---|
| 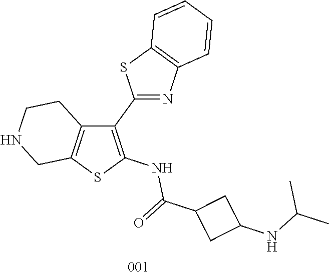 [SMILES: CC(C)NC1CC(C(=O)Nc2sc3c(c2-c2nc4ccccc4s2)CCNC3)C1] | N-(3-(benzo[d]thiazol-2-yl)- 4,5,6,7-tetrahydrothieno [2,3-c]pyridin-2-yl)-3- (isopropylamino)cyclobutane- 1-carboxamide |
| 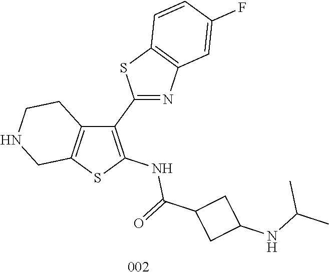 [SMILES: CC(C)NC1CC(C(=O)Nc2sc3c(c2-c2nc4cc(F)ccc4s2)CCNC3)C1] | N-(3-(5-fluorobenzo[d] thiazol-2-yl)-4,5,6,7- tetrahydrothieno [2,3-c]pyridin- 2-yl)-3-(isopropylamino) cyclobutane- 1-carboxamide |
| 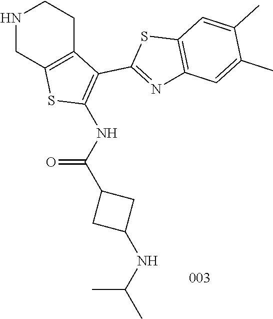 [SMILES: Cc1cc2nc(-c3c(NC(=O)C4CC(NC(C)C)C4)sc4c3CCNC4)sc2cc1C] | N-(3-(5,6-dimethylbenzo[d] thiazol-2-yl)-4,5,6,7- tetrahydrothieno[2,3-c]pyridin- 2-yl)-3-(isopropylamino) cyclobutane-1- carboxamide |
| 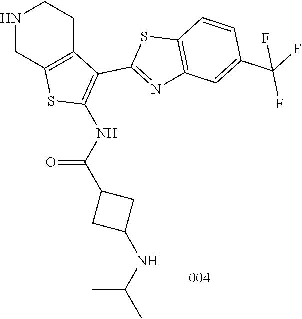 [SMILES: CC(C)NC1CC(C(=O)Nc2sc3c(c2-c2nc4cc(C(F)(F)F)ccc4s2)CCNC3)C1] | 3-(isopropylamino)-N-(3-(5- (trifluoromethyl)benzo [d]thiazol-2-yl)-4,5,6,7- tetrahydrothieno[2,3-c] pyridin-2-yl)cyclobutane- 1-carboxamide |
| 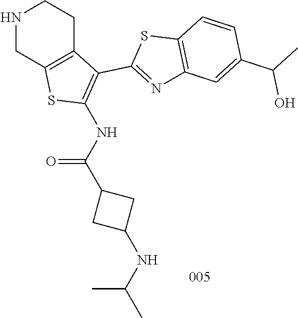 [SMILES: CC(C)NC1CC(C(=O)Nc2sc3c(c2-c2nc4cc(C(C)O)ccc4s2)CCNC3)C1] | N-(3-(5-(1-hydroxyethyl)benzo [d]thiazol-2-yl)-4,5,6,7- tetrahydrothieno [2,3-c]pyridin-2-yl)-3- (isopropylamino) cyclobutane-1-carboxamide |
| 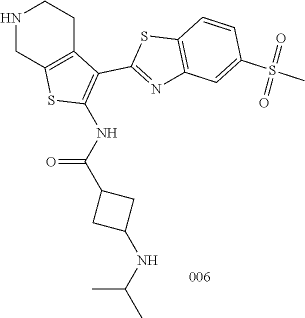 [SMILES: CC(C)NC1CC(C(=O)Nc2sc3c(c2-c2nc4cc(S(C)(=O)=O)ccc4s2)CCNC3)C1] | 3-(isopropylamino)-N-(3- (5-(methylsulfonyl)benzo [d]thiazol-2-yl)-4,5,6,7- tetrahydrothieno[2,3-c]pyridin- 2-yl)cyclobutane- 1-carboxamide |
| 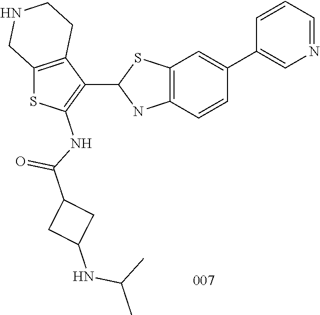 [SMILES: CC(C)NC1CC(C(=O)Nc2sc3c(c2C2Nc4ccc(-c5cccnc5)cc4S2)CCNC3)C1] | 3-(isopropylamino)-N-(3- (6-(pyridin-3-yl)benzo[d] thiazol-2-yl)-4,5,6,7- tetrahydrothieno[2,3-c]pyridin-2- yl)cyclobutane- 1-carboxamide |
| 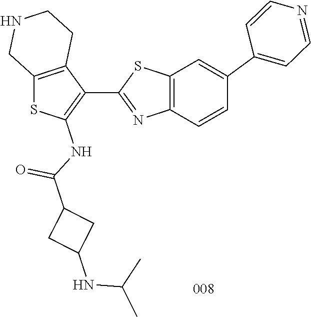 [SMILES: CC(C)NC1CC(C(=O)Nc2sc3c(c2-c2nc4ccc(-c5ccncc5)cc4s2)CCNC3)C1] | 3-(isopropylamino)-N-(3- (6-(pyridin-4-yl)benzo[d] thiazol-2-yl)-4,5,6,7- tetrahydrothieno[2,3-c]pyridin-2- yl)cyclobutane- 1-carboxamide |
| 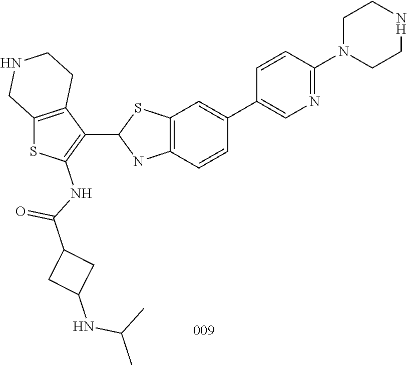 [SMILES: CC(C)NC1CC(C(=O)Nc2sc3c(c2C2Nc4ccc(-c5ccc(N6CCNCC6)nc5)cc4S2)CCNC3)C1] | 3-(isopropylamino)-N-(3-(6- (6-(piperazin-1-yl)pyridin- 3-yl)benzo[d]thiazol-2-yl)- 4,5,6,7-tetrahydrothieno [2,3-c]pyridin-2-yl) cyclobutane-1-carboxamide |
| 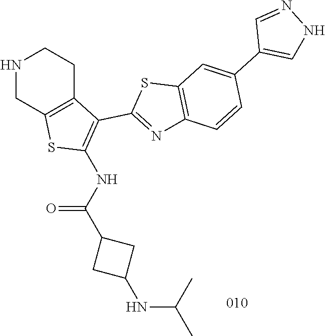 [SMILES: CC(C)NC1CC(C(=O)Nc2sc3c(c2-c2nc4ccc(-c5cn[nH]c5)cc4s2)CCNC3)C1] | N-(3-(6-(1H-pyrazol-4-yl) benzo[d]thiazol-2-yl)-4,5, 6,7-tetrahydrothieno [2,3-c]pyridin-2-yl)-3- (isopropylamino) cyclobutane-1-carboxamide |
| 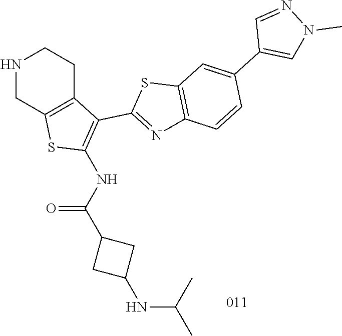 [SMILES: CC(C)NC1CC(C(=O)Nc2sc3c(c2-c2nc4ccc(-c5cnn(C)c5)cc4s2)CCNC3)C1] | 3-(isopropylamino)-N-(3-(6- (1-methyl-1H-pyrazol-4- yl)benzo[d]thiazol-2-yl)- 4,5,6,7-tetrahydrothieno [2,3-c]pyridin-2-yl) cyclobutane-1-carboxamide |
| 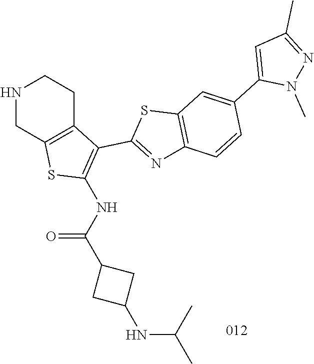 [SMILES: Cc1cc(-c2ccc3nc(-c4c(NC(=O)C5CC(NC(C)C)C5)sc5c4CCNC5)sc3c2)n(C)n1] | N-(3-(6-(1,3-dimethyl-1H- pyrazol-5-yl)benzo[d]thiazol- 2-yl)-4,5,6,7-tetrahydrothieno [2,3-c]pyridin-2-yl)- 3-(isopropylamino) cyclobutane-1-carboxamide |
| 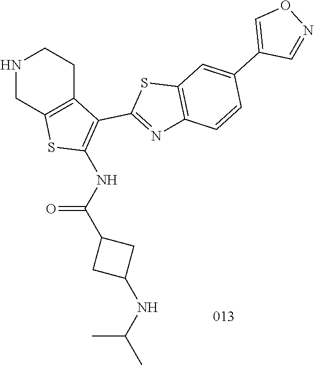 [SMILES: CC(C)NC1CC(C(=O)Nc2sc3c(c2-c2nc4ccc(-c5cnoc5)cc4s2)CCNC3)C1] | 3-(isopropylamino)-N-(3- (6-(isoxazol-4-yl)benzo[d] thiazol-2-yl)-4,5,6,7- tetrahydrothieno[2,3-c]pyridin-2- yl)cyclobutane- 1-carboxamide |
| 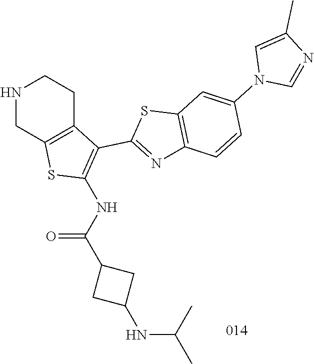 [SMILES: Cc1cn(-c2ccc3nc(-c4c(NC(=O)C5CC(NC(C)C)C5)sc5c4CCNC5)sc3c2)cn1] | 3-(isopropylamino)-N-(3- (6-(4-methyl-1H-imidazol- 1-yl)benzo[d]thiazol-2-yl)- 4,5,6,7-tetrahydrothieno [2,3-c]pyridin-2-yl) cyclobutane-1-carboxamide |
| 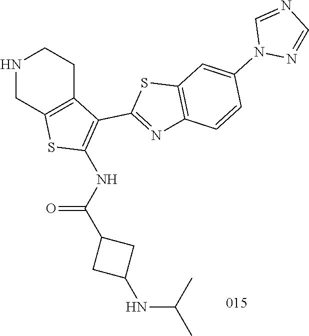 [SMILES: CC(C)NC1CC(C(=O)Nc2sc3c(c2-c2nc4ccc(-n5cncn5)cc4s2)CCNC3)C1] | N-(3-(6-(1H-1,2,4-triazol- 1-yl)benzo[d]thiazol-2-yl)- 4,5,6,7-tetrahydrothieno [2,3-c]pyridin-2-yl)-3- (isopropylamino) cyclobutane-1-carboxamide |
| 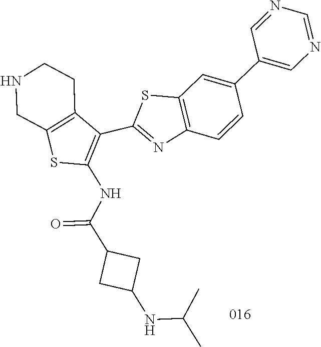 [SMILES: CC(C)NC1CC(C(=O)Nc2sc3c(c2-c2nc4ccc(-c5cncnc5)cc4s2)CCNC3)C1] | 3-(isopropylamino)-N-(3-(6- (pyrimidin-5-yl)benzo[d] thiazol-2-yl)-4,5,6,7- tetrahydrothieno[2,3-c]pyridin- 2-yl)cyclobutane- 1-carboxamide |
| 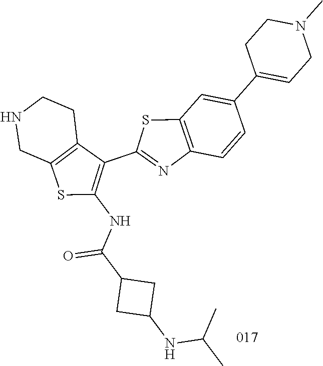 [SMILES: CC(C)NC1CC(C(=O)Nc2sc3c(c2-c2nc4ccc(C5=CCN(C)CC5)cc4s2)CCNC3)C1] | 3-(isopropylamino)-N- (3-(6-(1-methyl-1,2,3,6- tetrahydropyridin-4-yl) benzo[d]thiazol-2-yl)-4,5,6,7- tetrahydrothieno[2,3-c] pyridin-2-yl)cyclobutane-1- carboxamide |
| 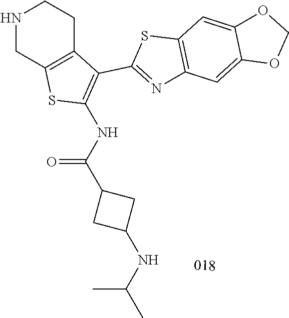 [SMILES: CC(C)NC1CC(C(=O)Nc2sc3c(c2-c2nc4cc5c(cc4s2)OCO5)CCNC3)C1] | N-(3-([1,3]dioxolo[4′,5′:4,5] benzo[1,2-d]thiazol-6-yl)- 4,5,6,7-tetrahydrothieno [2,3-c]pyridin-2-yl)-3- (isopropylamino) cyclobutane-1-carboxamide |
| 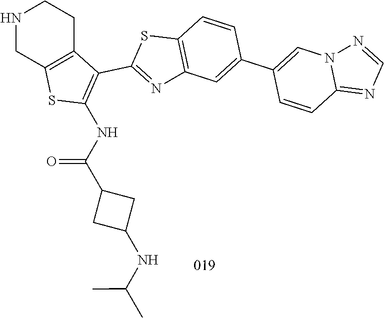 [SMILES: CC(C)NC1CC(C(=O)Nc2sc3c(c2-c2nc4cc(-c5ccc6ncnn6c5)ccc4s2)CCNC3)C1] | N-(3-(5-([1,2,4]triazolo[1,5- a]pyridin-6-yl)benzo[d] thiazol-2-yl)-4,5,6,7- tetrahydrothieno[2,3-c]pyridin-2- yl)-3-(isopropylamino) cyclobutane-1-carboxamide |
| 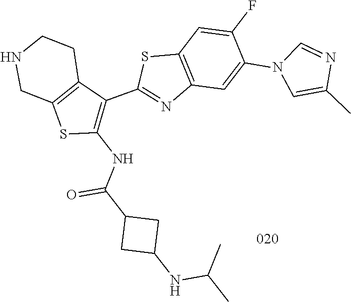 [SMILES: Cc1cn(-c2cc3nc(-c4c(NC(=O)C5CC(NC(C)C)C5)sc5c4CCNC5)sc3cc2F)cn1] | N-(3-(6-fluoro-5-(4-methyl- 1H-imidazol-1-yl)benzo [d]thiazol-2-yl)-4,5,6,7- tetrahydrothieno[2,3-c]pyridin- 2-yl)-3-(isopropylamino) cyclobutane-1-carboxamide |
| 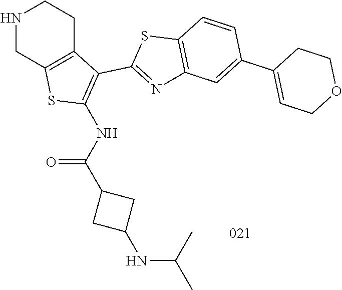 [SMILES: CC(C)NC1CC(C(=O)Nc2sc3c(c2-c2nc4cc(C5=CCOCC5)ccc4s2)CCNC3)C1] | N-(3-(5-(3,6-dihydro-2H- pyran-4-yl)benzo[d]thiazol- 2-yl)-4,5,6,7-tetrahydrothieno [2,3-c]pyridin-2-yl)-3- (isopropylamino) cyclobutane-1-carboxamide |
| 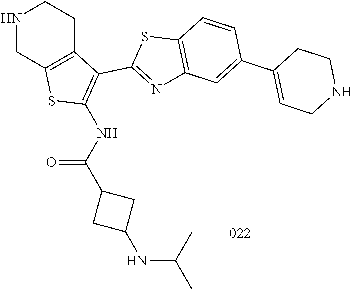 [SMILES: CC(C)NC1CC(C(=O)Nc2sc3c(c2-c2nc4cc(C5=CCNCC5)ccc4s2)CCNC3)C1] | 3-(isopropylamino)-N-(3-(5- (1,2,3,6-tetrahydropyridin- 4-yl)benzo[d]thiazol-2-yl)- 4,5,6,7-tetrahydrothieno [2,3-c]pyridin-2-yl) cyclobutane-1-carboxamide |
| 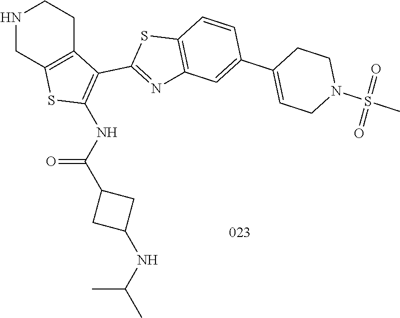 [SMILES: CC(C)NC1CC(C(=O)Nc2sc3c(c2-c2nc4cc(C5=CCN(S(C)(=O)=O)CC5)ccc4s2)CCNC3)C1] | 3-(isopropylamino)-N-(3-(5- (1-(methylsulfonyl)-1,2, 3,6-tetrahydropyridin-4-yl) benzo[d]thiazol-2-yl)-4,5, 6,7-tetrahydrothieno[2,3-c] pyridin-2-yl)cyclobutane- 1-carboxamide |
| 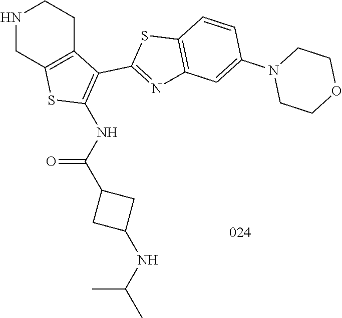 [SMILES: CC(C)NC1CC(C(=O)Nc2sc3c(c2-c2nc4cc(N5CCOCC5)ccc4s2)CCNC3)C1] | 3-(isopropylamino)-N-(3-(5- morpholinobenzo[d]thiazol- 2-yl)-4,5,6,7-tetrahydrothieno [2,3-c]pyridin-2-yl) cyclobutane- 1-carboxamide |
| 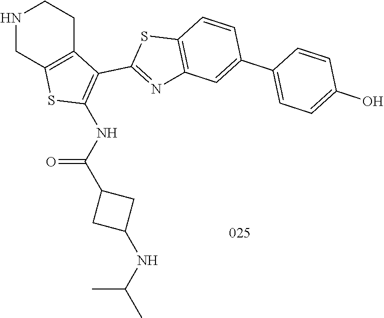 [SMILES: CC(C)NC1CC(C(=O)Nc2sc3c(c2-c2nc4cc(-c5ccc(O)cc5)ccc4s2)CCNC3)C1] | N-(3-(5-(4-hydroxyphenyl) benzo[d]thiazol-2-yl)-4,5, 6,7-tetrahydrothieno [2,3-c]pyridin-2-yl)-3- (isopropylamino) cyclobutane-1-carboxamide |
| 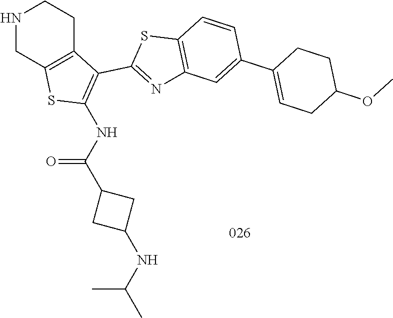 [SMILES: COC1CC=C(c2ccc3sc(-c4c(NC(=O)C5CC(NC(C)C)C5)sc5c4CCNC5)nc3c2)CC1] | 3-(isopropylamino)-N-(3-(5- (4-methoxyphenyl)benzo [d]thiazol-2-yl)-4,5,6,7- tetrahydrothieno[2,3-c] pyridin-2-yl)cyclobutane- 1-carboxamide |
| 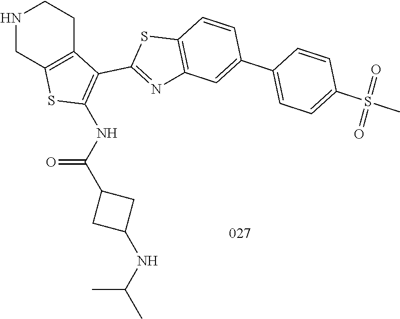 [SMILES: CC(C)NC1CC(C(=O)Nc2sc3c(c2-c2nc4cc(-c5ccc(S(C)(=O)=O)cc5)ccc4s2)CCNC3)C1] | 3-(isopropylamino)-N-(3-(5-(4- (methylsulfonyl)phenyl) benzo[d]thiazol-2-yl)-4,5,6,7- tetrahydrothieno[2,3-c] pyridin-2-yl)cyclobutane- 1-carboxamide |
| 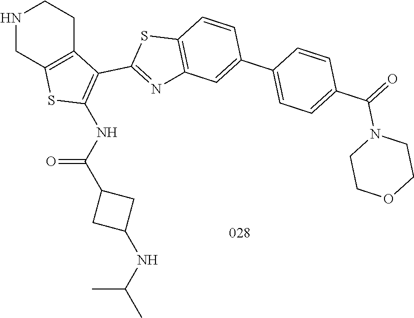 [SMILES: CC(C)NC1CC(C(=O)Nc2sc3c(c2-c2nc4cc(-c5ccc(C(=O)N6CCOCC6)cc5)ccc4s2)CCNC3)C1] | 3-(isopropylamino)-N-(3-(5-(4- (morpholine-4-carbonyl) phenyl)benzo[d]thiazol-2-yl)- 4,5,6,7-tetrahydrothieno [2,3-c]pyridin-2-yl)cyclobutane- 1-carboxamide |
| 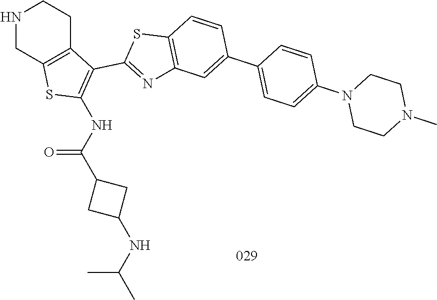 [SMILES: CC(C)NC1CC(C(=O)Nc2sc3c(c2-c2nc4cc(-c5ccc(N6CCN(C)CC6)cc5)ccc4s2)CCNC3)C1] | 3-(isopropylamino)-N-(3-(5- (4-(4-methylpiperazin-1- yl)phenyl)benzo[d]thiazol-2- yl)-4,5,6,7-tetrahydrothieno [2,3-c]pyridin-2-yl) cyclobutane-1-carboxamide |
| 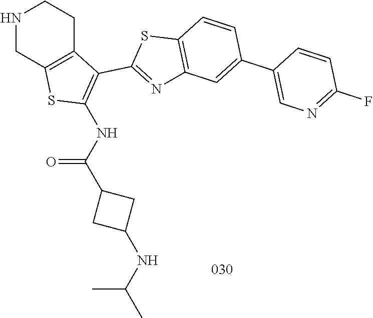 [SMILES: CC(C)NC1CC(C(=O)Nc2sc3c(c2-c2nc4cc(-c5ccc(F)nc5)ccc4s2)CCNC3)C1] | N-(3-(5-(6-fluoropyridin-3- yl)benzo[d]thiazol-2-yl)- 4,5,6,7-tetrahydrothieno [2,3-c]pyridin-2-yl)-3- (isopropylamino) cyclobutane-1-carboxamide |
| 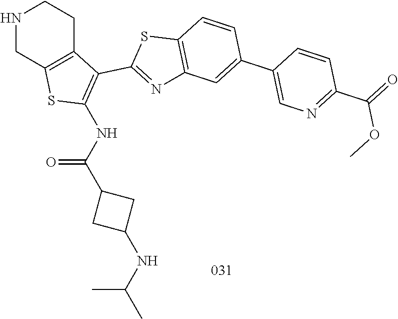 [SMILES: COC(=O)c1ccc(-c2ccc3sc(-c4c(NC(=O)C5CC(NC(C)C)C5)sc5c4CCNC5)nc3c2)cn1] | methyl 5-(2-(2-(3-(isopropylamino) cyclobutane-1-carboxamido)- 4,5,6,7-tetrahydrothieno [2,3-c]pyridin-3-yl) benzo[d]thiazol- 5-yl)picolinate |
| 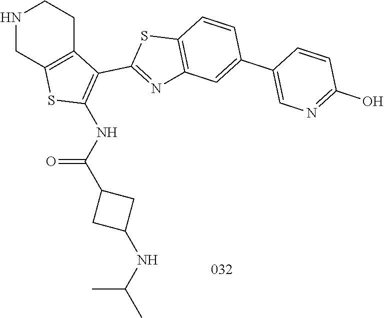 [SMILES: CC(C)NC1CC(C(=O)Nc2sc3c(c2-c2nc4cc(-c5ccc(O)nc5)ccc4s2)CCNC3)C1] | N-(3-(5-(6-hydroxypyridin- 3-yl)benzo[d]thiazol-2-yl)- 4,5,6,7-tetrahydrothieno [2,3-c]pyridin-2-yl)-3- (isopropylamino) cyclobutane-1-carboxamide |
| 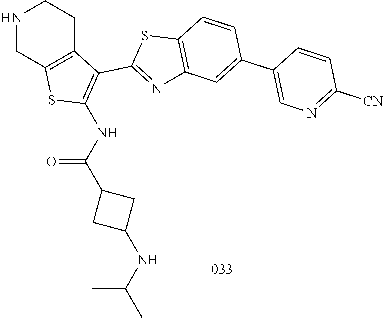 [SMILES: CC(C)NC1CC(C(=O)Nc2sc3c(c2-c2nc4cc(-c5ccc(C#N)nc5)ccc4s2)CCNC3)C1] | N-(3-(5-(6-cyanopyridin- 3-yl)benzo[d]thiazol-2-yl)- 4,5,6,7-tetrahydrothieno [2,3-c]pyridin-2-yl)-3- (isopropylamino) cyclobutane-1-carboxamide |
| 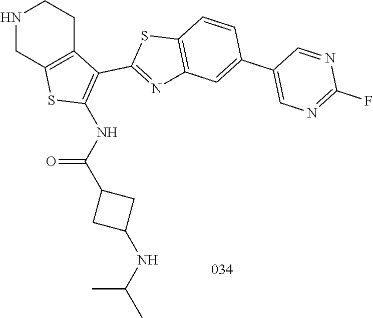 [SMILES: CC(C)NC1CC(C(=O)Nc2sc3c(c2-c2nc4cc(-c5cnc(F)nc5)ccc4s2)CCNC3)C1] | N-(3-(5-(2-fluoropyrimidin- 5-yl)benzo[d]thiazol-2-yl)- 4,5,6,7-tetrahydrothieno [2,3-c]pyridin-2-yl)-3- (isopropylamino) cyclobutane-1-carboxamide |
| 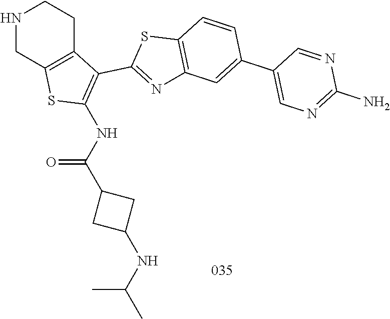 [SMILES: CC(C)NC1CC(C(=O)Nc2sc3c(c2-c2nc4cc(-c5cnc(N)nc5)ccc4s2)CCNC3)C1] | N-(3-(5-(2-aminopyrimidin- 5-yl)benzo[d]thiazol-2-yl)- 4,5,6,7-tetrahydrothieno [2,3-c]pyridin-2-yl)-3- (isopropylamino) cyclobutane-1-carboxamide |
| 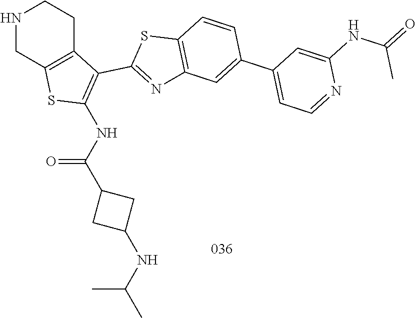 [SMILES: CC(=O)Nc1cc(-c2ccc3sc(-c4c(NC(=O)C5CC(NC(C)C)C5)sc5c4CCNC5)nc3c2)ccn1] | N-(3-(5-(2-acetamidopyridin- 4-yl)benzo[d]thiazol-2- yl)-4,5,6,7-tetrahydrothieno [2,3-c]pyridin-2-yl)-3- (isopropylamino) cyclobutane-1-carboxamide |
| 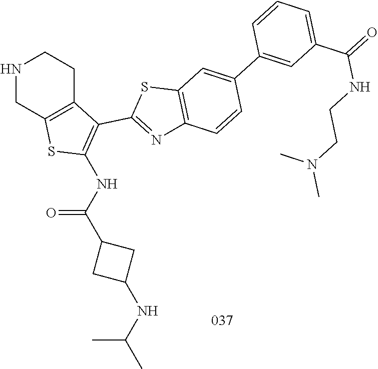 [SMILES: CC(C)NC1CC(C(=O)Nc2sc3c(c2-c2nc4ccc(-c5cccc(C(=O)NCCN(C)C)c5)cc4s2)CCNC3)C1] | N-(2-(dimethylamino)ethyl)-3- (2-(2-(3-(isopropylamino) cyclobutane-1-carboxamido)- 4,5,6,7-tetrahydrothieno [2,3-c]pyridin-3-yl)benzo [d]thiazol-6-yl)benzamide |
| 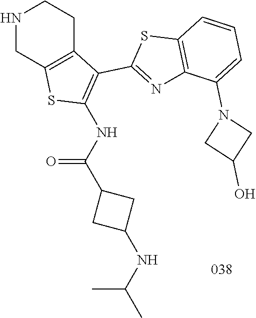 [SMILES: CC(C)NC1CC(C(=O)Nc2sc3c(c2-c2nc4c(N5CC(O)C5)cccc4s2)CCNC3)C1] | N-(3-(4-(3-hydroxyazetidin- 1-yl)benzo[d]thiazol-2-yl)- 4,5,6,7-tetrahydrothieno [2,3-c]pyridin-2-yl)-3- (isopropylamino) cyclobutane-1-carboxamide |
| 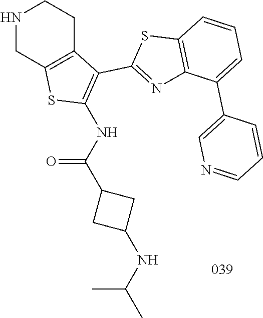 [SMILES: CC(C)NC1CC(C(=O)Nc2sc3c(c2-c2nc4c(-c5cccnc5)cccc4s2)CCNC3)C1] | 3-(isopropylamino)-N-(3-(4- (pyridin-3-yl)benzo[d] thiazol-2-yl)-4,5,6,7- tetrahydrothieno[2,3-c]pyridin-2- yl)cyclobutane- 1-carboxamide |
| 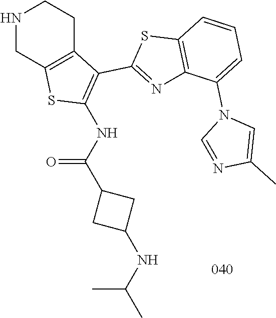 [SMILES: Cc1cn(-c2cccc3sc(-c4c(NC(=O)C5CC(NC(C)C)C5)sc5c4CCNC5)nc23)cn1] | 3-(isopropylamino)-N-(3-(4- (4-methyl-1H-imidazol- 1-yl)benzo[d]thiazol-2-yl)- 4,5,6,7-tetrahydrothieno [2,3-c]pyridin-2-yl) cyclobutane-1-carboxamide |
| 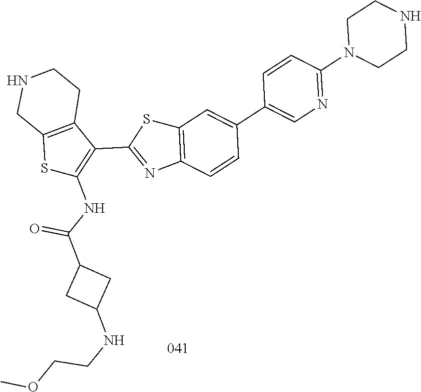 [SMILES: COCCNC1CC(C(=O)Nc2sc3c(c2-c2nc4ccc(-c5ccc(N6CCNCC6)nc5)cc4s2)CCNC3)C1] | 3-((2-methoxyethyl)amino)- N-(3-(6-(6-(piperazin-1- yl)pyridin-3-yl)benzo[d] thiazol-2-yl)-4,5,6,7- tetrahydrothieno[2,3-c] pyridin-2-yl)cyclobutane-1- carboxamide |
| 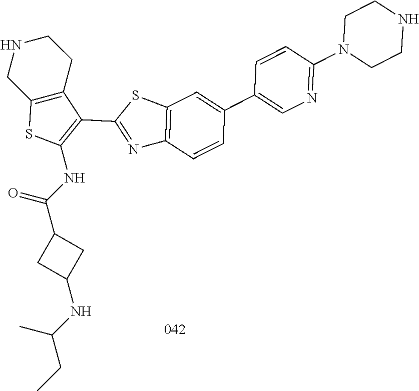 [SMILES: CCC(C)NC1CC(C(=O)Nc2sc3c(c2-c2nc4ccc(-c5ccc(N6CCNCC6)nc5)cc4s2)CCNC3)C1] | 3-(sec-butylamino)-N-(3-(6- (6-(piperazin-1-yl)pyridin- 3-yl)benzo[d]thiazol-2-yl)- 4,5,6,7-tetrahydrothieno [2,3-c]pyridin-2-yl) cyclobutane-1-carboxamide |
| 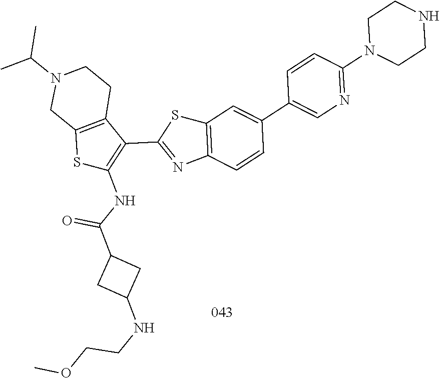 [SMILES: COCCNC1CC(C(=O)Nc2sc3c(c2-c2nc4ccc(-c5ccc(N6CCNCC6)nc5)cc4s2)CCN(C(C)C)C3)C1] | N-(6-isopropyl-3-(6-(6- (piperazin-1-yl)pyridin-3-yl) benzo[d]thiazol-2-yl)-4,5,6,7- tetrahydrothieno[2,3-c] pyridin-2-yl)-3-((2-methoxyethyl) amino)cyclobutane- 1-carboxamide |
| 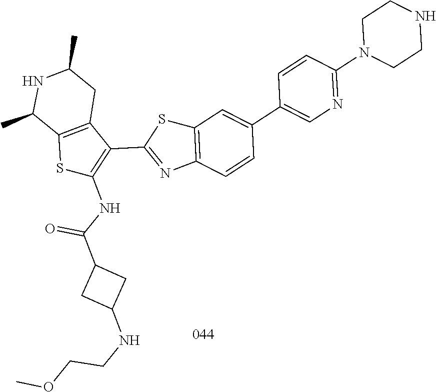 [SMILES: COCCNC1CC(C(=O)Nc2sc3c(c2-c2nc4ccc(-c5ccc(N6CCNCC6)nc5)cc4s2)C[C@H](C)N[C@@H]3C)C1] | N-((5S,7R)-5,7-dimethyl- 3-(6-(6-(piperazin-1-yl) pyridin-3-yl)benzo[d]thiazol-2- yl)-4,5,6,7-tetrahydrothieno [2,3-c]pyridin-2-yl)-3- ((2-methoxyethyl)amino) cyclobutane- 1-carboxamide |
| 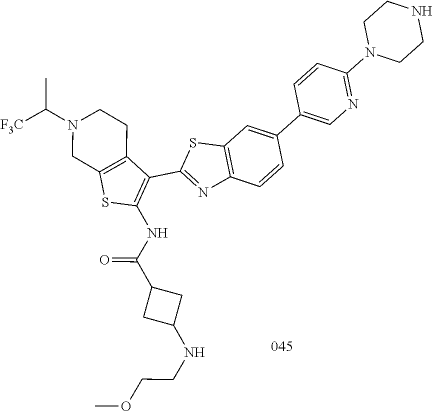 [SMILES: COCCNC1CC(C(=O)Nc2sc3c(c2-c2nc4ccc(-c5ccc(N6CCNCC6)nc5)cc4s2)CCN(C(C)C(F)(F)F)C3)C1] | 3-((2-methoxyethyl)amino)- N-(3-(6-(6-(piperazin-1- yl)pyridin-3-yl)benzo[d] thiazol-2-yl)-6-(1,1,1- trifluoropropan-2-yl)-4,5,6,7- tetrahydrothieno[2,3-c] pyridin-2-yl)cyclobutane- 1-carboxamide |
| 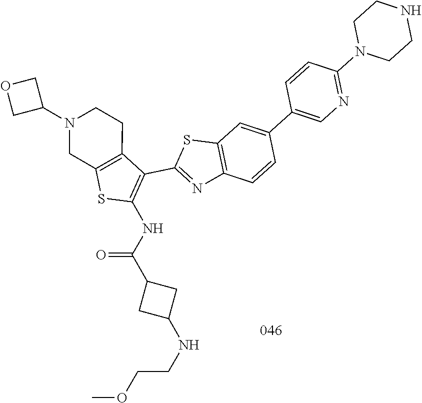 [SMILES: COCCNC1CC(C(=O)Nc2sc3c(c2-c2nc4ccc(-c5ccc(N6CCNCC6)nc5)cc4s2)CCN(C2COC2)C3)C1] | 3-((2-methoxyethyl)amino)- N-(6-(oxetan-3-yl)-3-(6- (6-(piperazin-1-yl)pyridin-3- yl)benzo[d]thiazol-2-yl)- 4,5,6,7-tetrahydrothieno [2,3-c]pyridin-2-yl) cyclobutane-1- carboxamide |
| 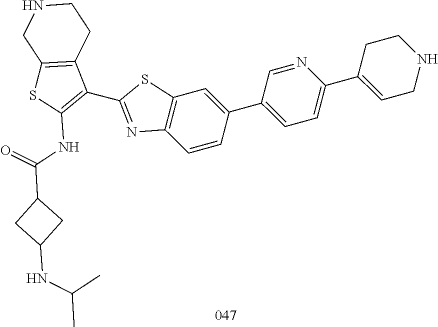 [SMILES: CC(C)NC1CC(C(=O)Nc2sc3c(c2-c2nc4ccc(-c5ccc(C6=CCNCC6)nc5)cc4s2)CCNC3)C1] | 3-(isopropylamino)-N-(3- (6-(1′,2′,3′,6′-tetrahydro-[2, 4′-bipyridin]-5-yl)benzo [d]thiazol-2-yl)-4,5,6,7- tetrahydrothieno[2,3-c] pyridin-2-yl)cyclobutane-1- carboxamide |
| 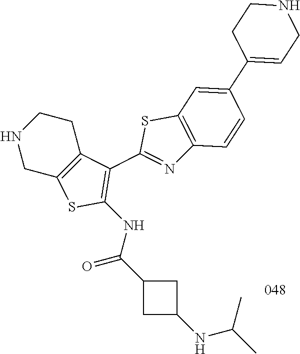 [SMILES: CC(C)NC1CC(C(=O)Nc2sc3c(c2-c2nc4ccc(C5=CCNCC5)cc4s2)CCNC3)C1] | 3-(isopropylamino)-N-(3-(6- (1,2,3,6-tetrahydropyridin- 4-yl)benzo[d]thiazol-2-yl)- 4,5,6,7-tetrahydrothieno [2,3-c]pyridin-2-yl) cyclobutane-1-carboxamide |
| 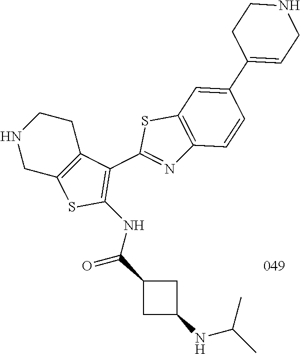 [SMILES: CC(C)N[C@H]1C[C@@H](C(=O)Nc2sc3c(c2-c2nc4ccc(C5=CCNCC5)cc4s2)CCNC3)C1] | (1s,3s)-3-(isopropylamino)- N-(3-(6-(1,2,3,6- tetrahydropyridin-4-yl)benzo [d]thiazol-2-yl)-4,5,6,7- tetrahydrothieno[2,3-c] pyridin-2-yl)cyclobutane-1- carboxamide |
| 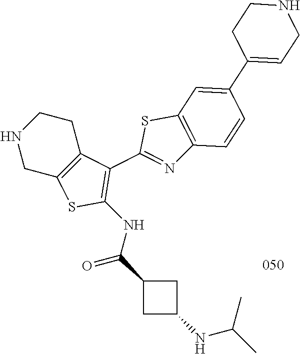 [SMILES: CC(C)N[C@H]1C[C@H](C(=O)Nc2sc3c(c2-c2nc4ccc(C5=CCNCC5)cc4s2)CCNC3)C1] | (1r,3r)-3-(isopropylamino)- N-(3-(6-(1,2,3,6- tetrahydropyridin-4-yl)benzo [d]thiazol-2-yl)-4,5,6,7- tetrahydrothieno[2,3-c] pyridin-2-yl)cyclobutane-1- carboxamide |
| 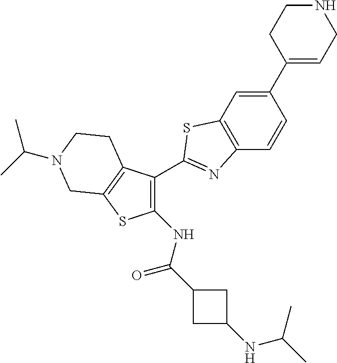 [SMILES: CC(C)NC1CC(C(=O)Nc2sc3c(c2-c2nc4ccc(C5=CCNCC5)cc4s2)CCN(C(C)C)C3)C1] 051 | N-(6-isopropyl-3-(6-(1,2,3,6- tetrahydropyridin-4-yl) benzo[d]thiazol-2-yl)-4,5,6,7- tetrahydrothieno[2,3-c] pyridin-2-yl)-3- (isopropylamino)cyclobutane-1- carboxamide |
| 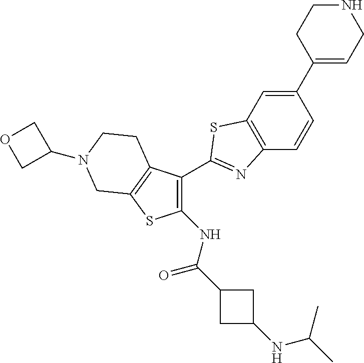 [SMILES: CC(C)NC1CC(C(=O)Nc2sc3c(c2-c2nc4ccc(C5=CCNCC5)cc4s2)CCN(C2COC2)C3)C1] 052 | 3-(isopropylamino)-N-(6- (oxetan-3-yl)-3-(6-(1,2,3,6- tetrahydropyridin-4-yl) benzo[d]thiazol-2-yl)-4,5,6,7- tetrahydrothieno[2,3-c] pyridin-2-yl)cyclobutane-1- carboxamide |
| 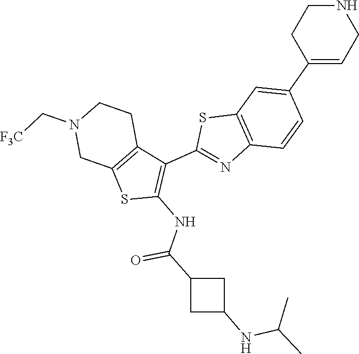 [SMILES: CC(C)NC1CC(C(=O)Nc2sc3c(c2-c2nc4ccc(C5=CCNCC5)cc4s2)CCN(CC(F)(F)F)C3)C1] 053 | 3-(isopropylamino)-N-(3-(6- (1,2,3,6-tetrahydropyridin- 4-yl)benzo[d]thiazol-2-yl)- 6-(2,2,2-trifluoroethyl)- 4,5,6,7-tetrahydrothieno [2,3-c]pyridin-2-yl) cyclobutane-1-carboxamide |
| 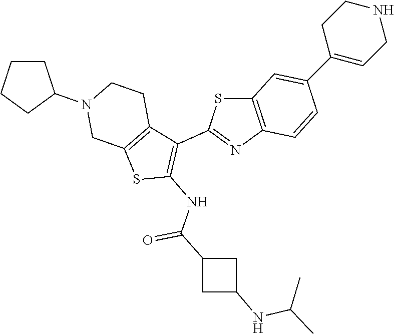 [SMILES: CC(C)NC1CC(C(=O)Nc2sc3c(c2-c2nc4ccc(C5=CCNCC5)cc4s2)CCN(C2CCCC2)C3)C1] 054 | N-(6-cyclopentyl-3-(6-(1,2,3,6- tetrahydropyridin-4-yl) benzo[d]thiazol-2-yl)- 4,5,6,7-tetrahydrothieno[2,3- c]pyridin-2-yl)-3- (isopropylamino)cyclobutane-1- carboxamide |
| 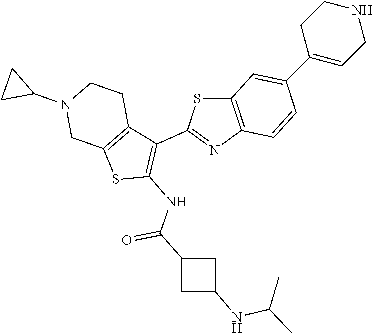 [SMILES: CC(C)NC1CC(C(=O)Nc2sc3c(c2-c2nc4ccc(C5=CCNCC5)cc4s2)CCN(C2CC2)C3)C1] 055 | N-(6-cyclopropyl-3-(6-(1,2,3,6- tetrahydropyridin-4- yl)benzo[d]thiazol-2-yl)-4,5,6,7- tetrahydrothieno[2,3- c]pyridin-2-yl)-3-(isopropylamino) cyclobutane-1- carboxamide |
| 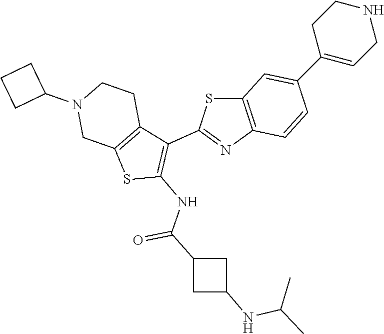 [SMILES: CC(C)NC1CC(C(=O)Nc2sc3c(c2-c2nc4ccc(C5=CCNCC5)cc4s2)CCN(C2CCC2)C3)C1] 056 | N-(6-cyclobutyl-3-(6-(1,2,3,6- tetrahydropyridin-4-yl) benzo[d]thiazol-2-yl)-4,5,6,7- tetrahydrothieno[2,3-c] pyridin-2-yl)-3-(isopropylamino) cyclobutane-1- carboxamide |
| 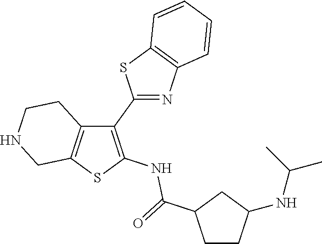 [SMILES: CC(C)NC1CCC(C(=O)Nc2sc3c(c2-c2nc4ccccc4s2)CCNC3)C1] 057 | N-(3-(benzo[d]thiazol-2-yl)- 4,5,6,7-tetrahydrothieno [2,3-c]pyridin-2-yl)-3- (isopropylamino)cyclopentane- 1-carboxamide |
| 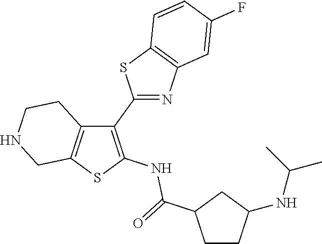 [SMILES: CC(C)NC1CCC(C(=O)Nc2sc3c(c2-c2nc4cc(F)ccc4s2)CCNC3)C1] 058 | N-(3-(5-fluorobenzo[d] thiazol-2-yl)-4,5,6,7- tetrahydrothieno[2,3-c]pyridin- 2-yl)-3-(isopropylamino) cyclopentane- 1-carboxamide |
| 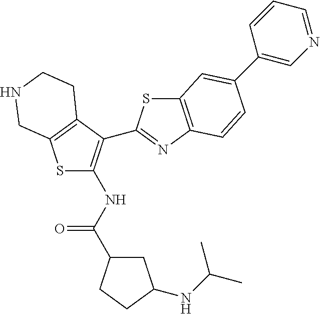 [SMILES: CC(C)NC1CCC(C(=O)Nc2sc3c(c2-c2nc4ccc(-c5cccnc5)cc4s2)CCNC3)C1] 059 | 3-(isopropylamino)-N-(3- (6-(pyridin-3-yl)benzo[d] thiazol-2-yl)-4,5,6,7- tetrahydrothieno[2,3-c]pyridin-2- yl)cyclopentane- 1-carboxamide |
| 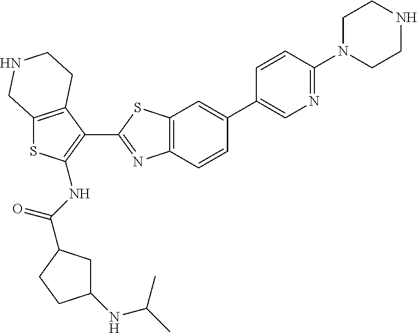 [SMILES: CC(C)NC1CCC(C(=O)Nc2sc3c(c2-c2nc4ccc(-c5ccc(N6CCNCC6)nc5)cc4s2)CCNC3)C1] 060 | 3-(isopropylamino)-N-(3- (6-(6-(piperazin-1-yl)pyridin- 3-yl)benzo[d]thiazol-2-yl)- 4,5,6,7-tetrahydrothieno [2,3-c]pyridin-2-yl) cyclopentane-1-carboxamide |
| 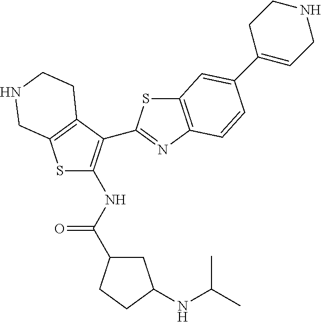 [SMILES: CC(C)NC1CCC(C(=O)Nc2sc3c(c2-c2nc4ccc(C5=CCNCC5)cc4s2)CCNC3)C1] 061 | 3-(isopropylamino)-N-(3-(6- (1,2,3,6-tetrahydropyridin- 4-yl)benzo[d]thiazol-2-yl)- 4,5,6,7-tetrahydrothieno [2,3-c]pyridin-2-yl) cyclopentane-1-carboxamide |
| 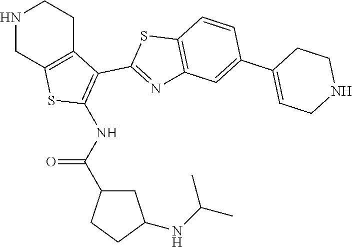 [SMILES: CC(C)NC1CCC(C(=O)Nc2sc3c(c2-c2nc4cc(C5=CCNCC5)ccc4s2)CCNC3)C1] 062 | 3-(isopropylamino)-N-(3-(5- (1,2,3,6-tetrahydropyridin- 4-yl)benzo[d]thiazol-2-yl)- 4,5,6,7-tetrahydrothieno [2,3-c]pyridin-2-yl) cyclopentane-1-carboxamide |
| 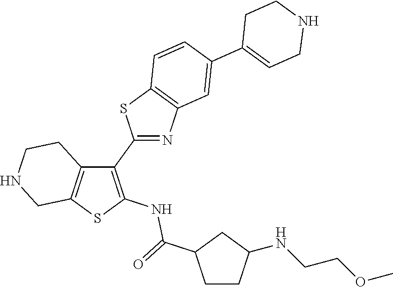 [SMILES: COCCNC1CCC(C(=O)Nc2sc3c(c2-c2nc4cc(C5=CCNCC5)ccc4s2)CCNC3)C1] 063 | 3-((2-methoxyethyl) amino)-N-(3-(5-(1,2,3,6- tetrahydropyridin-4-yl) benzo[d]thiazol-2-yl)-4,5,6,7- tetrahydrothieno[2,3-c] pyridin-2-yl)cyclopentane-1- carboxamide |
| 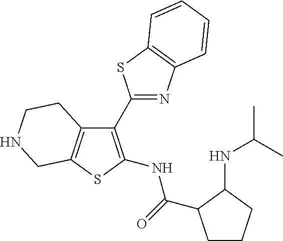 [SMILES: CC(C)NC1CCCC1C(=O)Nc1sc2c(c1-c1nc3ccccc3s1)CCNC2] 064 | N-(3-(benzo[d]thiazol-2-yl)- 4,5,6,7-tetrahydrothieno [2,3-c]pyridin-2-yl)-2- (isopropylamino)cyclopentane- 1-carboxamide |
| 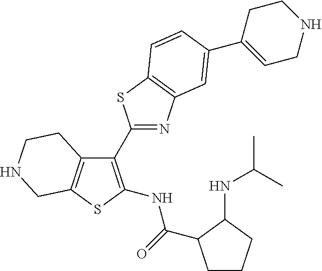 [SMILES: CC(C)NC1CCCC1C(=O)Nc1sc2c(c1-c1nc3cc(C4=CCNCC4)ccc3s1)CCNC2] 065 | 2-(isopropylamino)-N-(3-(5- (1,2,3,6-tetrahydropyridin- 4-yl)benzo[d]thiazol-2-yl)- 4,5,6,7-tetrahydrothieno [2,3-c]pyridin-2-yl) cyclopentane-1-carboxamide |
| 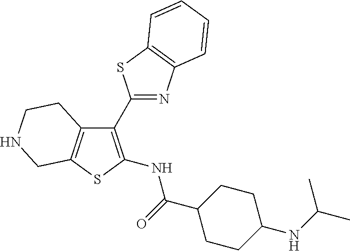 [SMILES: CC(C)NC1CCC(C(=O)Nc2sc3c(c2-c2nc4ccccc4s2)CCNC3)CC1] 066 | N-(3-(benzo[d]thiazol-2-yl)- 4,5,6,7-tetrahydrothieno [2,3-c]pyridin-2-yl)-4- (isopropylamino)cyclohexane- 1-carboxamide |
| 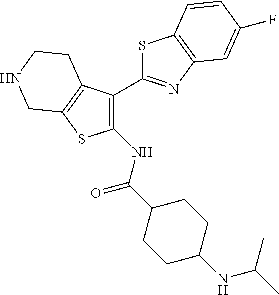 [SMILES: CC(C)NC1CCC(C(=O)Nc2sc3c(c2-c2nc4cc(F)ccc4s2)CCNC3)CC1] 067 | N-(3-(5-fluorobenzo[d] thiazol-2-yl)-4,5,6,7- tetrahydrothieno[2,3-c]pyridin- 2-yl)-4-(isopropylamino) cyclohexane- 1-carboxamide |
| 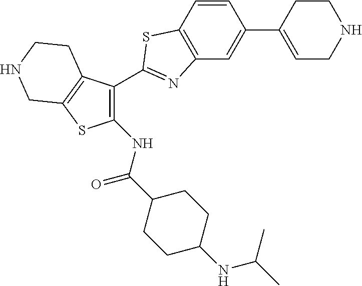 [SMILES: CC(C)NC1CCC(C(=O)Nc2sc3c(c2-c2nc4cc(C5=CCNCC5)ccc4s2)CCNC3)CC1] 068 | 4-(isopropylamino)-N-(3-(5- (1,2,3,6-tetrahydropyridin- 4-yl)benzo[d]thiazol-2-yl)- 4,5,6,7-tetrahydrothieno [2,3-c]pyridin-2-yl) cyclohexane-1-carboxamide |
| 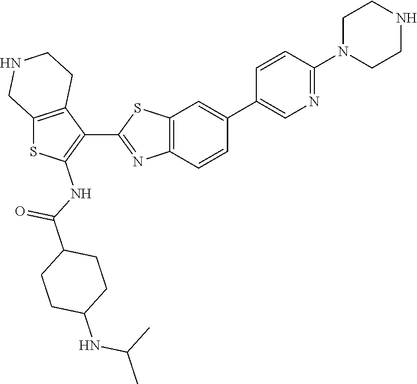 [SMILES: CC(C)NC1CCC(C(=O)Nc2sc3c(c2-c2nc4ccc(-c5ccc(N6CCNCC6)nc5)cc4s2)CCNC3)CC1] 069 | 4-(isopropylamino)-N-(3-(6- (6-(piperazin-1-yl)pyridin- 3-yl)benzo[d]thiazol-2-yl)- 4,5,6,7-tetrahydrothieno [2,3-c]pyridin-2-yl) cyclohexane-1-carboxamide |
| 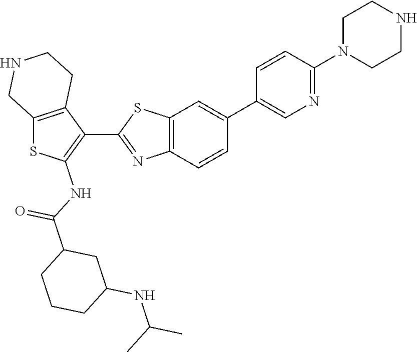 [SMILES: CC(C)NC1CCCC(C(=O)Nc2sc3c(c2-c2nc4ccc(-c5ccc(N6CCNCC6)nc5)cc4s2)CCNC3)C1] 070 | 3-(isopropylamino)-N-(3-(6- (6-(piperazin-1-yl)pyridin- 3-yl)benzo[d]thiazol-2-yl)- 4,5,6,7-tetrahydrothieno [2,3-c]pyridin-2-yl) cyclohexane-1-carboxamide |
| 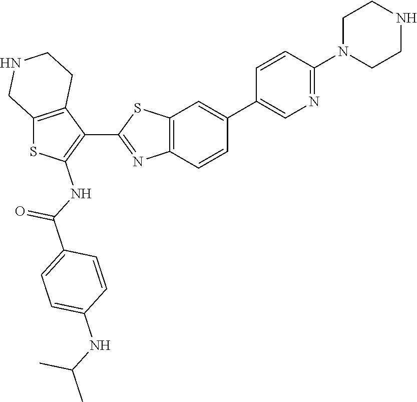 [SMILES: CC(C)Nc1ccc(C(=O)Nc2sc3c(c2-c2nc4ccc(-c5ccc(N6CCNCC6)nc5)cc4s2)CCNC3)cc1] 071 | 4-(isopropylamino)-N-(3-(6- (6-(piperazin-1-yl)pyridin- 3-yl)benzo[d]thiazol-2-yl)- 4,5,6,7-tetrahydrothieno [2,3-c]pyridin- 2-yl)benzamide |
| 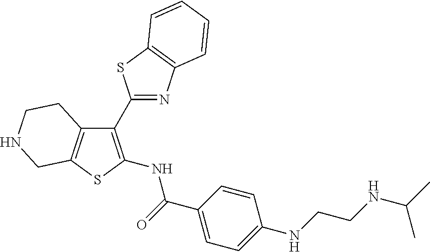 [SMILES: CC(C)NCCNc1ccc(C(=O)Nc2sc3c(c2-c2nc4ccccc4s2)CCNC3)cc1] 072 | N-(3-(benzo[d]thiazol-2-yl)- 4,5,6,7-tetrahydrothieno [2,3-c]pyridin-2-yl)-4-((2- (isopropylamino)ethyl) amino)benzamide |
| 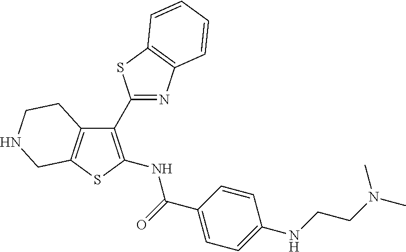 [SMILES: CN(C)CCNc1ccc(C(=O)Nc2sc3c(c2-c2nc4ccccc4s2)CCNC3)cc1] 073 | N-(3-(benzo[d]thiazol-2-yl)- 4,5,6,7-tetrahydrothieno [2,3-c]pyridin-2-yl)-4-((2- (dimethylamino)ethyl) amino)benzamide |
| 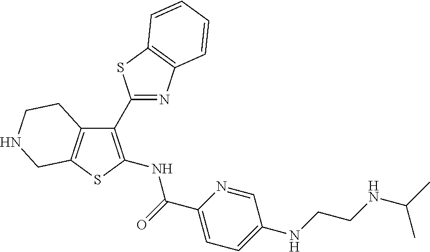 [SMILES: CC(C)NCCNc1ccc(C(=O)Nc2sc3c(c2-c2nc4ccccc4s2)CCNC3)nc1] 074 | N-(3-(benzo[d]thiazol-2-yl)- 4,5,6,7-tetrahydrothieno [2,3-c]pyridin-2-yl)-5-((2- (isopropylamino)ethyl) amino)picolinamide |
| 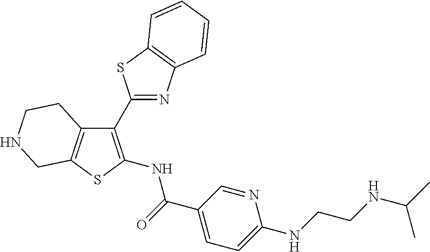 [SMILES: CC(C)NCCNc1ccc(C(=O)Nc2sc3c(c2-c2nc4ccccc4s2)CCNC3)cn1] 075 | N-(3-(benzo[d]thiazol-2-yl)- 4,5,6,7-tetrahydrothieno [2,3-c]pyridin-2-yl)-6-((2- (isopropylamino)ethyl) amino)nicotinamide |
| 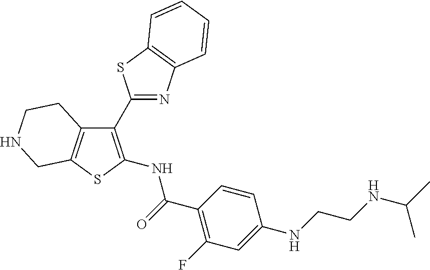 [SMILES: CC(C)NCCNc1ccc(C(=O)Nc2sc3c(c2-c2nc4ccccc4s2)CCNC3)c(F)c1] 076 | N-(3-(benzo[d]thiazol-2-yl)- 4,5,6,7-tetrahydrothieno [2,3-c]pyridin-2-yl)-2-fluoro- 4-((2-(isopropylamino) ethyl)amino)benzamide |
| 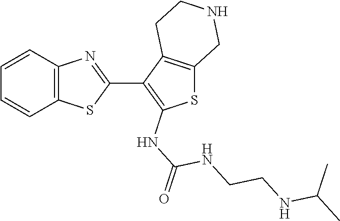 [SMILES: CC(C)NCCNC(=O)Nc1sc2c(c1-c1nc3ccccc3s1)CCNC2] 077 | 1-(3-(benzo[d]thiazol-2-yl)- 4,5,6,7-tetrahydrothieno [2,3-c]pyridin-2-yl)-3-(2- (isopropylamino)ethyl)urea |
| 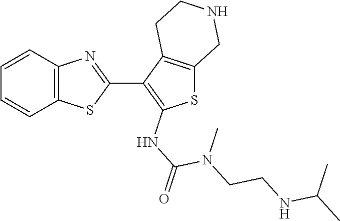 [SMILES: CC(C)NCCN(C)C(=O)Nc1sc2c(c1-c1nc3ccccc3s1)CCNC2] 078 | 3-(3-(benzo[d]thiazol-2-yl)- 4,5,6,7-tetrahydrothieno [2,3-c]pyridin-2-yl)-1-(2- (isopropylamino)ethyl)-1- methylurea |
| 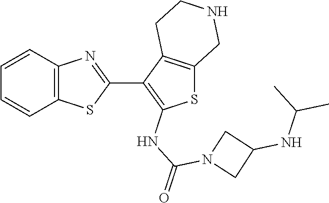 [SMILES: CC(C)NC1CN(C(=O)Nc2sc3c(c2-c2nc4ccccc4s2)CCNC3)C1] 079 | N-(3-(benzo[d]thiazol-2-yl)- 4,5,6,7-tetrahydrothieno [2,3-c]pyridin-2-yl)-3- (isopropylamino)azetidine-1- carboxamide |
| 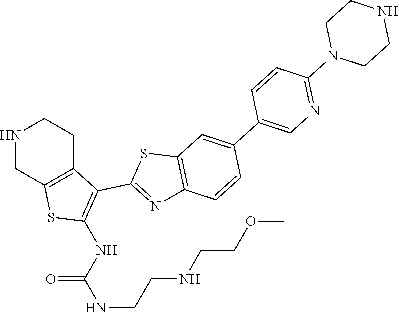 [SMILES: COCCNCCNC(=O)Nc1sc2c(c1-c1nc3ccc(-c4ccc(N5CCNCC5)nc4)cc3s1)CCNC2] 080 | 1-(2-((2-methoxyethyl) amino)ethyl)-3-(3-(6-(6- (piperazin-1-yl)pyridin-3-yl) benzo[d]thiazol-2-yl)-4,5,6,7- tetrahydrothieno[2,3-c] pyridin-2-yl)urea |
| 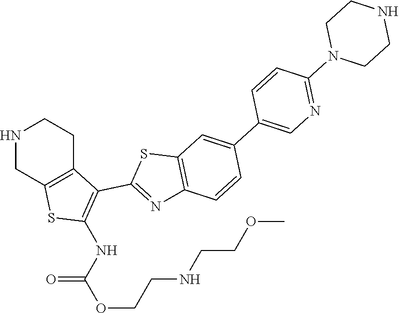 [SMILES: COCCNCCOC(=O)Nc1sc2c(c1-c1nc3ccc(-c4ccc(N5CCNCC5)nc4)cc3s1)CCNC2] 081 | 2-((2-methoxyethyl) amino)ethyl (3-(6-(6-(piperazin-1-yl)pyridin- 3-yl)benzo[d]thiazol- 2-yl)-4,5,6,7-tetrahydrothieno [2,3-c]pyridin-2-yl) carbamate |
| 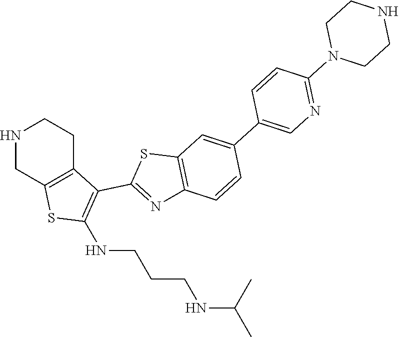 [SMILES: CC(C)NCCCNc1sc2c(c1-c1nc3ccc(-c4ccc(N5CCNCC5)nc4)cc3s1)CCNC2] 082 | N¹-isopropyl-N³-(3-(6-(6- (piperazin-1-yl)pyridin-3-yl) benzo[d]thiazol-2-yl)- 4,5,6,7-tetrahydrothieno[2,3- c]pyridin-2-yl) propane-1,3-diamine |
| 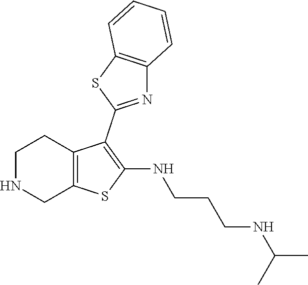 [SMILES: CC(C)NCCCNc1sc2c(c1-c1nc3ccccc3s1)CCNC2] 083 | N¹-(3-(benzo[d]thiazol-2-yl)- 4,5,6,7-tetrahydrothieno [2,3-c]pyridin-2-yl)-N³- isopropylpropane-1,3-diamine |
| 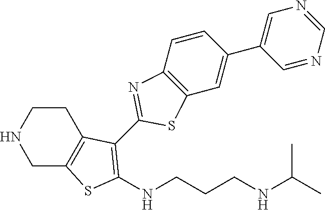 [SMILES: CC(C)NCCCNc1sc2c(c1-c1nc3ccc(-c4cncnc4)cc3s1)CCNC2] 084 | N¹-isopropyl-N³-(3-(6-(pyrimidin- 5-yl)benzo[d]thiazol- 2-yl)-4,5,6,7-tetrahydrothieno [2,3-c]pyridin-2-yl) propane-1,3-diamine |
| 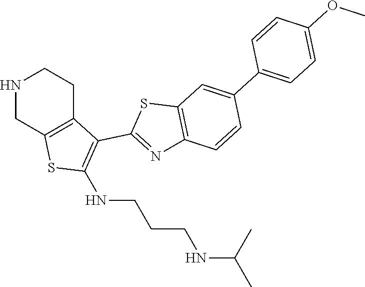 [SMILES: COc1ccc(-c2ccc3nc(-c4c(NCCCNC(C)C)sc5c4CCNC5)sc3c2)cc1] 085 | N¹-isopropyl-N³-(3-(6-(4- methoxyphenyl)benzo[d] thiazol-2-yl)-4,5,6,7- tetrahydrothieno[2,3-c]pyridin-2- yl)propane-1,3-diamine |
| 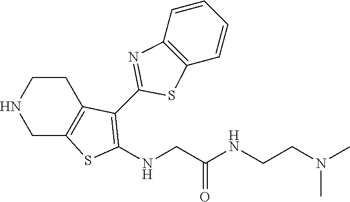 [SMILES: CN(C)CCNC(=O)CNc1sc2c(c1-c1nc3ccccc3s1)CCNC2] 086 | 2-((3-(benzo[d]thiazol-2-yl)- 4,5,6,7-tetrahydrothieno [2,3-c]pyridin-2-yl)amino)- N-(2-(dimethylamino) ethyl)acetamide |
| 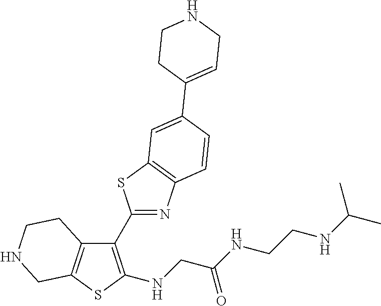 [SMILES: CC(C)NCCNC(=O)CNc1sc2c(c1-c1nc3ccc(C4=CCNCC4)cc3s1)CCNC2] 087 | N-(2-(isopropylamino)ethyl)- 2-((3-(6-(1,2,3,6- tetrahydropyridin-4-yl)benzo [d]thiazol-2-yl)-4,5,6,7- tetrahydrothieno[2,3-c]pyridin- 2-yl)amino)acetamide |
| 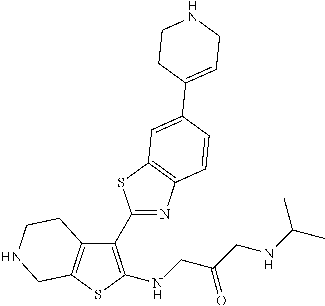 [SMILES: CC(C)NCC(=O)CNc1sc2c(c1-c1nc3ccc(C4=CCNCC4)cc3s1)CCNC2] 088 | 1-(isopropylamino)-3-((3-(6- (1,2,3,6-tetrahydropyridin- 4-yl)benzo[d]thiazol-2-yl)- 4,5,6,7-tetrahydrothieno [2,3-c]pyridin-2-yl) amino)propan-2-one |
| 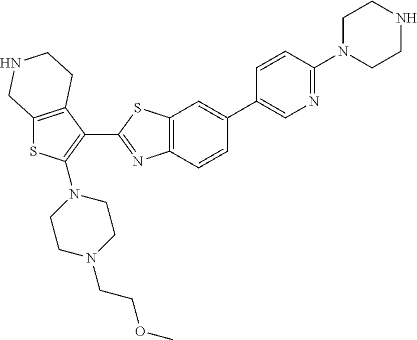 [SMILES: COCCN1CCN(c2sc3c(c2-c2nc4ccc(-c5ccc(N6CCNCC6)nc5)cc4s2)CCNC3)CC1] 089 | 2-(2-(4-(2-methoxyethyl) piperazin-1-yl)-4,5,6,7- tetrahydrothieno[2,3-c]pyridin- 3-yl)-6-(6-(piperazin-1- yl)pyridin-3-yl) benzo[d]thiazole |
| 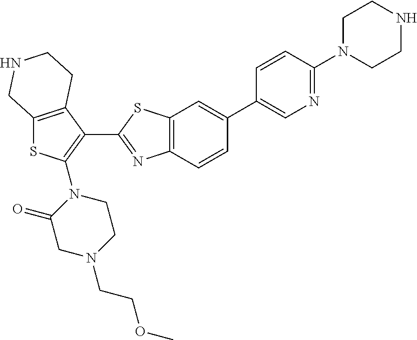 [SMILES: COCCN1CCN(c2sc3c(c2-c2nc4ccc(-c5ccc(N6CCNCC6)nc5)cc4s2)CCNC3)C(=O)C1] 090 | 4-(2-methoxyethyl)-1-(3-(6- (6-(piperazin-1-yl)pyridin- 3-yl)benzo[d]thiazol-2-yl)- 4,5,6,7-tetrahydrothieno [2,3-c]pyridin-2- yl)piperazin-2-one |
| 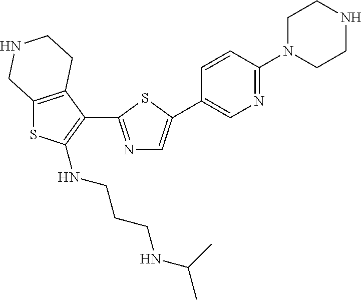 [SMILES: CC(C)NCCCNc1sc2c(c1-c1ncc(-c3ccc(N4CCNCC4)nc3)s1)CCNC2] 091 | N¹-isopropyl-N³-(3-(5-(6- (piperazin-1-yl)pyridin-3-yl) thiazol-2-yl)-4,5,6,7- tetrahydrothieno[2,3-c]pyridin- 2-yl)propane-1,3-diamine |
| 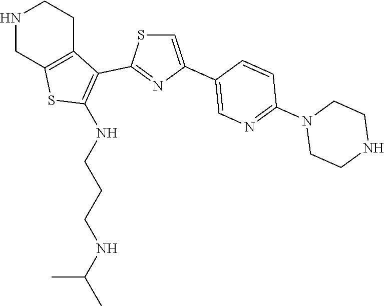 [SMILES: CC(C)NCCCNc1sc2c(c1-c1nc(-c3ccc(N4CCNCC4)nc3)cs1)CCNC2] 092 | N¹-isopropyl-N³-(3-(4-(6- (piperazin-1-yl)pyridin-3-yl) thiazol-2-yl)-4,5,6,7- tetrahydrothieno[2,3-c]pyridin- 2-yl)propane-1,3-diamine |
| 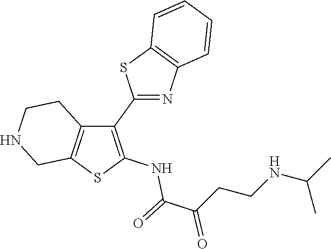 [SMILES: CC(C)NCCC(=O)C(=O)Nc1sc2c(c1-c1nc3ccccc3s1)CCNC2] 093 | N-(3-(benzo[d]thiazol-2-yl)- 4,5,6,7-tetrahydrothieno [2,3-c]pyridin-2-yl)-4- (isopropylamino)-2- oxobutanamide |
| 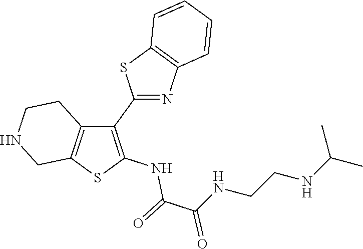 [SMILES: CC(C)NCCNC(=O)C(=O)Nc1sc2c(c1-c1nc3ccccc3s1)CCNC2] 094 | N¹-(3-(benzo[d]thiazol-2-yl)- 4,5,6,7-tetrahydrothieno [2,3-c]pyridin-2-yl)-N²- (2-(isopropylamino)ethyl) oxalamide |
| 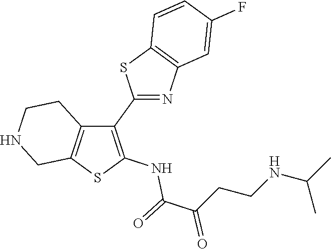 [SMILES: CC(C)NCCC(=O)C(=O)Nc1sc2c(c1-c1nc3cc(F)ccc3s1)CCNC2] 095 | N-(3-(5-fluorobenzo[d] thiazol-2-yl)-4,5,6,7- tetrahydrothieno[2,3-c]pyridin- 2-yl)-4-(isopropylamino)- 2-oxobutanamide |
| 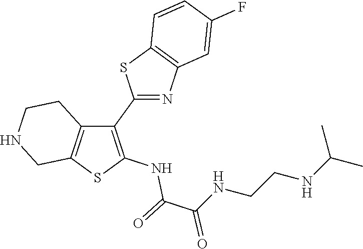 [SMILES: CC(C)NCCNC(=O)C(=O)Nc1sc2c(c1-c1nc3cc(F)ccc3s1)CCNC2] 096 | N¹-(3-(5-fluorobenzo[d] thiazol-2-yl)-4,5,6,7- tetrahydrothieno[2,3-c] pyridin-2-yl)-N²-(2- (isopropylamino) ethyl)oxalamide |
| 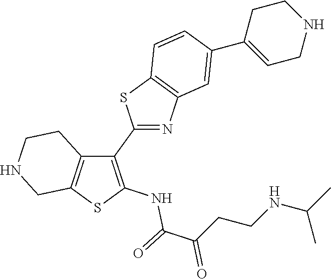 [SMILES: CC(C)NCCC(=O)C(=O)Nc1sc2c(c1-c1nc3cc(C4=CCNCC4)ccc3s1)CCNC2] 097 | 4-(isopropylamino)-2-oxo- N-(3-(5-(1,2,3,6- tetrahydropyridin-4-yl)benzo [d]thiazol-2-yl)-4,5,6,7- tetrahydrothieno[2,3-c] pyridin-2-yl)butanamide |
| 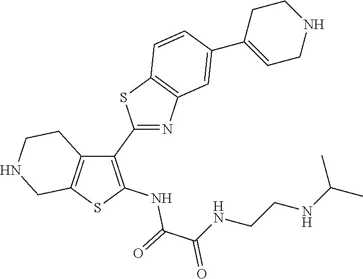 [SMILES: CC(C)NCCNC(=O)C(=O)Nc1sc2c(c1-c1nc3cc(C4=CCNCC4)ccc3s1)CCNC2] 098 | N¹-(2-(isopropylamino)ethyl)- N²-(3-(5-(1,2,3,6- tetrahydropyridin-4-yl)benzo [d]thiazol-2-yl)-4,5,6,7- tetrahydrothieno[2,3-c] pyridin-2-yl)oxalamide |
| 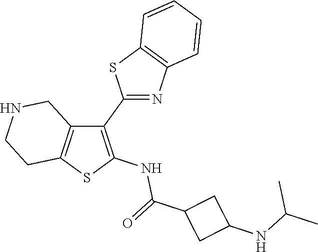 [SMILES: CC(C)NC1CC(C(=O)Nc2sc3c(c2-c2nc4ccccc4s2)CNCC3)C1] 099 | N-(3-(benzo[d]thiazol-2-yl)- 4,5,6,7-tetrahydrothieno [3,2-c]pyridin-2-yl)-3- (isopropylamino)cyclobutane- 1-carboxamide |
| 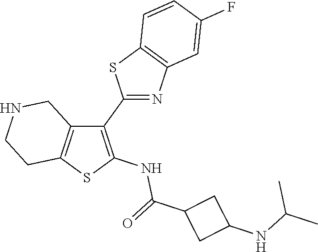 [SMILES: CC(C)NC1CC(C(=O)Nc2sc3c(c2-c2nc4cc(F)ccc4s2)CNCC3)C1] 100 | N-(3-(5-fluorobenzo[d] thiazol-2-yl)-4,5,6,7- tetrahydrothieno[3,2-c]pyridin- 2-yl)-3-(isopropylamino) cyclobutane- 1-carboxamide |
| 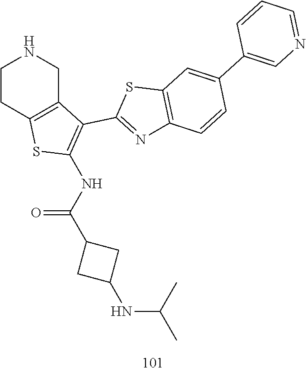 [SMILES: CC(C)NC1CC(C(=O)Nc2sc3c(c2-c2nc4ccc(-c5cccnc5)cc4s2)CNCC3)C1] | 3-(isopropylamino)-N-(3-(6- (pyridin-3-yl)benzo[d] thiazol-2-yl)-4,5,6,7- tetrahydrothieno[3,2-c]pyridin-2- yl)cyclobutane- 1-carboxamide |
| 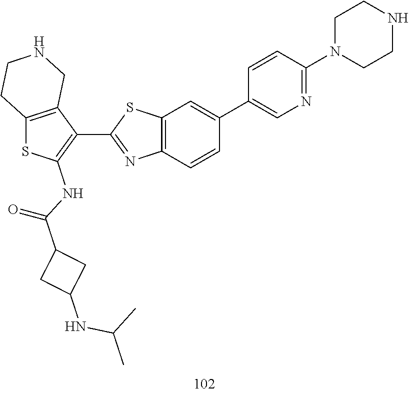 [SMILES: CC(C)NC1CC(C(=O)Nc2sc3c(c2-c2nc4ccc(-c5ccc(N6CCNCC6)nc5)cc4s2)CNCC3)C1] | 3-(isopropylamino)-N-(3-(6- (6-(piperazin-1-yl)pyridin- 3-yl)benzo[d]thiazol-2-yl)- 4,5,6,7-tetrahydrothieno [3,2-c]pyridin-2-yl) cyclobutane-1-carboxamide |
| 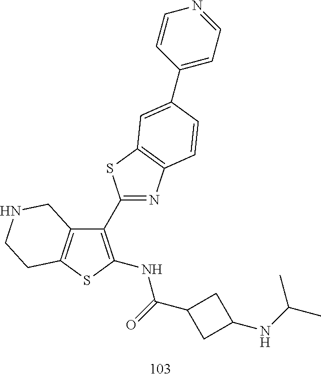 [SMILES: CC(C)NC1CC(C(=O)Nc2sc3c(c2-c2nc4ccc(-c5ccncc5)cc4s2)CNCC3)C1] | 3-(isopropylamino)-N-(3- (6-(pyridin-4-yl)benzo[d] thiazol-2-yl)-4,5,6,7- tetrahydrothieno[3,2-c]pyridin-2- yl)cyclobutane- 1-carboxamide |
| 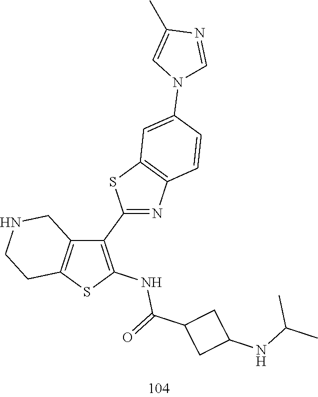 [SMILES: Cc1cn(-c2ccc3nc(-c4c(NC(=O)C5CC(NC(C)C)C5)sc5c4CNCC5)sc3c2)cn1] | 3-(isopropylamino)-N-(3-(6- (4-methyl-1H-imidazol- 1-yl)benzo[d]thiazol-2-yl)- 4,5,6,7-tetrahydrothieno [3,2-c]pyridin-2-yl) cyclobutane-1-carboxamide |
| 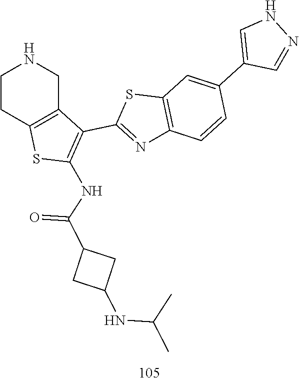 [SMILES: CC(C)NC1CC(C(=O)Nc2sc3c(c2-c2nc4ccc(-c5cn[nH]c5)cc4s2)CNCC3)C1] | N-(3-(6-(1H-pyrazol-4-yl) benzo[d]thiazol-2-yl)-4,5, 6,7-tetrahydrothieno [3,2-c]pyridin-2-yl)-3- (isopropylamino) cyclobutane-1-carboxamide |
| 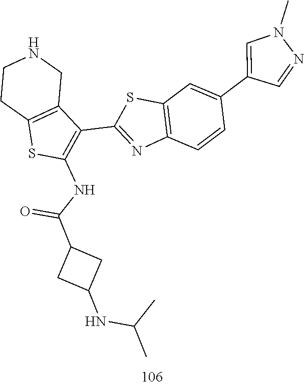 [SMILES: CC(C)NC1CC(C(=O)Nc2sc3c(c2-c2nc4ccc(-c5cnn(C)c5)cc4s2)CNCC3)C1] | 3-(isopropylamino)-N-(3- (6-(1-methyl-1H-pyrazol-4- yl)benzo[d]thiazol-2-yl)- 4,5,6,7-tetrahydrothieno[3, 2-c]pyridin-2-yl) cyclobutane-1-carboxamide |
| 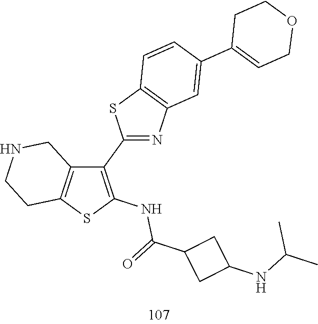 [SMILES: CC(C)NC1CC(C(=O)Nc2sc3c(c2-c2nc4cc(C5=CCOCC5)ccc4s2)CNCC3)C1] | N-(3-(5-(3,6-dihydro-2H- pyran-4-yl)benzo[d]thiazol- 2-yl)-4,5,6,7-tetrahydrothieno [3,2-c]pyridin-2-yl)-3- (isopropylamino) cyclobutane-1-carboxamide |
| 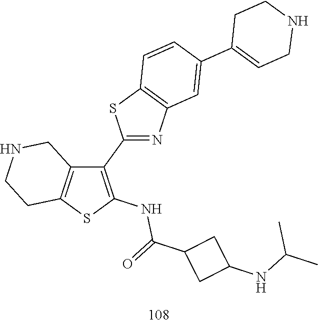 [SMILES: CC(C)NC1CC(C(=O)Nc2sc3c(c2-c2nc4cc(C5=CCNCC5)ccc4s2)CNCC3)C1] | 3-(isopropylamino)-N-(3-(5- (1,2,3,6-tetrahydropyridin- 4-yl)benzo[d]thiazol-2-yl)- 4,5,6,7-tetrahydrothieno [3,2-c]pyridin-2-yl) cyclobutane-1-carboxamide |
| 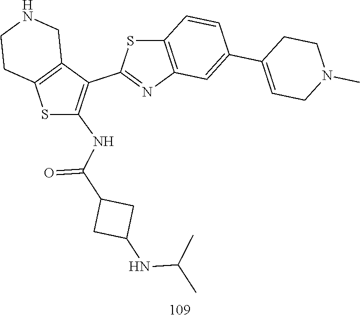 [SMILES: CC(C)NC1CC(C(=O)Nc2sc3c(c2-c2nc4cc(C5=CCN(C)CC5)ccc4s2)CNCC3)C1] | 3-(isopropylamino)-N- (3-(5-(1-methyl-1,2,3,6- tetrahydropyridin-4-yl) benzo[d]thiazol-2-yl)-4,5,6,7- tetrahydrothieno[3,2-c] pyridin-2-yl)cyclobutane-1- carboxamide |
| 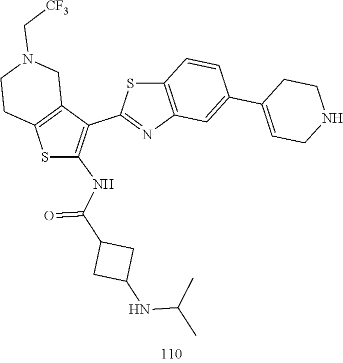 [SMILES: CC(C)NC1CC(C(=O)Nc2sc3c(c2-c2nc4cc(C5=CCNCC5)ccc4s2)CN(CC(F)(F)F)CC3)C1] | 3-(isopropylamino)-N-(3-(5- (1,2,3,6-tetrahydropyridin- 4-yl)benzo[d]thiazol-2-yl)- 5-(2,2,2-trifluoroethyl)- 4,5,6,7-tetrahydrothieno [3,2-c]pyridin-2-yl) cyclobutane- 1-carboxamide |
| 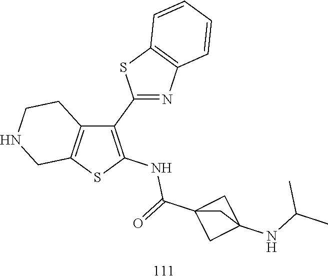 [SMILES: CC(C)NC12CC(C(=O)Nc3sc4c(c3-c3nc5ccccc5s3)CCNC4)(C1)C2] | N-(3-(benzo[d]thiazol-2-yl)- 4,5,6,7-tetrahydrothieno [2,3-c]pyridin-2-yl)-3- (isopropylamino)bicyclo[1.1.1] pentane-1-carboxamide |
| 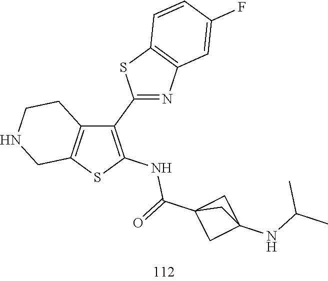 [SMILES: CC(C)NC12CC(C(=O)Nc3sc4c(c3-c3nc5cc(F)ccc5s3)CCNC4)(C1)C2] | N-(3-(5-fluorobenzo[d] thiazol-2-yl)-4,5,6,7- tetrahydrothieno[2,3-c]pyridin- 2-yl)-3-(isopropylamino) bicyclo[1.1.1]pentane- 1-carboxamide |
| 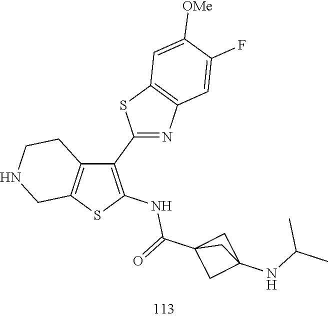 [SMILES: COc1cc2sc(-c3c(NC(=O)C45CC(NC(C)C)(C4)C5)sc4c3CCNC4)nc2cc1F] | N-(3-(5-fluoro-6-methoxybenzo [d]thiazol-2-yl)-4,5, 6,7-tetrahydrothieno [2,3-c]pyridin-2-yl)-3- (isopropylamino)bicyclo[1.1.1] pentane-1-carboxamide |
| 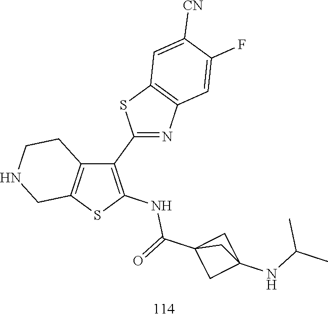 [SMILES: CC(C)NC12CC(C(=O)Nc3sc4c(c3-c3nc5cc(F)c(C#N)cc5s3)CCNC4)(C1)C2] | N-(3-(6-cyano-5-fluorobenzo [d]thiazol-2-yl)-4,5,6,7- tetrahydrothieno[2,3-c] pyridin-2-yl)-3- (isopropylamino)bicyclo[1.1.1] pentane-1-carboxamide |
| 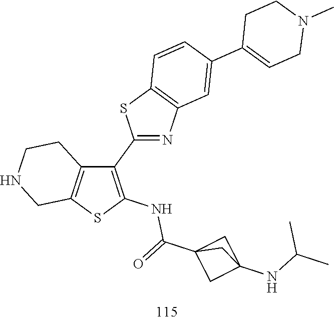 [SMILES: CC(C)NC12CC(C(=O)Nc3sc4c(c3-c3nc5cc(C6=CCN(C)CC6)ccc5s3)CCNC4)(C1)C2] | 3-(isopropylamino)-N-(3- (5-(1-methyl-1,2,3,6- tetrahydropyridin-4-yl)benzo [d]thiazol-2-yl)-4,5,6,7- tetrahydrothieno[2,3-c] pyridin-2-yl)bicyclo[1.1.1] pentane-1-carboxamide |
| 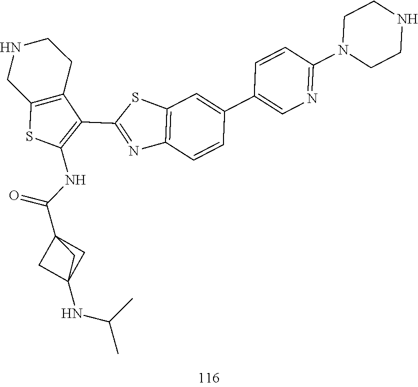 [SMILES: CC(C)NC12CC(C(=O)Nc3sc4c(c3-c3nc5ccc(-c6ccc(N7CCNCC7)nc6)cc5s3)CCNC4)(C1)C2] | 3-(isopropylamino)-N-(3-(6- (6-(piperazin-1-yl)pyridin- 3-yl)benzo[d]thiazol-2-yl)- 4,5,6,7-tetrahydrothieno [2,3-c]pyridin-2-yl)bicyclo [1.1.1]pentane-1-carboxamide |
| 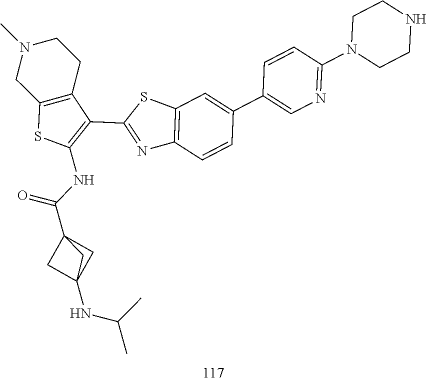 [SMILES: CC(C)NC12CC(C(=O)Nc3sc4c(c3-c3nc5ccc(-c6ccc(N7CCNCC7)nc6)cc5s3)CCN(C)C4)(C1)C2] | 3-(isopropylamino)-N-(6- methyl-3-(6-(6-(piperazin- 1-yl)pyridin-3-yl)benzo [d]thiazol-2-yl)-4,5,6,7- tetrahydrothieno[2,3-c] pyridin-2-yl)bicyclo[1.1.1] pentane-1-carboxamide |
| 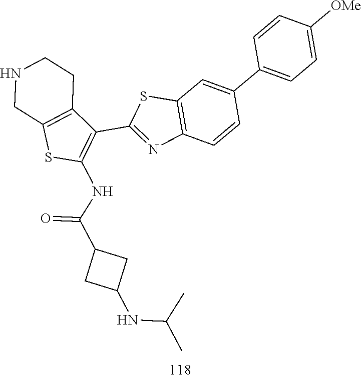 [SMILES: COc1ccc(-c2ccc3nc(-c4c(NC(=O)C5CC(NC(C)C)C5)sc5c4CCNC5)sc3c2)cc1] | 3-(isopropylamino)-N-(3-(6- (4-methoxyphenyl)benzo [d]thiazol-2-yl)-4,5,6,7- tetrahydrothieno[2,3-c] pyridin-2-yl)cyclobutane- 1-carboxamide |
| 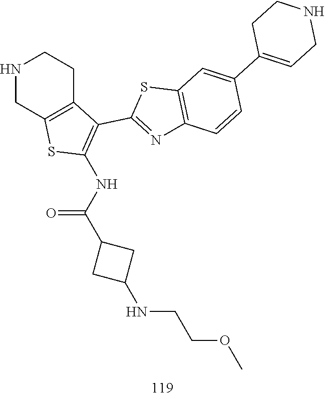 [SMILES: COCCNC1CC(C(=O)Nc2sc3c(c2-c2nc4ccc(C5=CCNCC5)cc4s2)CCNC3)C1] | 3-((2-methoxyethyl)amino)- N-(3-(6-(1,2,3,6- tetrahydropyridin-4-yl)benzo [d]thiazol-2-yl)-4,5,6,7- tetrahydrothieno[2,3-c] pyridin-2-yl)cyclobutane-1- carboxamide |
| 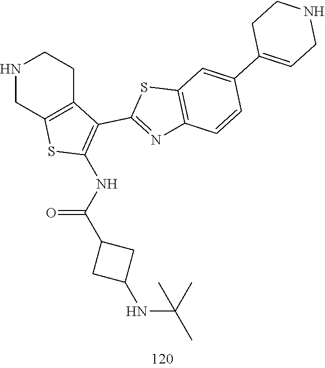 [SMILES: CC(C)(C)NC1CC(C(=O)Nc2sc3c(c2-c2nc4ccc(C5=CCNCC5)cc4s2)CCNC3)C1] | 3-(tert-butylamino)-N-(3-(6- (1,2,3,6-tetrahydropyridin- 4-yl)benzo[d]thiazol-2-yl)- 4,5,6,7-tetrahydrothieno [2,3-c]pyridin-2-yl) cyclobutane-1-carboxamide |
| 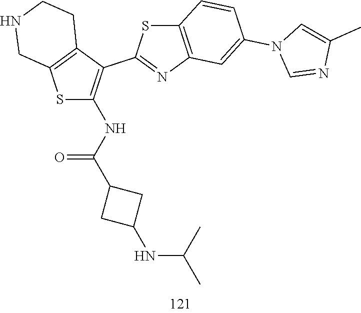 [SMILES: Cc1cn(-c2ccc3sc(-c4c(NC(=O)C5CC(NC(C)C)C5)sc5c4CCNC5)nc3c2)cn1] | 3-(isopropylamino)-N-(3-(5- (4-methyl-1H-imidazol- 1-yl)benzo[d]thiazol-2-yl)- 4,5,6,7-tetrahydrothieno [2,3-c]pyridin-2-yl) cyclobutane-1-carboxamide |
| 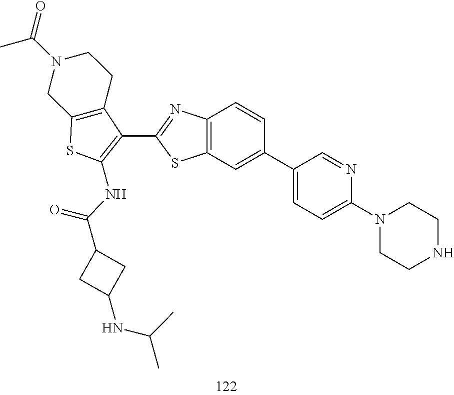 [SMILES: CC(=O)N1CCc2c(sc(NC(=O)C3CC(NC(C)C)C3)c2-c2nc3ccc(-c4ccc(N5CCNCC5)nc4)cc3s2)C1] | N-(6-acetyl-3-(6-(6- (piperazin-1-yl)pyridin-3-yl) benzo[d]thiazol-2-yl)- 4,5,6,7-tetrahydrothieno[2,3-c] pyridin-2-yl)-3- (isopropylamino)cyclobutane-1- carboxamide |
| 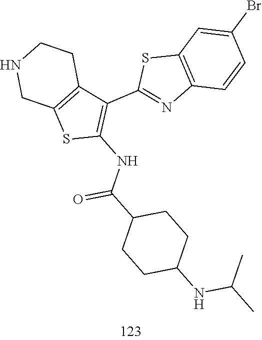 [SMILES: CC(C)NC1CCC(C(=O)Nc2sc3c(c2-c2nc4ccc(Br)cc4s2)CCNC3)CC1] | N-(3-(6-bromobenzo[d] thiazol-2-yl)-4,5,6,7- tetrahydrothieno[2,3-c]pyridin- 2-yl)-4-(isopropylamino) cyclohexane- 1-carboxamide |
| 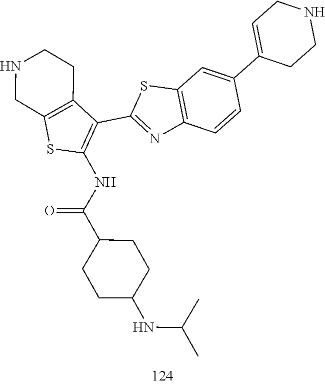 [SMILES: CC(C)NC1CCC(C(=O)Nc2sc3c(c2-c2nc4ccc(C5=CCNCC5)cc4s2)CCNC3)CC1] | 4-(isopropylamino)-N-(3-(6- (1,2,3,6-tetrahydropyridin- 4-yl)benzo[d]thiazol-2-yl)- 4,5,6,7-tetrahydrothieno [2,3-c]pyridin-2-yl) cyclohexane-1-carboxamide |
| 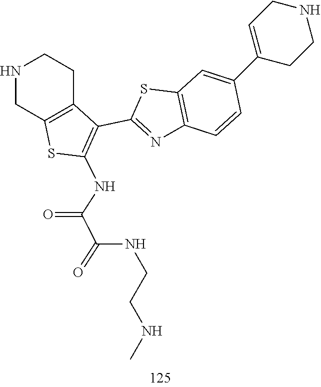 [SMILES: CNCCNC(=O)C(=O)Nc1sc2c(c1-c1nc3ccc(C4=CCNCC4)cc3s1)CCNC2] | N¹-(2-(methylamino)ethyl)- N²-(3-(6-(1,2,3,6- tetrahydropyridin-4-yl)benzo [d]thiazol-2-yl)-4,5,6,7- tetrahydrothieno[2,3-c] pyridin-2-yl)oxalamide |
| 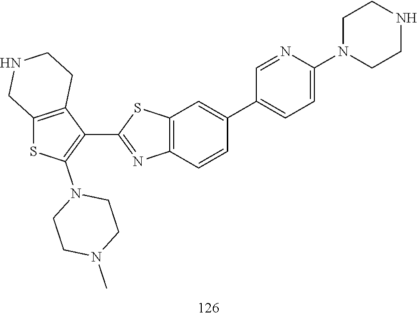 [SMILES: CN1CCN(c2sc3c(c2-c2nc4ccc(-c5ccc(N6CCNCC6)nc5)cc4s2)CCNC3)CC1] | 2-(2-(4-methylpiperazin-1- yl)-4,5,6,7-tetrahydrothieno [2,3-c]pyridin-3-yl)-6-(6- (piperazin-1-yl)pyridin-3- yl)benzo[d]thiazole |
| 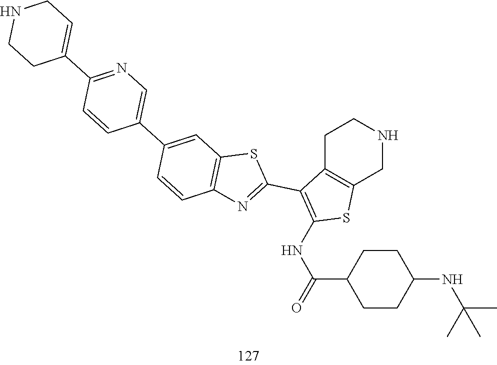 [SMILES: CC(C)(C)NC1CCC(C(=O)Nc2sc3c(c2-c2nc4ccc(-c5ccc(C6=CCNCC6)nc5)cc4s2)CCNC3)CC1] | 4-(tert-butylamino)-N-(3- (6-(1′,2′,3′,6′-tetrahydro- [2,4′-bipyridin]-5-yl)benzo [d]thiazol-2-yl)-4,5,6,7- tetrahydrothieno[2,3-c] pyridin-2-yl)cyclohexane-1- carboxamide |
| 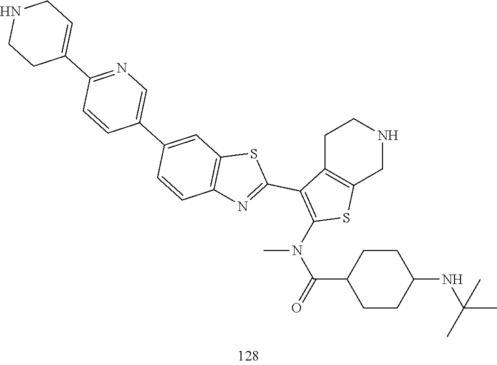 [SMILES: CN(C(=O)C1CCC(NC(C)(C)C)CC1)c1sc2c(c1-c1nc3ccc(-c4ccc(C5=CCNCC5)nc4)cc3s1)CCNC2] | 4-(tert-butylamino)-N- methyl-N-(3-(6-(1′,2′,3′,6′- tetrahydro-[2,4′-bipyridin]-5- yl)benzo[d]thiazol-2-yl)- 4,5,6,7-tetrahydrothieno [2,3-c]pyridin-2-yl) cyclohexane-1-carboxamide |
| 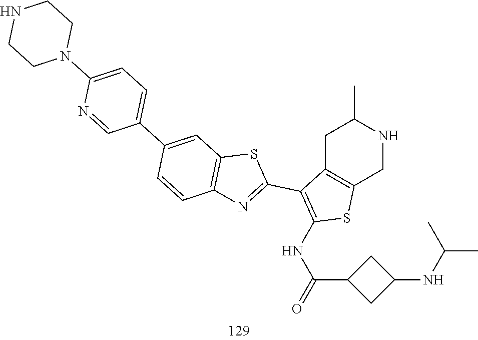 [SMILES: CC(C)NC1CC(C(=O)Nc2sc3c(c2-c2nc4ccc(-c5ccc(N6CCNCC6)nc5)cc4s2)CC(C)NC3)C1] | 3-(isopropylamino)-N-(5- methyl-3-(6-(6-(piperazin- 1-yl)pyridin-3-yl)benzo [d]thiazol-2-yl)-4,5,6,7- tetrahydrothieno[2,3-c] pyridin-2-yl)cyclobutane-1- carboxamide |
| 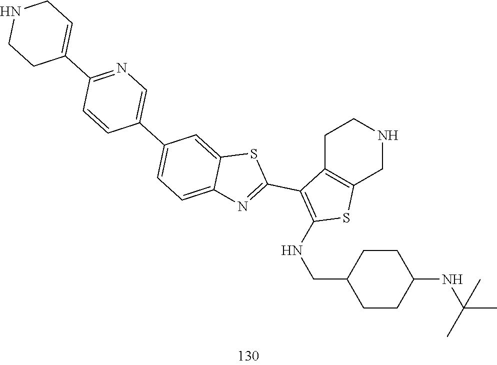 [SMILES: CC(C)(C)NC1CCC(CNc2sc3c(c2-c2nc4ccc(-c5ccc(C6=CCNCC6)nc5)cc4s2)CCNC3)CC1] | N-((4-(tert-butylamino) cyclohexyl)methyl)-3-(6-(1′, 2′,3′,6′-tetrahydro-[2,4′- bipyridin]-5-yl)benzo[d] thiazol-2-yl)-4,5,6,7- tetrahydrothieno[2,3-c]pyridin-2- amine |
| 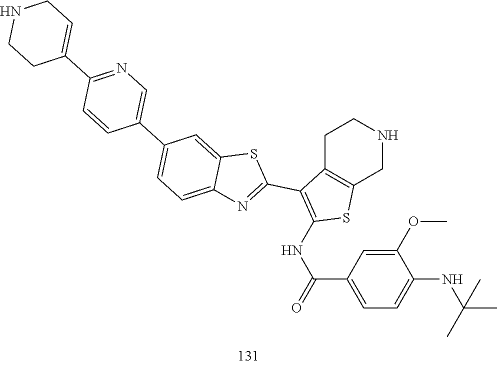 [SMILES: COc1cc(C(=O)Nc2sc3c(c2-c2nc4ccc(-c5ccc(C6=CCNCC6)nc5)cc4s2)CCNC3)ccc1NC(C)(C)C] | 4-(tert-butylamino)-3-methoxy- N-(3-(6-(1′,2′,3′,6′- tetrahydro-[2,4′-bipyridin]-5- yl)benzo[d]thiazol-2-yl)- 4,5,6,7-tetrahydrothieno[2,3-c] pyridin-2-yl)benzamide |
| 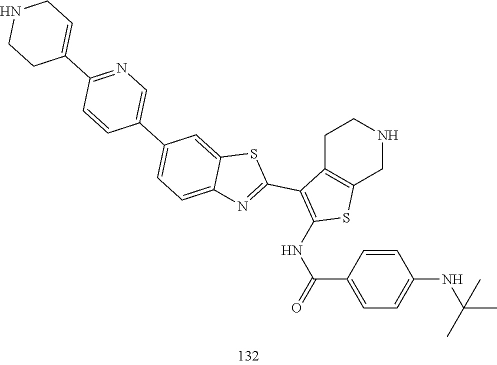 [SMILES: CC(C)(C)Nc1ccc(C(=O)Nc2sc3c(c2-c2nc4ccc(-c5ccc(C6=CCNCC6)nc5)cc4s2)CCNC3)cc1] | 5-(tert-butylamino)-N-(3- (6-(1′,2′,3′,6′-tetrahydro-[2, 4′-bipyridin]-5-yl)benzo [d]thiazol-2-yl)-4,5,6,7- tetrahydrothieno[2,3-c] pyridin-2-yl)benzamide |
| 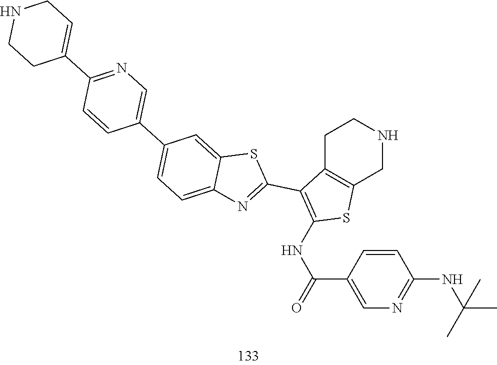 [SMILES: CC(C)(C)Nc1ccc(C(=O)Nc2sc3c(c2-c2nc4ccc(-c5ccc(C6=CCNCC6)nc5)cc4s2)CCNC3)cn1] | 6-(tert-butylamino)-N-(3- (6-(1′,2′,3′,6′-tetrahydro-[2, 4′-bipyridin]-5-yl)benzo [d]thiazol-2-yl)-4,5,6,7- tetrahydrothieno[2,3-c] pyridin-2-yl)nicotinamide |
| 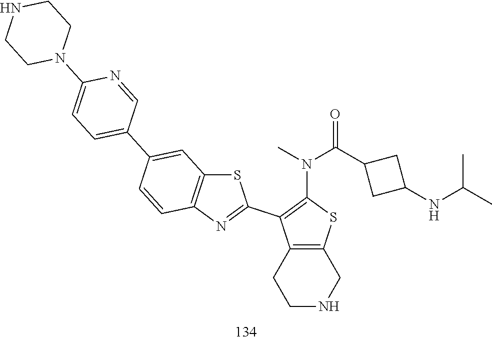 [SMILES: CC(C)NC1CC(C(=O)N(C)c2sc3c(c2-c2nc4ccc(-c5ccc(N6CCNCC6)nc5)cc4s2)CCNC3)C1] | 3-(isopropylamino)-N- methyl-N-(3-(6-(6-(piperazin- 1-yl)pyridin-3-yl)benzo [d]thiazol-2-yl)-4,5,6,7- tetrahydrothieno[2,3-c] pyridin-2-yl)cyclobutane-1- carboxamide |
| 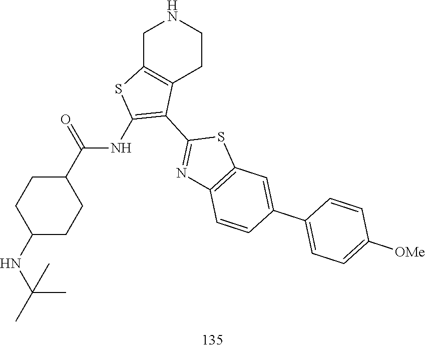 [SMILES: COc1ccc(-c2ccc3nc(-c4c(NC(=O)C5CCC(NC(C)(C)C)CC5)sc5c4CCNC5)sc3c2)cc1] | 4-(tert-butylamino)-N-(3- (6-(4-methoxyphenyl) benzo[d]thiazol-2-yl)-4,5,6,7- tetrahydrothieno[2,3-c] pyridin-2-yl)cyclohexane- 1-carboxamide |
| 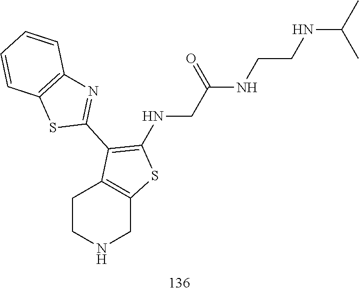 [SMILES: CC(C)NCCNC(=O)CNc1sc2c(c1-c1nc3ccccc3s1)CCNC2] | 2-((3-(benzo[d]thiazol-2-yl)- 4,5,6,7-tetrahydrothieno [2,3-c]pyridin-2-yl)amino)- N-(2-(isopropylamino) ethyl)acetamide |
| 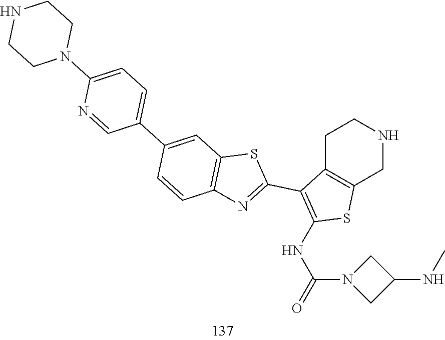 [SMILES: CNC1CN(C(=O)Nc2sc3c(c2-c2nc4ccc(-c5ccc(N6CCNCC6)nc5)cc4s2)CCNC3)C1] | 3-(methylamino)-N-(3-(6- (6-(piperazin-1-yl)pyridin- 3-yl)benzo[d]thiazol-2-yl)- 4,5,6,7-tetrahydrothieno [2,3-c]pyridin-2-yl) azetidine-1-carboxamide |
| 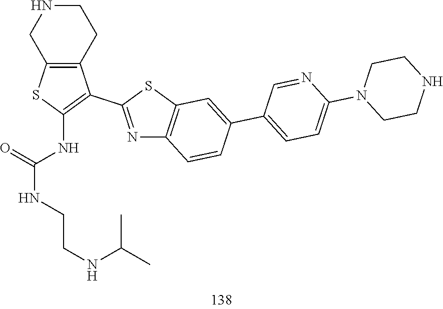 [SMILES: CC(C)NCCNC(=O)Nc1sc2c(c1-c1nc3ccc(-c4ccc(N5CCNCC5)nc4)cc3s1)CCNC2] | 1-(2-(isopropylamino)ethyl)- 3-(3-(6-(6-(piperazin-1- yl)pyridin-3-yl)benzo[d] thiazol-2-yl)-4,5,6,7- tetrahydrothieno[2,3-c] pyridin-2-yl)urea |
| 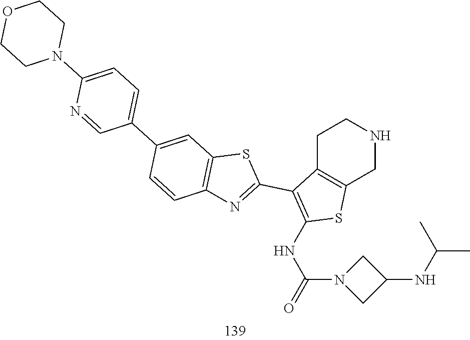 [SMILES: CC(C)NC1CN(C(=O)Nc2sc3c(c2-c2nc4ccc(-c5ccc(N6CCOCC6)nc5)cc4s2)CCNC3)C1] | 2-(isopropylamino)-N-(3- (6-(6-morpholinopyridin-3- yl)benzo[d]thiazol-2-yl)- 4,5,6,7-tetrahydrothieno[2, 3-c]pyridin-2-yl) cyclobutane-1-carboxamide |
| 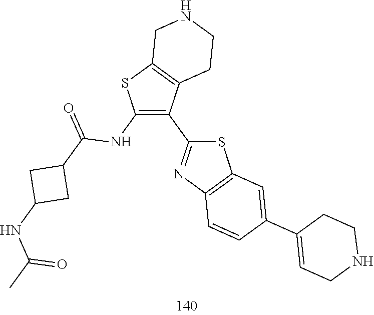 [SMILES: CC(=O)NC1CC(C(=O)Nc2sc3c(c2-c2nc4ccc(C5=CCNCC5)cc4s2)CCNC3)C1] | 3-(isopropylamino)-N-(3- (6-(6-morpholinopyridin-3- yl)benzo[d]thiazol-2-yl)- 4,5,6,7-tetrahydrothieno[2, 3-c]pyridin-2-yl) cyclobutane-1-carboxamide |
| 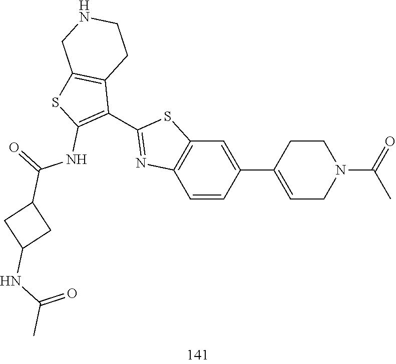 [SMILES: CC(=O)NC1CC(C(=O)Nc2sc3c(c2-c2nc4ccc(C5=CCN(C(C)=O)CC5)cc4s2)CCNC3)C1] | 4-acetamido-N-(3-(6-(1-acetyl- 1,2,3,6-tetrahydropyridin- 4-yl)benzo[d]thiazol-2-yl)- 4,5,6,7-tetrahydrothieno [2,3-c]pyridin-2-yl) cyclobutane-1-carboxamide |
| 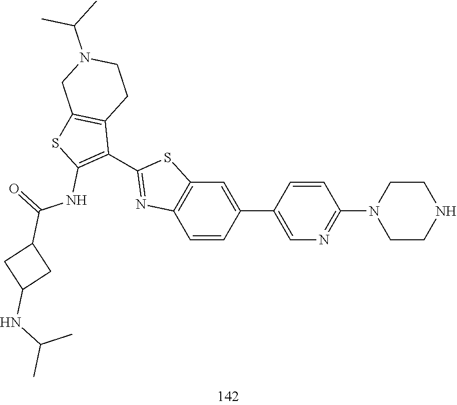 [SMILES: CC(C)NC1CC(C(=O)Nc2sc3c(c2-c2nc4ccc(-c5ccc(N6CCNCC6)nc5)cc4s2)CCN(C(C)C)C3)C1] | N-(6-isopropyl-3-(6-(6- (piperazin-1-yl)pyridin-3-yl) benzo[d]thiazol-2-yl)-4,5, 6,7-tetrahydrothieno[2,3-c] pyridin-2-yl)-3- (isopropylamino)cyclobutane-1- carboxamide |
| 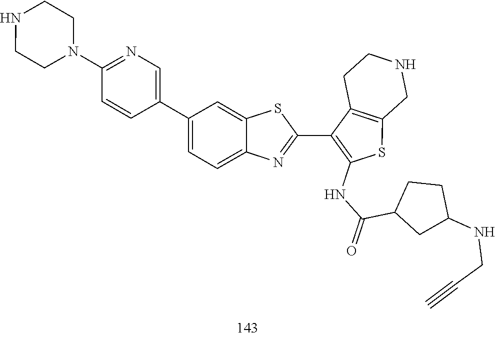 [SMILES: C#CCNC1CCC(C(=O)Nc2sc3c(c2-c2nc4ccc(-c5ccc(N6CCNCC6)nc5)cc4s2)CCNC3)C1] | O-(3-(6-(6-(piperazin-1-yl) pyridin-3-yl)benzo[d]thiazol- 2-yl)-4,5,6,7-tetrahydrothieno [2,3-c]pyridin-2-yl)- 3-(prop-2-yn-1-ylamino) cyclopentane-1-carboxamide |

or the stereoisomer, the tautomer or the pharmaceutically acceptable salt thereof.

Note: If there is a difference between a drawn structure and the name given to that structure, the drawn structure shall prevail.

Still further, the present disclosure provides a pharmaceutical composition, which comprises an effective amount of the compound represented by formula (I), (IIa), (IIb), (IIIa), (IIIb), (IVa), (IVb), (V), (VIa), (VIb), (VIc) or (VId), or the stereoisomer, the tautomer or the pharmaceutically acceptable salt thereof, and a pharmaceutically acceptable carrier, excipient or a combination thereof.

In the present disclosure, provided is use of the compound represented by formula (I), (IIa), (IIb), (IIIa), (IIIb), (IVa), (IVb), (V), (VIa), (VIb), (VIc) or (VId), or the stereoisomer, the tautomer or the pharmaceutically acceptable salt thereof, or the pharmaceutical composition thereof disclosed herein in the manufacture of a medicament for treating a disease mediated by Myc, wherein the medicament is preferably a Myc regulator. In some embodiments, further, the Myc regulator is preferably a c-Myc regulator.

In the present disclosure, provided is use of the compound represented by formula (I), (IIa), (IIb), (IIIa), (IIIb), (IVa), (IVb), (V), (VIa), (VIb), (VIc) or (VId), or the stereoisomer, the tautomer or the pharmaceutically acceptable salt thereof, or the pharmaceutical composition thereof disclosed herein in the manufacture of a medicament for treating a disease mediated by Myc, wherein the disease mediated by Myc is preferably a solid tumor or a malignant disease of blood system. Preferably, the disease mediated by Myc is selected from the group consisting of lymphoma, leukemia, osteosarcoma, liver cancer, kidney cancer, lung cancer, pancreatic cancer and breast cancer.

In the present disclosure, provided is use of the compound represented by formula (I), (IIa), (IIb), (IIIa), (IIIb), (IVa), (IVb), (V), (VIa), (VIb), (VIc) or (VId), or the stereoisomer, the tautomer or the pharmaceutically acceptable salt thereof, or the pharmaceutical composition thereof disclosed herein in the manufacture of a medicament for treating lymphoma, leukemia, osteosarcoma, liver cancer, kidney cancer, lung cancer, pancreatic cancer or breast cancer.

### Term Definitions

Unless otherwise stated, some terms used in the description and claims of the present disclosure are defined as follows:

“Bond” means that the indicated substituent does not exist, and the two ends of the substituent are directly connected to form a bond.

“Alkyl”, when used as a group or part of a group, refers to a C₁-C₂₀ linear or branched aliphatic hydrocarbon group. It is preferably C₁-C₁₀ alkyl, and more preferably C₁-C₈ alkyl. Examples of alkyl include, but are not limited to, methyl, ethyl, n-propyl, isopropyl, n-butyl, isobutyl, tert-butyl, sec-butyl, n-pentyl, 1,1-dimethylpropyl, 1,2-dimethylpropyl, 2,2-dimethylpropyl, 1-ethylpropyl, 2-methylbutyl, 3-methylbutyl, n-hexyl, 1-ethyl-2-methylpropyl, 1,1,2-trimethylpropyl, 1,1-dimethylbutyl, 1,2-dimethylbutyl, 2,2-dimethylbutyl, 1,3-dimethylbutyl, 2-ethylbutyl, 2-methylpentyl, 3-methylpentyl, 4-methylpentyl, 2,3-dimethylbutyl, etc. Alkyl may be substituted or unsubstituted.

“Alkenyl” refers to an aliphatic hydrocarbon group containing one carbon-carbon double bond, which may be straight or branched. It is preferably C₂-C₁₀ alkenyl, and more preferably C₂-C₈ alkenyl. Representative examples include, but are not limited to,

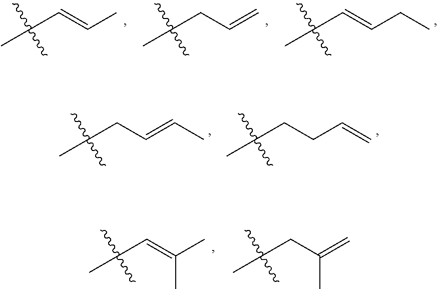

etc. Alkenyl may be substituted or unsubstituted.

“Alkynyl” refers to an aliphatic hydrocarbon group containing one carbon-carbon triple bond, which may be straight or branched. It is preferably C₂-C₁₀ alkynyl, more preferably C₂-C₈ alkynyl, and most preferably C₂-C₄ alkynyl. Examples of alkynyl include, but are not limited to, ethynyl,

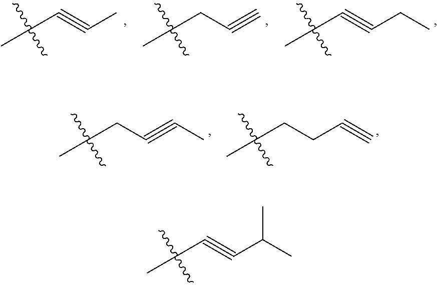

etc. Alkynyl may be substituted or unsubstituted.

“Cycloalkyl” refers to a saturated or partially saturated monocyclic, fused, bridged or spiro carbon ring. It is preferably C₃-C₁₂ cycloalkyl, more preferably C₃-C₈ cycloalkyl, and most preferably C₃-C₆ cycloalkyl. Examples of monocyclic cycloalkyl include, but are not limited to cyclopropyl, cyclobutyl, cyclopentyl, cyclopentenyl, cyclohexyl, cyclohexenyl, cyclohexadienyl, cycloheptyl, cycloheptatrienyl, cyclooctyl, and the like, preferably cyclopropyl and cyclohexenyl. Cycloalkyl may be substituted or unsubstituted.

“Spirocycloalkyl” refers to 5- to 18-membered polycyclic groups with two or more cyclic structures, in which the single rings share one carbon atom (called as spiro atom) with each other, and the ring contains one or more double bonds, but none of the rings is aromatic. It is preferably 6- to 14-membered, more preferably 7- to 10-membered. According to the number of shared spiro atoms between the rings, the spirocycloalkyl is divided into monospiro, bispiro or polyspiro cycloalkyl, preferably monospiro and bispiro cycloalkyl, preferably 4-membered/5-membered, 4-membered/6-membered, 5-membered/5-membered or 5-membered/6-membered. Examples of “spirocycloalkyl” include, but are not limited to, spiro[4.5]decyl, spiro[4.4]nonyl, spiro[3.5]nonyl, and spiro[2.4]heptyl.

“Fused cycloalkyl” refers to 5- to 18-membered all-carbon polycyclic groups, in which two or more cyclic structures share a pair of carbon atoms with each other, and one or more rings may contain one or more double bonds, but none of the rings is aromatic. It is preferably 6- to 12-membered, and more preferably 7- to 10-membered. According to the number of constituent rings, it can be divided into bicyclic, tricyclic, tetracyclic or polycyclic fused cycloalkyl, preferably or bicyclic tricyclic, and more preferably 5-membered/5-membered or 5-membered/6-membered bicyclic alkyl. Examples of “fused cycloalkyl” include, but are not limited to, bicyclo[3.1.0]hexyl, bicyclo[3.2.0]hept-1-enyl, bicyclo[3.2.0]heptyl, decalinyl or tetradecahydrophenanthyl.

“Bridged cycloalkyl” refers to 5- to 18-membered all-carbon polycyclic groups, in which two or more cyclic structures share two carbon atoms that are not directly attached with each other, and one or more rings may contain one or more double bonds, but none of the rings is aromatic. It is preferably 6- to 14-membered, more preferably 7- to 10-membered. According to the number of constituent rings, it can be divided into bicyclic, tricyclic, tetracyclic or polycyclic bridged cycloalkyl, preferably bicyclic, tricyclic or tetracyclic, and more preferably bicyclic or tricyclic. Examples of “bridged cycloalkyl” include, but are not limited to, (1s,4s)-bicyclo[2.2.1]heptyl, bicyclo[3.2.1]octyl, (1s,5s)-bicyclo[3.3.1]nonyl, bicyclo[2.2.2]octyl, (1r,5r)-bicyclo[3.3.2]decyl, and bicyclo[1.1.1]pentyl.

“Heterocyclyl”, “heterocycle” or “heterocyclic” can be used interchangeably in the present disclosure and all refer to a non-aromatic heterocyclic group, in which one or more atoms forming the ring are heteroatoms, such as N, O, S, P and Se. It includes a monocyclic ring, a fused ring, a bridged ring, and a spiro ring, and the ring may contain one or more double bonds. It preferably has 3 to 12 ring atoms, and is preferably a 4- to 7-membered monocyclic ring or a 7- to 10-membered bicyclic or tricyclic ring, which may contain 1, 2 or 3 atoms selected from the group consisting of N, O, S(O)ₙ (wherein n is selected from the group consisting of 0, 1 and 2), P(O)ₘ (wherein m is selected from the group consisting of 0 and 1), and Se. Examples of “heterocyclyl” include, but are not limited to, morpholinyl, oxetanyl, thiomorpholinyl, tetrahydropyranyl, 1,1-dioxo-thiomorpholinyl, piperidinyl, 2-oxo-piperidinyl, pyrrolidinyl, 2-oxo-pyrrolidinyl, piperazin-2-one, 8-oxa-3-aza-bicyclo[3.2.1]octyl, piperazinyl, 1,2,3,6-tetrahydropyridyl or 3,6-dihydro-2H-pyranyl. Heterocyclyl may be substituted or unsubstituted.

“Spiroheterocyclyl” refers to 5- to 18-membered polycyclic groups with two or more cyclic structures, in which single rings share one atom with each other, and the rings can contain one or more double bonds, but none of the ring is aromatic. One or more ring atoms therein are heteroatoms selected from the group consisting of N. O. S(O) n (wherein n is selected from the group consisting of 0, 1 and 2), P(O) m (wherein m is selected from the group consisting of 0 and 1) and Se, and the rest ring atoms are carbon. It is preferably 6- to 14-membered, and more preferably 7- to 10-membered. According to the number of shared spiro atoms between the rings, the spiroheterocyclyl is divided into monospiroheterocyclyl, bispiroheterocyclyl or polyspiroheterocyclyl, preferably monospiroheterocyclyl and bispiroheterocyclyl. It is more preferably a 4-membered/4-membered, 4-membered/5-membered, 4-membered/6-membered, 5-membered/5-membered or 5-membered/6-membered monospiroheterocyclyl. Examples of “spiroheterocyclyl” include, but are not limited to: 1,7-dioxaspiro[4.5]decyl, 2-oxa-7-azaspiro[4.4]nonyl, 7-oxaspiro[3.5]nonyl and 5-oxaspiro[2.4]heptyl.

“Fused heterocyclyl” refers to full-carbon polycyclic groups, in which two or more ring structures share a pair of atoms with each other, and one or more rings may contain one or more double bonds, but none of the rings is aromatic. One or more ring atoms therein are heteroatoms selected from the group consisting of N. O. S(O) n (wherein n is selected from the group consisting of 0, 1 and 2), P(O) m (wherein m is selected from the group consisting of 0 and 1) and Se, and the rest ring atoms are carbon. It is preferably 6- to 14-membered, and more preferably 7- to 10-membered. According to the number of constituent rings, it can be divided into bicyclic, tricyclic, tetracyclic or polycyclic fused heterocyclyl, preferably bicyclic or tricyclic, and more preferably 5-membered/5-membered or 5-membered/6-membered bicyclic fused heterocyclyl. Non-limiting examples of “fused heterocyclyl” include, but are not limited to, octahydropyrro [3,4-c]pyrrolyl, octahydro-1H-isoindolyl, 3-azabicyclo[3.1.0]hexyl, octahydrobenzo[b][1,4]dioxine.

“Bridged heterocyclyl” refers to 5- to 18-membered polycyclic groups, in which two or more cyclic structures share two atoms that are not directly attached to each other, and one or more rings may contain one or more double bonds, but none of the rings is aromatic. One or more ring atoms therein are heteroatoms selected from the group consisting of N. O. S(O) n (wherein n is selected from the group consisting of 0, 1 and 2), P(O) m (wherein m is selected from the group consisting of 0 and 1) and Se, and the rest ring atoms are carbon. It is preferably 6- to 14-membered, and more preferably 7- to 10-membered. According to the number of constituent rings, it can be divided into bicyclic, tricyclic, tetracyclic or polycyclic bridged heterocyclyl, preferably bicyclic, tricyclic or tetracyclic, and more preferably bicyclic or tricyclic. Examples of “bridged heterocyclyl” include, but are not limited to, 2-azabicyclo[2.2.1]heptyl, 2-azabicyclo[2.2.2]octyl, and 2-azabicyclo[3.3.2]decyl.

“Aryl” refers to a carbocyclic aromatic system containing one or two rings, wherein the rings may be connected together in a fused manner. “Aryl” includes monocyclic or bicyclic aryl, such as aromatic groups of phenyl, naphthyl, and tetrahydronaphthyl. Preferred aryl is C₆-C₁₀ aryl, more preferred aryl is phenyl and naphthyl, and most preferred aryl is phenyl. Aryl may be substituted or unsubstituted.

“Heteroaryl” and “heteroaromatic ring” can be used interchangeably in the present disclosure, and all refer to monocyclic or polycyclic aromatic ring groups containing 5 to 14 ring atoms, which may contain 1 to 4 atoms selected from the group consisting of N, O, S and Se. It preferably contains 5 to 12 ring atoms, and is more preferably 5- to 6-membered monocyclic heteroaryl or 8- to 10-membered bicyclic heteroaryl. Examples of “heteroaryl” include, but are not limited to, furyl, pyridyl, 2-oxo-1,2-dihydropyridyl, pyridazinyl, pyrimidinyl, pyrazinyl, thienyl, isoxazolyl, oxazolyl, oxadiazolyl, imidazolyl, pyrrolyl, pyrazolyl, triazolyl, tetrazolyl, thiazolyl, isothiazolyl, 1,2,3-thiadiazolyl, benzodioxolyl, benzothienyl, benzimidazolyl, indolyl, isoindolyl, 1,3-dioxo-isoindolyl, quinolinyl, indazolyl, benzisothiazolyl, benzoxazolyl, benzisoxazolyl,

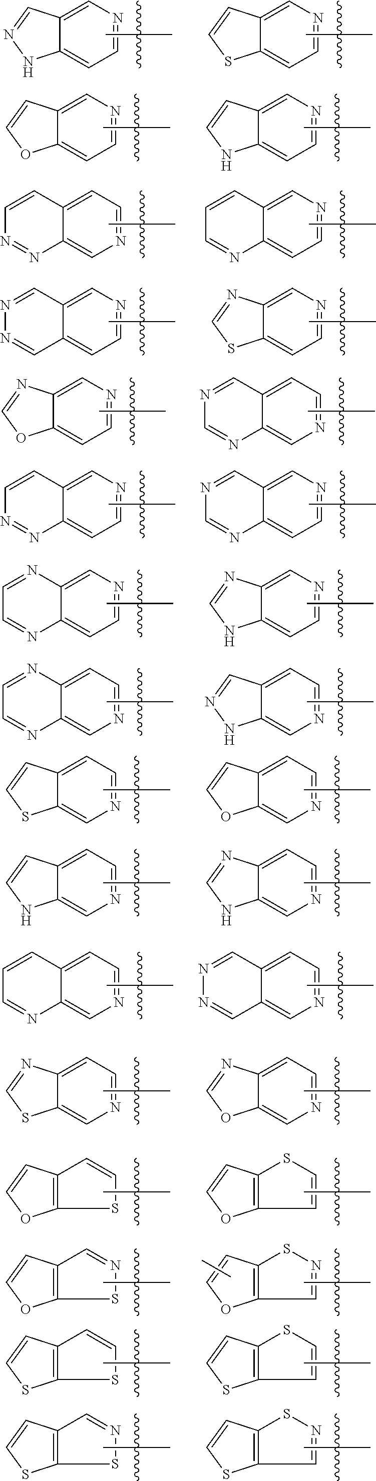

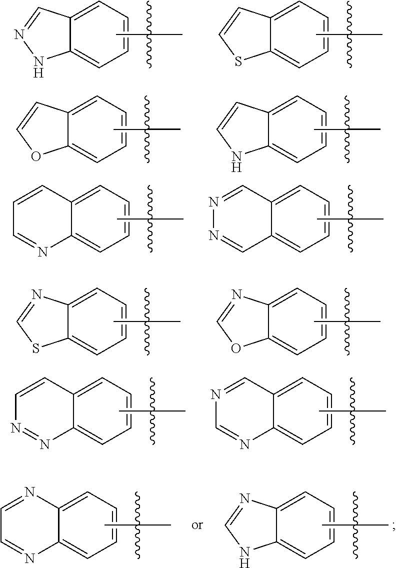

Heteroaryl may be substituted or unsubstituted.

“Fused ring” refers to a polycyclic group in which two or more cyclic structures share a pair of atoms with each other, and one or more rings may contain one or more double bonds, but at least one ring is not aromatic and at least one ring is aromatic at the same time, wherein zero, one or more ring atoms in the ring atoms are heteroatoms selected from the group consisting of N. O. S(O) n (wherein n is selected from the group consisting of 0, 1 and 2), P(O) m (wherein m is selected from the group consisting of 0 and 1) and Se, and the rest ring atoms are carbon. The fused ring is preferably a bicyclic or tricyclic fused ring, wherein the bicyclic fused ring is preferably a fused ring of aryl or heteroaryl with a monocyclic heterocyclyl or a monocyclic cycloalkyl. It is preferably 7- to 14-membered, more preferably 9- to 10-membered. Examples of “fused ring” include, but are not limited to:

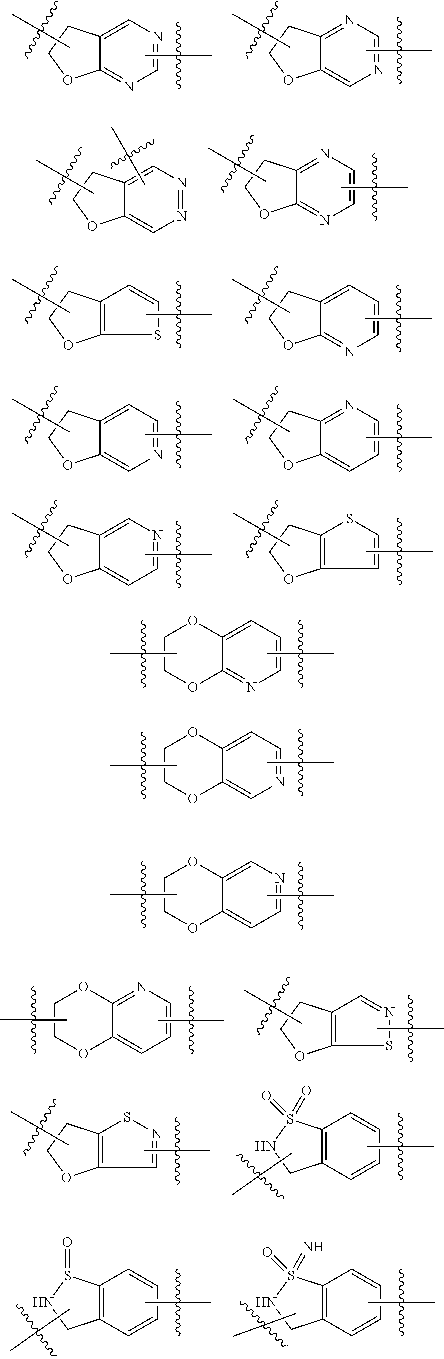

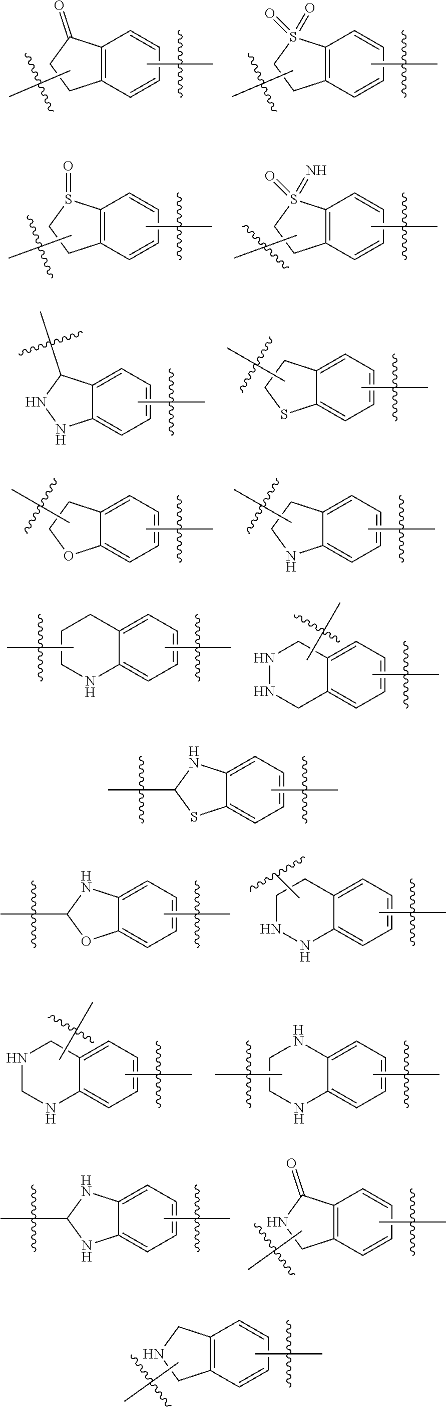

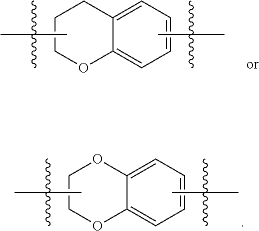

Fused ring may be substituted or unsubstituted.

“Alkoxy” refers to a group of (alkyl-O—). Wherein, the alkyl is as defined herein. C₁-C₈ alkoxy is preferred. Examples include, but are not limited to, methoxy, ethoxy, n-propoxy, isopropoxy, n-butoxy, isobutoxy, tert-butoxy, etc. Alkoxy may be substituted or unsubstituted.

“Alkenyloxy” refers to a group of (alkenyl-O—). Wherein, alkenyl is as defined herein. C₂-C₈ alkenyloxy is preferred. Alkenyloxy may be substituted or unsubstituted.

“Hydroxyalkyl” refers to a group of (-alkyl-OH). Wherein, the alkyl is as defined herein. C₁-C₈ hydroxyalkyl is preferred. Examples include, but are not limited to: hydroxymethyl, hydroxyethyl, hydroxypropyl, hydroxyisopropyl, hydroxybutyl, etc. Hydroxyalkyl may be substituted or unsubstituted.

“Alkylamino” refers to a group of (alkyl-NH—). Wherein, the alkyl is as defined herein. C₁-C₈ alkylamino is preferred. Examples include, but are not limited to, methylamino, ethylamino, n-propylamino, isopropylamino, n-butylamino, isobutoxy, tert-butoxy, etc. Alkylamino can be substituted or unsubstituted, and substituents can be on alkyl or N, such as dimethylamino and diethylamino.

“Aminoalkyl” refers to a group of (-alkyl-NH₂). Wherein, the alkyl is as defined herein. Examples include, but are not limited to, aminomethyl, aminoethyl, aminopropyl, aminoisopropyl, aminobutyl, aminopentyl, etc. Aminoalkyl can be substituted or unsubstituted, and substituents can be on alkyl groups or N, such as dimethylaminoalkyl.

“Alkylcarbonyl” refers to a group of (alkyl-C(O)—). Wherein, the alkyl is as defined herein. Examples include, but are not limited to, methylcarbonyl, ethylcarbonyl, n-propylcarbonyl, isopropylcarbonyl, n-butylcarbonyl, isobutylcarbonyl, etc. Alkylcarbonyl can be substituted or unsubstituted.

“Alkoxycarbonyl” refers to a group of (alkyl-O—C(O)—). Wherein, the alkyl is as defined herein. Examples include, but are not limited to, methoxycarbonyl, ethoxycarbonyl, n-propoxycarbonyl, isopropoxycarbonyl, etc. The alkoxycarbonyl may be substituted or unsubstituted.

“Haloalkyl” refers to alkyl substituted with halogen. Wherein, the halogen and alkyl are as defined herein.

“Haloalkoxy” refers to alkoxy substituted with halogen. Wherein, the halogen and alkoxy are as defined herein.

“Halohydroxyalkyl” refers to hydroxyalkyl substituted with halogen. Wherein, the halogen and hydroxyalkyl are as defined herein.

“Haloalkylamino” refers to alkylamino substituted with halogen. Wherein, the halogen and alkylamino are as defined herein.

“Cycloalkoxy” refers to a group of (cycloalkyl-O—). Wherein the cycloalkyl is as defined herein.

“Heteroepoxy” refers to a group of (heterocyclic-O—). Wherein the heterocyclyl is as defined herein.

“Hydroxyl” refers to the —OH group.

“Halogen” refers to fluorine, chlorine, bromine and iodine.

“Amino” refers to —NH₂.

“Cyano” refers to —CN.

“Nitro” refers to —NO₂.

“Carboxyl” refers to —C(O)OH.

“Amide” refers to —C(O)NH₂.

“Substituted” means that one or more hydrogen atoms, preferably 1 to 5, more preferably 1 to 3 hydrogen atoms in a group, are each independently substituted with a corresponding number of substituents. It goes without saying that the substituents are only in their possible chemical positions, and the person skilled in the art is able to determine (either experimentally or theoretically) possible or impossible substitutions without making undue effort. For example, an amino or hydroxyl group with a free hydrogen may be unstable when combined with a carbon atom with an unsaturated (e.g., olefinic) bond.

Unless otherwise specified, “substitution” or “substituted” described in this specification means that a group can be substituted by one or more groups selected from the group consisting of: H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heteroepoxy, aminosulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —OR_{g}, —SR_{g}, —C₁-C₈ alkylene-R_{g}, —OC(O)R_{g}, —C(O)R_{g}, —C(O)OR_{g}, —C(O)N(Rₓ)R_{y}, —NRₓR_{y}, —N(CH₃)R_{g}, —N(Rₓ)C(O)R_{y}, —N(Rₓ)C(O)NRₓR_{y}, —N(Rₓ)C(O)OR_{g}, —N(Rₓ)S(O)NRₓR_{y}, —N(Rₓ)S(O)₂NRₓR_{y}, —N(Rₓ)S(O)₂R_{g}, —S(O)R_{g}, —S(O)₂R_{g}, —S(O)₂NRₓR_{y} and —P(O)RₓR_{y}, wherein the alkyl, alkylene, alkoxy, alkenyl, alkynyl, alkenyloxy, cycloalkyl, cycloalkoxy, heterocyclyl, heterocycloalkoxy, aryl, heteroaryl, 3- to 12-membered ring, amino, hydroxyl or amide is optionally further substituted by one or more Rₒ.

When two Rₒ are attached to the same atom, two Rₒ form a 3- to 6-membered ring together with the atom to which they are attached, or when two Rₒ are attached to adjacent atoms, the two Rₒ form a 3- to 12-membered ring together with the atoms to which they are attached.
R_{g}, Rₓ, R_{y} and Rₒ are each independently selected from the group consisting of H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heterocyclyloxy, aminosulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —ORₛ, —SRₛ, —C₁-C₈ alkylene-Rₛ, —OC(O)Rₛ, —C(O)Rₛ, —C(O)ORₛ, —C(O)N(Rₛ)Rₜ, —NRₛRₜ, —N(CH₃)Rₛ, —N(Rₛ)C(O)Rₜ, —N(Rₛ)C(O)NRₛRₜ, —N(Rₛ)C(O)ORₜ, —N(Rₛ)S(O)NRₛRₜ, —N(Rₛ)S(O)₂NRₛRₜ, —N(Rₛ)S(O)₂Rₜ, —S(O)Rₛ, —S(O)₂Rₛ, —S(O)₂NRₛRₜ and —P(O)RₛRₜ, wherein the alkyl, alkylene, cycloalkyl, heterocyclyl, aryl or heteroaryl is optionally further substituted by one or more Rᵣ.

When two Rᵣ are attached to the same atom, the two Rᵣ form a 3- to 6-membered ring together with the atom to which they are attached, or when the two Rᵣ are attached to adjacent atoms, the two Rᵣ form a 3- to 12-membered ring together with the atoms to which they are attached.

Rᵣ, Rₛ and Rₜ are each independently selected from the group consisting of H, deuterium, C₁-C₈ alkyl, C₂-C₈ alkenyl, C₂-C₈ alkynyl, halogen, cyano, amino, nitro, hydroxyl, oxo, C₁-C₈ alkoxy, C₁-C₈ haloalkyl, hydroxyalkyl, aminoalkyl, C₁-C₈ alkylamino, alkylcarbonyl, alkoxycarbonyl, halohydroxyalkyl, C₁-C₈ haloalkylamino, C₃-C₁₂ cycloalkyl, 3- to 12-membered heterocyclyl, carboxyl, amide, C₆-C₁₀ aryl and 5- to 12-membered heteroaryl.

The compounds of the present disclosure can contain asymmetric centers or chiral centers and thus exist in different stereoisomeric forms. It is expected that all stereoisomeric forms of the compounds of the present disclosure, including but not limited to diastereomers, enantiomers, atropisomers, geometric (conformational) isomers and a mixture thereof, such as a racemic mixture, are within the scope of the present disclosure.

Unless otherwise indicated, the structures described in the present disclosure also include all isomeric forms of such structures (for example, diastereoisomers, enantiomers, atropisomers, and geometric (conformational) isomers), for example, R and S configurations of each asymmetric center, (Z) and (E) double bond isomers, and (Z) and (E) conformational isomers. Therefore, an individual stereoisomer, a mixture of enantiomers, a mixture of diastereoisomers and a mixture of geometric (conformational) isomers of the compounds of the present disclosure are within the scope of the present disclosure.

C, H, O, S, N, F, Cl, Br, I, etc. involved in the groups and compounds described in the present disclosure all include their isotopic forms. Meanwhile, C, H, O, S, N, F, Cl, Br and I involved in the groups and compounds described in the present disclosure can be optionally substituted by one or more of their corresponding isotopes, including but not limited to carbon isotopes ¹²C, ¹³C and ¹⁴C; hydrogen isotopes (H), deuterium (D), tritium (T); oxygen isotopes such as ¹⁶O, ¹⁷O, ¹⁸O; sulfur isotopes such as ³²S, ³³S, ³⁴S, ³⁶S; nitrogen isotopes such as ¹⁴N, ¹⁵N; fluorine isotopes such as ¹⁷F, ¹⁹F; chlorine isotopes such as ³⁵Cl, ³⁷Cl; bromine isotopes such as ⁷⁹Br, ⁸¹Br, and so on.

It is to be understood that both the foregoing general description and the following detailed description are exemplary and explanatory only, and do not limit any claims. It should be noted that in the specification and the appended claims, unless otherwise stated in the context, indication in singular forms such as “a”, “an” and “this”, includes plural forms. It should also be noted that “or” represents “and/or” unless otherwise specified. In addition, terms such as “comprising” and “including” are not limiting.

“Pharmaceutically acceptable salt” refers to certain salts of the above compounds that can maintain their original biological activity and are suitable for medical use. The pharmaceutically acceptable salt of the compound represented by formula (I) can be a metal salt, a salt formed with a suitable acid or a salt formed with a suitable base. A preferred salt is the salt formed by the compound of the present disclosure and an acid, and the acid suitable for forming the salt includes but is not limited to inorganic acids such as hydrochloric acid, hydrobromic acid, hydrofluoric acid, hydroiodic acid, sulfuric acid, nitric acid, phosphoric acid, carbonic acid, etc.; organic acids such as formic acid, acetic acid, trifluoroacetic acid, propionic acid, oxalic acid, malonic acid, succinic acid, fumaric acid, maleic acid, lactic acid, malic acid, tartaric acid, citric acid, picric acid, methanesulfonic acid, p-toluenesulfonic acid, ethanesulfonic acid, benzenesulfonic acid, camphorsulfonic acid, medlaric acid, isonicotinic acid, salicylic acid, ascorbic acid, gentisic acid, gluconic acid, pyruvic acid, naphthalenesulfonic acid, stearic acid, phenylacetic acid, p-aminobenzenesulfonic acid, hydroxyethyl sulfonic acid, dihydroxynaphthalene acid, tannic acid, etc.; and acidic amino acids such as aspartic acid and glutamic acid. A preferred salt is the salt formed by the compound of the present disclosure and a base, and bases suitable for forming the salt include but are not limited to inorganic bases such as sodium hydroxide, potassium hydroxide, sodium carbonate, sodium bicarbonate, sodium phosphate, etc. and organic bases such as ammonia water, triethylamine, diethylamine, piperazine, guanidine and diethanolamine.

### DETAILED DESCRIPTION

Chemical substances represented by some abbreviations in the present disclosure:
DCM: dichloromethane
TEA: triethylamine
DCE: dichloroethane
STAB: sodium triacetoxyborohydride
EDCI: carbodiimide hydrochloride
DMAP: 4-dimethylaminopyridine
DIAD: diisopropyl azodicarboxylate
TBAI: tetrabutylammonium iodide
EA: ethyl acetate
DIPEA: N,N-diisopropylethylamine
Pd(dppf)Cl₂: [1,1′-bis(diphenylphosphino)ferrocene]palladium dichloride
Me₄tBuXphos: 2-di-tert-butylphosphorus-3,4,5,6-tetramethyl-2′,4′,6′-triisopropyl biphenyl
Pd₂(dba)₃: tris(dibenzylideneacetone)dipalladium
DMP: dimethyl phthalate
TBD: 1,5,7-triazabicyclo[4.4.0]decyl-5-ene
EA/PE: ethyl acetate/petroleum ether
BINAP: (2R, 3S)-2.2′-diphenyl phosphine-1.1′-binaphthyl
Troc-Cl: chloroformate-2,2,2-trichloroethyl ester
(Boc)₂O: di-tert-butyl dicarbonate

Thereafter, the implementability of the present disclosure will be illustrated by the following examples. Those skilled in the art should understand that, in accordance with the teachings of the prior art, the modification or replacement of the corresponding technical features still falls within the protection scope claimed by the present disclosure.

### Example 1

Intermediate A1: tert-butyl 2-amino-3-(6-bromobenzo[d]thiazol-2-yl)-4,7-dihydrothieno[2,3-c]pyridin-6 (5H)-carboxylate

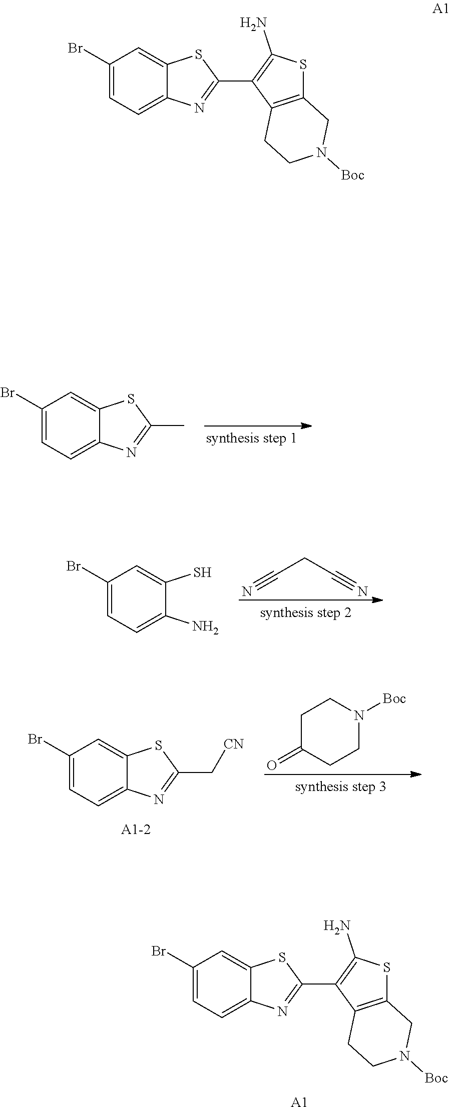

### Synthesis Step 1:2-amino-5-bromobenzenethiol (intermediate A1-1

6-Bromo-2-methylbenzo[d]thiazole (15.0 g, 65.79 mmol) was added into 45 mL of ethylene glycol, NaOH aqueous solution (11.5 mmol/mL, 45 mL) was added into the system, and the reaction was carried out at 140° C. for 5 h. After the reaction was completed, it was quenched by adding 50 mL of water and concentrated hydrochloric acid. The mixture was extracted three times with DCM, and the organic phases were combined, dried over anhydrous sodium sulfate, and concentrated under reduced pressure to obtain intermediate A1-1 (13 g) with a yield of 97%. ESI-MS (M+H)⁺=204.1.

### Synthesis Step 2:2-(6-bromobenzo[d]thiazol-2-yl) acetonitrile (intermediate A1-2)

Intermediate A1-1 (13.0 g, 51.38 mmol) was added into 40 mL of ethanol, malononitrile (3.7 g, 56.52 mmol) and acetic acid (40 mL) were added into the system in sequence, and the reaction was carried out at 90° C. for 3 h. After the reaction was completed, it was quenched by adding water, and the mixture was extracted three times with DCM. The organic phases were combined, dried over anhydrous sodium sulfate, concentrated under reduced pressure, slurried with methanol and petroleum ether in sequence, filtered and dried to obtain intermediate A1-2 (13 g) with a yield of 66%. ESI-MS (M+H)+=253.1.

### Synthesis Step 3: tert-butyl 2-amino-3-(6-bromobenzo[d]thiazol-2-yl)-4,7-dihydrothieno[2,3-c]pyridin-6 (5H)-carboxylate (intermediate A1)

Intermediate A1-2 (10.0 g, 39.68 mmol) was added into 35 mL of ethanol, and tert-butyl 4-oxopiperidin-1-carboxylate (8.7 g, 43.65 mmol) and morpholine (3.8 mL, 43.65 mmol) were added into the system in sequence, and the reaction was carried out at 40° C. for 2 h. After the reaction was completed, elemental sulfur (1.4 g, 43.56 mmol) was added into the system for reaction at 90° C. for 2 h. After the reaction was completed, it was quenched by adding water, and solids precipitated. The obtained mixture was filtered, slurried with ethanol, filtered and dried to obtain intermediate A1 (16 g) with a yield of 86%. ESI-MS (M+H)⁺=466.0.

### Example 2

### Intermediate A2: tert-butyl 2-amino-3-(benzo[d]thiazol-2-yl)-4,7-dihydrothieno[2,3-c]pyridin-6 (5H)-carboxylate

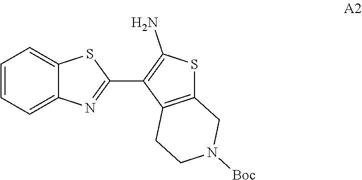 [SMILES: CC(C)(C)OC(=O)N1CCc2c(sc(N)c2-c2nc3ccccc3s2)C1]

Referring to the synthetic route and method of intermediate A1, in synthesis step 1, 6-bromo-2-methylbenzo[d]thiazole was replaced with 2-methylbenzo[d]thiazole to synthesize intermediate A2. ESI-MS (M+H)⁺-388.1.

### Example 3

### Intermediate A3: tert-butyl 2-amino-3-(5-bromobenzo[d]thiazol-2-yl)-4,7-dihydrothieno[2,3-c]pyridin-6 (5H)-carboxylate

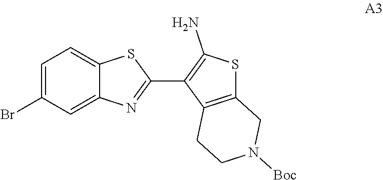 [SMILES: CC(C)(C)OC(=O)N1CCc2c(sc(N)c2-c2nc3cc(Br)ccc3s2)C1]

Referring to the synthetic route and method of intermediate A1, in synthesis step 1, 6-bromo-2-methylbenzo[d]thiazole was replaced with 5-bromo-2-methylbenzo[d]thiazole to synthesize intermediate A3. ESI-MS (M+H)⁺=466.0.

Referring to the synthetic routes and methods of intermediates A1 to A3, the following intermediate compounds were synthesized:

| Number of | | ESI-MS |
|---|---|---|
| intermediate | Structure of intermediate | (M + H)⁺ |
| A4 | 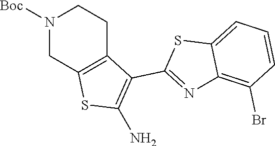 [SMILES: CC(C)(C)OC(=O)N1CCc2c(sc(N)c2-c2nc3c(Br)cccc3s2)C1] | 466.0 |
| A5 | 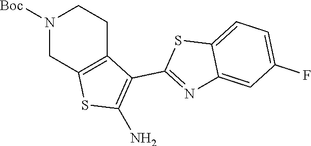 [SMILES: CC(C)(C)OC(=O)N1CCc2c(sc(N)c2-c2nc3cc(F)ccc3s2)C1] | 406.1 |
| A6 | 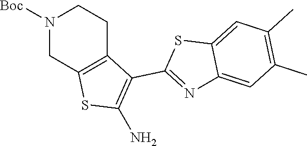 [SMILES: Cc1cc2nc(-c3c(N)sc4c3CCN(C(=O)OC(C)(C)C)C4)sc2cc1C] | 416.1 |
| A7 | 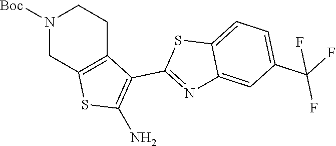 [SMILES: CC(C)(C)OC(=O)N1CCc2c(sc(N)c2-c2nc3cc(C(F)(F)F)ccc3s2)C1] | 456.1 |
| A8 | 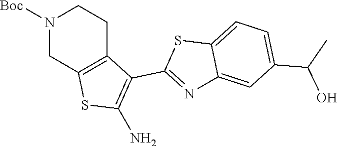 [SMILES: CC(O)c1ccc2sc(-c3c(N)sc4c3CCN(C(=O)OC(C)(C)C)C4)nc2c1] | 432.1 |
| A9 | 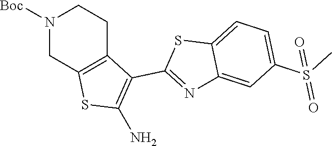 [SMILES: CC(C)(C)OC(=O)N1CCc2c(sc(N)c2-c2nc3cc(S(C)(=O)=O)ccc3s2)C1] | 466.1 |
| A10 | 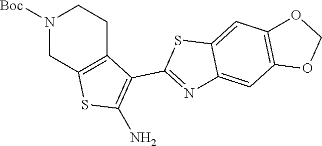 [SMILES: CC(C)(C)OC(=O)N1CCc2c(sc(N)c2-c2nc3cc4c(cc3s2)OCO4)C1] | 432.1 |
| A11 | 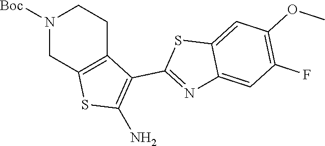 [SMILES: COc1cc2sc(-c3c(N)sc4c3CCN(C(=O)OC(C)(C)C)C4)nc2cc1F] | 436.1 |
| A12 | 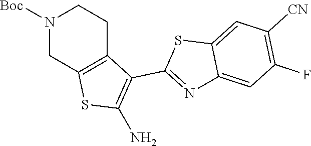 [SMILES: CC(C)(C)OC(=O)N1CCc2c(sc(N)c2-c2nc3cc(F)c(C#N)cc3s2)C1] | 431.1 |
| A13 | 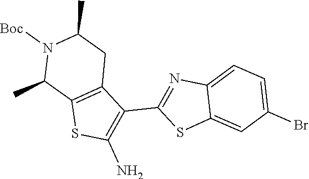 [SMILES: C[C@@H]1c2sc(N)c(-c3nc4ccc(Br)cc4s3)c2C[C@H](C)N1C(=O)OC(C)(C)C] | 494.0 |
| A14 | 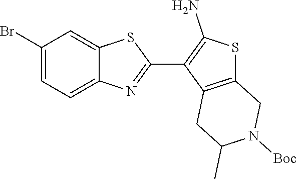 [SMILES: CC1Cc2c(sc(N)c2-c2nc3ccc(Br)cc3s2)CN1C(=O)OC(C)(C)C] | 480.0 |
| A15 | 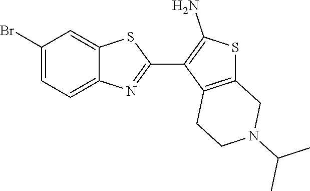 [SMILES: CC(C)N1CCc2c(sc(N)c2-c2nc3ccc(Br)cc3s2)C1] | 408.0 |

### Example 4

### Intermediate B1: tert-butyl 3-(6-bromobenzo[d]thiazol-2-yl)-2-(3-(isopropylamino)cyclobutane-1-formylamino)-4,7-dihydrothieno[2,3-c]pyridin-6 (5H)-carboxylate

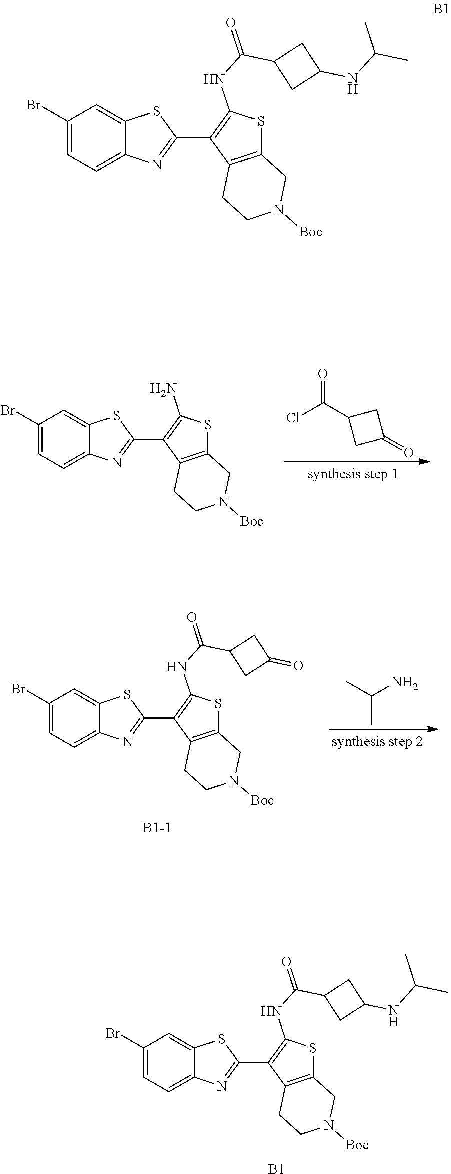

### Synthesis step 1: tert-butyl 3-(6-bromobenzo[d]thiazol-2-yl)-2-(3-oxocyclobutane-1-formylamino)-4,7-dihydrothieno[2,3-c]pyridin-6 (5H)-carboxylate (intermediate B1-1)

Intermediate A1 (2.0 g, 4.29 mmol) was dissolved in 20 mL of DCM, 3-oxocyclobutane-1-carbonyl chloride (1.4 g, 10.73 mmol) and TEA (866 mg, 8.58 mmol) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain intermediate B1-1 (1.9 g) with a yield of 79%. ESI-MS (M+H)⁺562.0.

### Synthesis Step 2: tert-butyl 3-(6-bromobenzo[d]thiazol-2-yl)-2-(3-(isopropylamino)cyclobutane-1-formylamino)-4,7-dihydrothieno[2,3-c]pyridin-6 (5H)-carboxylate (intermediate B1)

The intermediate B1-1 (1.9 g, 3.39 mmol) was dissolved in 10 mL of DCE, isopropylamine (1.4 mL, 16.93 mmol) and acetic acid (1.1 mL, 16.93 mmol) were added into the system in sequence, and the reaction was carried out at 80° C. for 2 h. Then STAB (1.4 g, 6.78 mmol) was added, and the reaction was continued at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain intermediate B1 (1.1 g) with a yield of 56%. ESI-MS (M+H)⁺605.1.

### Example 5

### Intermediate B2: tert-butyl 3-(5-bromobenzo[d]thiazol-2-yl)-2-(3-(isopropylamino)cyclobutane-1-formylamino)-4,7-dihydrothieno[2,3-c]pyridin-6 (5H)-carboxylate

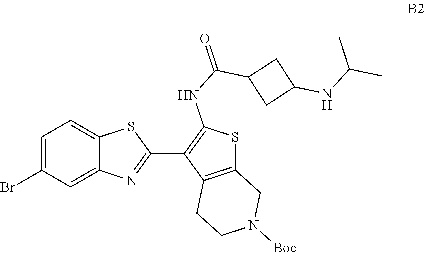 [SMILES: CC(C)NC1CC(C(=O)Nc2sc3c(c2-c2nc4cc(Br)ccc4s2)CCN(C(=O)OC(C)(C)C)C3)C1]

Referring to the synthesis route and method of intermediate B1, in synthesis step 1, intermediate A1 was replaced with intermediate A3 to synthesize intermediate B2. ESI-MS (M+H)⁺605.1.

### Example 6

### Intermediate B3: tert-butyl 3-(6-bromobenzo[d]thiazol-2-yl)-2-(3-((2-methoxyethyl)amino)cyclobutane-1-formamido)-4,7-dihydrothieno[2,3-c]pyridin-6 (5H)-carboxylate

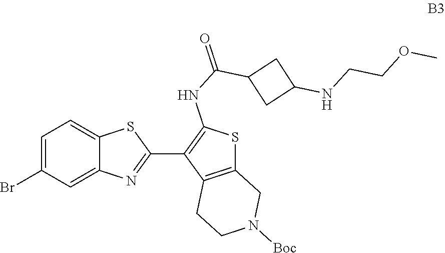 [SMILES: COCCNC1CC(C(=O)Nc2sc3c(c2-c2nc4cc(Br)ccc4s2)CCN(C(=O)OC(C)(C)C)C3)C1]

Referring to the synthetic route and method of intermediate B1, in synthesis step 2, the isopropylamine was replaced with 2-methoxyethane-1-amine to synthesize intermediate B3. ESI-MS (M+H)⁺621.1.

### Example 7

### Intermediate B4: tert-butyl 3-(6-bromobenzo[d]thiazol-2-yl)-2-(3-(isopropylamino)cyclopentane-1-formylamino)-4,7-dihydrothieno[2,3-c]pyridin-6 (5H)-carboxylate

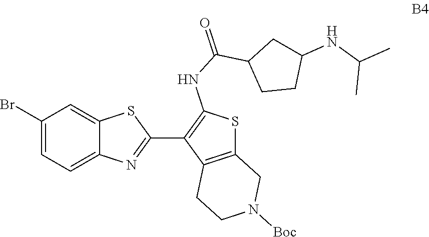 [SMILES: CC(C)NC1CCC(C(=O)Nc2sc3c(c2-c2nc4ccc(Br)cc4s2)CCN(C(=O)OC(C)(C)C)C3)C1]

Referring to the synthetic route and method of intermediate B1, in synthesis step 1, the 3-oxocyclobutane-1-carbonyl chloride was replaced with 3-oxocyclopentane-1-carbonyl chloride to synthesize intermediate B4. ESI-MS (M+H)⁺619.1.

Referring to the synthetic routes and methods of intermediates B1 to B4, the following intermediates were synthesized:

| Number of | | ESI-MS |
|---|---|---|
| intermediate | Structure of intermediate | (M + H)⁺ |
| B5 | 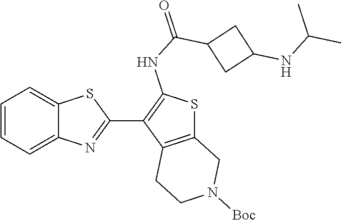 [SMILES: CC(C)NC1CC(C(=O)Nc2sc3c(c2-c2nc4ccccc4s2)CCN(C(=O)OC(C)(C)C)C3)C1] | 527.2 |
| B6 | 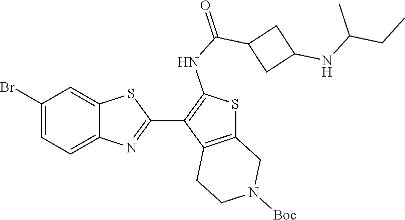 [SMILES: CCC(C)NC1CC(C(=O)Nc2sc3c(c2-c2nc4ccc(Br)cc4s2)CCN(C(=O)OC(C)(C)C)C3)C1] | 619.1 |
| B7 | 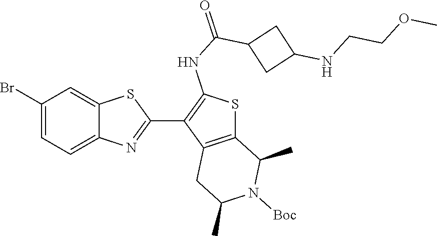 [SMILES: COCCNC1CC(C(=O)Nc2sc3c(c2-c2nc4ccc(Br)cc4s2)C[C@H](C)N(C(=O)OC(C)(C)C)[C@@H]3C)C1] | 649.1 |
| B8 | 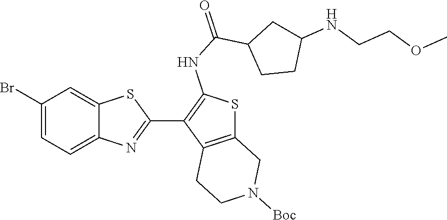 [SMILES: COCCNC1CCC(C(=O)Nc2sc3c(c2-c2nc4ccc(Br)cc4s2)CCN(C(=O)OC(C)(C)C)C3)C1] | 635.1 |
| B9 | 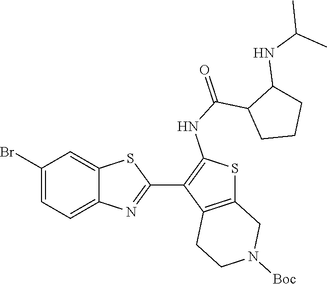 [SMILES: CC(C)NC1CCCC1C(=O)Nc1sc2c(c1-c1nc3ccc(Br)cc3s1)CCN(C(=O)OC(C)(C)C)C2] | 619.1 |
| B10 | 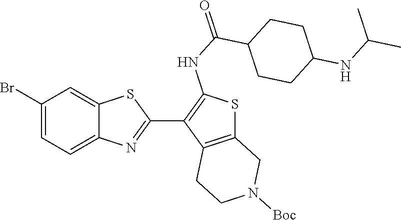 [SMILES: CC(C)NC1CCC(C(=O)Nc2sc3c(c2-c2nc4ccc(Br)cc4s2)CCN(C(=O)OC(C)(C)C)C3)CC1] | 633.1 |
| B11 | 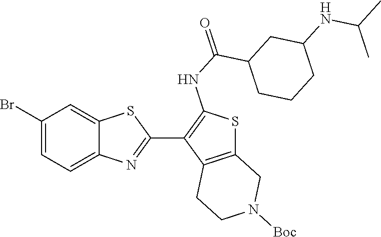 [SMILES: CC(C)NC1CCCC(C(=O)Nc2sc3c(c2-c2nc4ccc(Br)cc4s2)CCN(C(=O)OC(C)(C)C)C3)C1] | 633.1 |
| B18 | 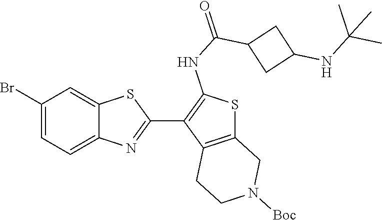 [SMILES: CC(C)(C)NC1CC(C(=O)Nc2sc3c(c2-c2nc4ccc(Br)cc4s2)CCN(C(=O)OC(C)(C)C)C3)C1] | 619.1 |
| B19 | 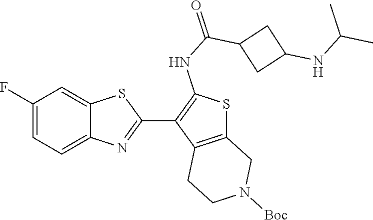 [SMILES: CC(C)NC1CC(C(=O)Nc2sc3c(c2-c2nc4ccc(F)cc4s2)CCN(C(=O)OC(C)(C)C)C3)C1] | 545.2 |
| B20 | 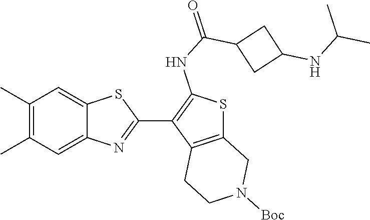 [SMILES: Cc1cc2nc(-c3c(NC(=O)C4CC(NC(C)C)C4)sc4c3CCN(C(=O)OC(C)(C)C)C4)sc2cc1C] | 555.2 |
| B21 | 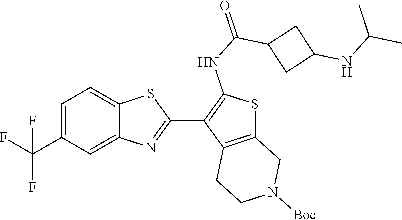 [SMILES: CC(C)NC1CC(C(=O)Nc2sc3c(c2-c2nc4cc(C(F)(F)F)ccc4s2)CCN(C(=O)OC(C)(C)C)C3)C1] | 595.2 |
| B22 | 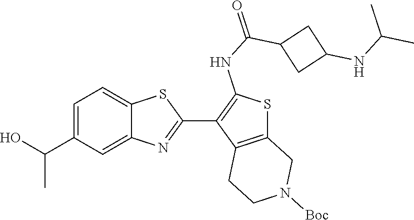 [SMILES: CC(C)NC1CC(C(=O)Nc2sc3c(c2-c2nc4cc(C(C)O)ccc4s2)CCN(C(=O)OC(C)(C)C)C3)C1] | 571.2 |
| B23 | 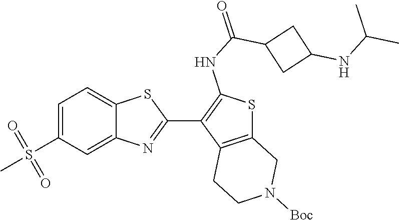 [SMILES: CC(C)NC1CC(C(=O)Nc2sc3c(c2-c2nc4cc(S(C)(=O)=O)ccc4s2)CCN(C(=O)OC(C)(C)C)C3)C1] | 605.2 |

### Example 8

### Intermediate B12: tert-butyl 3-(6-bromobenzo[d]thiazol-2-yl)-2-(3-((tert-butoxycarbonyl)(isopropyl)amino)bicyclo[1.1.1]pentane-1-formylamino]-4,7-dihydrothieno[2,3-c]pyridin-6 (5H)-carboxylate

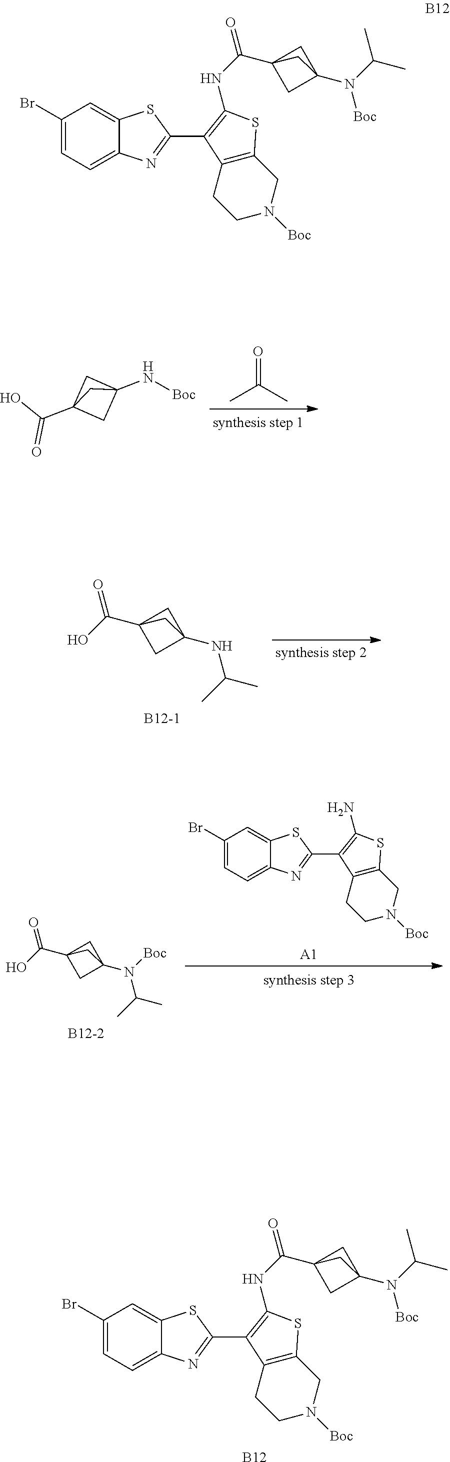

### Synthesis Step 1:3-(isopropylamino) bicyclo[1.1.1]pentane-1-carboxylic acid (intermediate B12-1)

3-((tert-butoxycarbonyl)amino) bicyclo[1.1.1]pentane-1-carboxylic acid (5 g, 22 mmol) was dissolved in methanol, 10 mL of concentrated hydrochloric acid was added, and the reaction was carried out at room temperature for 4 h. After the reaction was completed, methanol was removed by concentration, 20 ml of DCM was added as solvent, then acetone (1.4 g, 24.2 mmol) was added. After the reaction was carried out for 30 min at room temperature, STAB (5.6 g, 26.4 mmol) was added, and the reaction was continued at room temperature for 1 h. After the reaction was completed, the reaction solution was directly concentrated to obtain intermediate B12-1 (3.6 g), which was directly used in the next step without purification, with a yield of 98%. ESI-MS (M+H)⁺170.1.

### Synthesis Step 2:3-((tert-butoxycarbonyl)(isopropyl)amino) bicyclo[1.1.1]pentane-1-carboxylic acid (intermediate B12-2)

The intermediate B12-1 (3.6 g, 21.56 mmol) was dissolved in methanol, di-tert-butyl dicarbonate (7.06 g, 32.34 mmol) and saturated sodium bicarbonate aqueous solution (10 mL) were added, and the reaction was carried out at room temperature for 1 h. After the reaction was completed, methanol was removed by concentration, ethyl acetate and saturated ammonium chloride aqueous solution were added for extraction, and liquid separation was performed. The organic phase was purified by column chromatography to obtain intermediate B12-2 (3.95 g) with a yield of 68%, ESI-MS (M+H)⁺270.2.

### Synthesis Step 3: tert-butyl 3-(6-bromobenzo[d]thiazol-2-yl)-2-(3-((tert-butoxycarbonyl)(isopropyl)amino) bicyclo[1.1.1]pentane-1-formylamino]-4,7-dihydrothi eno [2,3-c]pyridin-6 (5H)-carboxylate (intermediate B12)

The intermediate A1 (330 mg, 0.71 mmol) was dissolved in 10 mL of dichloromethane, the intermediate B12-2 (288 mg, 1.07 mmol) and EDCI (205 mg, 1.07 mmol) were added to the system in sequence, and the reaction was carried out at room temperature for 14 h. After the reaction was completed, it was quenched by adding water, and the mixture was extracted with dichloromethane. The organic phases were combined, dried, then concentrated, and purified by column chromatography to obtain intermediate B12 (233 mg) with a yield of 10%. ESI-MS (M+H)⁺717.2.

### Example 9

### Intermediate B13: tert-butyl 3-(6-bromobenzo[d]thiazol-2-yl)-2-((tert-butoxycarbonyl)(3-(isopropylamino) propyl)amino)-4,7-dihydrothieno[2,3-c]pyridin-6 (5H) carboxylate

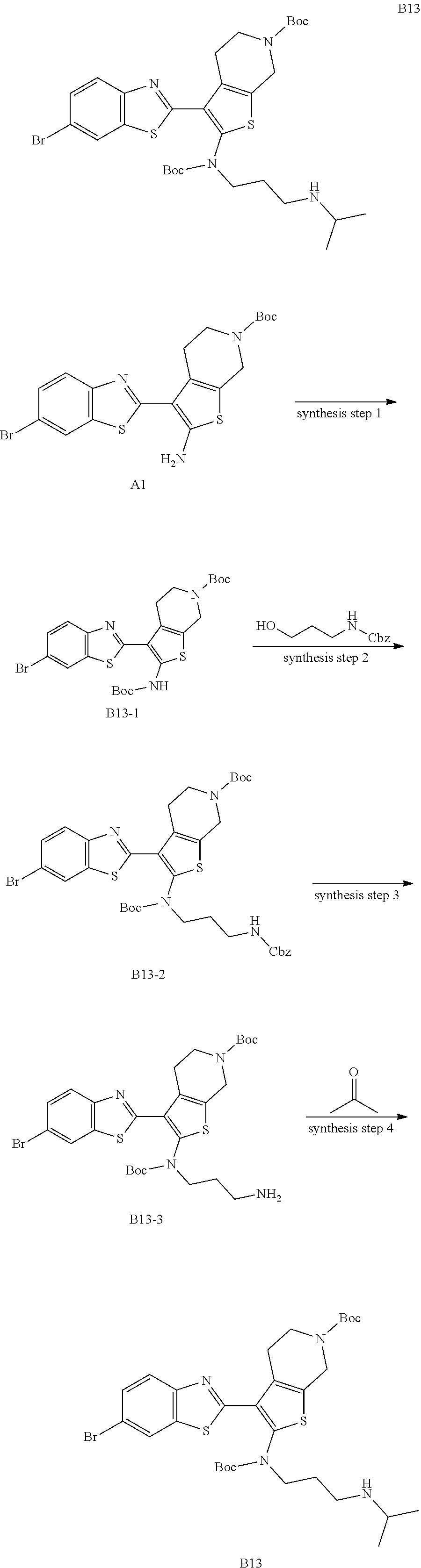

### Synthesis Step 1: tert-butyl 3-(6-bromobenzo[d]thiazol-2-yl)-2-((tert-butoxycarbonyl)amino)-4,7-dihydrothieno[2,3-c]pyridin-6 (5H) carboxylate (intermediate B13-1)

The intermediate A1 (66 g, 0.17 mol) was dissolved in 300 mL of THF, and the mixture was stirred at room temperature until a clear solution was obtained. Then (Boc)₂O (37 g, 0.17 mol) was slowly added and heated to 60° C. under stirring until a clear solution was obtained. 50 mL of a solution of DMAP(2 g, 0.017 mol) in THF was added dropwise, and a large amount of solid precipitated in the system along with the generation of a large number of bubbles. After the dropwise addition of DMAP was completed, the reaction was continued at 60° C. for 24 h. Then the reaction was monitored to be completed by TLC and LCMS, the system was cooled to room temperature, adjusted to pH 6 with 0.5N HCl, and extracted with ethyl acetate. The organic phase was extracted with saturated sodium chloride to remove water, dried over anhydrous sodium sulfate, and the crude product was purified by column chromatography with eluent of petroleum ether and ethyl acetate to obtain intermediate B13-1 (13 g) with a yield of 24%, ESI-MS (M+H)⁺566.1.

### Synthesis Step 2: tert-butyl 2-((3-((benzyloxy) carbonyl)amino) propyl)(tert-butoxycarbonyl)amino)-3-(6-bromobenzo[d]t hiazol-2-yl)-4,7-dihydrothieno[2,3-c]pyridin-6 (5H) carboxylate (intermediate B13-2)

The intermediate B13-1 (1.5 g, 3.075 mmol) was dissolved in 10 mL of THF, and the mixture was stirred at room temperature until a clear solution was obtained. Then, 3-(benzyloxycarbonylamino)-1-propanol (1.3 g, 6.15 mmol) and PPh₃ (1.6 g, 6.15 mmol) were added in sequence, the mixture was stirred until a clear solution was obtained, then DIAD (1.3 g, 6.15 mmol) was slowly added dropwise, and the reaction was continued at room temperature for 1 h. The reaction was monitored to be completed by TLC and LCMS, and quenched by adding water. THF was removed by concentration, and the mixture was extracted with ethyl acetate. The organic phase was extracted with saturated sodium chloride to remove water, dried over anhydrous sodium sulfate, and the crude product was purified by column chromatography with an eluent of petroleum ether and ethyl acetate to obtain intermediate B13-2 (1.5 g) with a yield of 64%. ESI-MS (M+H)⁺757.2.

### Synthesis Step 3: tert-butyl 2-((3-aminopropyl)(tert-butoxycarbonyl)amino)-3-(6-bromobenzo[d]thiazol-2-yl)-4,7-dihydrothieno[2,3-c]pyridin-6 (5H) carboxylate (intermediate B13-3)

Intermediate B13-2 (1.5 g, 1.98 mmol) was dissolved in 15 mL of methanol, and hydrogen (1 atm) was introduced to react at room temperature for 16 h. After the reaction was completed, the reaction solution was filtered, the filter cake was rinsed with methanol, and the mother liquor was concentrated to obtain intermediate B13-3 (1.1 g) with a yield of 89%, which can be directly used in the next reaction without further purification, ESI-MS (M+H)⁺623.1.

### Synthesis Step 4: tert-butyl 3-(6-bromobenzo[d]thiazol-2-yl)-2-((tert-butoxycarbonyl) (3-(isopropylamino) propyl)amino)-4,7-dihydrothieno[2,3-c]pyridin-6 (5H) carboxylate (intermediate B13)

The intermediate B13-3 (1.1 g, 1.76 mmol) was dissolved in 20 mL of anhydrous dichloromethane, acetone (130 μL, 1.76 mmol) was added, and the reaction was carried out at room temperature for 1 h. Then STAB (448 mg, 2.11 mmol) was added, and the reaction was continued at room temperature for 2 h. After the reaction was completed, the reaction solution was quenched by adding water, and extracted with dichloromethane. The organic phases were combined and concentrated, and purified by column chromatography to obtain intermediate B13 (841 mg) with a yield of 72%. ESI-MS (M+H)⁺665.2.

### Example 10

### Intermediate B14: tert-butyl 3-(benzo[d]thiazol-2-yl)-2-((tert-butoxycarbonyl) (3-(isopropylamino) propyl)amino)-4,7-dihydrothieno[2,3-c]pyridin-6 (5H)-carboxylate

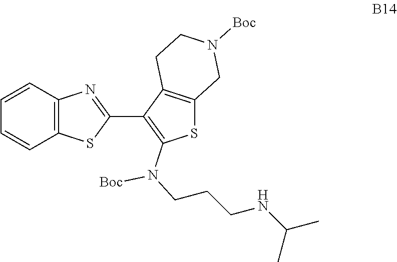 [SMILES: CC(C)NCCCN(C(=O)OC(C)(C)C)c1sc2c(c1-c1nc3ccccc3s1)CCN(C(=O)OC(C)(C)C)C2]

Referring to the synthetic route and method of intermediate B13, in synthesis step 1, intermediate A1 was replaced with intermediate A2 to synthesize the target intermediate B14, ESI-MS (M+H)⁺587.3.

### Example 11

### Intermediate B15: tert-butyl 3-(6-bromobenzo[d]thiazol-2-yl)-2-((tert-butoxycarbonyl)(2-((tert-butoxycarbonyl)(isopropyl)amino)ethyl)amino)-2-oxoethyl)amino)-4,7--dihydrothieno[2,3-c]pyridin-6 (5H)-carboxylate

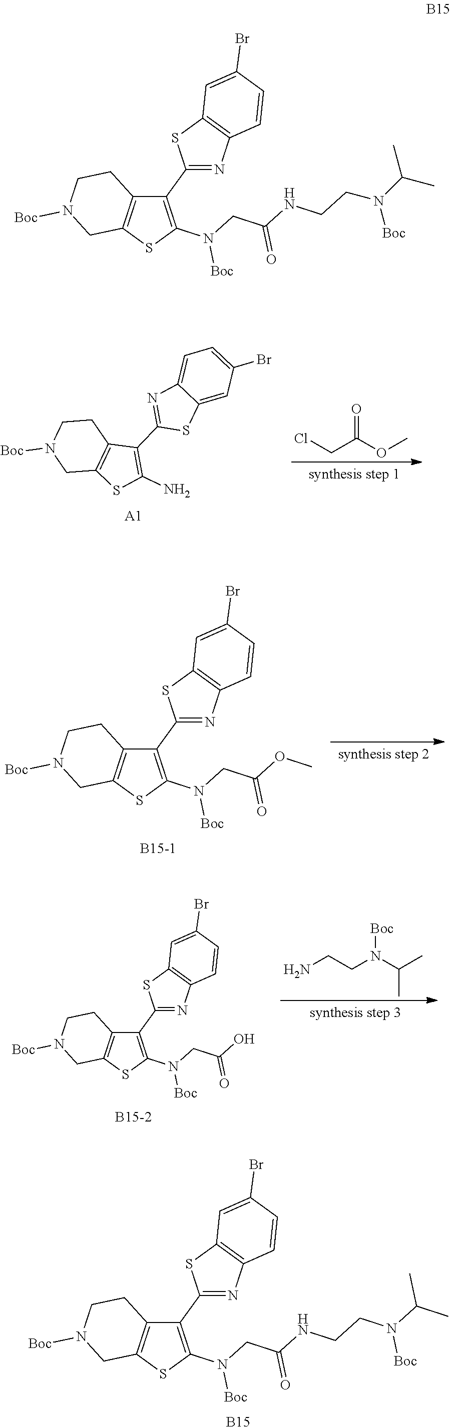

### Synthesis Step 1: tert-butyl 3-(6-bromobenzo[d]thiazol-2-yl)-2-((tert-butoxycarbonyl)(2-methoxy-2-ethoxy)amino)-4,7-dihydrothieno[2,3-c]pyridin-6 (5H) carboxylate (intermediate B15-1)

The intermediate A1 (4.5 g, 13.37 mmol) was dissolved in 5 mL of DMF, the mixture was stirred until a clear solution was obtained, then methyl chloroacetate (167 mg, 1.54 mmol), K₂CO₃ (212 mg, 1.54 mmol), KI (85 mg, 0.51 mmol) and TBAI (40 mg, 0.1025 mmol, 0.1 eq) were added in sequence, and the reaction was carried out at 80° C. for 16 h. After the reaction was completed, it was quenched by adding water, and the mixture was extracted with EA. The organic phases were combined, dried, concentrated, and purified by column chromatography to obtain intermediate B15-1 (460 mg) with a yield of 67%.

ESI-MS (M+H)⁺638.1.

### Synthesis Step 2: N-(3-(6-bromobenzo[d]thiazol-2-yl)-6-(tert-butoxycarbonyl)-4,5,6,7-tetrahydrothieno[2,3-c]pyridin-2-yl)-N-(tert-butoxycarbonyl)glycine (intermediate B15-2)

Intermediate B15-1 (460 mg, 0.823 mmol) was dissolved in 2 mL of MeOH and 2 ml of THE, the mixture was stirred and dissolved until a clear solution was obtained, then NaOH (3N, 2 ml) was added, and the reaction was carried out at room temperature for 16 h. After the reaction was completed, 2N HCl was added to adjust the pH to about 6, and the reaction mixture was concentrated and then added with EA for extraction. The crude product was purified by column chromatography to obtain intermediate B15-2 (400 mg) with a yield of 89%. ESI-MS (M+H)⁺624.1.

### Synthesis Step 3: tert-butyl 3-(6-bromobenzo[d]thiazol-2-yl)-2-((tert-butoxycarbonyl) (2-((2-((tert-butoxycarbonyl)(isopropyl)amino)ethyl)amino)-2-oxoethyl)amino)-4,7-dihydropthieno[2,3-c]pyridin-6 (5H)-carboxylate (intermediate B15)

The intermediate B15-2 (230 mg, 0.368 mmol) was dissolved in 2 mL of DCM, stirred and dissolved until a clear solution was obtained, then tert-butyl(2-aminoethyl)(isopropyl)carbamate (149 mg, 0.736 mmol) and EDCI (212 mg, 1.104 mmol) were added in sequence, and the reaction was carried out at room temperature for 2 h. After the reaction was completed, it was quenched by adding water, and the mixture was extracted with DCM. The organic phases were combined, dried and then concentrated, and the crude product was purified by column chromatography to obtain intermediate B15 (146 mg) with a yield of 52%. ESI-MS (M+H)⁺=808.2.

### Example 12

### Intermediate B16: tert-butyl 3-(benzo[d]thiazol-2-yl)-2-((tert-butoxycarbonyl) (2-((2-((tert-butoxycarbonyl)(isopropyl)amino)ethyl)amino)-2-oxoethyl)amino)-4,7-dihydrothieno[2,3-c]pyridin-6 (5H)-carboxylate

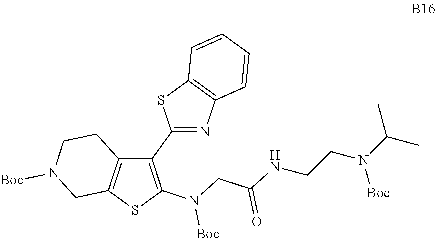 [SMILES: CC(C)N(CCNC(=O)CN(C(=O)OC(C)(C)C)c1sc2c(c1-c1nc3ccccc3s1)CCN(C(=O)OC(C)(C)C)C2)C(=O)OC(C)(C)C]

Referring to the synthetic route and method of intermediate B15, in synthesis step 1, intermediate A1 was replaced with intermediate A12 to synthesize the target intermediate B16, ESI-MS (M+H)⁺730.3.

### Example 13

### Intermediate B17: tert-butyl 3-(6-bromobenzo[d]thiazol-2-yl)-2-((tert-butoxycarbonyl)(3-(isopropylamino)-2-oxopropyl)amino)-4,7-dihydrothieno[2,3-c]pyridin-6 (5H)-carboxylate

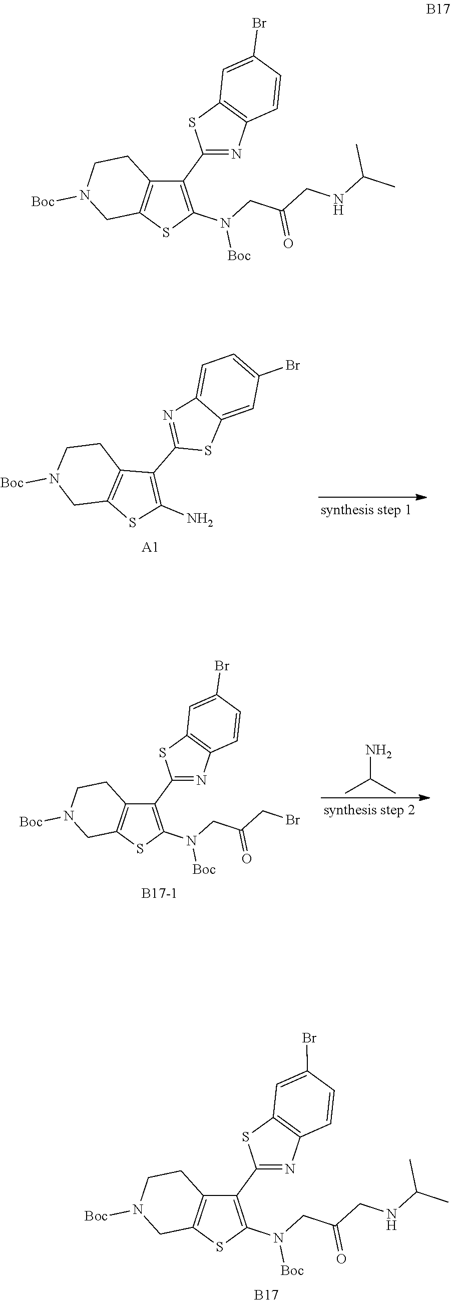

### Synthesis Step 1: tert-butyl 2-((3-bromo-2-oxopropyl)(tert-butoxycarbonyl)amino)-3-(6-bromobenzo[d]thiazol-2-yl)-4,7-dihydrothieno[2,3-c]pyridin-6 (5H) carboxylate (intermediate B17-1)

Referring to the synthesis route and method in synthesis step 1 of intermediate B15, in synthesis step 1, methyl chloroacetate was replaced with 1,3-dibromoacetone to synthesize intermediate B17-1, ESI-MS (M+H)⁺700.0.

### Synthesis Step 2: tert-butyl 3-(6-bromobenzo[d]thiazol-2-yl)-2-((tert-butoxycarbonyl) (3-(isopropylamino)-2-oxopropyl)amino)-4,7-dihydrothieno[2,3-c]pyridin-6 (5H) carboxylate (intermediate B17)

The intermediate B17-1 (1.3 g, 1.86 mmol) was dissolved in 10 mL of DMF, stirred and dissolved until a clear solution was obtained, then isopropyl amine (550 mg, 9.31 mmol) was added in sequence and the reaction was carried out at 50° C. for 6 h. After the reaction was completed, it was quenched by adding water, and then the mixture was extracted with EA. The organic phases were combined, dried and then concentrated, and the crude product was purified by column chromatography to obtain intermediate B17 (543 mg) with a yield of 43%.

ESI-MS (M+H)⁺679.2.

### Example 14

### Intermediate B24: tert-butyl (4-((3-(6-bromobenzo[d]thiazol-2-yl))-4,5,6,7-tetrahydrothieno[2,3-c]pyridin-2-yl) carbamoyl)cyclohexyl)(tert-butyl)carbamate

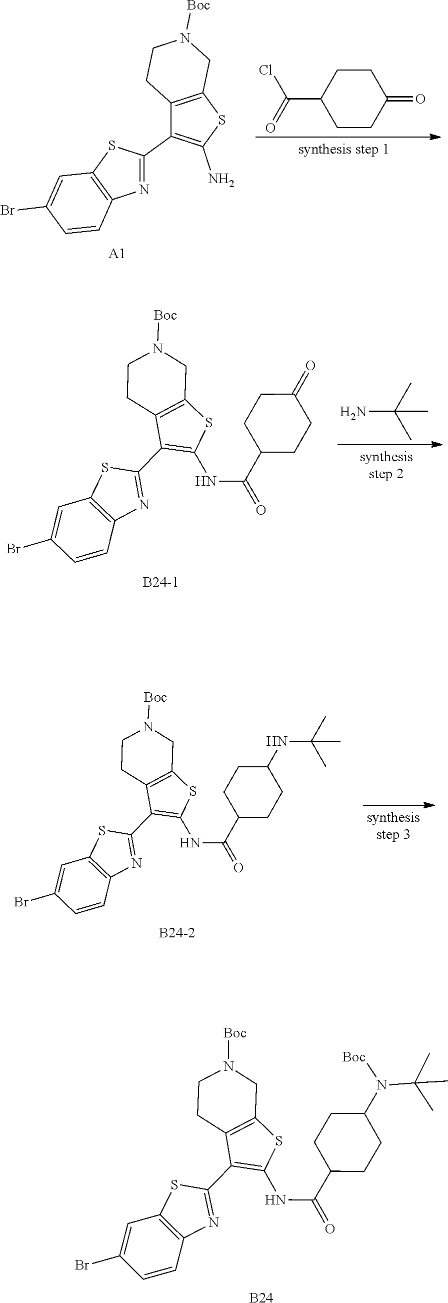

### Synthesis Step 1: N-(3-(6-bromobenzo[d]thiazol-2-yl)-4,5,6,7-tetrahydrothieno[2,3-c]pyridin-2-yl)-4-oxocyclohexane-1-formamide (intermediate B24-1)

3-(6-bromobenzo[d]thiazol-2-yl)-4,5,6,7-tetrahydrothieno[2,3-c]pyridin-2-amine (5.0 g, 13.70 mmol) was dissolved in 15 mL of DCM, 4-oxocyclohexane-1-carbonyl chloride (5.4 g, 34.25 mmol, 2.5 eq) and TEA (2.8 g, 27.40 mmol, 2.0 eq) were added into the system in sequence, and the reaction was carried out at room temperature for 1 h. After the reaction was completed, it was quenched by adding an aqueous solution of saturated ammonium chloride, and the mixture was extracted three times with DCM. The organic phases were combined, dried over anhydrous sodium sulfate, concentrated under reduced pressure, and purified by column chromatography (eluted with DCM/MeOH) to obtain intermediate B24-1, 5.3 g, with a yield of 79%. ESI-MS (M+H)⁺590.1.

### Synthesis Step 2: N-(3-(6-bromobenzo[d]thiazol-2-yl)-4,5,6,7-tetrahydrothieno[2,3-c]pyridin-2-yl)-4-(tert-butylamino)cyclohexane-1-carboxamide (intermediate B24-2)

N-(3-(6-bromobenzo[d]thiazol-2-yl)-4,5,6,7-tetrahydrothieno[2,3-c]pyridin-2-yl)-4-oxocyclohexane-1-carboxamide (5.3 g, 10.84 mmol) was dissolved in 50 mL of DCE, isopropylamine (4.5 mL, 54.20 mmol, 5.0 eq) and acetic acid (3.5 mL, 54.20 mmol, 5.0 eq) were added into the system in sequence, and the reaction was carried out at 80° C. for 2 h. Then STAB (4.6 g, 21.68 mmol, 2.0 eq) was added, and the reaction was continued at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain intermediate B24-2, 3.3 g, with a yield of 56%. ESI-MS (M+H)⁺647.2.

### Synthesis Step 3: tert-butyl (4-((3-(6-bromobenzo[d]thiazol-2-yl))-4,5,6,7-tetrahydrothieno[2,3-c]pyridin-2-yl) carbamoyl)cyclohexyl)(tert-butyl)carbamate (intermediate B24)

N-(3-(6-bromobenzo[d]thiazol-2-yl)-4,5,6,7-tetrahydrothieno[2,3-c]pyridin-2-yl)-4-(tert-butylamino)cyclohexane-1-carboxamide (3.3 g, 6.04 mmol) was dissolved in 30 mL of DCM, di-tert-butyl dicarbonate (2769 mL, 12.08 mmol) and triethylamine (2057 mL, 15.10 mmol) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain intermediate B24, 3.6 g, with a yield of 93%. ESI-MS (M+H)⁺747.2.

Referring to the synthetic route and method of intermediate B24, the following intermediates were synthesized:

| | | LC-MS(ESI-MS) |
|---|---|---|
| Intermediate | Structure of intermediate | [M + H]⁺ |
| B25 | 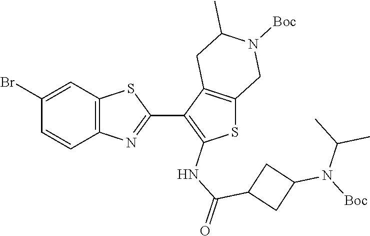 [SMILES: CC1Cc2c(sc(NC(=O)C3CC(N(C(=O)OC(C)(C)C)C(C)C)C3)c2-c2nc3ccc(Br)cc3s2)CN1C(=O)OC(C)(C)C] | 719.2 |
| B26 | 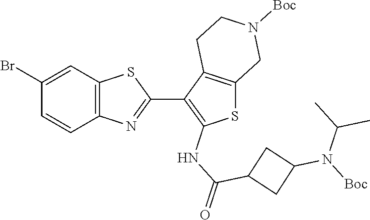 [SMILES: CC(C)N(C(=O)OC(C)(C)C)C1CC(C(=O)Nc2sc3c(c2-c2nc4ccc(Br)cc4s2)CCN(C(=O)OC(C)(C)C)C3)C1] | 705.2 |
| B27 | 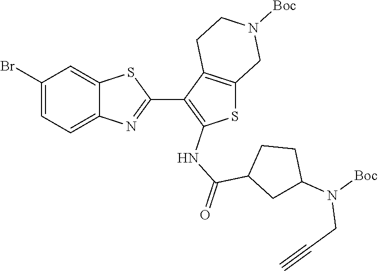 [SMILES: C#CCN(C(=O)OC(C)(C)C)C1CCC(C(=O)Nc2sc3c(c2-c2nc4ccc(Br)cc4s2)CCN(C(=O)OC(C)(C)C)C3)C1] | 715.2 |
| B28 | 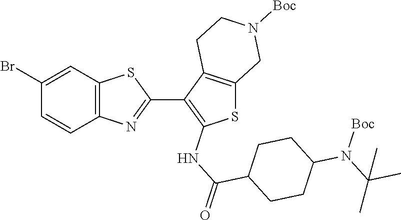 [SMILES: CC(C)(C)OC(=O)N1CCc2c(sc(NC(=O)C3CCC(N(C(=O)OC(C)(C)C)C(C)(C)C)CC3)c2-c2nc3ccc(Br)cc3s2)C1] | 747.2 |

### Example 15

### Synthesis Route of Intermediate B29

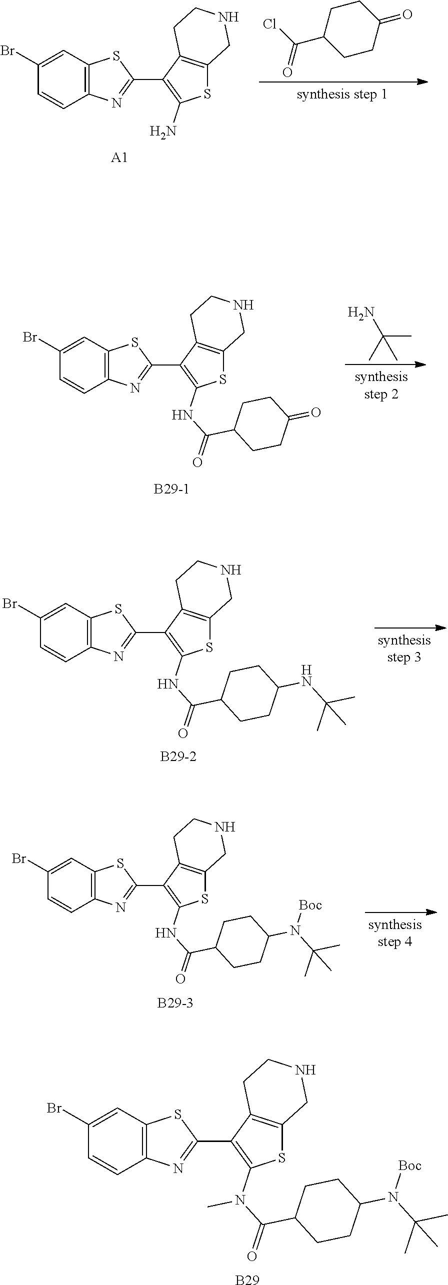

### Synthesis Step 1: N-(3-(6-bromobenzo[d]thiazol-2-yl)-4,5,6,7-tetrahydrothieno[2,3-c]pyridin-2-yl)-4-oxocyclohexane-1-formamide (intermediate B29-1)

3-(6-bromobenzo[d]thiazol-2-yl)-4,5,6,7-tetrahydrothieno[2,3-c]pyridin-2-amine (5.0 g, 13.70 mmol) was dissolved in 15 mL of DCM, 4-oxocyclohexane-1-carbonyl chloride (5.4 g, 34.25 mmol, 2.5 eq) and TEA (2.8 g, 27.40 mmol, 2.0 eq) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain B29-1 (5.3 g) with a yield of 79%. ESI-MS (M+H)⁺490.0.

### Synthesis Step 2: N-(3-(6-bromobenzo[d]thiazol-2-yl)-4,5,6,7-tetrahydrothieno[2,3-c]pyridin-2-yl)-4-(tert-butylamino)cyclohexane-1-carboxamide (intermediate B29-2)

B29-1 (5.3 g, 10.84 mmol) was dissolved in 50 mL of DCE, isopropylamine (4.5 mL, 54.20 mmol, 5.0 eq) and acetic acid (3.5 mL, 54.20 mmol, 5.0 eq) were added into the system in sequence, and the reaction was carried out at 80° C. for 2 h. Then STAB (4.6 g, 21.68 mmol, 2.0 eq) was added, and the reaction was continued at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain B29-2 (3.3 g) with a yield of 56%. ESI-MS (M+H)⁺547.1.

### Synthesis Step 3: tert-butyl (4-((3-(6-bromobenzo[d]thiazol-2-yl))-4,5,6,7-tetrahydrothieno[2,3-c]pyridin-2-yl) carbamoyl)cyclohexyl)(tert-butyl)carbamate (intermediate B29-3)

B29-2 (3.3 g, 6.04 mmol) was dissolved in 30 mL of DCM, di-tert-butyl dicarbonate (2769 mL, 12.08 mmol) and triethylamine (2057 mL, 15.10 mmol) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain B29-3 (3.6 g) with a yield of 93%. ESI-MS (M+H)⁺647.2.

### Synthesis Step 4: tert-butyl (4-((3-(6-bromobenzo[d]thiazol-2-yl))-4,5,6,7-tetrahydrothieno[2,3-c]pyridin-2-yl)(methyl)carbamoyl)cyclohexyl)(tert-butyl)carbamate (intermediate B29)

B29-3 (3.6 g, 4.82 mmol) was dissolved in 20 mL of DMF, sodium hydride (193 mg, 4.82 mmol) and methyl iodide (616 mg, 4.34 mmol) were added into the system at 0° C. in sequence, and the reaction was carried out in ice bath for 4 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain B29 (2.2 g) with a yield of 61%. ESI-MS (M+H)⁺661.2.

Referring to the synthetic route and method of intermediate B29, the following intermediate was synthesized.

| Inter- | | LC-MS(ESI-MS) |
|---|---|---|
| mediate | Structure of intermediate | [M + H]⁺ |
| B30 | 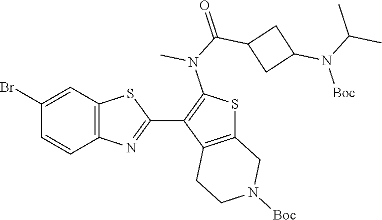 [SMILES: CC(C)N(C(=O)OC(C)(C)C)C1CC(C(=O)N(C)c2sc3c(c2-c2nc4ccc(Br)cc4s2)CCN(C(=O)OC(C)(C)C)C3)C1] | 719.2 |

### Example 16

### Synthesis Route of Intermediate B31

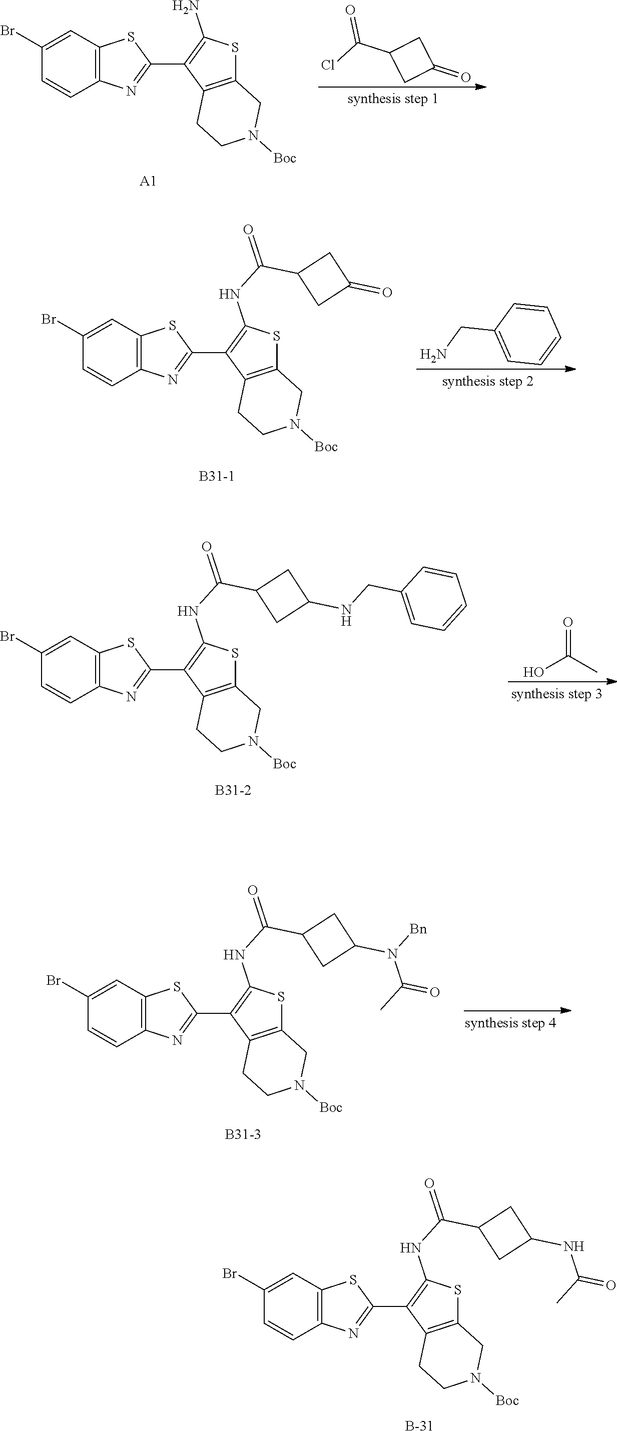

Synthesis step 1: tert-butyl 3-(6-bromobenzo[d]thiazol-2-yl)-2-(3-oxocyclobutane-1-formamidyl)-4,7-dihydrothieno[2,3-c]pyridin-6 (5H)-carboxylate (intermediate B31-1)

Tert-butyl 2-amino-3-(6-bromobenzo[d]thiazol-2-yl)-4,7-dihydrothieno[2,3-c]pyridin-6 (5H)-carboxylate (2.0 g, 4.29 mmol) was dissolved in 20 mL of DCM, 3-oxocyclobutane-1-carbonyl chloride (1.4 g, 10.73 mmol) and TEA (866 mg, 8.58 mmol) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure, and subjected to column chromatography (eluted with DCM/MeOH) to obtain B31-1 (1.9 g) with a yield of 79%. ESI-MS (M+H)⁺562.0.

### Synthesis Step 2: tert-butyl 2-(3-(benzylamino)cyclobutane-1-formamido)-3-(6-bromobenzo[d]thiazol-2-yl)-4,7-dihydrothieno[2,3-c]pyridin-6 (5H)-carboxylate (intermediate B31-2)

B31-1 (1.9 g, 3.39 mmol) was dissolved in 10 mL of DCE, benzylamine (1.8 g, 16.93 mmol) and acetic acid (1.1 mL, 16.93 mmol) were added into the system in sequence, and the reaction was carried out at 80° C. for 2 h, then STAB (1.4 g, 6.78 mmol) was added, and the reaction was carried out at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain B31-2 (1.2 g) with a yield of 56%. ESI-MS (M+H)⁺653.1.

### Synthesis Step 3: tert-butyl (3-(N-benzylacetamido)cyclobutane-1-formamido)-3-(6-bromobenzo[d]thiazol-2-yl)-4,7-dihydrothieno[2,3-c]pyridin-6 (5H)-carboxylate (intermediate B31-3)

B31-2 (1.2 g, 1.84 mmol) was dissolved in 10 mL of DCM, acetic acid (166 mg, 2.76 mmol), HATU (1.0 g, 2.76 mmol) and triethylamine (559 mg, 5.52 mmol) were added into the system in sequence, and the reaction was carried out at 80° C. for 2 h, then STAB (1.4 g, 6.78 mmol) was added, and the reaction was continued at room temperature for 1 h. After the reaction was completed, it was quenched by adding saturated sodium bicarbonate aqueous solution, the mixture was extracted three times with DCM, and the organic phases were combined, dried over anhydrous sodium sulfate, concentrated under reduced pressure, and subjected to column chromatography (eluted with DCM/MeOH) to obtain B31-3 (996 mg) with a yield of 78%. ESI-MS (M+H)⁺695.1.

### Synthesis Step 4: tert-butyl 2-(3-acetylaminocyclobutane-1-carboxamido)-3-(6-bromobenzo[d]thiazol-2-yl)-4,7-dihydrothieno[2,3-c]pyridin-6 (5H)-carboxylate (intermediate B31)

B31-3 (996 mg, 1.43 mmol) was dissolved in 10 mL of THF, Pd/C (100 mg) and hydrogen gas were added into the system in sequence, and the reaction was carried out at 50° C. under three atmospheric pressures for 4 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain B31 (587 mg) with a yield of 68%. ESI-MS (M+H)⁺605.1.

### Example 17

Intermediate B32: tert-butyl 3-(6-bromobenzo[d]thiazol-2-yl)-2-(4-(tert-butylamino)-3-methoxybenzoylamino)-4,7-dihydrothieno[2,3-c]pyridin-6 (5H)-carboxylate

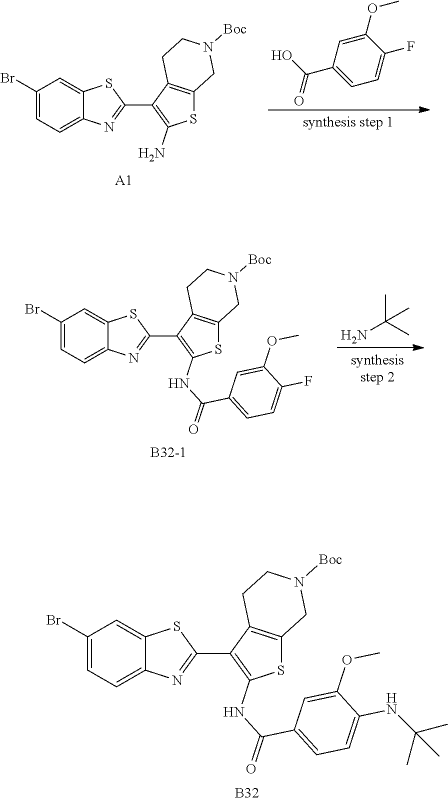

### Synthesis step 1: tert-butyl 3-(6-bromobenzo[d]thiazol-2-yl)-2-(4-fluoro-3-methoxybenzoylamino)-4,7-dihydrothieno[2,3-c]pyridin-6 (5H)-carboxylate (intermediate B32-1)

The compound tert-butyl 2-amino-3-(6-bromobenzo[d]thiazol-2-yl)-4,7-dihydrothieno[2,3-c]pyridin-6 (5H)-carboxylate (4 g, 8.60 mmol) was dissolved in 50 mL of N,N-dimethylformamide, 2-(7-azabenzotriazol)-N,N,N′,N′-tetramethylurea hexafluorophosphate (4.9 g, 12.90 mmol) and triethylamine (2.6 g, 25.80 mmol) were added into the system, and the reaction was carried out at room temperature for 2 h. After the reaction was completed, it was quenched by adding water, and the mixture was extracted with dichloromethane. The organic phases were combined, dried over anhydrous sodium sulfate, concentrated under reduced pressure, and subjected to column chromatography to obtain B32-1 (3) g with a yield of 56% and ESI-MS (M+H)⁺618.1.

### Synthesis step 2: tert-butyl 3-(6-bromobenzo[d]thiazol-2-yl)-2-(4-(tert-butylamino)-3-methoxybenzoylamino)-4,7-dihydrothieno[2,3-c]pyridin-6 (5H)-carboxylate (intermediate B32)

Compound B32-1 (2 g, 3.23 mmol) was dissolved in 20 ml of N,N-dimethylformamide, cesium carbonate (3.1 g, 9.70 mmol) and tert-butylamine (306 mg, 4.19 mmol) were added into the system, and the reaction was carried out at 80° C. for 12 h. After the reaction was completed, it was quenched by adding water, and the mixture was extracted with dichloromethane. The organic phases were combined, dried over anhydrous sodium sulfate, concentrated under reduced pressure, and subjected to column chromatography to obtain B32 (1.5) g with a yield of 69%. ESI-MS (M+H)⁺671.1.

Referring to the synthetic route of intermediate B32, the following intermediates were synthesized:

| Number of | | LC-MS ESI-MS |
|---|---|---|
| intermediate | Structure of intermediate | (M + H)⁺ |
| B33 | 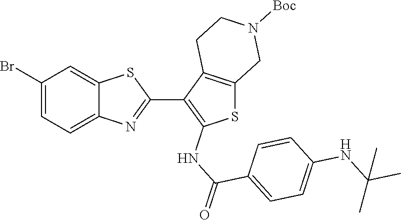 [SMILES: CC(C)(C)Nc1ccc(C(=O)Nc2sc3c(c2-c2nc4ccc(Br)cc4s2)CCN(C(=O)OC(C)(C)C)C3)cc1] | 641.1 |
| B34 | 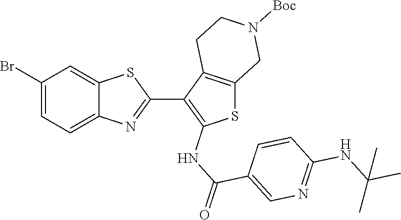 [SMILES: CC(C)(C)Nc1ccc(C(=O)Nc2sc3c(c2-c2nc4ccc(Br)cc4s2)CCN(C(=O)OC(C)(C)C)C3)cn1] | 642.1 |

### Example 18

### Intermediate B35: tert-butyl 3-(6-bromobenzo[d]thiazol-2-yl)-2-((4-(tert-butylamino)cyclohexyl)methyl)amino)-4,7-dihydrothieno[2,3-c]pyridin-6 (5H)-carboxylate

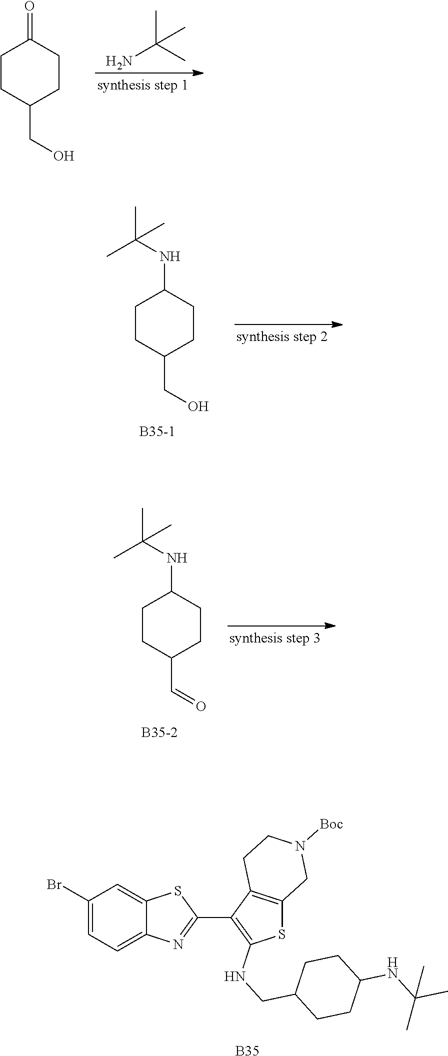

### Synthesis Step 1:(4-(tert-butylamino)cyclohexyl)methanol (intermediate B35-1)

4-(Hydroxymethyl)cyclohexane-1-one (5 g, 39.04 mmol, 1.0 eq) was dissolved in 50 mL of dichloromethane, 2-methylpropane-2-amine (2.8 g, 39.04 mmol, 1.0 eq) and sodium triacetoxyborohydride (8.3 g, 78.07 mmol, 2.0 eq) were added in sequence, and the reaction was carried out at room temperature for 4 h. After the reaction was completed, water was added for extraction, and the organic phases were subjected to column chromatography to obtain B35-1 (5.2) g, ESI-MS (M+H)⁺186.2.

### Synthesis Step 2:4-(tert-butylamino)cyclohexane-1-carboxylic acid (intermediate B35-2)

B35-1 (5 g, 27.00 mmol, 1.0 eq) was dissolved in 50 mL of dichloromethane, desmartin (23 g, 54.00 mmol, 2.0 eq) was added in ice bath for 10 times at intervals of 2 min each time. After the addition was completed, the reaction was transferred to room temperature and carried out for 12 h. After the reaction was completed, water was added for extraction, and the organic phases were subjected to column chromatography to obtain B35-2 (2.1) g, ESI-MS (M+H)⁺184.2.

### Synthesis step 3: tert-butyl 3-(6-bromobenzo[d]thiazol-2-yl)-2-((4-(tert-butylamino)cyclohexyl)methyl)amino)-4,7-dihydrothieno[2,3-c]pyridin-6 (5H)-carboxylate (intermediate B35)

B35-2 (2 g, 10.92 mmol, 1.0 eq) was dissolved in 20 mL of dichloromethane, intermediate A-1 (5 g, 10.92 mmol, 1.0 eq) and sodium triacetoxyborohydride (4.6 g, 21.84 mmol, 2.0 eq) were added in sequence, and the reaction was carried out at room temperature for 4 h. After the reaction was completed, water was added for extraction, and the organic phases were subjected to column chromatography to obtain intermediate B35, 7 g, ESI-MS (M+H)⁺633.2.

### Example 19

### Intermediate C1: tert-butyl 2-amino-3-(4,4,5,5-tetramethyl-1,3,2-dioxaborolane-2-yl)-6,7-dihydrothieno[3,2-c]pyridin-5 (4H)-carboxylate

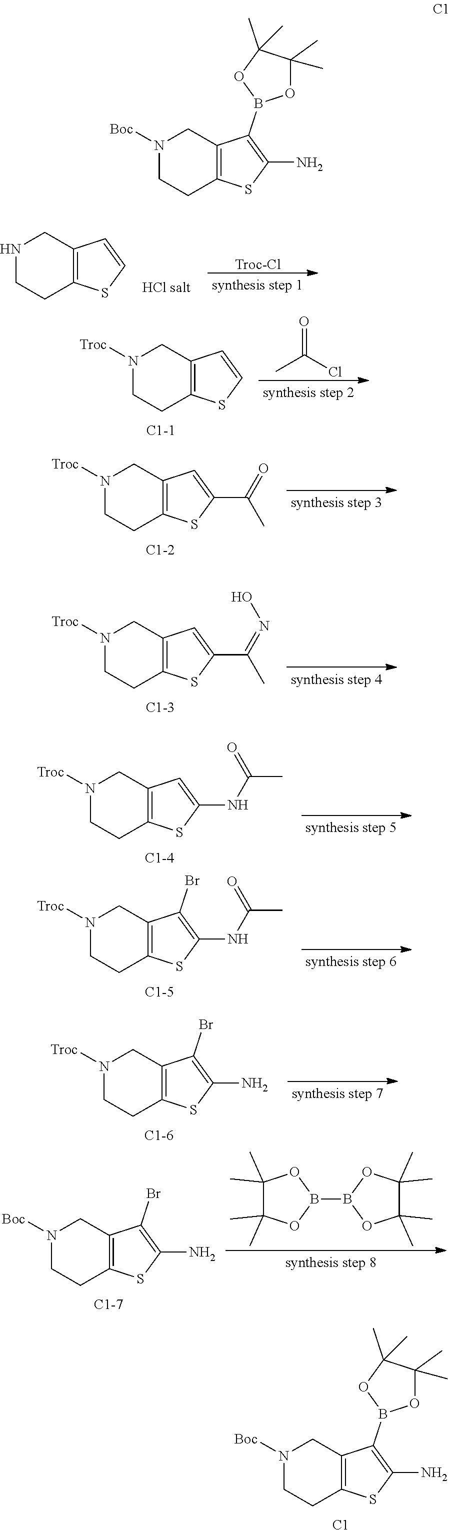

### Synthesis Step 1. 2,2,2-trichloroethyl 6,7-dihydrothieno[3,2-c]pyridin-5 (4H)-carboxylate (intermediate C1-1)

4,5,6,7-tetrahydrothieno[3,2-c]pyridine hydrochloride (100 g, 718.3 mmol) was added into 500 mL of dichloromethane. The system was cooled to 0° C., triethylamine (182 g, 1796 mmol) was added, and then 2,2,2-trichloroethyl chloroformate (152 g, 718.3 mmol) was added slowly. The reaction was carried out at 0° C. for 4 h. After the reaction was completed, it was quenched by adding water, and the mixture was extracted with DCM. The organic phases were combined, then washed with 0.1N hydrochloric acid, dried and concentrated, and subjected to column chromatography to obtain intermediate C1-1 (214 g) with a yield of 95%. ESI-MS (M+H)⁺314.0.

### Synthesis Step 2:2,2,2-trichloroethyl 2-acetyl-6,7-dihydrothieno[3,2-c]pyridin-5 (4H)-carboxylate (intermediate C1-2)

The intermediate C1-1 (214 g, 680 mmol) was dissolved in 2000 mL of dichloromethane, and cooled to 0° C. Titanium tetrachloride (193 g, 1020 mmol) was added, and then acetyl chloride (64 g, 816 mmol) was added slowly. After the dropwise addition was completed, the system was transferred to room temperature and reacted for 13 h. After the reaction was completed, it was quenched by adding water, and the mixture was extracted with DCM. The organic phases were combined, dried and concentrated, and then subjected to column chromatography to obtain intermediate C1-2 (174 g) with a yield of 72%. ESI-MS (M+H)⁺356.0.

### Synthesis Step 3:2,2,2-trichloroethyl (Z)-2-(1-(hydroxyimino)ethyl)-6,7-dihydrothieno[3,2-c]pyridin-5 (4H)-carboxylate (intermediate C1-3)

The intermediate C1-2 (174 g, 489 mmol) was dissolved in 1000 mL of absolute ethanol, hydroxylamine hydrochloride (51 g, 734 mmol) was added, triethylamine (99 g, 979 mmol) and 200 mL of water were finally added, and the reaction was carried out under reflux for 4 h. After the reaction was completed, the mixture was cooled to room temperature, and a product precipitated, which was filtered to obtain intermediate C1-3 (156 g) with a yield of 86%. ESI-MS (M+H)⁺371.0.

### Synthesis Step 4:2,2,2-trichloroethyl 2-acetamido-6,7-dihydrothieno[3,2-c]pyridin-5 (4H)-carboxylate (intermediate C1-4)

Intermediate C₁-3 (58 g, 0.15 mol) was dissolved in 1000 mL of tetrahydrofuran solution, stirred at 0° C. for 10 min, then phosphorus pentachloride (81 g, 0.39 mol) was added, and the reaction was stirred at 0° C. for 2 h, and monitored by TLC. After the reaction was completed, the reaction solution was poured into a large amount of ice water to quench the reaction. The mixture was then extracted with EA, and washed with saturated sodium chloride aqueous solution. The organic phase was dried over anhydrous sodium sulfate, filtered, and concentrated under reduced pressure. The obtained crude product was purified by column chromatography to obtain intermediate C1-4 (21 g) with a yield of 37%, ESI-MS (M+H)⁺371.0.

### Synthesis Step 5:2,2,2-trichloroethyl 2-acetamido-3-bromo-6,7-dihydrothieno[3,2-c]pyridin-5 (4H)-carboxylate (intermediate C1-5)

The intermediate C1-4 (21 g, 56.5 mmol) was dissolved in 210 mL of tetrahydrofuran solution. After a clear solution was obtained, it was stirred at 0° C. for 5 min. NBS dissolved in THF (11 g NBS/50 mL THF) was added dropwise to the system. After the dropwise addition was completed, the system was transferred to room temperature and the stirring was continued for 30 min. The reaction was monitored by TLC. After the reaction was completed, it was quenched by adding water, concentrated under reduced pressure, and then extracted with EA. The organic phases were combined, dried, concentrated, and purified by column chromatography to obtain intermediate C1-5 (22 g) with a yield of 87%, ESI-MS (M+H)⁺448.9.

### Synthesis Step 6:2,2,2-trichloroethyl 2-amino-3-bromo-6,7-dihydrothieno[3,2-c]pyridin-5 (4H)-carboxylate (intermediate C1-6)

The intermediate C1-5 (22 g, 48.8 mmol) was dissolved in 100 mL of methanol solution, 60 mL of concentrated hydrochloric acid was added under stirring, and the reaction was stirred at 60° C. for 2 h, and monitored by LC-MS. After the reaction was completed, the reaction solution was cooled to room temperature and slowly poured into saturated sodium bicarbonate aqueous solution, and the mixture was extracted with EA. The organic phases were combined, dried and then concentrated, and the crude product was purified by column chromatography to obtain intermediate C1-6 (15.5 g) with a yield of 78%, which was directly used in the next step without purification. ESI-MS (M+H)⁺406.9.

### Synthesis Step 7: tert-butyl 2-amino-3-bromo-6,7-dihydrothieno[3,2-c]pyridin-5 (4H)-carboxylate (intermediate C1-7)

The intermediate C1-6 (15.5 g, 37.9 mmol) was dissolved in 100 mL of dichloromethane, 50 mL of ice acetic acid was added, zinc powder (12.4 g, 189.5 mmol) was added slowly, and the reaction was carried out at room temperature for 6 h. After the reaction was completed, the reaction solution was filtered, and the filter cake was washed with methanol. After the mother liquor was concentrated, the concentrate was dissolved in methanol, triethylamine (11.5 g, 113.7 mmol) was added. After the system was dissolved completely and became clear, di-tert-butyl dicarbonate (12.4 g, 56.8 mmol) was added slowly, and the reaction was carried out at room temperature for 1 h. After the reaction was completed, the methanol reaction solution was concentrated, appropriate amount of water was added, and the mixture was extracted with DCM. The organic phases were combined, dried and concentrated, and the crude product was purified by column chromatography to obtain intermediate C1-7 (7.8 g), with a yield of 62%, ESI-MS (M+H)⁺333.0.

### Synthesis Step 8: tert-butyl 2-amino-3-(4,4,5,5-tetramethyl-1,3,2-dioxaborolane-2-yl)-6,7-dihydrothieno[3,2-c]pyridin-5 (4H)-carboxylate (intermediate C1)

The intermediate C1-7 (3 g, 9.0 mmol) was dissolved in 30 mL of dioxane, pinacol diboronate (3.4 g, 13.5 mmol), potassium acetate (1.32 g, 13.5 mmol) and Pd(dppf)Cl₂ (653 mg, 0.9 mmol) were added in sequence, and the reaction was carried out at 100° C. for 4 h. After the reaction was completed, the mixture was cooled to room temperature, the reaction solution was concentrated, appropriate amount of water was added, and the mixture was extracted with EA. The organic phases were combined, dried and concentrated, and the crude product was purified by column chromatography to obtain intermediate C1 (958 mg) with a yield of 28%, ESI-MS (M+H)⁺381.2.

### Example 20

### Intermediate C2: tert-butyl 5-(4,4,5,5-tetramethyl-1,3,2-dioxaborolane-2-yl)-3′,6′-dihydro-[2,4′-bipyridine]-1′ (2′H)-carboxylate

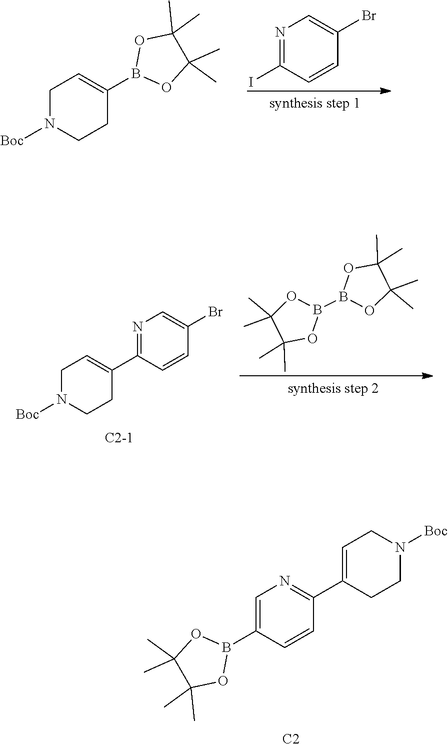

### Synthesis Step 1: tert-butyl 5-bromo-3′, 6′-dihydro-[2,4′-bipyridine]-1′ (2′H)-carboxylate (intermediate C2-1)

Tert-butyl 4-(4,4,5,5-tetramethyl-1,3,2-dioxaborolane-2-yl)-3,6-dihydropyridin-1 (2H)-carboxylate (2.0 g, 6.47 mmol) was added into a mixed solution (dioxane: water=3:1, 20 mL), 5-bromo-2-iodopyridine (2.1 g, 7.54 mmol), Pd(dppf)Cl₂ (450 mg, 0.64 mmol) and Na₂CO₃ (2.0 g, 19.41 mmol) were added into the system in sequence, and the reaction was carried out at 80° C. for 5 h. After the reaction was completed, it was quenched by adding water, and the mixture was extracted three times with DCM. The organic phases were combined, dried over anhydrous sodium sulfate, concentrated under reduced pressure, and subjected to column chromatography to obtain intermediate C2-1 (1.5 g) with a yield of 68%.

ESI-MS (M+H)⁺339.1.

### Synthesis step 2: tert-butyl 5-(4,4,5,5-tetramethyl-1,3,2-dioxaborolane-2-yl)-3′,6′-dihydro-[2,4′-bipyridine]-1′ (2′H)-carb oxylate (intermediate C2)

The intermediate C2-1 (1.5 g, 6.63 mmol) was added into dioxane, 4,4,4′,4′,5,5,5′,5′-octamethyl-2,2′-bis (1,3,2-dioxaborolane)(1.6 g, 6.63 mmol), Pd (dppf) C₁₂ (350 mg, 0.44 mmol) and potassium acetate (1.3 g, 13.26 mmol) were added into the system in sequence, and the reaction was carried out at 80° C. for 5 h. After the reaction was completed, it was quenched by adding water, and the mixture was extracted three times with DCM. The organic phases were combined, dried over anhydrous sodium sulfate, concentrated under reduced pressure, and subjected to column chromatography to obtain intermediate C2 (1.2 g) with a yield of 70%. ESI-MS (M+H)⁺387.2.

### Example 21

### Intermediate C3:1-(4-(4,4,5,5-tetramethyl-1,3,2-dioxaborolane-2-yl)-3,6-dihydropyridin-1 (2H)-yl)ethyl-1-one

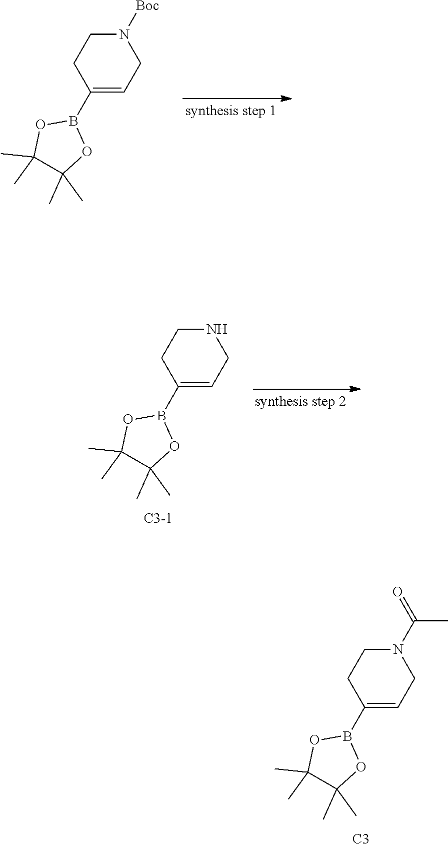

### Synthesis Step 1:4-(4,4,5,5-tetramethyl-1,3,2-dioxaborolane-2-yl)-1,2,3,6-tetrahydropyridine (intermediate C3-1)

Tert-butyl 4-(4,4,5,5-tetramethyl-1,3,2-dioxaborolane-2-yl)-3,6-dihydropyridin-1 (2H)-carboxylate (2.3 g, 7.44 mmol) was added into dichloromethane (20 ml), trifluoroacetic acid (3.0 ml, 37.20 mmol) was added into the system, and the reaction was carried out at room temperature for 2 h. After the reaction was completed, the mixture was spin-dried, and dichloromethane was added for successive spin-drying three times to obtain intermediate C3-1 (1.5 g) with a yield of 96%. ESI-MS (M+H)⁺210.2.

### Synthesis Step 2:1-(4-(4,4,5,5-tetramethyl-1,3,2-dioxaborolane-2-yl)-3,6-dihydropyridin-1 (2H)-yl)ethyl-1-one (intermediate C₃)

Intermediate C3-1 (1.5 g, 7.17 mmol) was added, N,N-diisopropylethylamine (4.0 ml, 21.51 mmol) was added into the system, and acetyl chloride (0.8 ml, 10.75 mmol) was added dropwise under ice bath. After the reaction was completed, it was quenched by adding water, and the mixture was extracted three times with DCM. The organic phases were combined, dried over anhydrous sodium sulfate, concentrated under reduced pressure, and subjected to column chromatography to obtain intermediate C3 (1.3 g) with a yield of 69%. ESI-MS (M+H)⁺2522.

### Example 22

### Intermediate D1: tert-butyl 4-(5-(2-bromobenzo[d]thiazol-6-yl)pyridin-2-yl) piperazin-1-carboxylate

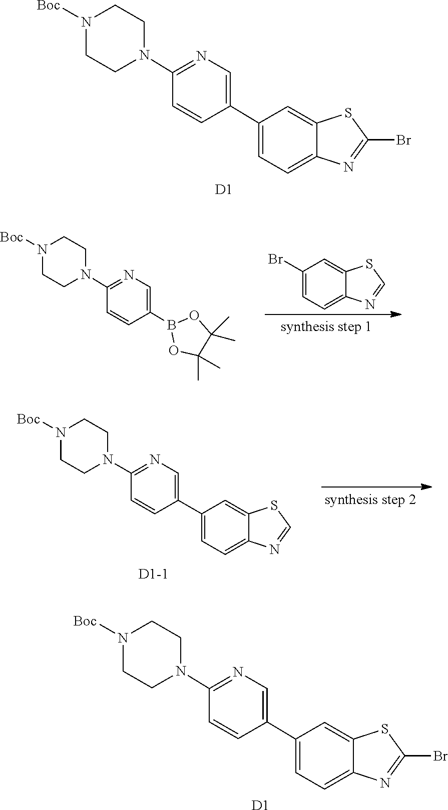

### Synthesis Step 1: tert-butyl 4-(5-(benzo[d]thiazol-6-yl)pyridin-2-yl) piperazin-1-carboxylate (intermediate D1-1)

6-Bromo-1,3-benzothiazole (20 g, 93.42 mmol) was dissolved in 200 mL of dioxane, tert-butyl 4-(5-(4,4,5,5-tetramethyl-1,3,2-dioxaborolane-2-yl)pyridin-2-yl) piperazin-1-carboxylate (40 g, 102.76 mmol), sodium carbonate (9.7 g, 91.46 mmol), Pd(dppf)Cl₂ (3.4 g, 4.67 mmol) and water (40 mL) were added in sequence, and the reaction was carried out at 100° C. for 2 h. After the reaction was completed, the mixture was cooled to room temperature, the reaction solution was concentrated, an appropriate amount of water was added, and the mixture was extracted with EA. The organic phases were combined, dried, concentrated, and subjected to column chromatography to obtain intermediate D1-1 (30.3 g) with a yield of 82%. ESI-MS (M+H)⁺397.2.

### Synthesis Step 2: tert-butyl 4-(5-(2-bromobenzo[d]thiazol-6-yl)pyridin-2-yl) piperazin-1-carboxylate (intermediate D1)

The intermediate D1-1 (10 g, 21.08 mmol) was dissolved in 100 mL of tetrahydrofuran. Under nitrogen protection, the system was cooled to −78° C., and n-butyllithium (2.5 mol/L, 9.3 mL, 23.19 mmol) was added. The reaction was carried out at −60° C. to −70° C. for 1 h, and then carbon tetrabromide (9.08 g, 27.40 mmol) was added. After 30 minutes of reaction, water was added to quench the reaction, and the mixture was extracted with EA. The organic phases were combined, dried, concentrated, and purified by column chromatography to obtain 4.6 g of intermediate D1 with a yield of 46%.

ESI-MS (M+H)⁺475.1.

### Example 23

### Intermediate D2:2-bromo-6-(pyridin-3-yl)benzo[d]thiazole

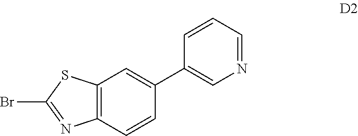 [SMILES: Brc1nc2ccc(-c3cccnc3)cc2s1]

Referring to the synthetic route and method of intermediate D1, in synthesis step 1, tert-butyl 4-(5-(4,4,5,5-tetramethyl-1,3,2-dioxaborolane-2-yl)pyridin-2-yl) piperazin-1-carboxylate was replaced with 3-(4,4,5,5-tetramethyl-1,3,2-dioxaborolane-2-yl)pyridine to synthesize intermediate D2, ESI-MS (M+H)⁺291.0.

Referring to the synthetic routes and methods of intermediates D1 to D2, the following intermediate compounds were synthesized:

| Number of | | |
|---|---|---|
| intermediate | Structure of intermediate | ESI-MS(M + H)⁺ |
| D3 | 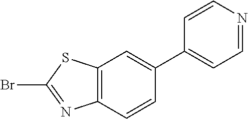 [SMILES: Brc1nc2ccc(-c3ccncc3)cc2s1] | 291.0 |
| D4 | 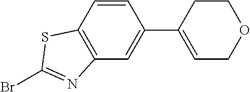 [SMILES: Brc1nc2cc(C3=CCOCC3)ccc2s1] | 296.0 |
| D5 | 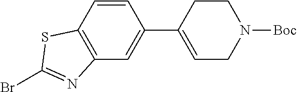 [SMILES: CC(C)(C)OC(=O)N1CC=C(c2ccc3sc(Br)nc3c2)CC1] | 395.0 |
| D6 | 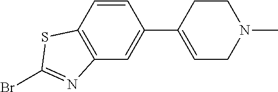 [SMILES: CN1CC=C(c2ccc3sc(Br)nc3c2)CC1] | 309.0 |
| D7 | 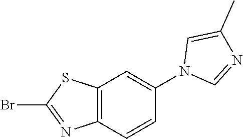 [SMILES: Cc1cn(-c2ccc3nc(Br)sc3c2)cn1] | 294.0 |
| D8 | 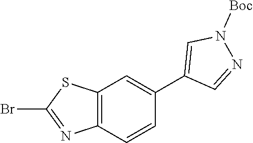 [SMILES: CC(C)(C)OC(=O)n1cc(-c2ccc3nc(Br)sc3c2)cn1] | 380.0 |
| D9 | 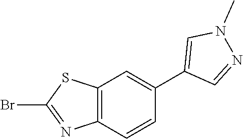 [SMILES: Cn1cc(-c2ccc3nc(Br)sc3c2)cn1] | 294.0 |

### Example 24

Intermediate E1: benzyl 3-(benzo[d]thiazol-2-yl)-2-isocyano-4,7-dihydrothieno[2,3-c]pyridin-6 (5H)-carboxylate

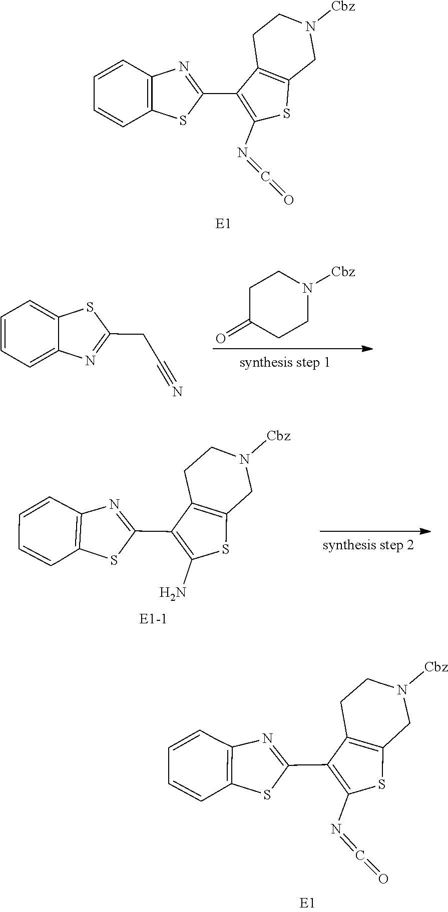 [SMILES: O=C=Nc1sc2c(c1-c1nc3ccccc3s1)CCN(C(=O)OCc1ccccc1)C2 ; N#CCc1nc2ccccc2s1 ; O=C1CCN(C(=O)OCc2ccccc2)CC1 ; Nc1sc2c(c1-c1nc3c(s1)CC=CC=C3)CCN(C(=O)OCc1ccccc1)C2 ; O=C=Nc1sc2c(c1-c1nc3c(s1)C=CC=CC3)CCCN(C(=O)OCc1ccccc1)C2]

### Synthesis Step 1: benzyl 2-amino-3-(benzo[d]thiazol-2-yl)-4,7-dihydrothieno[2,3-c]pyridin-6 (5H)-carboxylate (intermediate E1-1)

2-(Benzo[d]thiazol-2-yl) acetonitrile (3.64 g, 21.0 mmol) was dissolved in 100 mL of ethanol solution, benzyl 4-oxopiperidin-1-carboxylate (5.8 g, 25.2 mmol) and morpholine (2.18 mL, 25.1 mmol) were added into the system in sequence, and the mixture was stirred at 40° C. for 15 min. After the solution became clear, elemental sulfur (804 mg, 25.1 mmol) was added, and the temperature was raised to 90° C. for reaction for 2 h. After the reaction was completed, the mixture was cooled to room temperature and filtered. The filter cake was dried to obtain intermediate E1-1 (8.4 g) with a yield of 94.8%, which was directly used in the next step without purification. ESI-MS (M+H)⁺422.1.

### Synthesis Step 2: benzyl 3-(benzo[d]thiazol-2-yl)-2-isocyano-4,7-dihydrothieno[2,3-c]pyridin-6 (5H)-carboxylate (intermediate E1)

The intermediate E1-1 (5 g, 11.8 mmol) was dissolved in 50 mL of tetrahydrofuran solution, DIPEA (3 g, 23.6 mmol) and triphosgene (1.2 g, 3.9 mmol) were added in sequence at 0° C., and then the mixture was reacted at room temperature for 3 h. After that, the reaction solution was concentrated, slurried with petroleum ether and filtered to obtain 3.8 g of intermediate E1 as product with a yield of 73%, which was directly used in the next step without purification. ESI-MS (M+H)⁺448.1.

Referring to the synthetic route and method of intermediate E1, the following intermediate compound was synthesized:

| Number of | | |
|---|---|---|
| intermediate | Structure of intermediate | ESI-MS(M + H)⁺ |
| E2 | 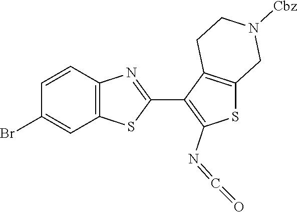 [SMILES: O=C=Nc1sc2c(c1-c1nc3ccc(Br)cc3s1)CCN(C(=O)OCc1ccccc1)C2] | 526.0 |

### Example 25: N-(3-(benzo[d]thiazol-2-yl)-4,5,6,7-tetrahydrothieno[2,3-c]pyridin-2-yl)-3-(isopropylamino)cyclobutane-1-carboxamide (001)

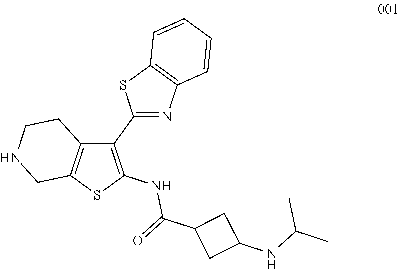 [SMILES: CC(C)NC1CC(C(=O)Nc2sc3c(c2-c2nc4ccccc4s2)CCNC3)C1]

The intermediate B5 (105 mg, 0.20 mmol) was dissolved in 5 mL of DCM, hydrogen chloride (dissolved in dioxane, 4M, 2 mL) was added to the system, and the reaction was carried out at room temperature for 1 h. After the reaction was completed, the mixture was concentrated under reduced pressure, adjusted to pH of weak alkalinity by adding saturated sodium bicarbonate aqueous solution, and extracted with dichloromethane. The organic phase was concentrated, then separated and purified by reverse-phase chromatography, and freeze-dried to obtain compound 001 (40 mg). ESI-MS (M+H)⁺427.2.

¹H-NMR (400 MHz, DMSO-d₆) δ 9.11-9.62 (m, 3H), 8.18-8.21 (m, 1H), 8.10-8.14 (m, 1H), 7.58-7.63 (m, 1H), 7.48-7.53 (m, 1H), 4.32-4.34 (m, 2H), 3.66-3.85 (m, 1H), 3.47-3.49 (m, 2H), 3.37-3.39 (m, 1H), 3.18-3.29 (m, 3H), 2.53-2.71 (m, 4H), 1.20-1.24 (m, 6H).

Referring to the synthetic route and method of Example 25, the following compounds were synthesized:

| Structure and number of compound | Characterization data |
|---|---|
| 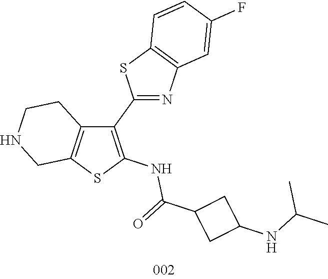 [SMILES: CC(C)NC1CC(C(=O)Nc2sc3c(c2-c2nc4cc(F)ccc4s2)CCNC3)C1] | ESI-MS(M + H)⁺ = 445.1 |
| 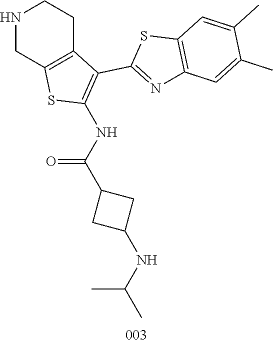 [SMILES: Cc1cc2nc(-c3c(NC(=O)C4CC(NC(C)C)C4)sc4c3CCNC4)sc2cc1C] | ESI-MS(M + H)⁺ = 455.2 |
| 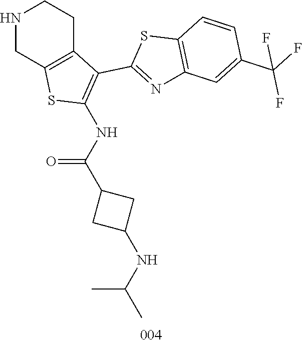 [SMILES: CC(C)NC1CC(C(=O)Nc2sc3c(c2-c2nc4cc(C(F)(F)F)ccc4s2)CCNC3)C1] | ESI-MS(M + H)⁺ = 495.1 |
| 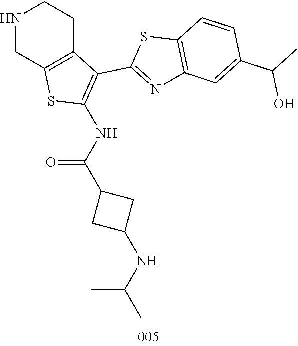 [SMILES: CC(C)NC1CC(C(=O)Nc2sc3c(c2-c2nc4cc(C(C)O)ccc4s2)CCNC3)C1] | ESI-MS(M + H)⁺ = 471.2 |
| 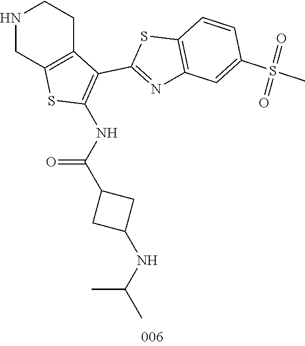 [SMILES: CC(C)NC1CC(C(=O)Nc2sc3c(c2-c2nc4cc(S(C)(=O)=O)ccc4s2)CCNC3)C1] | ESI-MS(M + H)⁺ = 505.1 |

### Example 26 3-(isopropylamino)-N-(3-(6-(1,2,3,6-tetrahydropyridin-4-yl)tetrahydrothieno[2,3-c]pyridin-2-yl)cyclobutane-1-benzo[d]thiazol-2-yl)-4,5,6,7-formylamino (048)

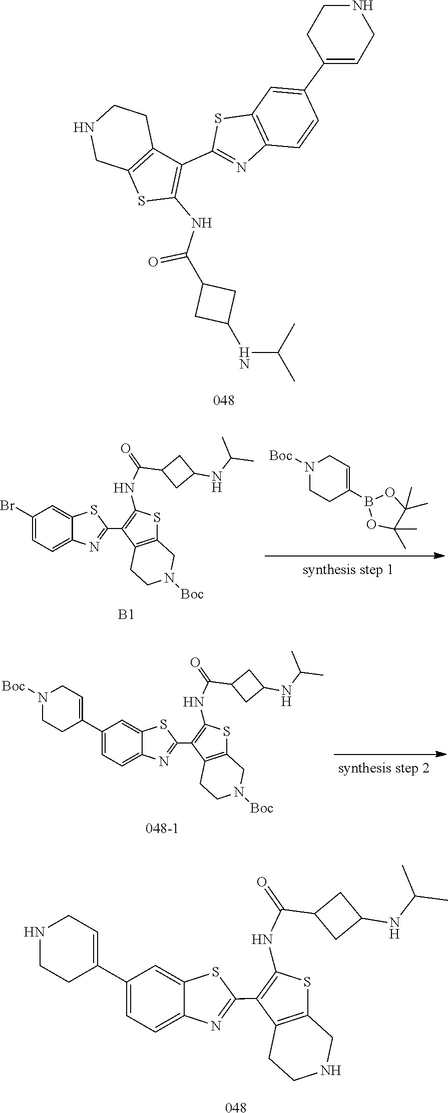

### Synthesis Step 1: tert-butyl 3-(6-(1-(tert-butoxycarbonyl)-1,2,3,6-tetrahydropyridin-4-yl)benzo[d]thiazol-2-yl)-2-(3-(isopropylamino)cyclobutane-1-formylamino)-4,7-dihydropthieno[2,3-c]pyridin-6 (5H)-carboxylate (048-1)

The intermediate B1 (130 mg, 0.21 mmol) was added to a mixed solution (dioxane: water=3:1, 5 mL), tert-butyl 4-(4,4,5,5-tetramethyl-1,3,2-dioxaborolane-2-yl)-3,6-dihydropyridin-1 (2H)-carboxylate (80 mg, 0.25 mmol), Pd(dppf)Cl₂ (15 mg, 0.02 mmol) and Na₂CO₃ (70 mg, 0.63 mmol) were added into the system in sequence, and the reaction was carried out at 80° C. for 5 h. After the reaction was completed, it was quenched by adding water and extracted three times with DCM. The organic phases were combined, dried over anhydrous sodium sulfate, concentrated under reduced pressure, and subjected to column chromatography to obtain 048-1 (140 mg) with a yield of 92%. ESI-MS (M+H)⁺708.3.

### Synthesis Step 2:3-(isopropylamino)-N-(3-(6-(1,2,3,6-tetrahydropyridin-4-yl)benzo[d]thiazol-2-yl)-4,5,6,7-tetr ahydrothieno[2,3-c]pyridin-2-yl)cyclobutane-1-formylamino (048)

048-1 (140 mg, 0.20 mmol) was dissolved in 5 mL of DCM, hydrogen chloride (dissolved in dioxane, 4M, 2 mL) was added into the system, and the reaction was carried out at room temperature for 1 h. After the reaction was completed, the mixture was concentrated under reduced pressure, adjusted to pH of weak alkalinity by adding saturated sodium bicarbonate aqueous solution, and extracted with dichloromethane. The organic phase was concentrated, then separated and purified by reverse-phase chromatography, and freeze-dried to obtain compound 048 (70 mg). ESI-MS (M+H)⁺508.2. ESI-MS (M+H)⁺508.2.

¹H-NMR (400 MHz, DMSO-d₆) δ 9.13-9.64 (m, 4H), 8.31-8.32 (m, 1H), 8.09-8.14 (m, 1H), 7.76-7.78 (m, 1H), 6.36 (brs, 1H), 4.32-4.34 (m, 2H), 3.78-3.81 (m, 2H), 3.66-3.71 (m, 1H), 3.45-3.50 (m, 3H), 3.15-3.24 (m, 5H), 2.77-2.80 (m, 2H), 2.52-2.68 (m, 4H), 1.20-1.24 (m, 6H).

### Example 27:3-(isopropylamino)-N-(3-(6-(pyrimidin-5-yl)benzo[d]thiazol-2-yl)-4,5,6,7-tetrahydrothieno[2,3-c]pyridin-2-yl)cyclobutane-1-carboxamide (016)

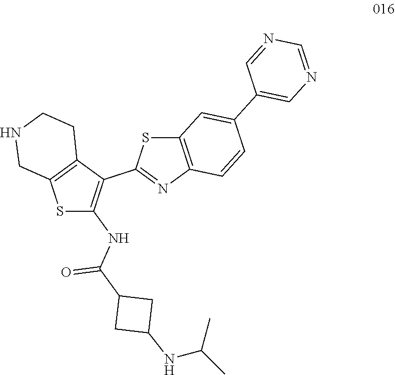 [SMILES: CC(C)NC1CC(C(=O)Nc2sc3c(c2-c2nc4ccc(-c5cncnc5)cc4s2)CCNC3)C1]

Referring to the synthesis route and method of Example 26, in synthesis step 1, tert-butyl 4-(4,4,5,5-tetramethyl-1,3,2-dioxaborolane-2-yl)-3,6-dihydropyridin-1 (2H)-carboxylate was replaced with 5-(4,4,5,5-tetramethyl-1,3,2-dioxaborolane-2-yl)pyrimidine to synthesize and obtain the target compound 016.

ESI-MS (M+H)⁺505.2.

¹H-NMR (400 MHz, DMSO-d₆) δ 9.64 (brs, 2H), 9.26-9.27 (m, 2H), 9.23-9.24 (m, 1H), 9.10 (brs, 1H), 8.68-8.69 (m, 1H), 8.23-8.26 (m, 1H), 8.04-8.08 (m, 1H), 4.32-4.34 (m, 2H), 3.78-3.83 (m, 1H), 3.48-3.50 (m, 2H), 3.18-3.30 (m, 3H), 2.52-2.72 (m, 4H), 1.95-1.99 (m, 1H), 1.20-1.22 (m, 6H).

### Example 28:3-(isopropylamino)-N-(3-(6-(1,2,3,6-tetrahydropyridin-4-yl)benzo[d]thiazol-2-yl)-4,5,6,7-tetrahydrothieno[2,3-c]pyridin-2-yl)cyclobutane-1-carboxamide (061)

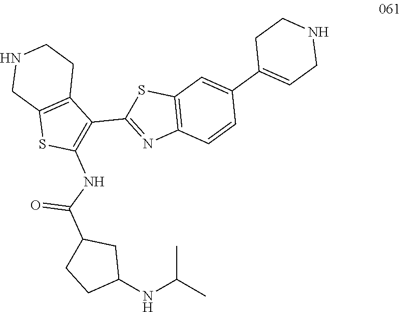 [SMILES: CC(C)NC1CCC(C(=O)Nc2sc3c(c2-c2nc4ccc(C5=CCNCC5)cc4s2)CCNC3)C1]

Referring to the synthesis route and method of Example 26, in synthesis step 1, intermediate B1 was replaced with intermediate B4 to obtain the target compound 061. ESI-MS (M+H)⁺522.2.

Referring to the synthetic routes and methods of Examples 26 to 28, the following target compounds were synthesized:

| Structure and number of compound | Characterization data |
|---|---|
| 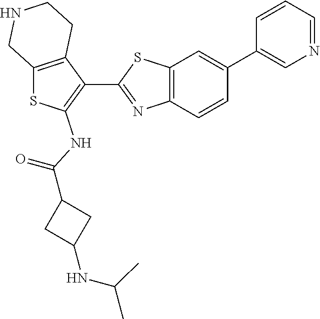 [SMILES: CC(C)NC1CC(C(=O)Nc2sc3c(c2-c2nc4ccc(-c5cccnc5)cc4s2)CCNC3)C1] | ESI-MS (M + H)⁺ = 504.2 |
| 007 | |
| 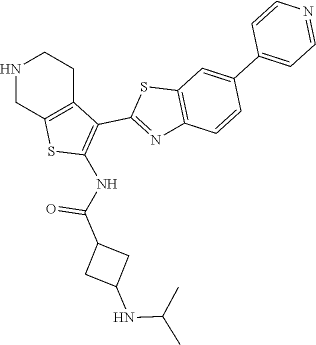 [SMILES: CC(C)NC1CC(C(=O)Nc2sc3c(c2-c2nc4ccc(-c5ccncc5)cc4s2)CCNC3)C1] | ESI-MS (M + H)⁺ = 504.2 |
| 008 | |
| 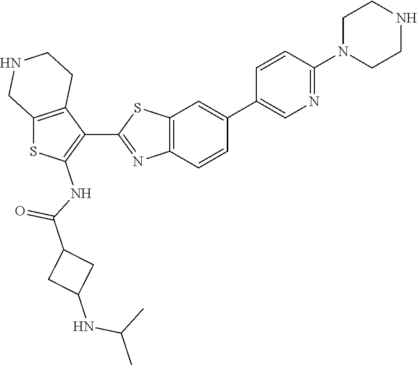 [SMILES: CC(C)NC1CC(C(=O)Nc2sc3c(c2-c2nc4ccc(-c5ccc(N6CCNCC6)nc5)cc4s2)CCNC3)C1] | ESI-MS (M + H)⁺ = 588.3 ¹H-NMR (400 MHz, DMSO-d₆) δ 9.21-9.74 (m, 4H), 8.59-8.60 (m, 1H), 8.49-8.51 (m, 1H), 8.11-8.20 (m, 2H), 7.89-7.92 (m, 1H), 7.13- 7.15 (m, 1H), 4.32-4.34 (m, 2H), 3.85-3.86 (m, 7H), 3.37-3.51 (m, 3H), 3.18-3.29 (m, 5H), 2.53-2.74 (m, 4H), 1.20-1.24 (m, 6H). |
| 009 | |
| 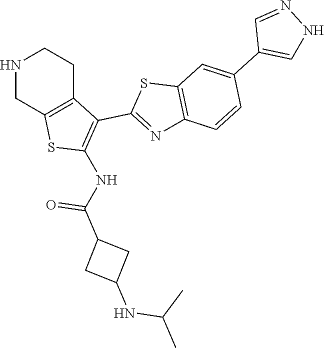 [SMILES: CC(C)NC1CC(C(=O)Nc2sc3c(c2-c2nc4ccc(-c5cn[nH]c5)cc4s2)CCNC3)C1] | ESI-MS (M + H)⁺ = 493.2 |
| 010 | |
| 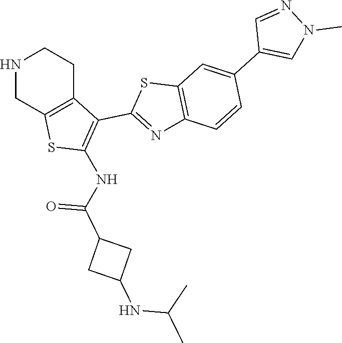 [SMILES: CC(C)NC1CC(C(=O)Nc2sc3c(c2-c2nc4ccc(-c5cnn(C)c5)cc4s2)CCNC3)C1] | ESI-MS (M + H)⁺ = 507.2 |
| 011 | |
| 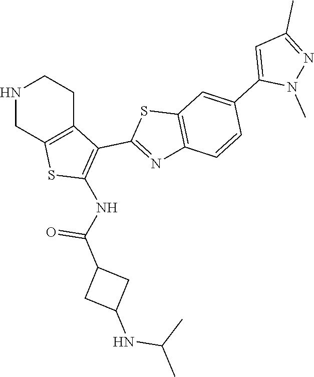 [SMILES: Cc1cc(-c2ccc3nc(-c4c(NC(=O)C5CC(NC(C)C)C5)sc5c4CCNC5)sc3c2)n(C)n1] | ESI-MS (M + H)⁺ = 521.2 |
| 012 | |
| 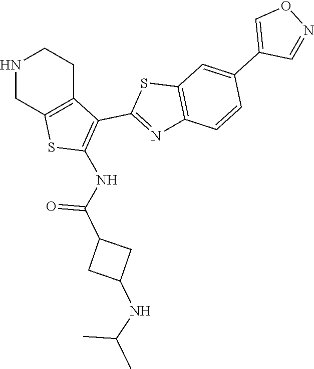 [SMILES: CC(C)NC1CC(C(=O)Nc2sc3c(c2-c2nc4ccc(-c5cnoc5)cc4s2)CCNC3)C1] | ESI-MS (M + H)⁺ = 494.2 |
| 013 | |
| 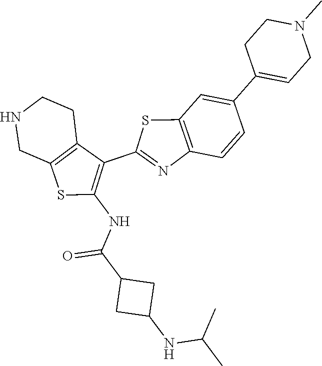 [SMILES: CC(C)NC1CC(C(=O)Nc2sc3c(c2-c2nc4ccc(C5=CCN(C)CC5)cc4s2)CCNC3)C1] | ESI-MS (M + H)⁺ = 522.2 ¹H-NMR (400 MHz, DMSO-d₆) δ 9.19-9.74 (m, 3H), 8.30-8.32 (m, 1H), 8.07-8.11 (m, 1H), 7.74-7.77 (m, 1H), 6.35 (s, 1H), 4.30-4.32 (m, 2H), 3.69-3.77 (m, 3H), 3.45-3.59 (m, 6H), 3.16-3.18 (m, 5H), 2.54-2.72 (m, 6H), 1.12- 1.13 (m, 6H). |
| 017 | |
| 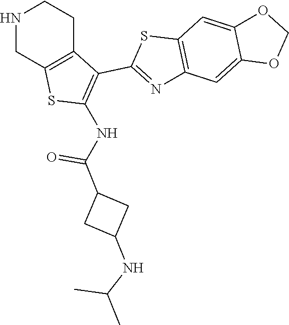 [SMILES: CC(C)NC1CC(C(=O)Nc2sc3c(c2-c2nc4cc5c(cc4s2)OCO5)CCNC3)C1] | ESI-MS (M + H)⁺ = 471.1 |
| 018 | |
| 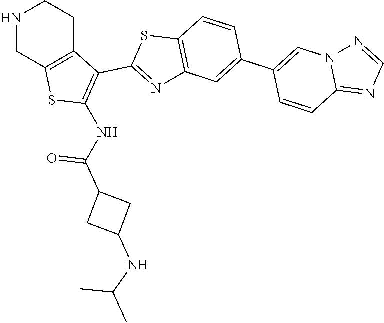 [SMILES: CC(C)NC1CC(C(=O)Nc2sc3c(c2-c2nc4cc(-c5ccc6ncnn6c5)ccc4s2)CCNC3)C1] | ESI-MS (M + H)⁺ = 544.2 |
| 019 | |
| 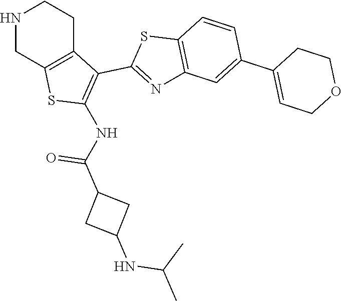 [SMILES: CC(C)NC1CC(C(=O)Nc2sc3c(c2-c2nc4cc(C5=CCOCC5)ccc4s2)CCNC3)C1] | ESI-MS (M + H)⁺ = 509.2 |
| 021 | |
| 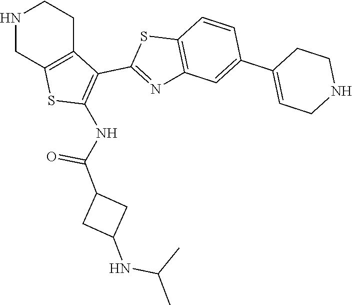 [SMILES: CC(C)NC1CC(C(=O)Nc2sc3c(c2-c2nc4cc(C5=CCNCC5)ccc4s2)CCNC3)C1] | ESI-MS (M + H)⁺ = 508.2 ¹H-NMR (400 MHz, DMSO-d₆) δ 9.28-9.61 (m, 4H), 8.14-8.22 (m, 2H), 7.64-7.67 (m, 1H), 6.40-6.42 (m, 1H), 4.32-4.33 (m, 2H), 3.78- 3.79 (m, 3H), 3.45-3.49 (m, 3H), 3.15-3.21 (m, 5H), 2.58-2.83 (m, 6H), 1.23-1.25 (m, 6H). |
| 022 | |
| 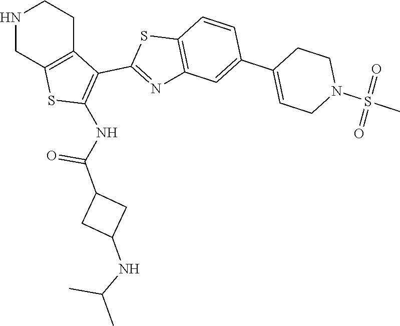 [SMILES: CC(C)NC1CC(C(=O)Nc2sc3c(c2-c2nc4cc(C5=CCN(S(C)(=O)=O)CC5)ccc4s2)CCNC3)C1] | ESI-MS (M + H)⁺ = 586.2 |
| 023 | |
| 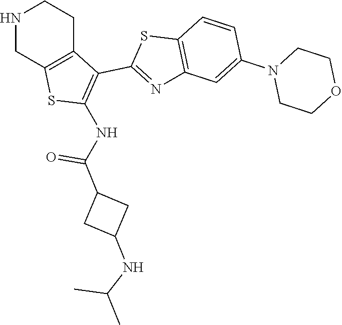 [SMILES: CC(C)NC1CC(C(=O)Nc2sc3c(c2-c2nc4cc(N5CCOCC5)ccc4s2)CCNC3)C1] | ESI-MS (M + H)⁺ = 512.2 |
| 024 | |
| 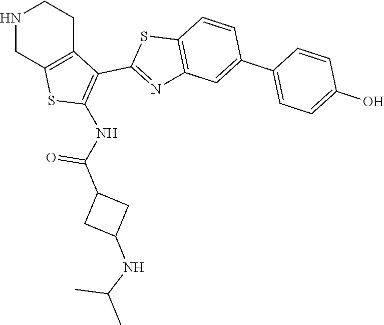 [SMILES: CC(C)NC1CC(C(=O)Nc2sc3c(c2-c2nc4cc(-c5ccc(O)cc5)ccc4s2)CCNC3)C1] | ESI-MS (M + H)⁺ = 519.2 |
| 025 | |
| 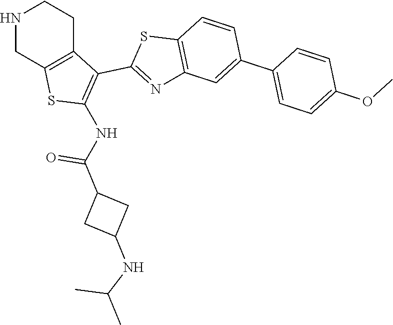 [SMILES: COc1ccc(-c2ccc3sc(-c4c(NC(=O)C5CC(NC(C)C)C5)sc5c4CCNC5)nc3c2)cc1] | ESI-MS (M + H)⁺ = 533.2 |
| 026 | |
| 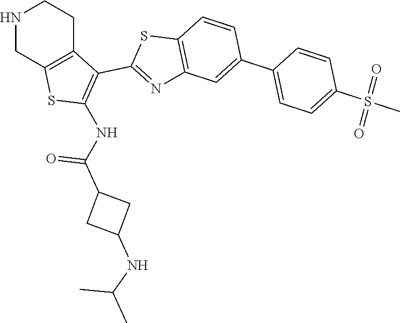 [SMILES: CC(C)NC1CC(C(=O)Nc2sc3c(c2-c2nc4cc(-c5ccc(S(C)(=O)=O)cc5)ccc4s2)CCNC3)C1] | ESI-MS (M + H)⁺ = 581.2 |
| 027 | |
| 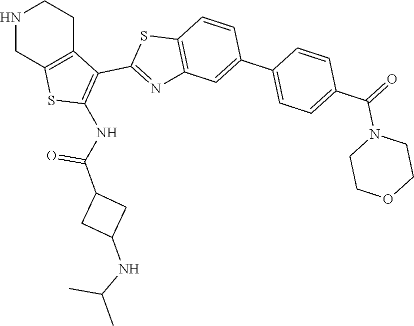 [SMILES: CC(C)NC1CC(C(=O)Nc2sc3c(c2-c2nc4cc(-c5ccc(C(=O)N6CCOCC6)cc5)ccc4s2)CCNC3)C1] | ESI-MS (M + H)⁺ = 616.2 |
| 028 | |
| 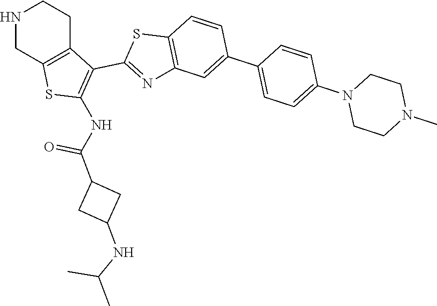 [SMILES: CC(C)NC1CC(C(=O)Nc2sc3c(c2-c2nc4cc(-c5ccc(N6CCN(C)CC6)cc5)ccc4s2)CCNC3)C1] | ESI-MS (M + H)⁺ = 601.3 |
| 029 | |
| 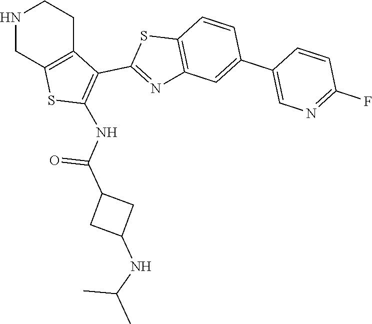 [SMILES: CC(C)NC1CC(C(=O)Nc2sc3c(c2-c2nc4cc(-c5ccc(F)nc5)ccc4s2)CCNC3)C1] | ESI-MS (M + H)⁺ = 522.2 |
| 030 | |
| 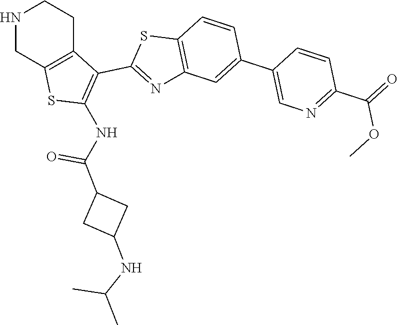 [SMILES: COC(=O)c1ccc(-c2ccc3sc(-c4c(NC(=O)C5CC(NC(C)C)C5)sc5c4CCNC5)nc3c2)cn1] | ESI-MS (M + H)⁺ = 562.2 |
| 031 | |
| 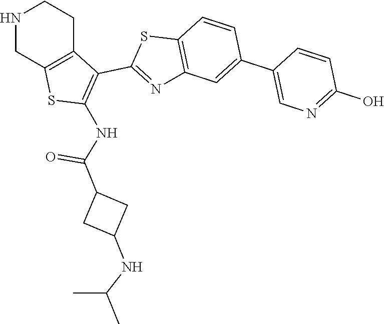 [SMILES: CC(C)NC1CC(C(=O)Nc2sc3c(c2-c2nc4cc(-c5ccc(O)nc5)ccc4s2)CCNC3)C1] | ESI-MS (M + H)⁺ = 520.2 |
| 032 | |
| 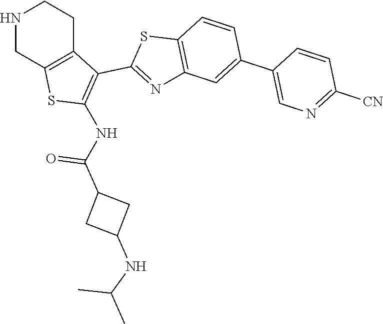 [SMILES: CC(C)NC1CC(C(=O)Nc2sc3c(c2-c2nc4cc(-c5ccc(C#N)nc5)ccc4s2)CCNC3)C1] | ESI-MS (M + H)⁺ = 529.2 |
| 033 | |
| 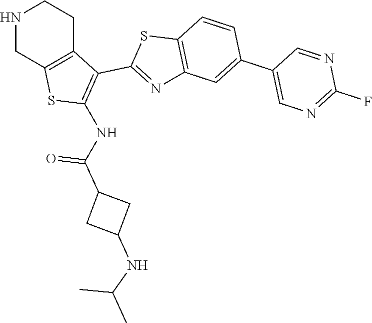 [SMILES: CC(C)NC1CC(C(=O)Nc2sc3c(c2-c2nc4cc(-c5cnc(F)nc5)ccc4s2)CCNC3)C1] | ESI-MS (M + H)⁺ = 523.2 |
| 034 | |
| 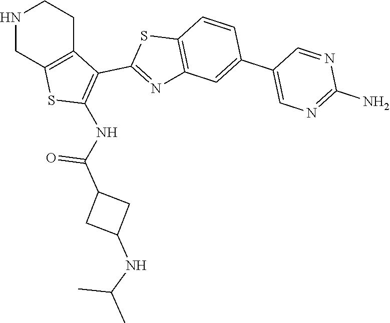 [SMILES: CC(C)NC1CC(C(=O)Nc2sc3c(c2-c2nc4cc(-c5cnc(N)nc5)ccc4s2)CCNC3)C1] | ESI-MS (M + H)⁺ = 520.2 |
| 035 | |
| 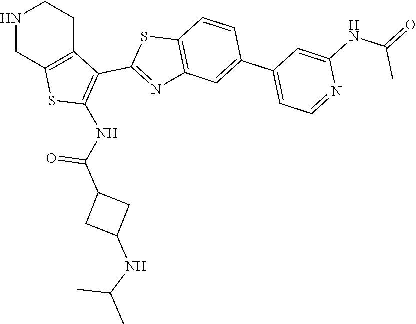 [SMILES: CC(=O)Nc1cc(-c2ccc3sc(-c4c(NC(=O)C5CC(NC(C)C)C5)sc5c4CCNC5)nc3c2)ccn1] | ESI-MS (M + H)⁺ = 561.2 |
| 036 | |
| 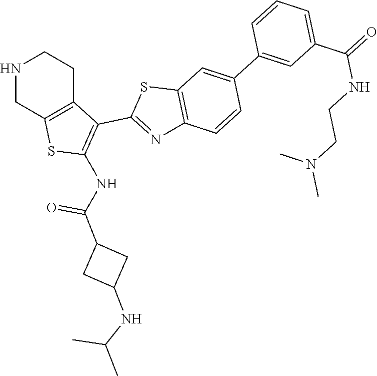 [SMILES: CC(C)NC1CC(C(=O)Nc2sc3c(c2-c2nc4ccc(-c5cccc(C(=O)NCCN(C)C)c5)cc4s2)CCNC3)C1] | ESI-MS (M + H)⁺ = 617.3 |
| 037 | |
| 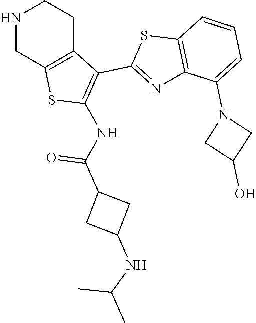 [SMILES: CC(C)NC1CC(C(=O)Nc2sc3c(c2-c2nc4c(N5CC(O)C5)cccc4s2)CCNC3)C1] | ESI-MS (M + H)⁺ = 498.2 |
| 038 | |
| 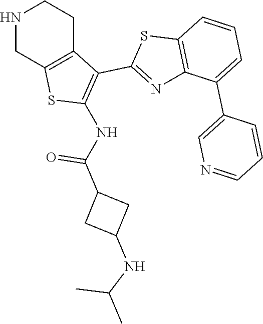 [SMILES: CC(C)NC1CC(C(=O)Nc2sc3c(c2-c2nc4c(-c5cccnc5)cccc4s2)CCNC3)C1] | ESI-MS (M + H)⁺ = 504.2 |
| 039 | |
| 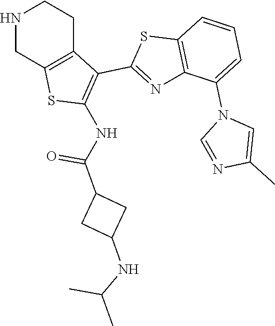 [SMILES: Cc1cn(-c2cccc3sc(-c4c(NC(=O)C5CC(NC(C)C)C5)sc5c4CCNC5)nc23)cn1] | ESI-MS (M + H)⁺ = 507.2 |
| 040 | |
| 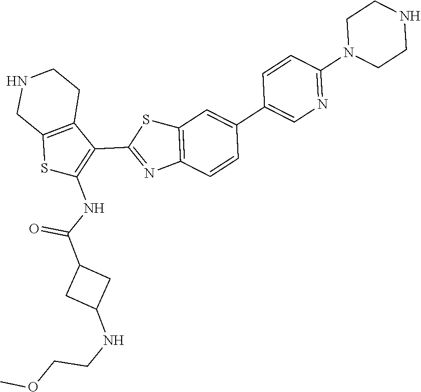 [SMILES: COCCNC1CC(C(=O)Nc2sc3c(c2-c2nc4ccc(-c5ccc(N6CCNCC6)nc5)cc4s2)CCNC3)C1] | ESI-MS (M + H)⁺ = 604.2 |
| 041 | |
| 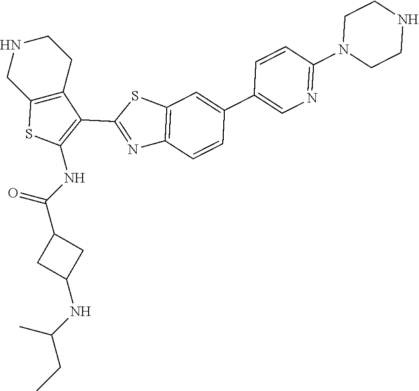 [SMILES: CCC(C)NC1CC(C(=O)Nc2sc3c(c2-c2nc4ccc(-c5ccc(N6CCNCC6)nc5)cc4s2)CCNC3)C1] | ESI-MS (M + H)⁺ = 602.3 |
| 042 | |
| 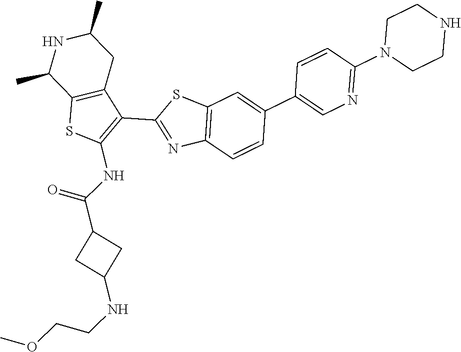 [SMILES: COCCNC1CC(C(=O)Nc2sc3c(c2-c2nc4ccc(-c5ccc(N6CCNCC6)nc5)cc4s2)C[C@H](C)N[C@@H]3C)C1] | ESI-MS (M + H)⁺ = 632.3 |
| 044 | |
| 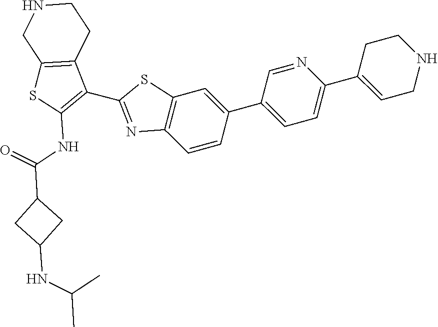 [SMILES: CC(C)NC1CC(C(=O)Nc2sc3c(c2-c2nc4ccc(-c5ccc(C6=CCNCC6)nc5)cc4s2)CCNC3)C1] | ESI-MS (M + H)⁺ = 585.2 ¹H-NMR (400 MHz, DMSO-d₆) δ 9.15-9.68 (m, 4H), 9.02-9.03 (m, 1H), 8.64-8.65 (m, 1H), 8.20-8.26 (m, 2H), 8.02-8.04 (m, 1H), 7.78- 7.81 (m, 1H), 6.83 (s, 1H), 4.31-4.33 (m, 2H), 3.82-3.83 (m, 2H), 3.60-3.70 (m, 4H), 3.15- 3.25 (m, 5H), 2.81-2.82 (m, 2H), 2.56-2.70 (m, 4H), 1.12-1.13 (m, 6H). |
| 047 | |
| 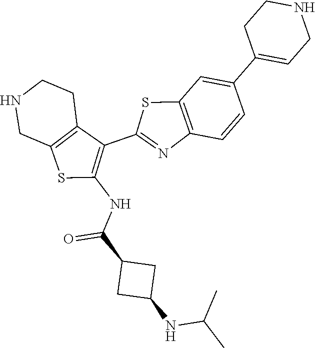 [SMILES: CC(C)N[C@H]1C[C@@H](C(=O)Nc2sc3c(c2-c2nc4ccc(C5=CCNCC5)cc4s2)CCNC3)C1] | ESI-MS (M + H)⁺ = 508.2 |
| 049 | |
| 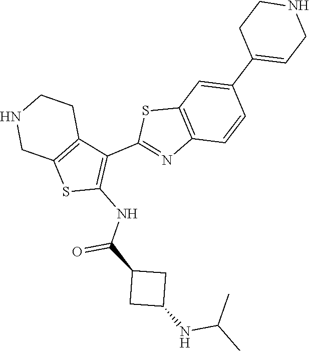 [SMILES: CC(C)N[C@H]1C[C@H](C(=O)Nc2sc3c(c2-c2nc4ccc(C5=CCNCC5)cc4s2)CCNC3)C1] | ESI-MS (M + H)⁺ = 508.2 |
| 050 | |
| 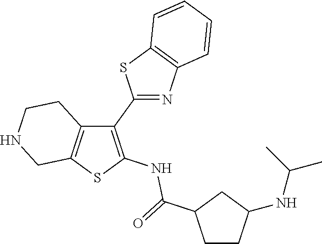 [SMILES: CC(C)NC1CCC(C(=O)Nc2sc3c(c2-c2nc4ccccc4s2)CCNC3)C1] | ESI-MS (M + H)⁺ = 441.2 |
| 057 | |
| 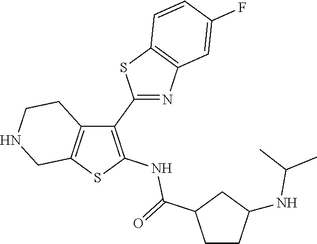 [SMILES: CC(C)NC1CCC(C(=O)Nc2sc3c(c2-c2nc4cc(F)ccc4s2)CCNC3)C1] | ESI-MS (M + H)⁺ = 459.2 |
| 058 | |
| 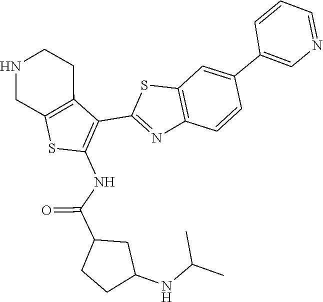 [SMILES: CC(C)NC1CCC(C(=O)Nc2sc3c(c2-c2nc4ccc(-c5cccnc5)cc4s2)CCNC3)C1] | ESI-MS (M + H)⁺ = 518.2 |
| 059 | |
| 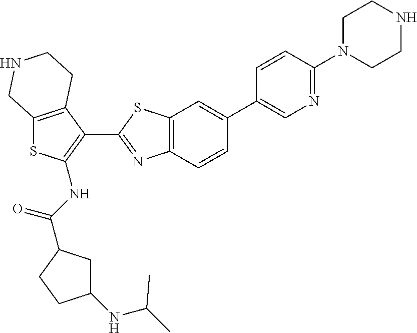 [SMILES: CC(C)NC1CCC(C(=O)Nc2sc3c(c2-c2nc4ccc(-c5ccc(N6CCNCC6)nc5)cc4s2)CCNC3)C1] | ESI-MS (M + H)⁺ = 602.3 ¹H-NMR (400 MHz, DMSO-d₆) δ 9.03-9.77 (m, 4H), 8.59-8.60 (m, 2H), 8.49-8.50 (m, 1H), 8.19-8.34 (m, 1H), 8.11-8.14 (m, 1H), 7.88- 7.92 (m, 1H), 4.32-4.33 (m, 2H), 3.85-3.87 (m, 4H), 3.64-3.66 (m, 2H), 3.18-3.31 (m, 9H), 1.87-2.23 (m, 6H), 1.28-1.30 (m, 6H). |
| 060 | |
| 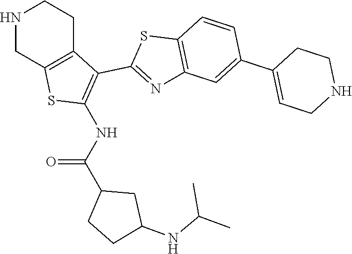 [SMILES: CC(C)NC1CCC(C(=O)Nc2sc3c(c2-c2nc4cc(C5=CCNCC5)ccc4s2)CCNC3)C1] | ESI-MS (M + H)⁺ = 522.2 |
| 062 | |
| 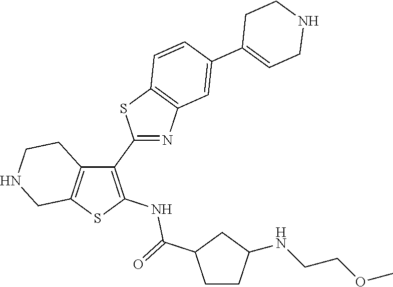 [SMILES: COCCNC1CCC(C(=O)Nc2sc3c(c2-c2nc4cc(C5=CCNCC5)ccc4s2)CCNC3)C1] | ESI-MS (M + H)⁺ = 538.2 |
| 063 | |
| 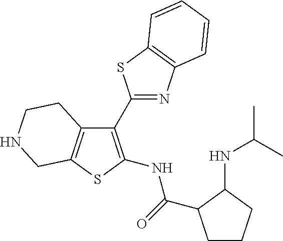 [SMILES: CC(C)NC1CCCC1C(=O)Nc1sc2c(c1-c1nc3ccccc3s1)CCNC2] | ESI-MS (M + H)⁺ = 441.2 |
| 064 | |
| 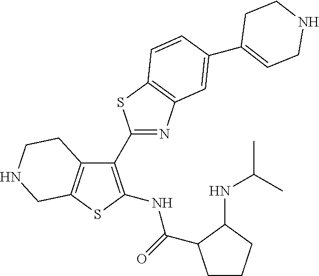 [SMILES: CC(C)NC1CCCC1C(=O)Nc1sc2c(c1-c1nc3cc(C4=CCNCC4)ccc3s1)CCNC2] | ESI-MS (M + H)⁺ = 522.2 |
| 065 | |
| 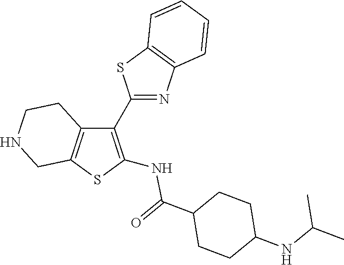 [SMILES: CC(C)NC1CCC(C(=O)Nc2sc3c(c2-c2nc4ccccc4s2)CCNC3)CC1] | ESI-MS (M + H)⁺ = 455.2 |
| 066 | |
| 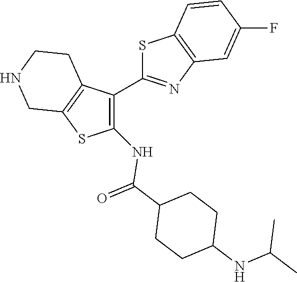 [SMILES: CC(C)NC1CCC(C(=O)Nc2sc3c(c2-c2nc4cc(F)ccc4s2)CCNC3)CC1] | ESI-MS (M + H)⁺ = 473.2 |
| 067 | |
| 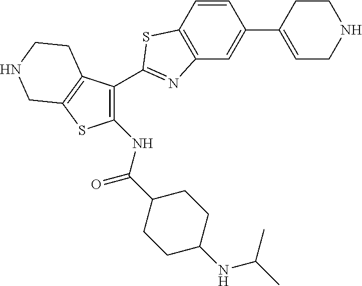 [SMILES: CC(C)NC1CCC(C(=O)Nc2sc3c(c2-c2nc4cc(C5=CCNCC5)ccc4s2)CCNC3)CC1] | ESI-MS (M + H)⁺ = 536.2 |
| 068 | |
| 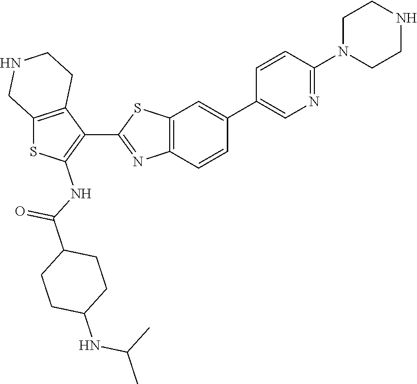 [SMILES: CC(C)NC1CCC(C(=O)Nc2sc3c(c2-c2nc4ccc(-c5ccc(N6CCNCC6)nc5)cc4s2)CCNC3)CC1] | ESI-MS (M + H)⁺ = 616.3 ¹H-NMR (400 MHz, DMSO-d₆) δ 9.48-9.91 (m, 3H), 8.91-8.94 (m, 1H), 8.49-8.56 (m, 2H), 8.04-8.16 (m, 2H), 7.89-7.92 (m, 1H), 7.16- 7.18 (m, 1H), 4.32-4.33 (m, 2H), 3.87-3.89 (m, 4H), 3.59-3.66 (m, 3H), 3.18-3.20 (m, 8H), 2.59-3.02 (m, 1H), 2.19-2.38 (m, 3H), 1.81- 2.02 (m, 2H), 1.52-1.63 (m, 2H), 1.18-1.28 (m, 6H). |
| 069 | |
| 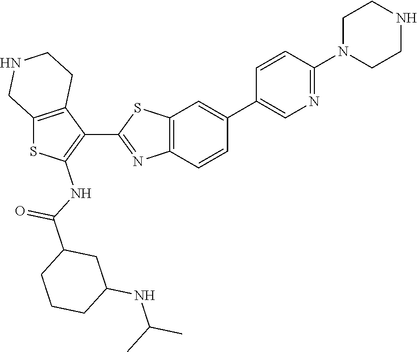 [SMILES: CC(C)NC1CCCC(C(=O)Nc2sc3c(c2-c2nc4ccc(-c5ccc(N6CCNCC6)nc5)cc4s2)CCNC3)C1] | ESI-MS (M + H)⁺ = 616.3 |
| 070 | |
| 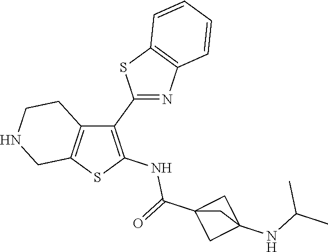 [SMILES: CC(C)NC12CC(C(=O)Nc3sc4c(c3-c3nc5ccccc5s3)CCNC4)(C1)C2] | ESI-MS (M + H)⁺ = 439.2 |
| 111 | |
| 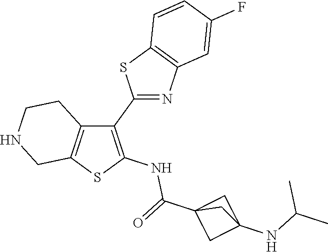 [SMILES: CC(C)NC12CC(C(=O)Nc3sc4c(c3-c3nc5cc(F)ccc5s3)CCNC4)(C1)C2] | ESI-MS (M + H)⁺ = 457.1 |
| 112 | |
| 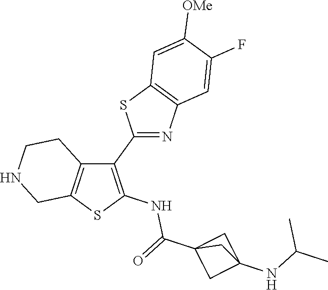 [SMILES: COc1cc2sc(-c3c(NC(=O)C45CC(NC(C)C)(C4)C5)sc4c3CCNC4)nc2cc1F] | ESI-MS (M + H)⁺ = 487.2 |
| 113 | |
| 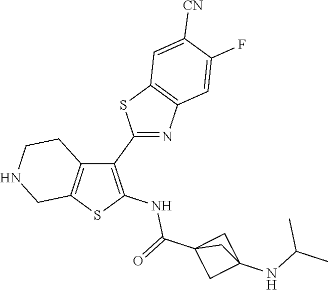 [SMILES: CC(C)NC12CC(C(=O)Nc3sc4c(c3-c3nc5cc(F)c(C#N)cc5s3)CCNC4)(C1)C2] | ESI-MS (M + H)⁺ = 482.1 |
| 114 | |
| 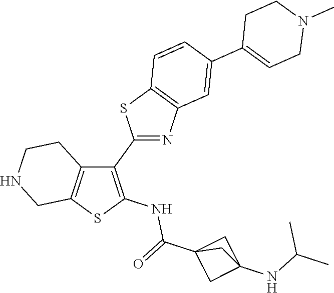 [SMILES: CC(C)NC12CC(C(=O)Nc3sc4c(c3-c3nc5cc(C6=CCN(C)CC6)ccc5s3)CCNC4)(C1)C2] | ESI-MS (M + H)⁺ = 534.2 |
| 115 | |
| 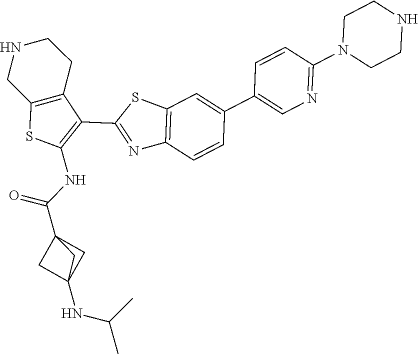 [SMILES: CC(C)NC12CC(C(=O)Nc3sc4c(c3-c3nc5ccc(-c6ccc(N7CCNCC7)nc6)cc5s3)CCNC4)(C1)C2] | ESI-MS (M + H)⁺ = 600.3 |
| 116 | |
| 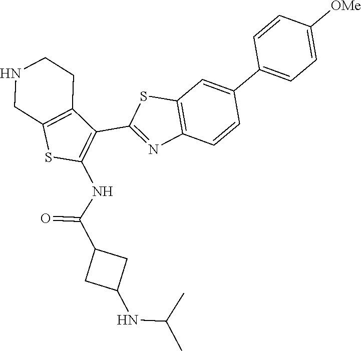 [SMILES: COc1ccc(-c2ccc3nc(-c4c(NC(=O)C5CC(NC(C)C)C5)sc5c4CCNC5)sc3c2)cc1] | ESI-MS (M + H)⁺ = 533.2 ¹H-NMR (400 MHz, DMSO-d₆) δ 9.70 (brs, 2H), 9.15 (brs, 1H), 8.44-8.45 (m, 1H), 8.13-8.18 (m, 1H), 7.85-7.89 (m, 1H), 7.72-7.74 (m, 2H), 7.06-7.09 (m, 2H), 4.32-4.37 (m, 2H), 3.82 (s, 3H), 3.66-3.71 (m, 1H), 3.42-3.50 (m, 3H), 3.18-3.29 (m, 3H), 2.54-2.72 (m, 4H), 1.21- 1.24 (m, 6H). |
| 118 | |
| 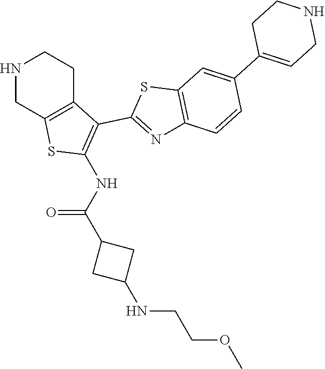 [SMILES: COCCNC1CC(C(=O)Nc2sc3c(c2-c2nc4ccc(C5=CCNCC5)cc4s2)CCNC3)C1] | ESI-MS (M + H)⁺ = 524.2 1H-NMR (400 MHz, DMSO-d₆) δ 9.22-9.73 (m, 4H), 8.29-8.31 (m, 1H), 8.09-8.14 (m, 1H), 7.74-7.78 (m, 1H), 6.35 (s, 1H), 4.32-4.33 (m, 2H), 3.79-3.80 (m, 2H), 3.56-3.59 (m, 2H), 3.45-3.49 (m, 3H), 3.16-3.28 (m, 8H), 3.02- 3.05 (m, 2H), 2.78-2.80 (m, 2H), 2.54-2.68 (m, 4H). |
| 119 | |
| 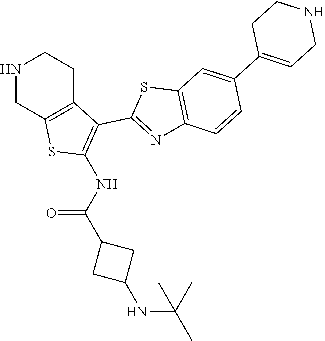 [SMILES: CC(C)(C)NC1CC(C(=O)Nc2sc3c(c2-c2nc4ccc(C5=CCNCC5)cc4s2)CCNC3)C1] | ESI-MS (M + H)⁺ = 522.2 ¹H-NMR (400 MHz, DMSO-d₆) δ 9.16-9.63 (m, 4H), 8.31-8.32 (m, 1H), 8.09-8.12 (m, 1H), 7.74-7.77 (m, 1H), 6.35-6.36 (m, 1H), 4.32- 4.33 (m, 2H), 3.78-3.79 (m, 2H), 3.64-3.71 (m, 1H), 3.45-3.49 (m, 4H), 3.15-3.21 (m, 3H), 2.58-2.79 (m, 6H), 1.27-1.28 (m, 9H). |
| 120 | |
| 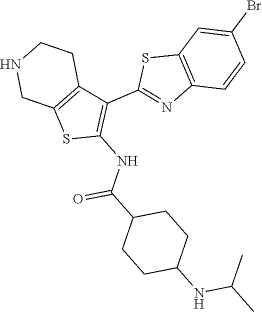 [SMILES: CC(C)NC1CCC(C(=O)Nc2sc3c(c2-c2nc4ccc(Br)cc4s2)CCNC3)CC1] | ESI-MS (M + H)⁺ = 533.1 ¹H-NMR (400 MHz, DMSO-d₆) δ 9.72-9.80 (m, 2H), 8.82 (s, 1H), 8.50-8.51 (m, 2H), 7.96-8.01 (m, 1H), 7.74-7.78 (m, 1H), 4.32-4.33 (m, 2H), 3.40-3.48 (m, 3H), 3.15-3.19 (m, 3H), 2.60- 3.02 (m, 1H), 2.14-2.35 (m, 3H), 1.80-2.01 (m, 2H), 1.54-1.60 (m, 2H), 1.21-1.28 (m, 6H). |
| 123 | |
| 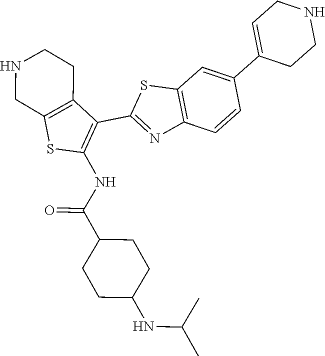 [SMILES: CC(C)NC1CCC(C(=O)Nc2sc3c(c2-c2nc4ccc(C5=CCNCC5)cc4s2)CCNC3)CC1] | ESI-MS (M + H)⁺ = 536.2 ¹H-NMR (400 MHz, DMSO-d₆) δ 9.40-9.83 (m, 3H), 8.55-8.89 (m, 1H), 8.31-8.32 (m, 1H), 8.00-8.05 (m, 1H), 7.74-7.78 (m, 1H), 6.35- 6.36 (m, 1H), 4.32-4.33 (m, 2H), 3.78-3.79 (m, 2H), 3.41-3.50 (m, 5H), 3.15-3.25 (m, 3H), 2.59-3.02 (m, 3H), 2.17-2.35 (m, 2H), 1.51- 2.01 (m, 6H), 1.18-1.28 (m, 6H). |
| 124 | |

### Example 29:3-(isopropylamino)-N-(3-(6-(4-methyl-1H-imidazol-1-yl)benzo[d]thiazol-2-yl)-4,5,6,7-tetrahydrothieno[2,3-c]pyridin-2-yl)cyclobutane-1-carboxamide (014)

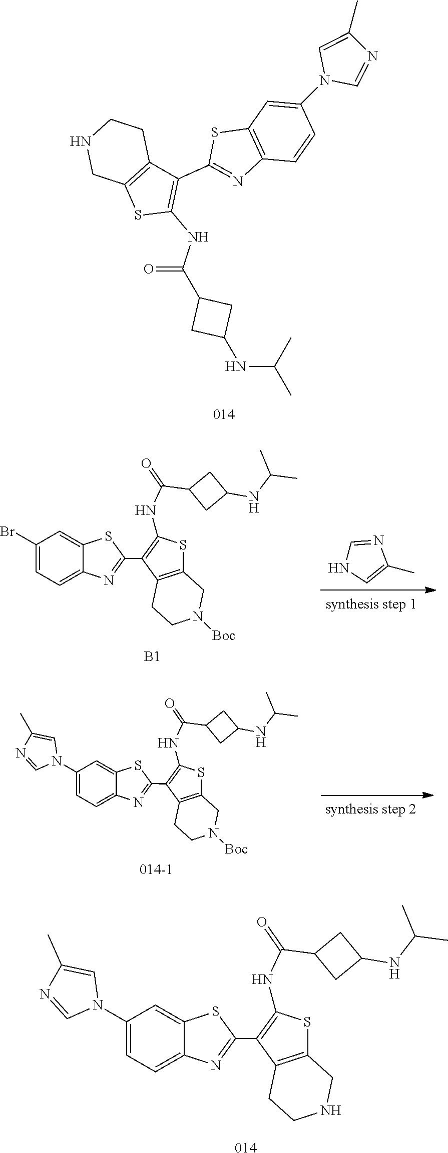

### Synthesis Step 1: tert-butyl 2-(3-(isopropylamino)cyclobutane-1-formylamino)-3-(6-(4-methyl-1H-imidazol-1-yl)benzo[d]thiazol-2-yl)-4,7-dihydrothieno[2,3-c]pyridin-6 (5H) carboxylate (014-1)

MeatBuXphos (95 mg, 0.20 mmol) and Pd₂(dba)₃ (180 mg, 0.20 mmol) were added into a mixed solution (dioxane: toluene=4:1, 15 mL) and reacted at 120° C. for 5 min. The intermediate B1 (600 mg, 0.99 mmol), 4-methyl-1H-imidazole (70 mg, 0.85 mmol) and K₃PO₄ (420 mg, 1.98 mmol) were added into the system in sequence, and the reaction was carried out at 120° C. for 4 h. After the reaction was completed, it was quenched by adding water, and the mixture was extracted three times with DCM. The organic phases were combined, dried over anhydrous sodium sulfate, concentrated under reduced pressure, and subjected to column chromatography to obtain 014-1 (480 mg) with a yield of 80%. ESI-MS (M+H)⁺607.7.

### Synthesis Step 2:3-(isopropylamino)-N-(3-(6-(4-methyl-1H-imidazol-1-yl)benzo[d]thiazol-2-yl)-4,5,6,7-tetrahydrothieno[2,3-c]pyridin-2-yl)cyclobutane-1-carboxamide (014)

014-1 (100 mg, 0.16 mmol) was dissolved in 5 mL of DCM, hydrogen chloride (dissolved in dioxane, 4M, 2 mL) was added into the system, and the reaction was carried out at room temperature for 1 h. After the reaction was completed, the mixture was concentrated under reduced pressure, adjusted to pH of weak alkalinity by adding saturated sodium bicarbonate aqueous solution, and extracted with dichloromethane. The organic phase was concentrated, then separated and purified by reverse-phase chromatography, and freeze-dried to obtain compound 014 (30 mg). ESI-MS (M+H)⁺507.6.

Referring to the synthetic route and method of Example 29, the following target compounds were synthesized:

| Structure of compound | Characterization data |
|---|---|
| 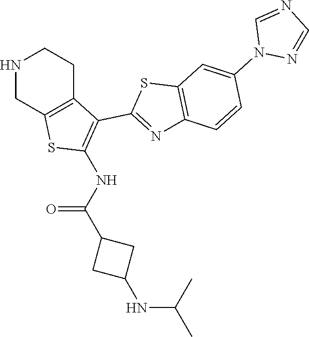 [SMILES: CC(C)NC1CC(C(=O)Nc2sc3c(c2-c2nc4ccc(-n5cncn5)cc4s2)CCNC3)C1] | ESI-MS (M + H)⁺ = 494.2 |
| 015 | |
| 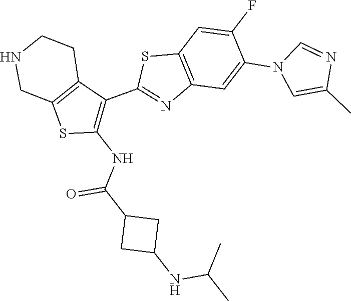 [SMILES: Cc1cn(-c2cc3nc(-c4c(NC(=O)C5CC(NC(C)C)C5)sc5c4CCNC5)sc3cc2F)cn1] | ESI-MS (M + H)⁺ = 525.2 |
| 020 | |
| 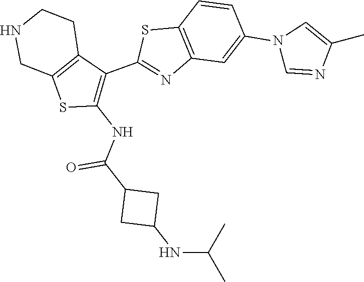 [SMILES: Cc1cn(-c2ccc3sc(-c4c(NC(=O)C5CC(NC(C)C)C5)sc5c4CCNC5)nc3c2)cn1] | ESI-MS (M + H)⁺ = 507.2 ¹H-NMR (400 MHz, DMSO-d₆) δ 9.52-9.91 (m, 3H), 8.29-8.46 (m, 3H), 7.82-7.88 (m, 1H), 4.32-4.33 (m, 2H), 3.78-3.82 (m, 1H), 3.47- 3.49 (m, 3H), 3.12-3.25 (m, 3H), 2.56-2.78 (m, 4H), 2.37 (s, 3H), 1.23-1.25 (m, 6H). |
| 121 | |

### Example 30: N-(6-isopropyl-3-(6-(1,2,3,6-tetrahydropyridin-4-yl)benzo[d]thiazol-2-yl)-4,5,6,7-tetrahydrothieno[2,3-c]pyridin-2-yl)-3-(isopropylamino)cyclobutane-1-carboxamide (051)

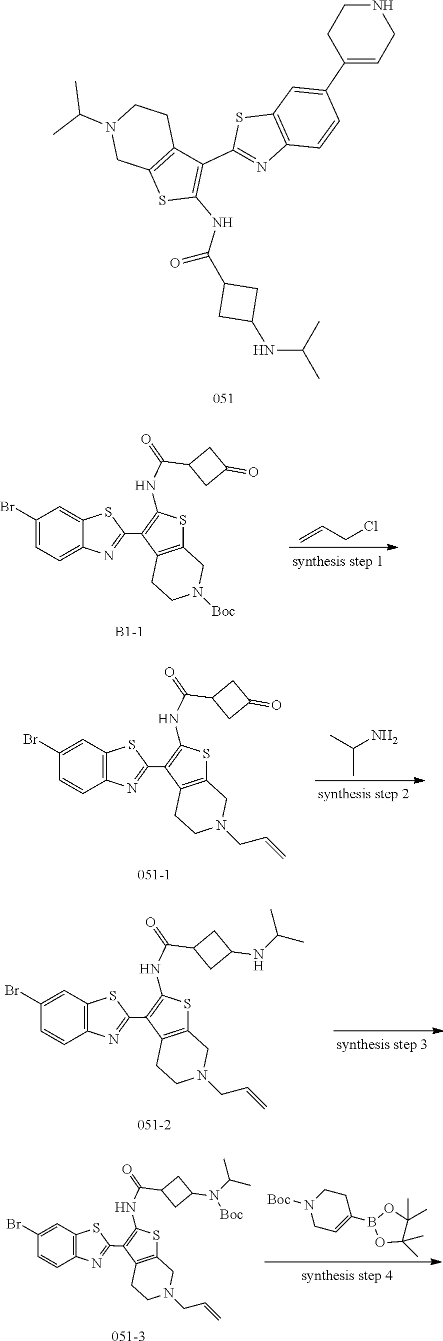

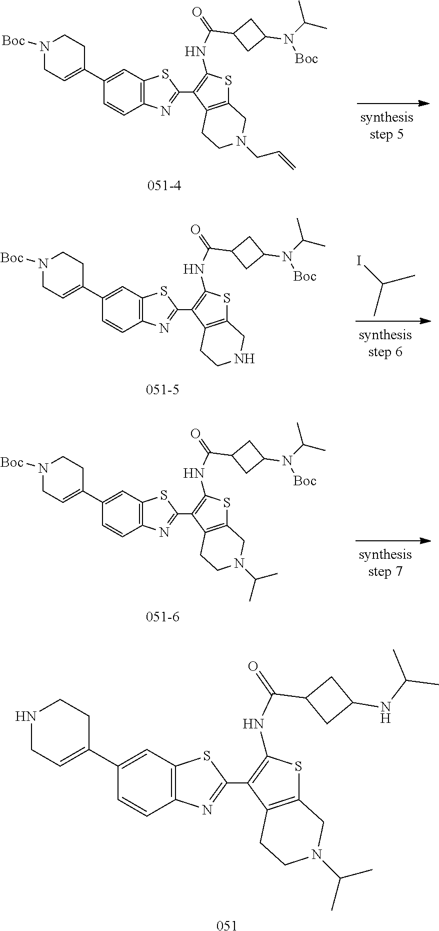

### Synthesis Step 1: N-(6-allyl-3-(6-bromobenzo[d]thiazol-2-yl)-4,5,6,7-tetrahydrothieno[2,3-c]pyridin-2-yl)-3-oxocyclobutane-1-carboxamide (051-1)

The intermediate B1-1 (5 g, 8.91 mmol) was dissolved in 50 mL of DCM, hydrogen chloride (dissolved in dioxane, 4M, 5 mL) was added to the system, and the reaction was carried out at room temperature for 1 h, concentrated under reduced pressure and directly used in the next step. The concentrate was dissolved in 40 mL of DCM, 3-chloropropyl-1-ene (988 mg, 13.00 mmol) and TEA (2.6 g, 26.01 mmol) were added into the system in sequence, and the reaction was carried out at room temperature for 1 h. After the reaction was completed, it was quenched by adding water, and the mixture was extracted three times with DCM. The organic phases was combined, dried over anhydrous sodium sulfate, and concentrated under reduced pressure to obtain 051-1 (4.1 g), with a yield of 94%, ESI-MS (M+H)⁺502.0.

### Synthesis Step 2: N-(6-allyl-3-(6-bromobenzo[d]thiazol-2-yl)-4,5,6,7-tetrahydrothieno[2,3-c]pyridin-2-yl)-3-(isopropylamino)cyclobutane-1-carboxamide (051-2)

051-1 (4.1 g, 8.18 mmol) was dissolved in 40 mL of DCE, isopropylamine (4.8 g, 81.83 mmol) and ice acetic acid (4.9 g, 81.83 mmol) were added into the system in sequence, and the reaction was carried out at 90° C. for 4 h. The system was cooled to room temperature, and added with STAB (4.3 g, 20.45 mmol), and the reaction was carried out at 90° C. for 2 h until the reaction was completed. The reaction was quenched by adding water, and extracted three times with DCM. The organic phases were combined, dried over anhydrous sodium sulfate, concentrated under reduced pressure, and subjected to column chromatography (DCM/MeOH) to obtain 051-2 (2 g), with a yield of 44%, ESI-MS (M+H)⁺545.1.

### Synthesis Step 3: tert-butyl (3-((6-allyl-3-(6-bromobenzo[d]thiazol-2-yl)-4,5,6,7-tetrahydrothieno[2,3-c]pyridin-2-yl) carbamoyl)cyclobutyl)(isopropyl)carbamate (051-3)

051-2 (2 g, 3.67 mmol) was dissolved in 20 mL of DCM, (Boc) 20 (1.6 g, 7.35 mmol) and TEA (1.1 g, 11.01 mmol) were added into the system in sequence, and the reaction was carried out at room temperature for 1 h. After the reaction was completed, it was quenched by adding water, and the mixture was extracted three times with DCM. The organic phases were combined, dried over anhydrous sodium sulfate, concentrated under reduced pressure, and subjected to column chromatography to obtain 051-3 (2.2 g) with a yield of 92%. ESI-MS (M+H)⁺645.2.

### Synthesis Step 4: tert-butyl 4-(2-(6-allyl-2-(3-((tert-butoxycarbonyl)(isopropyl) amino)cyclobutane-1-formamido)-4,5,6,7-tetrahydrothieno[2,3-c]pyridin-3-yl)benzo[d]thiazol-6-yl)-3,6-dihydropyridin-1 (2H)-carboxylate (051-4)

051-3 (2.2 g, 3.41 mmol) was added to the mixed solution (dioxane: water-3:1, 20 mL), tert-butyl 4-(4,4,5,5-tetramethyl-1,3,2-dioxaborolane-2-yl)-3,6-dihydropyridin-1 (2H)-carboxylate (1.6 g, 5.12 mmol), Pd(dppf)Cl₂ (248 mg, 0.34 mmol) and Na₂CO₃ (1.3 g, 11.23 mmol) were added into the system in sequence, and the reaction was carried out at 80° C. for 5 h. After the reaction was completed, it was quenched by adding water, and the mixture was extracted three times with DCM. The organic phases were combined, dried over anhydrous sodium sulfate, concentrated under reduced pressure and subjected to column chromatography to obtain 051-4 (2 g) with a yield of 78%. ESI-MS (M+H)⁺748.3.

### Synthesis Step 5: tert-butyl 4-(2-(2-(3-((tert-butoxycarbonyl)(isopropyl)amino)cyclobutane-1-carboxamido)-4,5,6,7-tetrahydrothieno[2,3-c]pyridin-3-yl)benzo[d]thiazol-6-yl)-3,6-dihydropyridin-1 (2H)-carboxylate (051-5)

051-4 (1 g, 1.34 mmol) was dissolved in 10 ml of 1,4-dioxane, Pd(PPh₃)₄ (154 mg, 0.01 mmol) and piperidine (227 mg, 2.67 mmol) were added into the system in sequence, and the reaction was carried out at 80° C. for 5 h until the completion. The reaction was quenched by adding water, the solvent was removed under reduced pressure, and the mixture was extracted three times with DCM. The organic phases were combined, dried over anhydrous sodium sulfate, concentrated under reduced pressure, and subjected to column chromatography to obtain 051-5 (500 mg) with a yield of 52%, ESI-MS (M+H)⁺708.3.

### Synthesis Step 6: tert-butyl 4-(2-(2-(3-((tert-butoxycarbonyl)(isopropyl)amino)cyclobutane-1-carboxamido)-6-isopropyl-4,5,6,7-tetrahydrothieno[2,3-c]pyridin-3-yl)benzo[d]thiazol-6-yl)-3,6-dihydropyridin-1 (2H)-carboxylate (051-6)

051-5 (200 mg, 0.28 mmol) was dissolved in 2 mL of DMF, potassium carbonate (120 mg, 0.84 mmol) and 2-iodopropane (70 mg, 0.42 mmol) were added into the system, and the reaction was completed at 80° C. for 1 h until the reaction was completed. The reaction was quenched by adding water and extracted three times with DCM. The organic phases were combined, concentrated under reduced pressure, and subjected to column chromatography to obtain 051-6 (120 mg) with a yield of 57%, ESI-MS (M+H)⁺750.4.

### Synthesis Step 7: N-(6-isopropyl-3-(6-(1,2,3,6-tetrahydropyridin-4-yl)benzo[d]thiazol-2-yl)-4,5,6,7-tetrahydrothieno[2,3-c]pyridin-2-yl)-3-(isopropylamino)cyclobutane-1-carboxamide (051)

051-6 (120 mg, 0.16 mmol) was dissolved in 2 mL of DCM, hydrogen chloride (dissolved in dioxane, 4M, 2 mL) was added to the system, and the reaction was carried out at room temperature for 1 h. After the reaction was completed, the mixture was concentrated under reduced pressure, adjusted to pH of weak alkalinity by adding saturated sodium bicarbonate aqueous solution, and extracted with dichloromethane. The organic phase was concentrated, then separated and purified by reverse-phase chromatography, and freeze-dried to obtain compound 051 (48 mg), ESI-MS (M+H)⁺550.3.

### Example 31: N-(6-isopropyl-3-(6-(6-(piperazin-1-yl)pyridin-3-yl)benzo[d]thiazol-2-yl)-4,5,6,7-tetrahydrothieno[2,3-c]pyridin-2-yl)-3-((2-methoxyethyl)amino)cyclobutane-1-carboxamide (043)

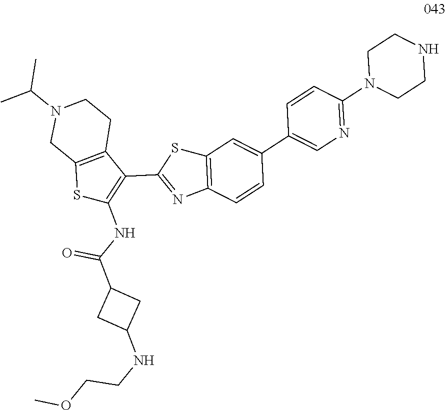 [SMILES: COCCNC1CC(C(=O)Nc2sc3c(c2-c2nc4ccc(-c5ccc(N6CCNCC6)nc5)cc4s2)CCN(C(C)C)C3)C1]

Referring to the synthesis route and method of Example 30, isopropyl amine in synthesis step 2 was replaced with 2-methoxyethane-1-amine, and tert-butyl 4-(4,4,5,5-tetramethyl-1,3,2-dioxaborolane-2-yl)-3,6-dihydropyridin-1 (2H)-carboxylate in synthesis 4 step was replaced with tert-butyl 4-(5-(4,4,5,5-tetramethyl-1,3,2-dioxaborolane-2-yl)pyridin-2-yl) piperazin-1-carboxylate, to synthesize compound 043. ESI-MS (M+H)⁺646.3.

### Example 32 3-(isopropylamino)-N-(6-(oxetane-3-yl)-3-(6-(1,2,3,6-tetrahydropyridin-4-yl)benzo[d]thiazol-2-yl)-4,5,6,7-tetrahydrothieno[2,3-c]pyridin-2-yl)cyclobutane-1-carboxamide (052)

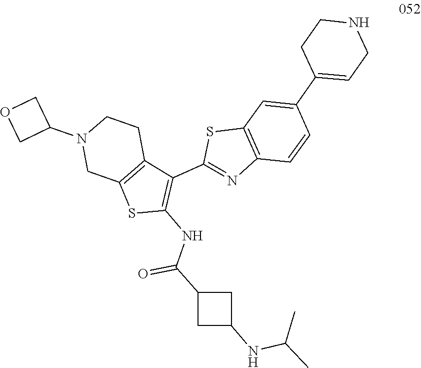 [SMILES: CC(C)NC1CC(C(=O)Nc2sc3c(c2-c2nc4ccc(C5=CCNCC5)cc4s2)CCN(C2COC2)C3)C1]

Referring to the synthesis route and method of Example 30, in synthesis step 6, 2-iodopropane was replaced with oxetane-3-one to synthesize compound 052. ESI-MS (M+H)⁺564.2.

Referring to the synthetic routes and methods of Examples 30 to 32, the following target compounds were synthesized:

| Structure of compound | Characterization data |
|---|---|
| 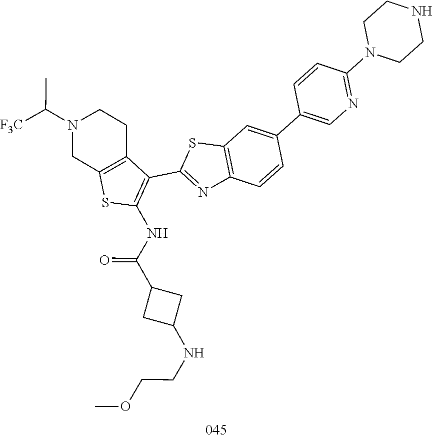 [SMILES: COCCNC1CC(C(=O)Nc2sc3c(c2-c2nc4ccc(-c5ccc(N6CCNCC6)nc5)cc4s2)CCN(C(C)C(F)(F)F)C3)C1] | ESI-MS (M + H)⁺ = 700.3 |
| 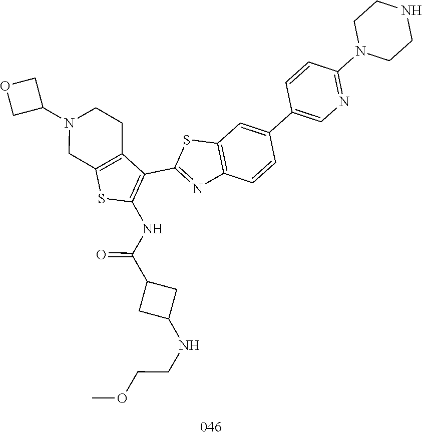 [SMILES: COCCNC1CC(C(=O)Nc2sc3c(c2-c2nc4ccc(-c5ccc(N6CCNCC6)nc5)cc4s2)CCN(C2COC2)C3)C1] | ESI-MS (M + H)⁺ = 660.3 |
| 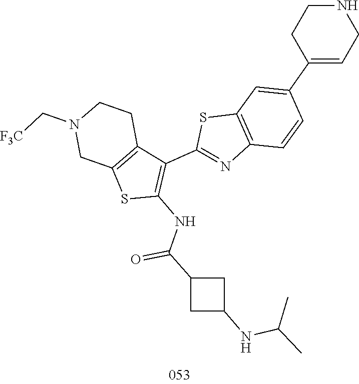 [SMILES: CC(C)NC1CC(C(=O)Nc2sc3c(c2-c2nc4ccc(C5=CCNCC5)cc4s2)CCN(CC(F)(F)F)C3)C1] | ESI-MS (M + H)⁺ = 590.2 ¹H-NMR (400 MHz, DMSO-d₆) δ 9.18-9.38 (m, 3H), 8.29-8.30 (m, 1H), 8.06-8.08 (m, 1H), 7.73-7.76 (m, 1H), 6.34-6.36 (m, 1H), 3.86- 3.88 (m, 4H), 3.32-3.49 (m, 5H), 3.08-3.10 (m, 2H), 2.96-2.98 (m, 3H), 2.78-2.80 (m, 3H), 2.56-2.70 (m, 4H), 1.21-1.23 (m, 6H). |
| 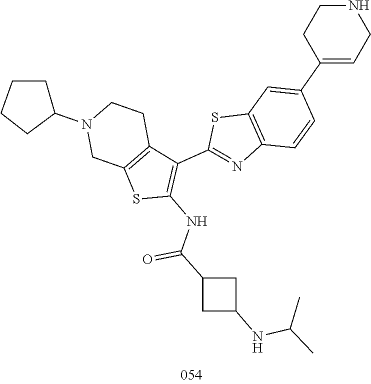 [SMILES: CC(C)NC1CC(C(=O)Nc2sc3c(c2-c2nc4ccc(C5=CCNCC5)cc4s2)CCN(C2CCCC2)C3)C1] | ESI-MS (M + H)⁺ = 576.3 ¹H-NMR (400 MHz, DMSO-d₆) δ 9.13-9.36 (m, 3H), 8.32-8.33 (m, 1H), 8.09-8.13 (m, 1H), 7.74-7.79 (m, 1H), 6.35-6.36 (m, 1H), 4.61- 4.65 (m, 1H), 4.32-4.38 (m, 1H), 3.66-3.85 (m, 6H), 3.23-3.30 (m, 6H), 2.54-2.79 (m, 6H), 1.86-2.15 (m, 4H), 1.56-1.78 (m, 4H), 1.21- 1.23 (m, 6H). |
| 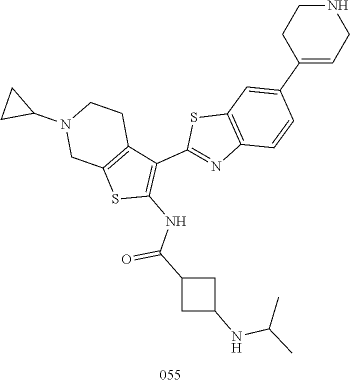 [SMILES: CC(C)NC1CC(C(=O)Nc2sc3c(c2-c2nc4ccc(C5=CCNCC5)cc4s2)CCN(C2CC2)C3)C1] | ESI-MS (M + H)⁺ = 548.2 |
| 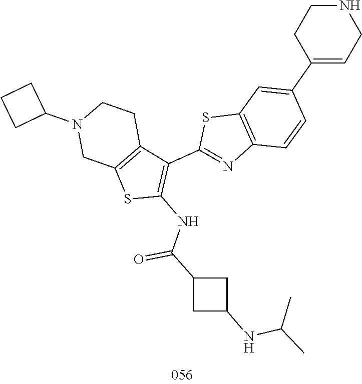 [SMILES: CC(C)NC1CC(C(=O)Nc2sc3c(c2-c2nc4ccc(C5=CCNCC5)cc4s2)CCN(C2CCC2)C3)C1] | ESI-MS (M + H)⁺ = 562.3 |
| 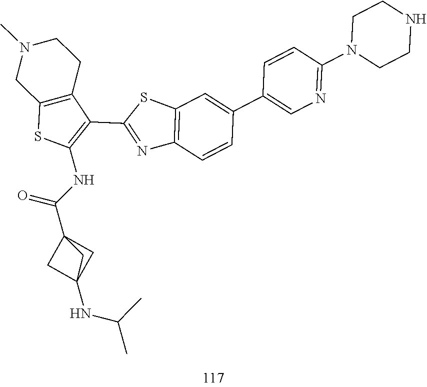 [SMILES: CC(C)NC12CC(C(=O)Nc3sc4c(c3-c3nc5ccc(-c6ccc(N7CCNCC7)nc6)cc5s3)CCN(C)C4)(C1)C2] | ESI-MS (M + H)⁺ = 614.3 |
| 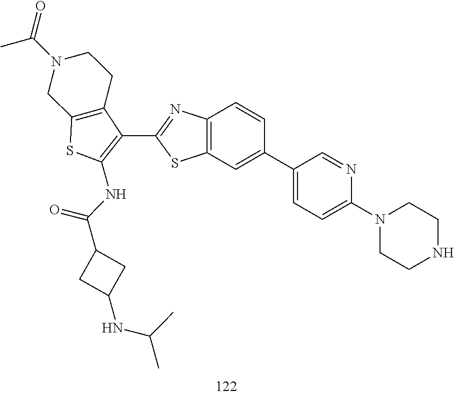 [SMILES: CC(=O)N1CCc2c(sc(NC(=O)C3CC(NC(C)C)C3)c2-c2nc3ccc(-c4ccc(N5CCNCC5)nc4)cc3s2)C1] | ESI-MS (M + H)⁺ = 630.3 ¹H-NMR (400 MHz, DMSO-d₆) δ 9.24-9.48 (m, 3H), 8.55-8.56 (m, 1H), 8.47-8.48 (m, 1H), 8.10-8.18 (m, 2H), 7.87-7.89 (m, 1H), 7.18- 7.21 (m, 1H), 4.63-4.69 (m, 2H), 3.81-3.90 (m, 7H), 3.19-3.38 (m, 6H), 2.92-3.04 (m, 2H), 2.55-2.71 (m, 4H), 2.09-2.15 (m, 3H), 1.21- 1.23 (m, 6H). |

### Example 33: N-(3-(benzo[d]thiazol-2-yl)-4,5,6,7-tetrahydrothieno[2,3-c]pyridin-2-yl)-4-((2-(isopropylamino)ethyl)amino)benzamide (072)

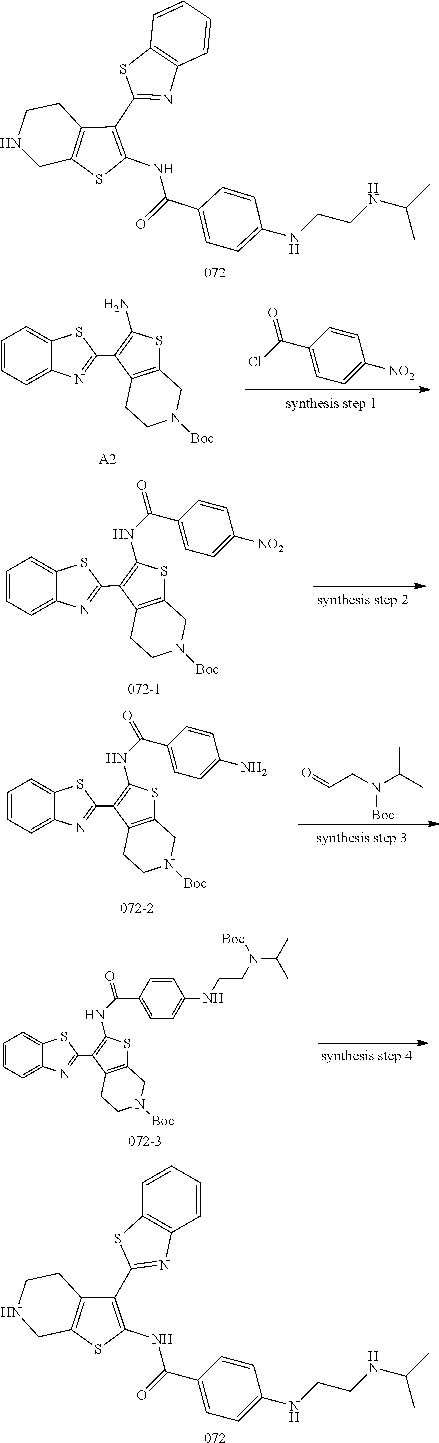

### Synthesis Step 1: tert-butyl 3-(benzo[d]thiazol-2-yl)-2-(4-nitroformamidyl)-4,7-dihydrothieno[2,3-c]pyridin-6 (5H)-carboxylate (072-1)

The intermediate A2 (5.0 g, 12.92 mmol) was dissolved in 25 mL of DCM, 4-nitrobenzoyl chloride (3.6 g, 19.38 mmol) and triethylamine (3.5 mL) were added into the system in sequence, and the reaction was carried out at room temperature for 1 h. After the reaction was completed, it was quenched by adding saturated ammonium chloride aqueous solution and the mixture was extracted three times with DCM. The organic phases were combined, dried over anhydrous sodium sulfate, concentrated under reduced pressure, and subjected to column chromatography to obtain 072-1 (4.5 g) with a yield of 65%. ESI-MS (M+H)⁺537.1.

### Synthesis Step 2: tert-butyl 2-(4-aminobenzoylamino)-3-(benzo[d]thiazol-2-yl)-4,7-dihydrothieno[2,3-c]pyridin-6 (5H)-carboxylate (072-2)

072-1 (4.5 g, 8.39 mmol) was dissolved in 20 mL of THF, ammonium chloride (4.5 g, 83.95 mmol), iron powder (4.7 g, 83.95 mmol), H₂O (5 mL) and tetrahydrofuran (15 mL) were added into the system in sequence, and the reaction was carried out at 60° C. for 5 h. After the reaction was completed, the mixture was filtered while being hot, concentrated under reduced pressure, and subjected to column chromatography to obtain 072-2 (4.1 g) with a yield of 95%. ESI-MS (M+H)⁺507.6.

### Synthesis Step 3: tert-butyl 3-(benzo[d]thiazol-2-yl)-2-(4-((2-((tert-butoxycarbonyl) (isopropyl)amino)ethyl)amino)benzamido)-4,7-dihydrothieno[2,3-c]pyridin-6 (5H)-carboxylate (072-3)

072-2 (1.0 g, 1.98 mmol) was dissolved in 10 mL DCE, tert-butyl isopropyl(2-oxoethyl)carbamate (600 mg, 2.97 mmol) and acetic acid (315 μL, 1.98 mmol) were added to the system in sequence, the reaction was carried out at 80° C. for 2 h, then sodium borohydride (150 mg, 3.96 mmol) was added, and the reaction was continued at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure, and subjected to column chromatography (eluted with DCM/MeOH) to obtain 072-3 (820 mg) with a yield of 63%. ESI-MS (M+H)⁺692.3.

### Synthesis Step 4: N-(3-(benzo[d]thiazol-2-yl)-4,5,6,7-tetrahydrothieno[2,3-c]pyridin-2-yl)-4-((2-(isopropylamino)ethyl)amino)benzamide (072)

Referring to the synthetic route and method of Example 25, intermediate B5 was replaced with 072-3 to synthesize the target compound 072. ESI-MS (M+H)⁺492.2.

### Example 34 4-(isopropylamino)-N-(3-(6-(6-(piperazin-1-yl)pyridin-3-yl)benzo[d]thiazol-2-yl)-4,5,6,7-tetrahydrothieno[2,3-c]pyridin-2-yl)benzamide (071)

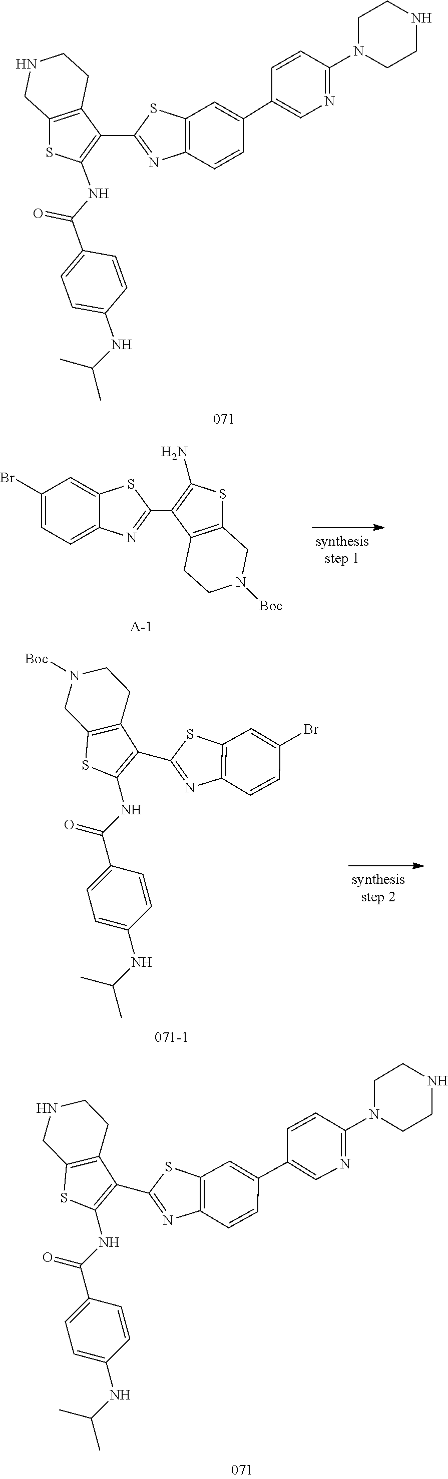 [SMILES: CC(C)Nc1ccc(C(=O)Nc2sc3c(c2-c2nc4ccc(-c5ccc(N6CCNCC6)nc5)cc4s2)CCNC3)cc1 ; CC(C)(C)OC(=O)N1CCc2c(sc(N)c2-c2nc3ccc(Br)cc3s2)C1 ; "A-1" ; CC(C)Nc1ccc(C(=O)Nc2sc3c(c2-c2nc4ccc(Br)cc4s2)CCN(C(=O)OC(C)(C)C)C3)cc1 ; CC(C)Nc1ccc(C(=O)Nc2sc3c(c2-c2nc4ccc(-c5ccc(N6CCNCC6)nc5)cc4s2)CCNC3)cc1]

### Synthesis step 1: tert-butyl 3-(6-bromobenzo[d]thiazol-2-yl)-2-(4-(isopropylamino)benzamido)-4,7-dihydrothieno[2,3-c]pyridin-6 (5H)-carboxylate (071-1)

Referring to the synthetic route and method of synthesis steps 1 to 3 in Example 33, intermediate A2 was replaced with intermediate A1 in synthesis step 1, and tert-butyl isopropyl(2-oxoethyl)carbamate was replaced with propyl-2-one in synthesis step 3, to synthesize 071-1, ESI-MS (M+H)⁺627.1.

### Synthesis Step 2:4-(isopropylamino)-N-(3-(6-(6-(piperazin-1-yl)pyridin-3-yl)benzo[d]thiazol-2-yl)-4,5,6,7-dihydrothieno[2,3-c]pyridin-2-yl)benzamide (071)

Referring to the synthesis route and method of Example 26, in synthesis step 1, intermediate B1 replaced was with 071-1, tert-butyl 4-(4,4,5,5-tetramethyl-1,3,2-dioxaborolane-2-yl)-3,6-dihydropyridin-1 (2H)-carboxylate was replaced with tert-butyl 4-(5-(4,4,5,5-tetramethyl-1,3,2-dioxaborolane-2-yl)pyridin-2-yl) piperazin-1-carboxylate to synthesize the target compound 071. ESI-MS (M+H)⁺710.3.

Referring to the synthetic routes and methods of Examples 33 to 34, the following target compounds were synthesized:

| Structure of compound | Characterization data |
|---|---|
| 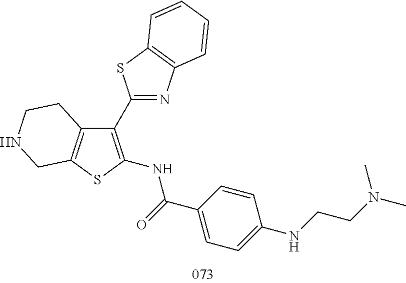 [SMILES: CN(C)CCNc1ccc(C(=O)Nc2sc3c(c2-c2nc4ccccc4s2)CCNC3)cc1] | ESI-MS (M + H)⁺ = 478.2 |
| 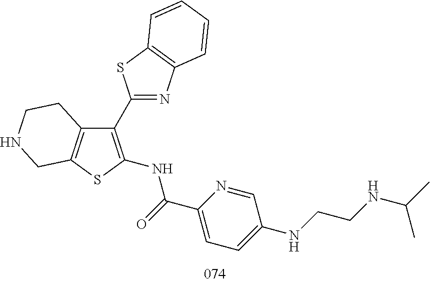 [SMILES: CC(C)NCCNc1ccc(C(=O)Nc2sc3c(c2-c2nc4ccccc4s2)CCNC3)nc1] | ESI-MS (M + H)⁺ = 493.2 |
| 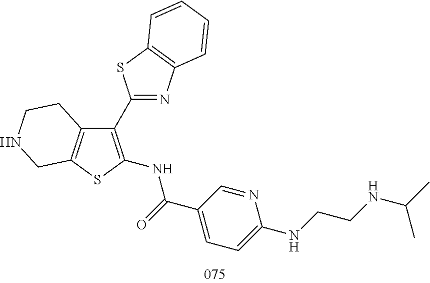 [SMILES: CC(C)NCCNc1ccc(C(=O)Nc2sc3c(c2-c2nc4ccccc4s2)CCNC3)cn1] | ESI-MS (M + H)⁺ = 493.2 |
| 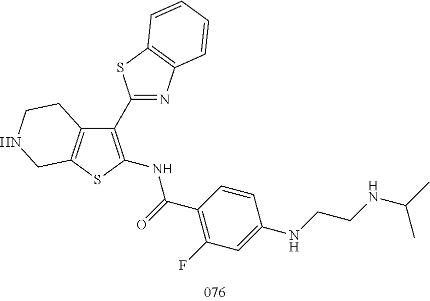 [SMILES: CC(C)NCCNc1ccc(C(=O)Nc2sc3c(c2-c2nc4ccccc4s2)CCNC3)c(F)c1] | ESI-MS (M + H)⁺ = 510.2 |

### Example 35: N-(3-(benzo[d]thiazol-2-yl)-4,5,6,7-tetrahydrothieno[2,3-c]pyridin-2-yl)-4-(isopropylamino)-2-oxobutanamide (093)

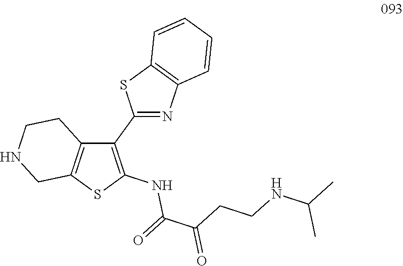 [SMILES: CC(C)NCCC(=O)C(=O)Nc1sc2c(c1-c1nc3ccccc3s1)CCNC2]

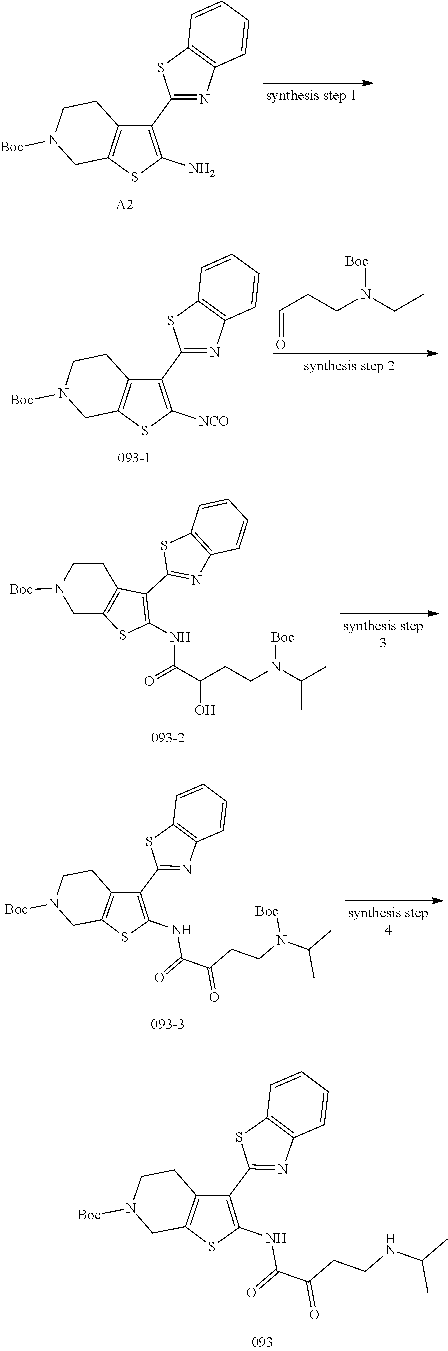

### Synthesis Step 1: tert-butyl 3-(benzo[d]thiazol-2-yl)-2-isocyanate-4,7-dihydrothieno[2,3-c]pyridin-6 (5H)-carboxylate (093-1)

The intermediate A2 (2 g, 4.30 mmol) was dissolved in 20 mL of ethyl acetate, triphosgene (0.8 g, 2.69 mmol) was introduced into the system at low temperature, and the reaction was carried out at 80° C. for 1 h. After the reaction was completed, it was concentrated under reduced pressure to obtain 093-1 (1.8 g) with a yield of 99%, ESI-MS (M+H)⁺414.1.

### Synthesis Step 2: tert-butyl 3-(benzo[d]thiazol-2-yl)-2-(4-((tert-butoxycarbonyl) (isopropyl)amino)-2-hydroxybutanamido)-4,7-dihydrothieno[2,3-c]pyridin-6 (5H)-carboxylate (093-2)

093-1 (1.1 g, 3.25 mmol) was dissolved in 20 mL of DCM, chloroacetic acid (337 mg, 3.57 mmol) and tert-butyl isopropyl (3-oxopropyl)carbamate (768 mg, 3.57 mmol) were added to the system in sequence at 0° C., and the reaction was carried out at room temperature for 12 h, and a solid precipitated. The solid was dissolved in a mixed solvent (MeOH: H₂O=1:1, 10 mL), potassium carbonate (1.1 g, 8.12 mmol) was added to the system, and the reaction was carried out at room temperature for 5 h. After the reaction was completed, methanol was removed under reduced pressure, and the mixture was extracted three times with EA. The organic phases were combined, dried over anhydrous sodium sulfate and concentrated under reduced pressure to obtain 093-2 (1.07 g), with a yield of 52%, ESI-MS (M+H)⁺631.3.

### Synthesis Step 3: tert-butyl 3-(benzo[d]thiazol-2-yl)-2-(4-((tert-butoxycarbonyl) (isopropyl)amino)-2-oxobutanamido)-4,7-dihydrothieno[2,3-c]pyridin-6 (5H)-carboxylate (093-3)

093-2 (1.07 g, 1.7 mmol) was dissolved in 15 mL of DCM, DMP(1 g, 2.54 mmol) was added to the system, and the reaction was carried out at room temperature for 1 h. After the reaction was completed, it was quenched by adding water. The mixture was extracted three times with DCM, and the organic phases were combined, dried over anhydrous sodium sulfate, concentrated under reduced pressure, and subjected to column chromatography to obtain 093-3 (716 mg) with a yield of 67%, ESI-MS (M+H)⁺629.2.

### Synthesis Step 4: N-(3-(benzo[d]thiazol-2-yl)-4,5,6,7-tetrahydrothieno[2,3-c]pyridin-2-yl)-4-(isopropylamino)-2-oxobutanamide (093)

093-3 (88 mg, 0.14 mmol) was dissolved in 2 mL of DCM, hydrogen chloride (dissolved in dioxane, 4M, 1 mL) was added to the system, and the reaction was carried out at room temperature for 1 h. After the reaction was completed, the mixture was concentrated under reduced pressure, adjusted to a pH of weak alkalinity by adding saturated sodium bicarbonate aqueous solution, and extracted with dichloromethane. The organic phase was concentrated, then separated and purified by reverse-phase chromatography, and freeze-dried to obtain compound 093 (38 mg). ESI-MS (M+H)⁺429.1.

Referring to the synthetic route and method of Example 35, the following target compounds were synthesized:

| Structure of compound | Characterization data |
|---|---|
| 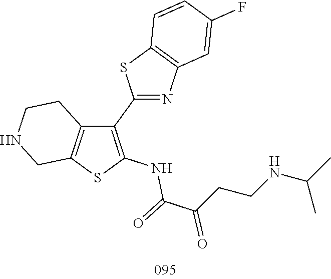 [SMILES: CC(C)NCCC(=O)C(=O)Nc1sc2c(c1-c1nc3cc(F)ccc3s1)CCNC2] | ESI-MS (M + H)⁺ = 447.1 |
| 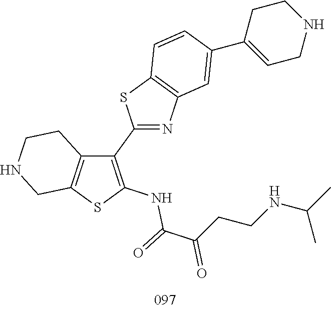 [SMILES: CC(C)NCCC(=O)C(=O)Nc1sc2c(c1-c1nc3cc(C4=CCNCC4)ccc3s1)CCNC2] | ESI-MS (M + H)⁺ = 510.2 |

### Example 36: N¹-(3-(benzo[d]thiazol-2-yl)-4,5,6,7-tetrahydrothieno[2,3-c]pyridin-2-yl)-N²-(2-(isopropylamino)ethyl)oxalamide (094)

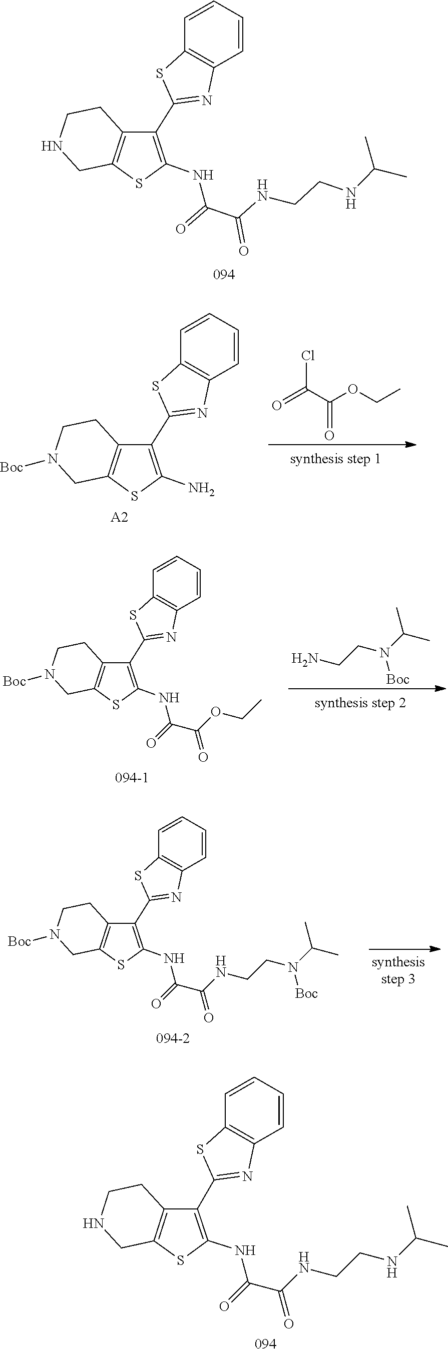

### Synthesis Step 1: tert-butyl 3-(benzo[d]thiazol-2-yl)-2-(2-ethoxy-2-oxoacetylamino)-4,7-dihydrothieno[2,3-c]pyridin-6 (5H)-carboxylate (094-1)

The intermediate A2 (120 mg, 0.31 mmol) was dissolved in 5 mL of DCM, ethyl 2-chloro-2-oxoacetate (110 mg, 0.77 mmol) and TEA (100 mg, 0.93 mmol) were added into the system in sequence, and the reaction was carried out at room temperature for 1 h. After the reaction was completed, it was quenched by adding saturated ammonium chloride aqueous solution, and the mixture was extracted three times with DCM. The organic phases were combined, dried over anhydrous sodium sulfate, concentrated under reduced pressure and subjected to column chromatography (eluted with DCM/MeOH) to obtain 094-1 (140 mg) with a yield of 92%. ESI-MS (M+H)⁺488.1.

### Synthesis Step 2: tert-butyl 3-(benzo[d]thiazol-2-yl)-2-(2-((2-((tert-butoxycarbonyl) (isopropyl)amino)ethyl)amino)-2-oxoacetamido)-4,7-dihydrothieno[2,3-c]pyridin-6 (5H)-carb oxylate (094-2)

094-1 (140 mg, 0.28 mmol) was dissolved in 5 mL of THE, tert-butyl (2-aminoethyl)(isopropyl)carbamate (70 mg, 0.33 mmol) and 1,5,7-triazabicyclo[4.4.0]dec-5-ene (TBD, 40 mg, 0.28 mmol) were added into the system in sequence, and the reaction was carried out at 50° C. for 10 h. After the reaction was completed, it was quenched by adding water, and the mixture was extracted three times with DCM. The organic phases were combined, dried over anhydrous sodium sulfate, concentrated under reduced pressure, and subjected to column chromatography (eluted with EA/PE) to obtain 094-2 (130 mg) with a yield of 70%. ESI-MS (M+H)⁺644.3.

### Synthesis step 3: N¹-(3-(benzo[d]thiazol-2-yl)-4,5,6,7-tetrahydrothieno[2,3-c]pyridin-2-yl)-N₂-(2-(isopropylamino)ethyl) oxalamide (094)

094-2 (170 mg, 0.26 mmol) was dissolved in 5 mL of DCM, hydrogen chloride (dissolved in dioxane, 4M, 2 mL) was added into the system, and the reaction was carried out at room temperature for 1 h. After the reaction was completed, the mixture was concentrated under reduced pressure, adjusted to the pH of weak alkalinity by adding saturated sodium bicarbonate aqueous solution, and extracted with dichloromethane. The organic phase was concentrated, then separated and purified by reverse-phase chromatography, and freeze-dried to obtain compound 094 (75 mg). ESI-MS (M+H)⁺444.1.

Referring to the synthetic route and method of Example 36, the following target compounds were synthesized:

| Structure of compound | Characterization data |
|---|---|
| 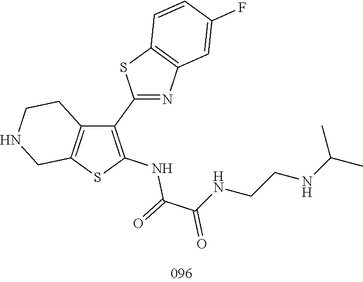 [SMILES: CC(C)NCCNC(=O)C(=O)Nc1sc2c(c1-c1nc3cc(F)ccc3s1)CCNC2] | ESI-MS (M + H)⁺ = 462.1 |
| 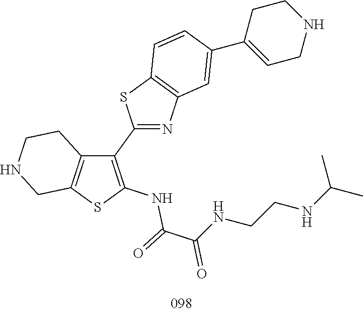 [SMILES: CC(C)NCCNC(=O)C(=O)Nc1sc2c(c1-c1nc3cc(C4=CCNCC4)ccc3s1)CCNC2] | ESI-MS (M + H)⁺ = 525.2 |
| 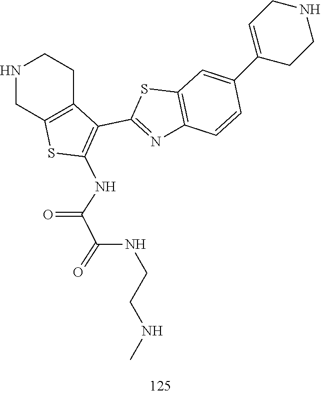 [SMILES: CNCCNC(=O)C(=O)Nc1sc2c(c1-c1nc3ccc(C4=CCNCC4)cc3s1)CCNC2] | ESI-MS (M + H)⁺ = 497.2 ¹H-NMR (400 MHz, DMSO-d₆) δ 9.83 (brs, 1H), 9.35-9.38 (m, 3H), 8.81 (brs, 1H), 8.32-8.33 (m, 1H), 7.96-7.99 (m, 1H), 7.80-7.82 (m, 1H), 6.34-6.36 (m, 1H), 4.36-4.37 (m, 2H), 3.78- 3.79 (m, 2H), 3.57-3.59 (m, 2H), 3.50-3.52 (m, 2H), 3.10-3.24 (m, 6H), 2.78-2.79 (m, 2H), 2.56-2.59 (m, 3H). |

### Example 37: N-(3-(benzo[d]thiazol-2-yl)-4,5,6,7-tetrahydrothieno[3,2-c]pyridin-2-yl)-3-(isopropylamino)cyclobutane-1-carboxamide (099)

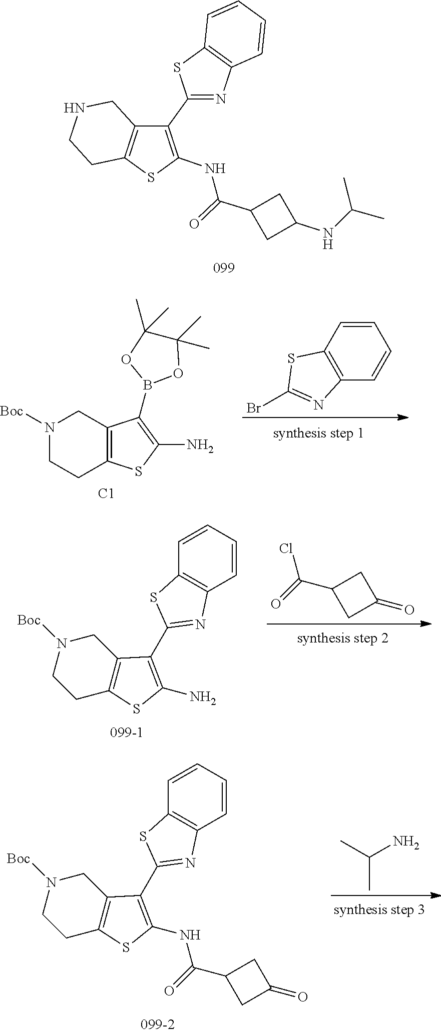

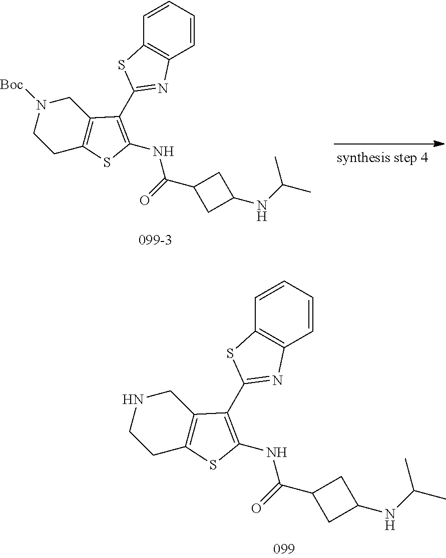 [SMILES: CC(C)NC1CC(C(=O)Nc2sc3c(c2-c2nc4ccccc4s2)CN(C(=O)OC(C)(C)C)CC3)C1 ; CC(C)NC1CC(C(=O)Nc2sc3c(c2-c2nc4ccccc4s2)CNCC3)C1]

### Synthesis Step 1: tert-butyl 2-amino-3-(benzo[d]thiazol-2-yl)-6,7-dihydrothieno[3,2-c]pyridin-5 (4H)-carboxylate (099-1)

The intermediate C₁ (300 mg, 0.789 mmol) was dissolved in 15 mL of dioxane, 2-bromobenzo[d]thiazole (168 mg, 0.789 mmol), sodium carbonate (125 mg, 1.18 mmol), Pd(dppf)Cl₂ (58 mg, 0.08 mmol) and water (3 mL) were added in sequence, and the reaction was carried out at 100° C. for 4 h. After the reaction was completed, the mixture was cooled to room temperature, and dioxane was removed by concentration. An appropriate amount of water was added, and the mixture was extracted with EA. The organic phases were combined, dried and concentrated, and the crude product was purified by column chromatography to obtain 099-1 (226 mg) with a yield of 74%, ESI-MS (M+H)⁺388.1.

### Synthesis Step 2: tert-butyl 3-(benzo[d]thiazol-2-yl)-2-(3-oxocyclobutane-1-carboxamido)-6,7-dihydrothieno[3,2-c]pyridin-5 (4H)-carboxylate (099-2)

099-1 (249 mg, 0.644 mmol) was dissolved in 10 mL of DCM, TEA (98 mg, 0.966 mmol) and 3-oxocyclobutane-1-carbonyl chloride (111 mg, 0.837 mmol) were added into the system in sequence, and the reaction was carried out at room temperature for 1 h. After the reaction was completed, it was quenched by adding saturated ammonium chloride aqueous solution and the mixture was extracted three times with DCM. The organic phases were combined, dried over anhydrous sodium sulfate, concentrated under reduced pressure, and subjected to column chromatography (eluted with DCM/MeOH) to obtain 099-2 (246 mg) with a yield of 79%. ESI-MS (M+H)⁺484.1.

### Synthesis Step 3: tert-butyl 3-(benzo[d]thiazol-2-yl)-2-(3-(isopropylamino)cyclobutane-1-carboxamido)-6,7-dihydrothieno[3,2-c]pyridin-5 (4H)-carboxylate (099-3)

099-2 (046 mg, 0.509 mmol) was dissolved in 5 mL of DCE, isopropylamine (300 mg, 5.09 mmol) and acetic acid (305 mg, 5.09 mmol) were added into the system in sequence, and the reaction was carried out at 80° C. for 2 h. Then sodium triacetoxyborohydride (STAB, 323 mg, 1.53 mmol) was added, and the reaction was continued at 80° C. for 1 h. After the reaction was completed, it was quenched by adding saturated sodium bicarbonate aqueous solution, and the mixture was extracted three times with DCM. The organic phases were combined, dried over anhydrous sodium sulfate, concentrated under reduced pressure and subjected to column chromatography (eluted with DCM/MeOH) to obtain 099-3 (150 mg) with a yield of 56%. ESI-MS (M+H)⁺527.2.

### Synthesis Step 4: N-(3-(benzo[d]thiazol-2-yl)-4,5,6,7-tetrahydrothieno[3,2-c]pyridin-2-yl)-3-(isopropylamino)cyclobutane-1-carboxamide (099)

099-3 (150 mg, 0.285 mmol) was dissolved in 5 mL of dioxane, 4 mol/L hydrogen chloride (dissolved in dioxane, 4M, 3 mL) was added into the system, and the reaction was carried out at room temperature for 14 h. After the reaction was completed, the mixture was concentrated under reduced pressure, adjusted to a pH of weak alkalinity by adding saturated sodium bicarbonate aqueous solution, and extracted with dichloromethane. The organic phase was concentrated, then separated and purified by reverse-phase chromatography, and freeze-dried to obtain compound 099 (70 mg) with a yield of 58%. ESI-MS (M+H)⁺427.2.

### Example 38:3-(isopropylamino)-N-(3-(6-(6-(piperazin-1-yl)pyridin-3-yl)benzo[d]thiazol-2-yl)-4,5,6,7-tetrahydrothieno[3,2-c]pyridin-2-yl)cyclobutane-1-carboxamide (102)

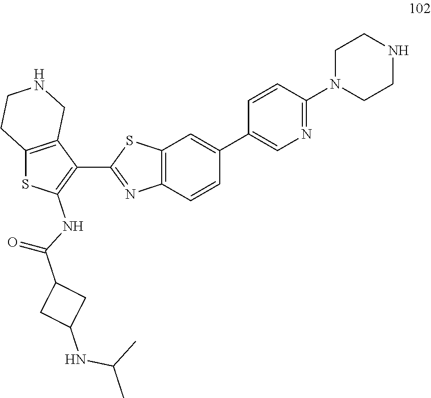 [SMILES: CC(C)NC1CC(C(=O)Nc2sc3c(c2-c2nc4ccc(-c5ccc(N6CCNCC6)nc5)cc4s2)CNCC3)C1]

Referring to the synthesis route and method of Example 37, in the synthesis step 1, 2-bromobenzo[d]thiazole was replaced with intermediate D1 to synthesize and obtain the compound 102. ESI-MS (M+H)⁺588.3.

Referring to the synthetic routes and methods of Examples 37 to 38, the following target compounds were synthesized and obtained:

| Structure of compound | Characterization data |
|---|---|
| 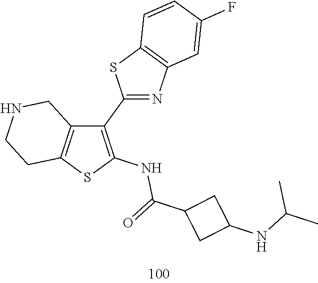 [SMILES: CC(C)NC1CC(C(=O)Nc2sc3c(c2-c2nc4cc(F)ccc4s2)CNCC3)C1] | ESI-MS (M + H)⁺ = 445.1 |
| 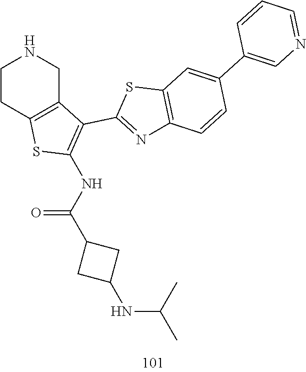 [SMILES: CC(C)NC1CC(C(=O)Nc2sc3c(c2-c2nc4ccc(-c5cccnc5)cc4s2)CNCC3)C1] | ESI-MS (M + H)⁺ = 504.2 |
| 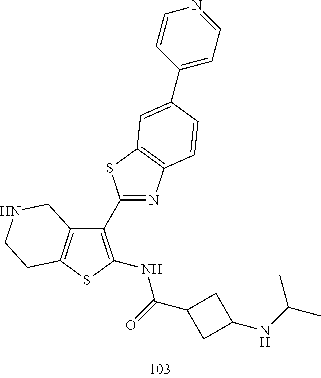 [SMILES: CC(C)NC1CC(C(=O)Nc2sc3c(c2-c2nc4ccc(-c5ccncc5)cc4s2)CNCC3)C1] | ESI-MS (M + H)⁺ = 504.2 |
| 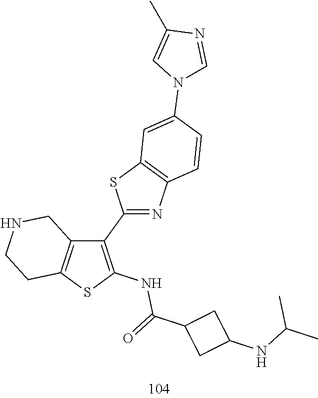 [SMILES: Cc1cn(-c2ccc3nc(-c4c(NC(=O)C5CC(NC(C)C)C5)sc5c4CNCC5)sc3c2)cn1] | ESI-MS (M + H)⁺ = 507.2 |
| 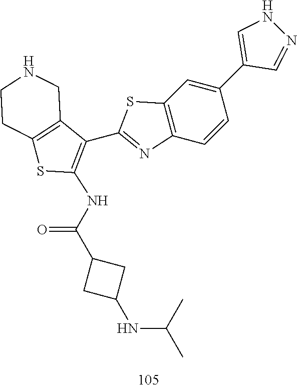 [SMILES: CC(C)NC1CC(C(=O)Nc2sc3c(c2-c2nc4ccc(-c5cn[nH]c5)cc4s2)CNCC3)C1] | ESI-MS (M + H)⁺ = 493.2 |
| 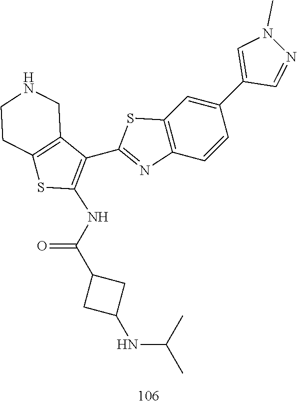 [SMILES: CC(C)NC1CC(C(=O)Nc2sc3c(c2-c2nc4ccc(-c5cnn(C)c5)cc4s2)CNCC3)C1] | ESI-MS (M + H)⁺ = 507.2 |
| 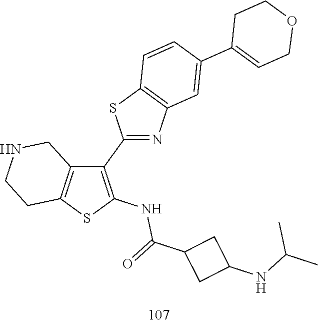 [SMILES: CC(C)NC1CC(C(=O)Nc2sc3c(c2-c2nc4cc(C5=CCOCC5)ccc4s2)CNCC3)C1] | ESI-MS (M + H)⁺ = 509.2 |
| 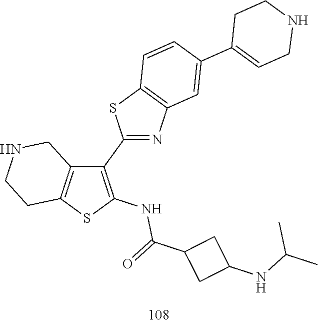 [SMILES: CC(C)NC1CC(C(=O)Nc2sc3c(c2-c2nc4cc(C5=CCNCC5)ccc4s2)CNCC3)C1] | ESI-MS (M + H)⁺ = 508.2 |
| 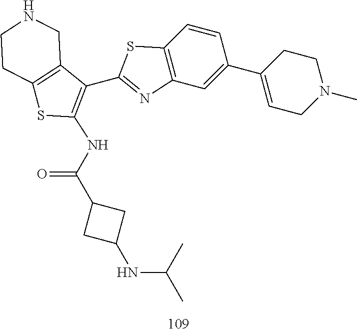 [SMILES: CC(C)NC1CC(C(=O)Nc2sc3c(c2-c2nc4cc(C5=CCN(C)CC5)ccc4s2)CNCC3)C1] | ESI-MS (M + H)⁺ = 522.2 |
| 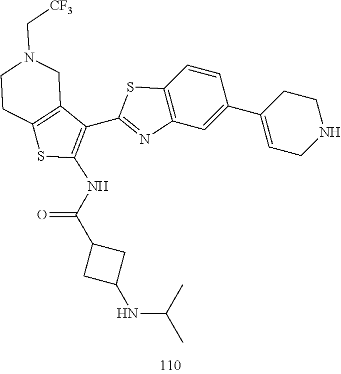 [SMILES: CC(C)NC1CC(C(=O)Nc2sc3c(c2-c2nc4cc(C5=CCNCC5)ccc4s2)CN(CC(F)(F)F)CC3)C1] | ESI-MS (M + H)⁺ = 590.2 |

### Example 39:1-(3-(benzo[d]thiazol-2-yl)-4,5,6,7-tetrahydrothieno[2,3-c]pyridin-2-yl)-3-(2-(isopropylamino)ethyl) urea (077)

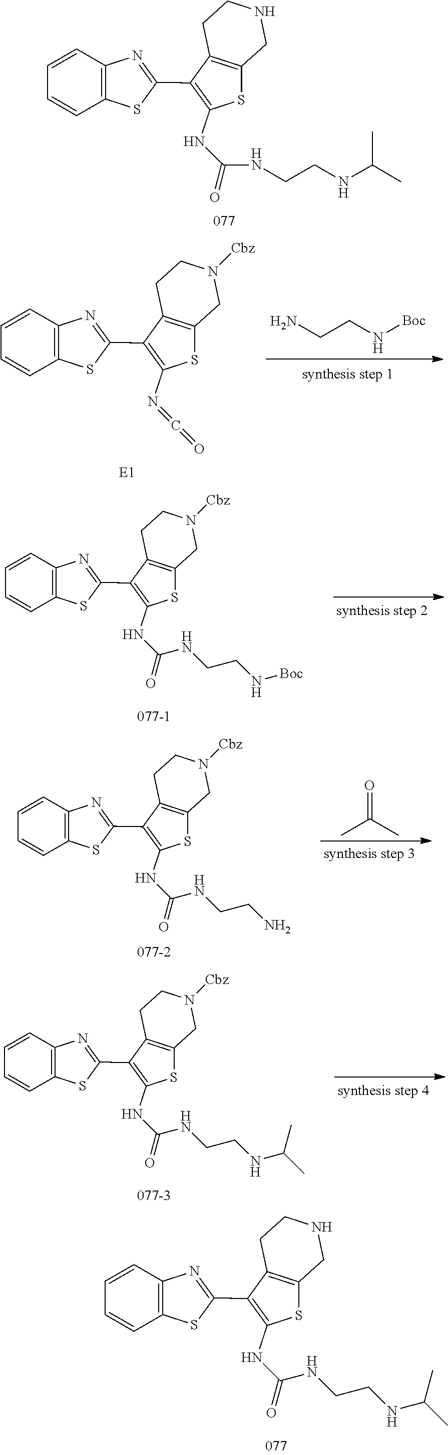

### Synthesis Step 1: benzyl 3-(benzo[d]thiazol-2-yl)-2-(3-(2-((tert-butoxycarbonyl)amino)ethyl)ureido)-4,7-dihydrothieno[2,3-c]pyridin-6 (5H)-carboxylate (077-1)

The intermediate E1 (400 mg, 0.89 mmol) was dissolved in 10 mL of tetrahydrofuran solution, N-tert-butoxycarbonyl-1,2-ethylenediamine (143 mg, 0.89 mmol) was added at room temperature, the reaction was carried out at room temperature for 1 h, and then tetrahydrofuran was removed by concentration. An appropriate amount of water was added, and the mixture was extracted with DCM. The organic phases were combined, dried and then concentrated, and the crude product was separated and purified by column chromatography to obtain product 077-1 (263 mg) with a yield of 48.6%. ESI-MS (M+H)⁺608.2.

### Synthesis Step 2: benzyl 2-(3-(2-aminoethyl) ureido)-3-(benzo[d]thiazol-2-yl)-4,7-dihydrothieno[2,3-c]pyridin-6 (5H)-carboxylate (077-2)

077-1 (263 mg, 0.43 mmol) was dissolved in 15 mL of dichloromethane, trifluoroacetic acid (165 μL, 2.15 mmol) was added, and the reaction was carried out at room temperature for 2 h, then dichloromethane and trifluoroacetic acid were removed by concentration to obtain 077-2 (261 mg) with a yield of 97.9%, which was directly used in the next reaction without further purification. ESI-MS (M+H)⁺508.1.

### Synthesis Step 3: benzyl 3-(benzo[d]thiazol-2-yl)-2-(3-(2-(isopropylamino)ethyl) ureido)-4,7-dihydrothieno[2,3-c]pyridin-6 (5H)-carboxylate (077-3)

077-2 (261 mg, 0.42 mmol) was suspended in 10 mL of anhydrous dichloromethane, and triethylamine (128 mg, 1.26 mmol) was added. After the substrate was dissolved and became clear, acetone (34 μL, 0.46 mmol) was added, then STAB (136 mg, 0.63 mmol) was added, and the reaction was carried out at room temperature overnight. After the reaction was completed, dichloromethane and saturated aqueous sodium bicarbonate solution were added for extraction. The dichloromethane phase was recovered and purified by column chromatography to obtain 077-3 (166 mg) with a yield of 71.9%. ESI-MS (M+H)⁺550.2.

### Synthesis Step 4:1-(3-(benzo[d]thiazol-2-yl)-4,5,6,7-tetrahydrothieno[2,3-c]pyridin-2-yl)-3-(2-(isopropylamino)ethyl) urea (077)

To a high-pressure reaction flask, 077-3 (166 mg, 0.30 mmol) was added, then 8 mL of methanol and 5% palladium on carbon (83 mg) were added in sequence, then hydrogen gas was introduced (3 atm), and the reaction was carried out at room temperature overnight. After the reaction was completed as monitored by LC-MS, the reaction solution was filtered, and the filter cake was rinsed with tetrahydrofuran. The mother liquor was recovered and concentrated, and then dissolved with methyl tert-butyl ether. 4 mol/L hydrogen chloride in dioxane solution (500 μL) was slowly added, and the reaction was carried out at room temperature for 3 h, after which a solid precipitated. The solid was dissolved to saturated sodium bicarbonate aqueous solution, and the pH was adjusted to weak alkalinity. The mixture was extracted with dichloromethane, and the organic phase was concentrated, then separated and purified by reverse-phase chromatography and freeze-dried to obtain compound 077 (55 mg) with a yield of 44%. ESI-MS (M+H)⁺416.2.

¹H NMR (400 MHz, DMSO-d₆) δ 12.08 (s, 1H), 9.69 (s, 2H), 8.79 (s, 1H), 8.49 (d, J=8.2 Hz, 1H), 8.08 (dd, J=8.1, 1.2 Hz, 1H), 7.52 (ddd, J=8.3, 7.1, 1.2 Hz, 1H), 7.40 (ddd, J=8.3, 7.2, 1.2 Hz, 1H), 4.22 (s, 2H), 3.50 (q, J=6.0 Hz, 3H), 3.35-3.26 (m, 2H), 3.12 (s, 2H), 3.07 (dd, J=11.0, 5.3 Hz, 2H), 1.24 (d, J=6.5 Hz, 6H).

Referring to the synthetic route and method of Example 39, the following target compound was synthesized:

| Number of | | Characterization |
|---|---|---|
| compound | Structure of compound | data |
| 078 | 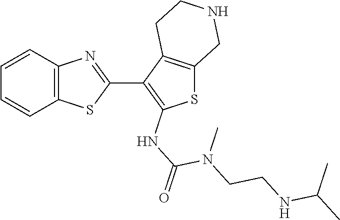 [SMILES: CC(C)NCCN(C)C(=O)Nc1sc2c(c1-c1nc3ccccc3s1)CCNC2] | ESI-MS (M + H)⁺ = 430.2 |
| | 078 | |

### Example 40: N-(3-(benzo[d]thiazol-2-yl)-4,5,6,7-tetrahydrothieno[2,3-c]pyridin-2-yl)-3-(isopropylamino) azetidine-1-carboxamide (079)

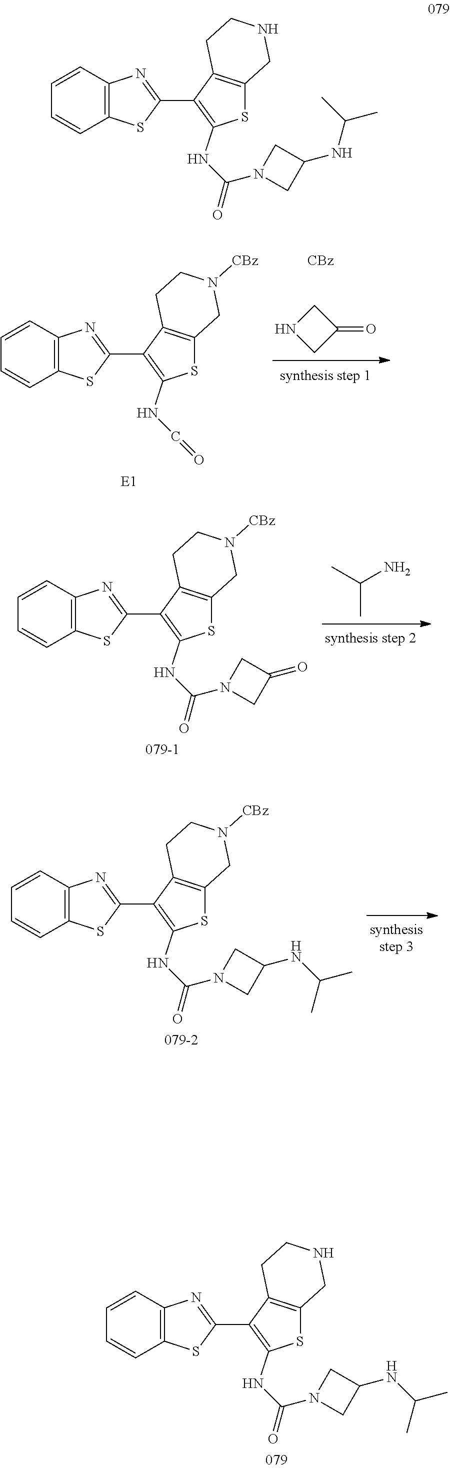

### Synthesis Step 1: benzyl 3-(benzo[d]thiazol-2-yl)-2-(3-oxoazetidine-1-carboxamido)-4,7-dihydrothieno[2,3-c]pyridin-6 (5H)-carboxylate (079-1)

Referring to the synthesis route and method of synthesis step 1 in Example 39, in synthesis step 1, N-tert-butoxycarbonyl-1,2-ethylenediamine was replaced with azetidine-3-one to synthesize 079-1. ESI-MS (M+H)⁺519.1.

### Synthesis Step 2: benzyl 3-(benzo[d]thiazol-2-yl)-2-(3-(isopropylamino) azetidine-1-carboxamido)-4,7-dihydrothieno[2,3-c]pyridin-6 (5H)-carboxylate (079-2)

079-1 (160 mg, 0.31 mmol) was dissolved in 12 mL of anhydrous dichloromethane, and isopropylamine (30 μL, 0.34 mmol) was added. The mixture was stirred at room temperature for 30 min, and added with sodium triacetoxyborohydride (STAB, 98 mg, 0.46 mmol), and the reaction was continued at room temperature for 1 h. After the reaction was completed, the mixture was extracted by adding dichloromethane and saturated sodium bicarbonate. The organic phase was recovered and separated and purified by column chromatography to obtain 079-2 (80 mg) with a yield of 46.2%. and ESI-MS (M+H)⁺562.2.

### Synthesis Step 3: N-(3-(benzo[d]thiazol-2-yl)-4,5,6,7-tetrahydrothieno[2,3-c]pyridin-2-yl)-3-(isopropylamino) azetidine-1-carboxamide (079)

Referring to the operation and method of synthesis step 4 in Example 39, in synthesis step 4, 077-3 was replaced with 079-2 to synthesize and obtain compound 079, ESI-MS (M+H)⁺428.2.

### Example 41:1-(2-((2-Methoxyethyl)amino)ethyl)-3-(3-(6-(6-(piperazin-1-yl) pyridin-3-yl)benzo[d]thiazol-2-yl)-4,5,6,7-tetrahydrothieno[2,3-c]pyridin-2-yl)urea (080) (080)

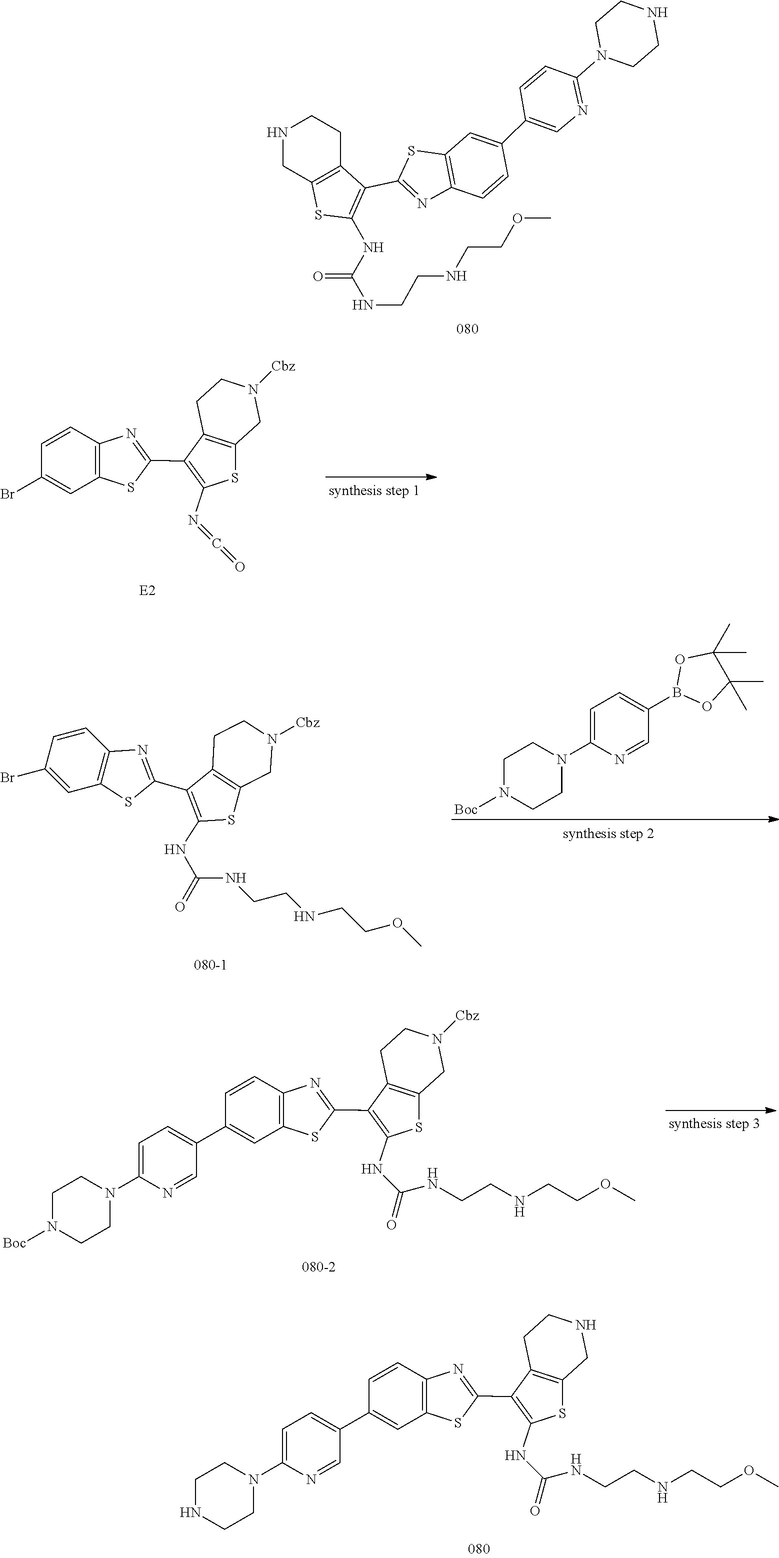 [SMILES: COCCNCCNC(=O)Nc1sc2c(c1-c1nc3ccc(-c4ccc(N5CCNCC5)nc4)cc3s1)CCNC2 ; O=C=Nc1sc2c(c1-c1nc3ccc(Br)cc3s1)CCN(C(=O)OCc1ccccc1)C2 ; COCCNCCNC(=O)Nc1sc2c(c1-c1nc3ccc(Br)cc3s1)CCN(C(=O)OCc1ccccc1)C2 ; CC(C)(C)OC(=O)N1CCN(c2ccc(B3OC(C)(C)C(C)(C)O3)cn2)CC1 ; COCCNCCNC(=O)Nc1sc2c(c1-c1nc3ccc(-c4ccc(N5CCN(C(=O)OC(C)(C)C)CC5)nc4)cc3s1)CCN(C(=O)OCc1ccccc1)C2 ; COCCNCCNC(=O)Nc1sc2c(c1-c1nc3ccc(-c4ccc(N5CCNCC5)nc4)cc3s1)CCNC2]

### Synthesis Step 1: benzyl 3-(6-bromobenzo[d]thiazol-2-yl)-2-(3-(2-((2-methoxyethyl)amino)ethyl) ureido)-4,7-dihydrothieno[2,3-c]pyridin-6 (5H)-carboxylate (080-1)

Referring to the routes and methods of synthesis steps 1, 2 and 3 in Example 39, intermediate E1 was replaced with intermediate E2 in synthesis step 1, and acetone was replaced with 1,1,2-trimethoxyethane in synthesis step 3, to synthesize and obtain 080-1, ESI-MS (M+H)⁺644.1.

### Synthesis Step 2: benzyl 3-(6-(6-(4-(tert-butoxycarbonyl) piperazin-1-yl)pyridin-3-yl)benzo[d]thiazol-2-yl)-2-(3-(2-((2-methoxyethyl)amino)ethyl) ureido)-4,7-dihydrothieno[2,3-c]pyridin-6 (5H)-carboxylate (080-2)

080-1 (420 mg, 0.65 mmol) was dissolved in 12 mL of anhydrous dioxane, at room temperature, tert-butyl 4-(5-(4,4,5,5-tetramethyl-1,3,2-dioxaborolane-2-yl)pyridin-2-yl) piperazin-1-carboxylate (331 mg, 0.85 mmol), sodium carbonate (136 mg, 1.28 mmol), Pd(dppf)Cl₂ (47 mg, 0.065 mmol) were added in sequence, and finally water (3 mL) was added. The reaction was carried out at 100° C. for 6 h. After the reaction was completed, dioxane was removed by concentration, an appropriate amount of water was added, and the mixture was extracted with EA. The organic phases were combined, dried and then concentrated. The crude product was purified by column chromatography to obtain 080-2 (395 mg) with a yield of 73.6%, ESI-MS (M+H)⁺827.3.

### Synthesis Step 3:1-(2-((2-methoxyethyl)amino)ethyl)-3-(3-(6-(6-(piperazin-1-yl) pyridin-3-yl)benzo[d]thiazol-2-yl)-4,5,6,7-tetrahydrothieno[2,3-c]pyridin-2-yl) urea (080)

Referring to the synthetic route and method of synthesis step 4 in Example 39, in synthesis step 4, 077-3 was replaced with 080-2 to synthesize compound 080, ESI-MS (M+H)⁺593.2.

### Example 42:2-((2-methoxyethyl)amino)ethyl (3-(6-(6-(piperazin-1-yl)pyridin-3-yl)benzo[d]thiazol-2-yl)-4,5,6,7-tetrahydrothieno[2,3-c]pyridin-2-yl)carbamate (081)

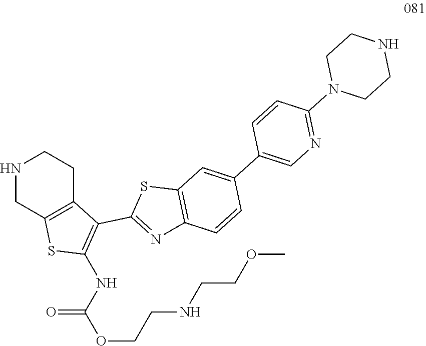 [SMILES: COCCNCCOC(=O)Nc1sc2c(c1-c1nc3ccc(-c4ccc(N5CCNCC5)nc4)cc3s1)CCNC2]

Referring to the synthesis routes and methods of Example 39 and Example 41, in synthesis step 1 of Example 32, N-tert-butoxycarbonyl-1,2-ethylenediamine was replaced with N-(tert-butoxycarbonyl) ethanolamine to synthesize compound 081, ESI-MS (M+H)⁺594.2.

### Example 43: N¹-isopropyl-N³-(3-(6-(6-(piperazin-1-yl)pyridin-3-yl)benzo[d]thiazol-2-yl)-4,5,6,7-tetrahydrothieno[2,3-c]pyridin-2-yl) propane-1,3-diamine (082)

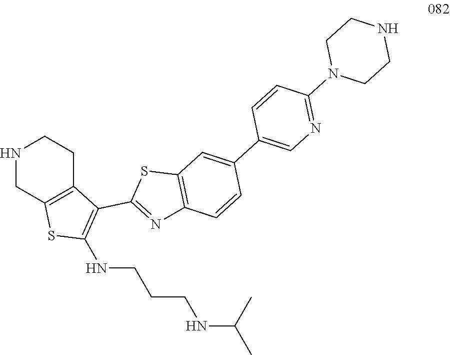 [SMILES: CC(C)NCCCNc1sc2c(c1-c1nc3ccc(-c4ccc(N5CCNCC5)nc4)cc3s1)CCNC2]

Referring to the synthesis route and method of Example 26, in the synthesis step 1, intermediate B1 was replaced with intermediate B13 and tert-butyl 4-(4,4,5,5-tetramethyl-1,3,2-dioxaborolane-2-yl)-3,6-dihydropyridin-1 (2H)-carboxylate was replaced with tert-butyl 4-(5-(4,4,5,5-tetramethyl-1,3,2-dioxaborolane-2-yl)pyridin-2-yl) piperazin-1-carboxylate, to synthesize compound 082, ESI-MS (M+H)⁺548.3.

¹H NMR (400 MHz, DMSO-d₆) δ 9.77 (s, 1H), 9.48 (s, 2H), 9.14 (s, 1H), 8.49 (d, J=2.5 Hz, 1H), 8.33 (d, J=1.9 Hz, 1H), 8.14 (d, J=9.0 Hz, 1H), 8.06 (d, J=8.4 Hz, 1H), 7.76 (dd, J=8.5, 1.9 Hz, 1H), 7.18 (d, J=9.2 Hz, 1H), 4.16 (s, 2H), 3.86 (d, J=5.3 Hz, 6H), 3.47 (s, 2H), 3.41 (d, J=6.1 Hz, 2H), 3.24 (d, J=6.1 Hz, 1H), 3.18 (d, J=5.4 Hz, 4H), 2.97 (d, J=13.1 Hz, 2H), 2.07 (q, J=7.2 Hz, 2H), 1.22 (d, J=6.5 Hz, 6H).

Referring to the synthetic route and method of Example 43, the following target compounds were synthesized:

| Number of | | |
|---|---|---|
| compound | Structure of compound | Characterization data |
| 083 | 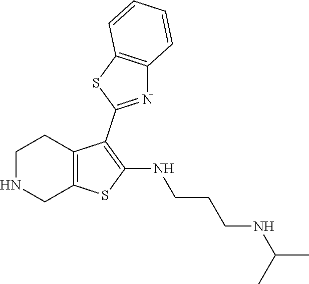 | ESI-MS(M + H)⁺ = 587.2 ¹H NMR (400 MHZ, DMSO-d₆) δ 9.77 (s, 1H), 9.41 (s, 1H), 9.13 (s, 1H), 8.03-7.95 (m, 2H), 7.45 (ddd, J = 8.2, 7.2, 1.3 Hz, 1H), 7.30 (td, J = 7.6, 1.2 Hz, 1H), 4.14 (d, J = 5.8 Hz, 2H), 3.46 (s, 2H), 3.43-3.34 (m, 2H), 3.23 (dt, J = 12.5, 6.2 Hz, 1H), 3.08 (t, J = 6.1 Hz, 2H), 2.93 (d, J = 8.5 Hz, 2H), 2.19-2.00 (m, 2H), 1.21 (d, J = 6.5 Hz, 6H). |
| | 083 | |
| 084 | 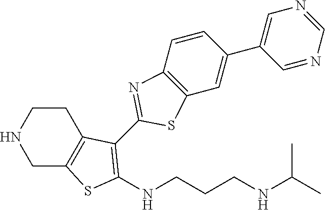 [SMILES: CC(C)NCCCNc1sc2c(c1-c1nc3ccc(-c4cncnc4)cc3s1)CCNC2] | ESI-MS(M + H)⁺ = 465.2 ¹H NMR (400 MHZ, DMSO-d₆) δ 9.54-9.22 (m, 5H), 8.53 (d, J = 1.9 Hz, 1H), 8.19 (d, J = 8.5 Hz, 1H), 7.94 (dd, J = 8.5, 1.9 Hz, 1H), 4.17 (s, 2H), 3.52 (t, J = 6.5 Hz, 2H), 3.45 (s, 2H), 3.15-3.10 (m, 3H), 2.98 (s, 2H), 2.13 (q, J = 7.1 Hz, 2H), 1.26 (d, J = 6.4 Hz, 6H). |
| | 084 | |
| 085 | 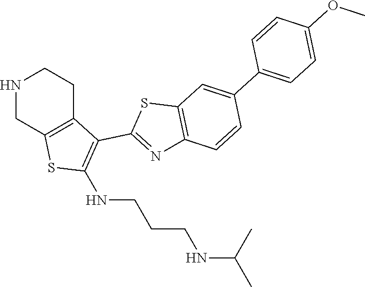 [SMILES: COc1ccc(-c2ccc3nc(-c4c(NCCCNC(C)C)sc5c4CCNC5)sc3c2)cc1] | ESI-MS(M + H)⁺ = 493.2 ¹H NMR (400 MHZ, DMSO-d₆) δ 9.82 (s, 1H), 9.47 (s, 1H), 9.18 (s, 1H), 8.31-8.25 (m, 1H), 8.06 (d, J = 8.6 Hz, 1H), 7.76-7.72 (m, 1H), 7.69 (d, J = 8.8 Hz, 2H), 7.05 (d, J = 8.9 Hz, 2H), 4.19 (s, 2H), 3.81 (s, 3H), 3.50 (t, J = 6.6 Hz, 2H), 3.45 (d, J = 6.7 Hz, 2H), 3.26 (dq, J = 12.4, 6.2 Hz, 1H), 3.13 (s, 2H), 2.99 (s, 2H), 2.19-2.07 (m, 2H), 1.26 (d, J = 6.6 Hz, 6H). |
| | 085 | |
| 091 | 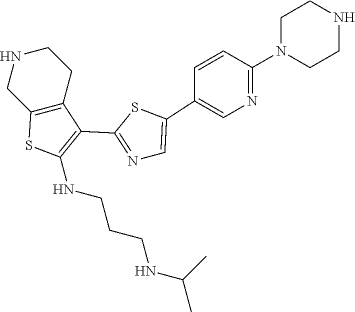 [SMILES: CC(C)NCCCNc1sc2c(c1-c1ncc(-c3ccc(N4CCNCC4)nc3)s1)CCNC2] | ESI-MS(M + H)⁺ = 498.2 |
| | 091 | |
| 092 | 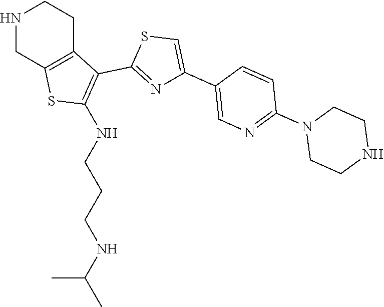 [SMILES: CC(C)NCCCNc1sc2c(c1-c1nc(-c3ccc(N4CCNCC4)nc3)cs1)CCNC2] | ESI-MS(M + H)⁺ = 498.2 |
| | 092 | |

### Example 44: N-(2-(isopropylamino)ethyl)-2-((3-(6-(1,2,3,6-tetrahydropyridin-4-yl)benzo[d]thiazol-2-yl)-4,5,6,7-tetrahydrothieno[2,3-c]pyridin-2-yl)amino) acetamide (087)

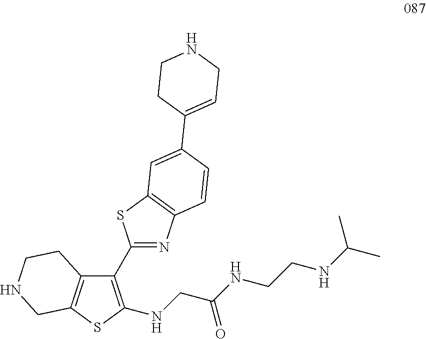 [SMILES: CC(C)NCCNC(=O)CNc1sc2c(c1-c1nc3ccc(C4=CCNCC4)cc3s1)CCNC2]

Referring to the synthesis route and method of Example 26, in synthesis step 1, intermediate B1 was replaced with intermediate B15 to synthesize compound 087. ESI-MS (M+H)⁺511.2.

Referring to the synthetic route and method of Example 44, the following target compounds were synthesized and obtained:

| Example | Structure of compound | Characterization data |
|---|---|---|
| 086 | 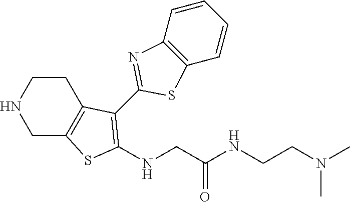 [SMILES: CN(C)CCNC(=O)CNc1sc2c(c1-c1nc3ccccc3s1)CCNC2] | ESI-MS(M + H)⁺ = 416.2 |
| | 086 | |
| 088 | 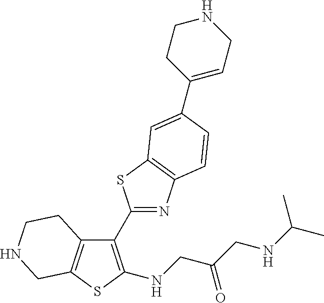 [SMILES: CC(C)NCC(=O)CNc1sc2c(c1-c1nc3ccc(C4=CCNCC4)cc3s1)CCNC2] | ESI-MS(M + H)⁺ = 482.2 |
| | 088 | |

### Example 45:2-(2-(4-(2-methoxyethyl) piperazin-1-yl)-4,5,6,7-tetrahydrothieno[2,3-c]pyridin-3-yl)-6-(6-(piperazin-1-yl)pyridin-3-yl)benzo[d]thiazole (089)

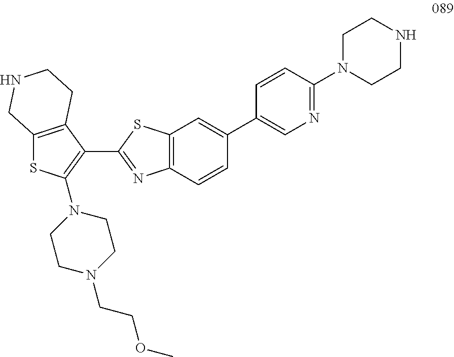 [SMILES: COCCN1CCN(c2sc3c(c2-c2nc4ccc(-c5ccc(N6CCNCC6)nc5)cc4s2)CCNC3)CC1]

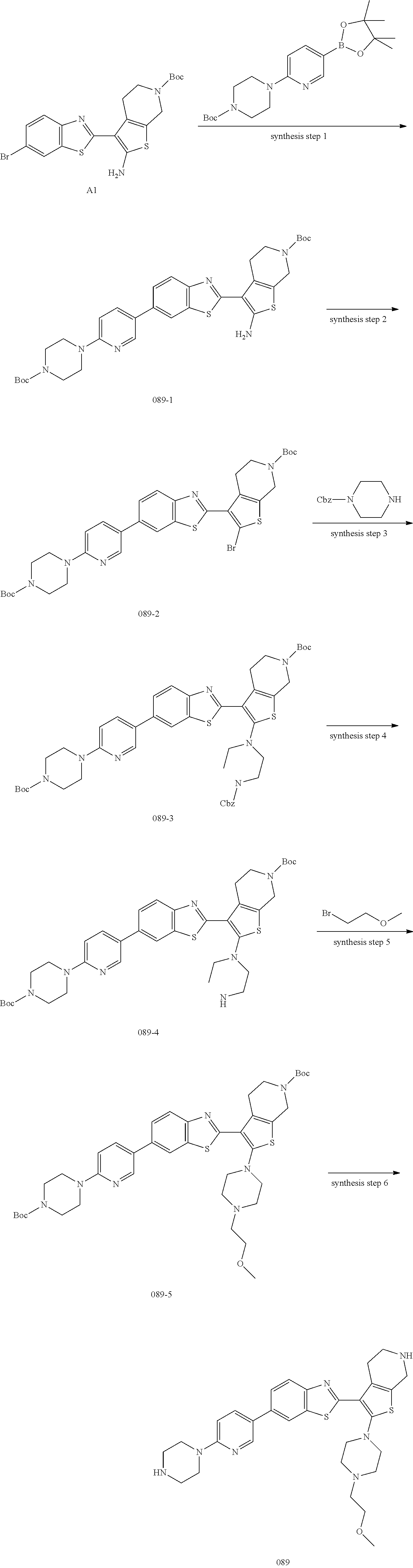

### Synthesis Step 1: tert-butyl 2-amino-3-(6-(6-(4-(tert-butoxycarbonyl) piperazin-1-yl)pyridin-3-yl)benzo[d]thiazol-2-yl)-4,7-dihydrothieno[2,3-c]pyridin-6 (5H)-carboxylate (089-1)

The intermediate A1 (2 g, 4.29 mmol) was dissolved in 20 mL of dioxane, tert-butyl 4-(5-(4,4,5,5-tetramethyl-1,3,2-dioxaborolane-2-yl)pyridin-2-yl) piperazin-1-carboxylate (1.67 g, 4.29 mmol), sodium carbonate (455 mg, 4.29 mmol), Pd(dppf)Cl₂ (312 mg, 0.43 mmol) and water (4 mL) were added in sequence, and the reaction was carried out at 100° C. for 2 h. After the reaction was completed, the mixture was cooled to room temperature, and dioxane was removed by concentration. An appropriate amount of water was added, and the mixture was extracted with EA. The organic phases were combined, dried and then concentrated. The crude product was purified by column chromatography to obtain 089-1 (494 mg) with a yield of 76%. ESI-MS (M+H)⁺649.3.

### Synthesis Step 2: tert-butyl 2-bromo-3-(6-(6-(4-(tert-butoxycarbonyl) piperazin-1-yl) pyridin-3-yl)benzo[d]thiazol-2-yl)-4,7-dihydrothieno[2,3-c]pyridin-6 (5H)-carboxylate (089-2)

Copper (I) bromide (530 mg, 3.70 mmol) was dissolved in 20 mL of acetonitrile, tert-butyl nitrite (317 mg, 3.08 mmol) was added, the mixture was cooled to 0° C., and mixed under stirring for 10 min, and 089-1 (2 g, 3.08 mmol) was added. The reaction was continued for 10 min, and the mixture was transferred to room temperature and reacted for 6 h. After the reaction was completed, it was quenched by adding water, and the mixture was extracted with EA. The organic phases were combined, dried and then concentrated. The obtained crude product was purified by column chromatography to obtain 089-2 (966 mg) with a yield of 44%. ESI-MS (M+H)⁺712.2.

### Synthesis Step 3: tert-butyl 2-(4-((benzyloxy) carbonyl) piperazin-1-yl)-3-(6-(6-(4-(tert-butoxycarbonyl) piperazin-1-yl)pyridin-3-yl)benzo[d]thiazol-2-yl)-4,7-dihydrothieno[2,3-c]pyridin-6 (5H)-carboxylate (089-3)

In a sealed tube, 089-2 (200 mg, 0.308 mmol), benzyl-1-piperazine carboxylate (81 mg, 0.370 mmol), tris(dibenzylideneacetone) dipalladium (29 mg, 0.031 mmol), BINAP(192 mg, 0.308 mmol) and cesium carbonate (121 mg, 0.370 mmol) were added in sequence, and 15 mL of toluene was added. The reaction was carried out in the sealed tube at 110° C. overnight. After the reaction was completed, the system was cooled to room temperature, quenched by adding water, and extracted with dichloromethane. The organic phase was extracted with saturated sodium chloride solution to remove water, and dried over anhydrous sodium sulfate. The obtained crude product was purified by column chromatography using eluent of petroleum ether and ethyl acetate to obtain 089-3 (128 mg) with a yield of 49%. ESI-MS (M+H)⁺852.4.

### Synthesis Step 4: tert-butyl 3-(6-(6-(4-(tert-butoxycarbonyl) piperazin-1-yl) pyridin-3-yl)benzo[d]thiazol-2-yl)-2-(piperazin-1-yl)-4,7-dihydrothieno[2,3-c]pyridin-6 (5H)-carboxylate (089-4)

To a high-pressure reaction flask, 089-3 (128 mg, 0.151 mmol) was added, and 10 mL of methanol and 5% palladium on carbon (38 mg) were added. Hydrogen gas was then introduced (3 atm), and the reaction was carried out at room temperature overnight. After the reaction was completed as monitored by LC-MS, the reaction solution was filtered, and the filter cake was rinsed with tetrahydrofuran. The mother liquor was recovered and concentrated to obtain 089-4 (89 mg) with a yield of 82%. ESI-MS (M+H)⁺718.3.

### Synthesis Step 5: tert-butyl 3-(6-(6-(4-(tert-butoxycarbonyl) piperazin-1-yl) pyridin-3-yl)benzo[d]thiazol-2-yl)-2-(4-(2-methoxyethyl) piperazin-1-yl)-4,7-dihydrothieno[2, 3-c]pyridin-6 (5H)-carboxylate (089-5)

To a reaction flask, 089-4 (89 mg, 0.124 mmol) was added, and 5 mL of anhydrous DMF, 2-bromoethyl methyl ether (18 mg, 0.124 mmol), potassium carbonate (21 mg, 0.149 mmol) and potassium iodide (21 mg, 0.124 mmol) were added in sequence. The reaction was carried out at 50° C. for 4 h. After the reaction was completed, the mixture was cooled to room temperature, quenched by adding water, and extracted with EA. The organic phases were combined, dried and then concentrated. The crude product was purified by column chromatography to obtain 089-5 (75 mg) with a yield of 78%. ESI-MS (M+H)⁺776.4.

### Synthesis Step 6:2-(2-(4-(2-Methoxyethyl) piperazin-1-yl)-4,5,6,7-tetrahydrothieno[2,3-c]pyridin-3-yl)-6-(6-(piperazin-1-yl)pyridin-3-yl)benzo[d]thiazole (089)

089-5 (75 mg, 0.097 mol) was dissolved in 5 mL of dioxane, 5 mL of 4 mol/L solution of hydrogen chloride in dioxane was added, and the reaction was carried out at room temperature for 7 h. After the reaction was completed, the mixture was concentrated under reduced pressure, adjusted to a pH of weak alkalinity by adding saturated sodium bicarbonate aqueous solution, and extracted with dichloromethane. The organic phase was concentrated, then separated and purified by reverse-phase chromatography, and freeze-dried to obtain compound 089 (35 mg) with a yield of 63%. ESI-MS (M+H)⁺576.3.

Referring to the synthetic route and method of Example 45, the following target compound was synthesized:

| Example | Structure of compound | Characterization data |
|---|---|---|
| 126 | 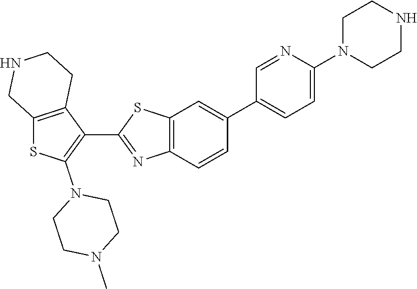 [SMILES: CN1CCN(c2sc3c(c2-c2nc4ccc(-c5ccc(N6CCNCC6)nc5)cc4s2)CCNC3)CC1] | ESI-MS(M + H)⁺ = 532.2 ¹H NMR (400 MHZ, DMSO-d₆) δ 9.91 (s, 1H), 9.63 (s, 1H), 8.49 (d, J = 2.5 Hz, 1H), 8.38 (d, J = 1.9 Hz, 1H), 8.30 (d, J = 9.3 Hz, 1H), 8.07 (d, J = 8.6 Hz, 1H), 7.85 (dd, J = 8.5, 1.9 Hz, 1H), 7.34 (d, J = 9.4 Hz, 1H), 4.33 (m, 2H), 3.97 (m, 5H), 3.56 (s, 3H), 3.47-3.31 (m, 9H), 3.24 (m, 4H), 2.89 (m, 2H). |
| | 126 | |

### Example 46:4-(2-Methoxyethyl)-1-(3-(6-(6-(piperazin-1-yl)pyridin-3-yl)benzo[d]thiazol-2-yl)-4,5,6,7-tetrahydrothieno[2,3-c]pyridin-2-yl) piperazin-2-one (090)

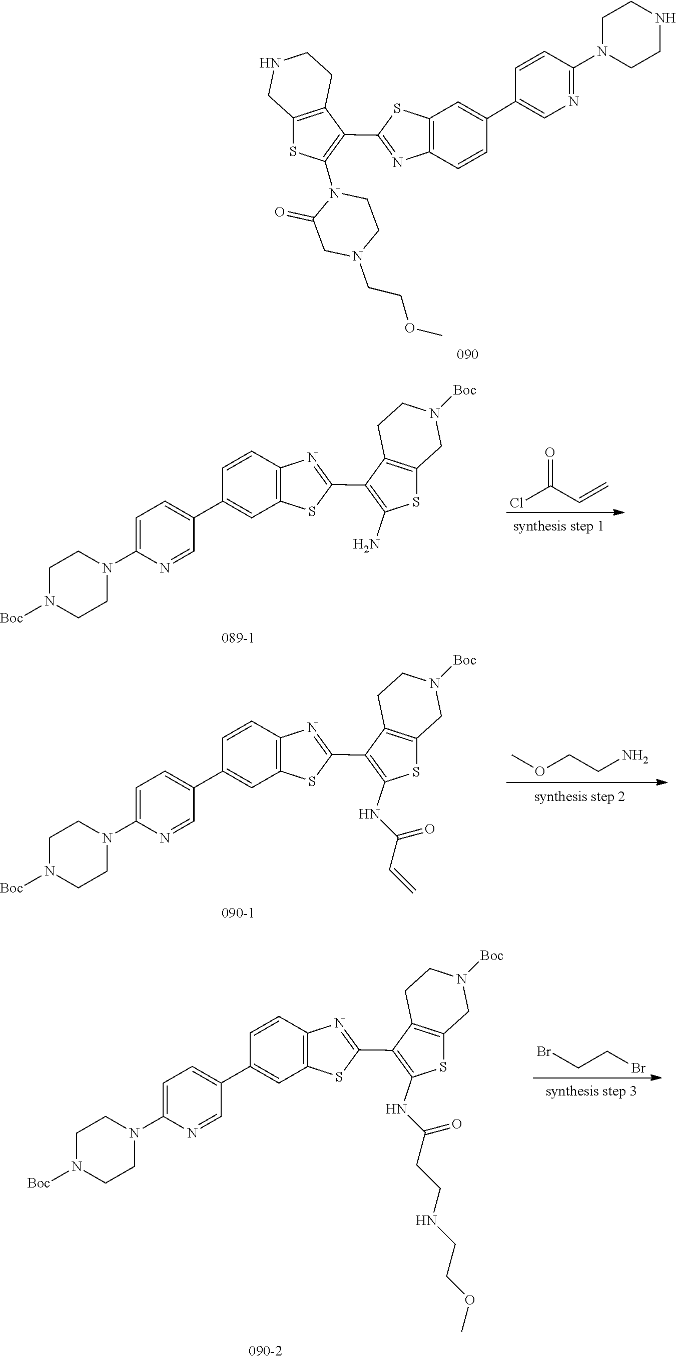

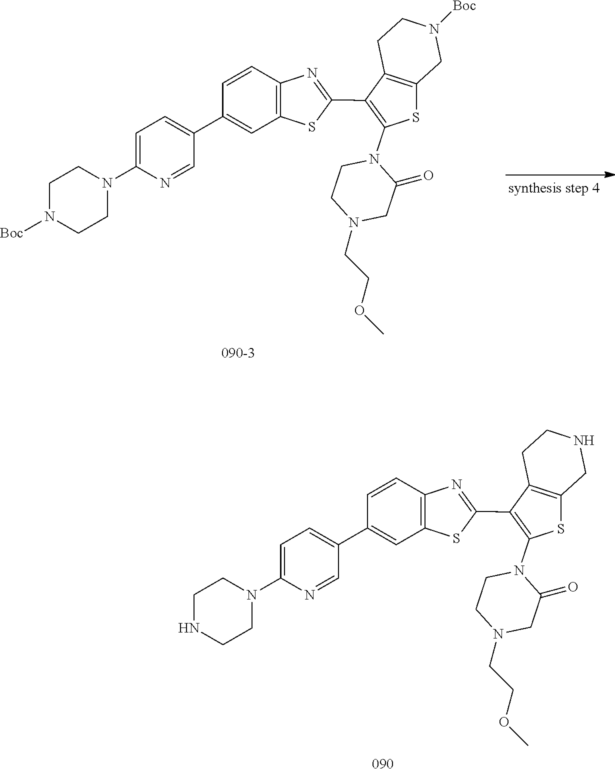 [SMILES: COCCN1CCN(c2sc3c(c2-c2nc4ccc(-c5ccc(N6CCN(C(=O)OC(C)(C)C)CC6)nc5)cc4s2)CCN(C(=O)OC(C)(C)C)C3)C(=O)C1 ; COCCN1CCN(c2sc3c(c2-c2nc4ccc(-c5ccc(N6CCNCC6)nc5)cc4s2)CCNC3)C(=O)C1]

### Synthesis Step 1: tert-butyl 2-acrylamido-3-(6-(6-(4-(tert-butoxycarbonyl) piperazin-1-yl)pyridin-3-yl)benzo[d]thiazol-2-yl)-4,7-dihydrothieno[2,3-c]pyridin-6 (5H)-carboxylate (090-1)

089-1 (2 g, 3.08 mmol) was dissolved in 30 mL of dichloromethane, and triethylamine (468 mg, 4.62 mmol) was added. The mixture was cooled to 0° C., and then acryloyl chloride (307 mg, 3.38 mmol) was slowly added dropwise. After the dropwise addition was completed, the mixture was transferred to room temperature and reacted for 2 h. After the reaction was completed, it was quenched by adding water, and the mixture was extracted with DCM. The organic phases were combined, dried and then concentrated. The crude product was purified by column chromatography to obtain 090-1 (1.86 g) with a yield of 86%. ESI-MS (M+H)⁺703.3.

### Synthesis Step 2: tert-butyl 3-(6-(6-(4-(tert-butoxycarbonyl) piperazin-1-yl)pyridin-3-yl)benzo[d]thiazol-2-yl)-2-(3-((2-methoxyethyl)amino) propanamido)-4,7-dihydrothieno[2, 3-c]pyridin-6 (5H)-carboxylate (090-2)

2-Methoxyethylamine (199 mg, 2.65 mmol) was dissolved in a mixed solvent of 10 mL of anhydrous ethanol and 10 mL of tetrahydrofuran. The system was cooled to 0° C., then 090-1 (1.86 g, 2.65 mmol) was added. After the dropwise addition was completed, the mixture was transferred to room temperature and reacted for 2 h. After the reaction was completed, ethanol and tetrahydrofuran were removed by concentration under reduced pressure. An appropriate amount of water was added, and the mixture was extracted with EA. The organic phases were combined, dried and then concentrated. The crude product was purified by column chromatography to obtain 090-2 (1.9 g) with a yield of 92%. ESI-MS (M+H)⁺778.3.

### Synthesis Step 3: tert-butyl 3-(6-(6-(4-(tert-butoxycarbonyl) piperazin-1-yl)pyridin-3-yl)benzo[d]thiazol-2-yl)-2-(4-(2-methoxyethyl)-2-oxopiperazin-1-yl)-4,7-dihydrothieno[2, 3-c]pyridin-6 (5H)-carboxylate (090-3)

090-2 (1.9 g, 2.438 mmol) was dissolved in 20 mL of anhydrous DMF, potassium iodide (404 mg, 2.438 mmol) and 1,2-dibromoethane (458 mg, 2.438 mmol) were added in sequence, and the reaction was carried out at 60° C. for 15 h. After the reaction was completed, it was quenched by adding water, and the mixture was extracted with EA. The organic phases were combined, dried and then concentrated. The crude product was purified by column chromatography to obtain 090-3 (423 mg) with a yield of 22%. ESI-MS (M+H)⁺790.3.

### Synthesis Step 4:4-(2-Methoxyethyl)-1-(3-(6-(6-(piperazin-1-yl)pyridin-3-yl)benzo[d]thiazol-2-yl)-4,5,6,7-tetrahydrothieno[2,3-c]pyridin-2-yl) piperazin-2-one (090)

090-3 (423 mg, 0.536 mmol) was dissolved in 5 mL of dioxane, a solution of 4 mol/L hydrogen chloride in dioxane was added, and the reaction was carried out at room temperature for 11 h. After the reaction was completed, the mixture was concentrated under reduced pressure, adjusted to a pH of weak alkalinity by adding saturated sodium bicarbonate aqueous solution, and extracted with dichloromethane. The organic phase was concentrated, then separated and purified by reverse-phase chromatography, and freeze-dried to obtain compound 090 (146 mg) with a yield of 46%. ESI-MS (M+H)⁺590.2.

### Example 47:4-(Tert-butylamino)-N-(3-(6-(1′,2′,3′,6′-tetrahydro-[2,4′-bipyridin]-5-yl)benzo[d]thiazol-2-yl)-4,5,6,7-tetrahydrothieno[2,3-c]pyridin-2-yl)cyclohexane-1-carboxamide (127)

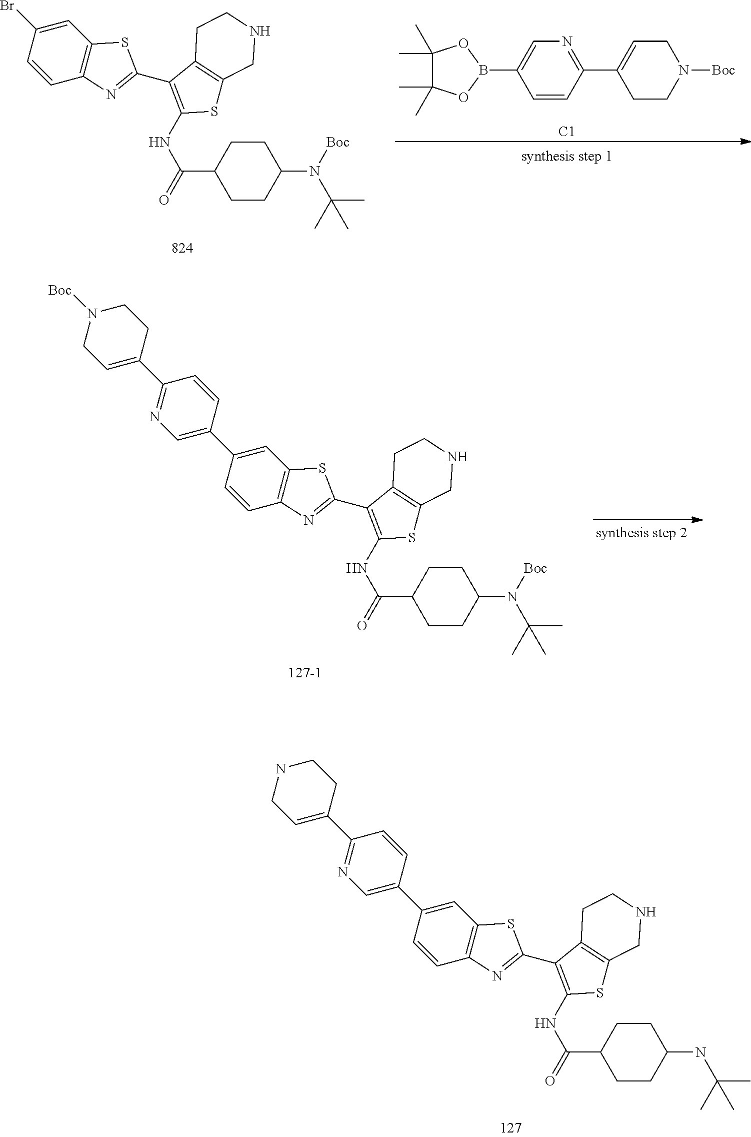

### Synthesis Step 1: tert-butyl 5-(2-(2-(4-((tert-butoxycarbonyl)(tert-butyl)amino)cyclohexane-1-carboxamido)-4,5,6,7-tetrahydrothieno[2,3-c]pyridin-3-yl)benzo[d]thiazol-6-y 1)-3′,6′-dihydro-[2,4′-bipyridin]-1′ (2′H)-carboxylate (127-1)

B1 (130 mg, 0.21 mmol) was added to a mixed solution (dioxane: water=3:1, 5 mL), then C1 (78 mg, 0.25 mmol), [1,1′-bis(diphenylphosphino) ferrocene]dichloropalladium (15 mg, 0.02 mmol) and sodium carbonate (70 mg, 0.63 mmol) were added into the system in sequence, and the reaction was carried out at 80° C. for 5 h. After the reaction was completed, it was quenched by adding water, and the mixture was extracted three times with DCM. The organic phases were combined, dried over anhydrous sodium sulfate, concentrated under reduced pressure, and subjected to column chromatography to obtain 127-1 (140 mg). ESI-MS (M+H)⁺827.4.

### Synthesis Step 2:4-(tert-butylamino)-N-(3-(6-(1′,2′,3′,6′-tetrahydro-[2,4′-bipyridin]-5-yl)benzo[d]thiazol-2-yl)-4,5,6,7-tetrahydrothieno[2,3-c]pyridin-2-yl)cyclohexane-1-carboxamide (127)

D1-1 was dissolved in 5 mL of dichloromethane, hydrogen chloride (dissolved in dioxane, 4 M, 2 mL) was added into the system, and the reaction was carried out at room temperature for 1 h. After the reaction was completed, the mixture was concentrated under reduced pressure, stirred with petroleum ether, then concentrated under reduced pressure and dried to obtain compound 127 (100 mg). ESI-MS (M+H)⁺627.3.

Referring to the synthetic route and method of Example 47, the following target compounds were synthesized:

| | |
|---|---|
| Structure and number of compound | Characterization data |

| | |
|---|---|
| 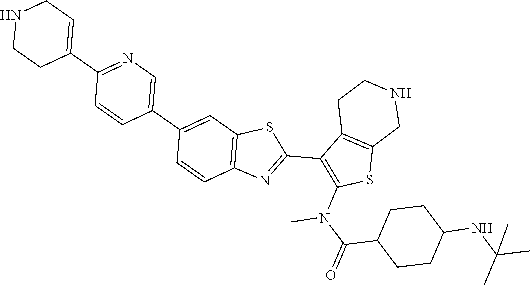 [SMILES: CN(C(=O)C1CCC(NC(C)(C)C)CC1)c1sc2c(c1-c1nc3ccc(-c4ccc(C5=CCNCC5)nc4)cc3s1)CCNC2] | ESI-MS(M + H)⁺ = 641.30 |

128

| | |
|---|---|
| 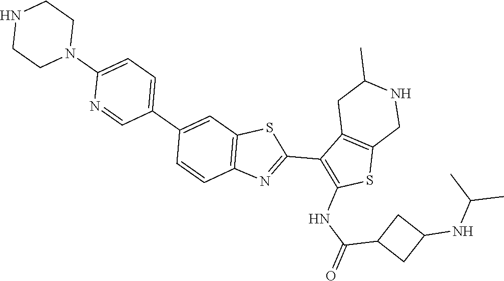 [SMILES: CC(C)NC1CC(C(=O)Nc2sc3c(c2-c2nc4ccc(-c5ccc(N6CCNCC6)nc5)cc4s2)CC(C)NC3)C1] | ESI-MS(M + H)⁺ = 602.3 |

129

| | |
|---|---|
| 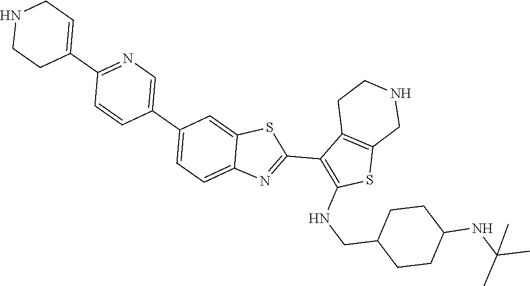 [SMILES: CC(C)(C)NC1CCC(CNc2sc3c(c2-c2nc4ccc(-c5ccc(C6=CCNCC6)nc5)cc4s2)CCNC3)CC1] | ESI-MS(M + H)⁺ = 613.3 |

130

| | |
|---|---|
| 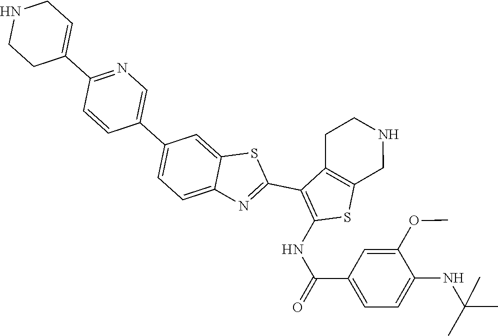 [SMILES: COc1cc(C(=O)Nc2sc3c(c2-c2nc4ccc(-c5ccc(C6=CCNCC6)nc5)cc4s2)CCNC3)ccc1NC(C)(C)C] | ESI-MS(M + H)⁺ = 651.3 |

131

| | |
|---|---|
| 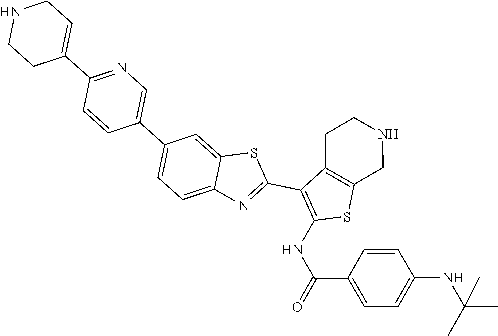 [SMILES: CC(C)(C)Nc1ccc(C(=O)Nc2sc3c(c2-c2nc4ccc(-c5ccc(C6=CCNCC6)nc5)cc4s2)CCNC3)cc1] | ESI-MS(M + H)⁺ = 621.24 |

132

| | |
|---|---|
| 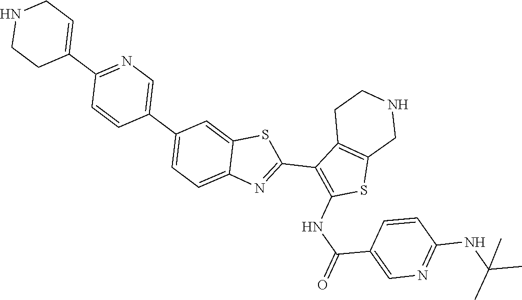 [SMILES: CC(C)(C)Nc1ccc(C(=O)Nc2sc3c(c2-c2nc4ccc(-c5ccc(C6=CCNCC6)nc5)cc4s2)CCNC3)cn1] | ESI-MS(M + H)⁺ = 622.2 |

133

| | |
|---|---|
| 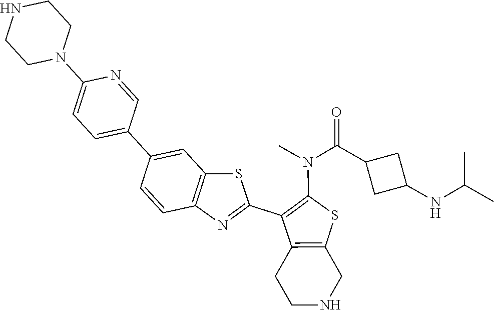 [SMILES: CC(C)NC1CC(C(=O)N(C)c2sc3c(c2-c2nc4ccc(-c5ccc(N6CCNCC6)nc5)cc4s2)CCNC3)C1] | ESI-MS(M + H)⁺ = 602.3 |

134

| | |
|---|---|
| 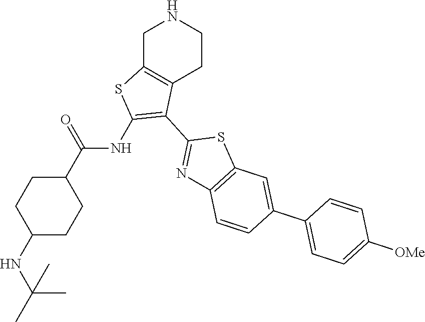 [SMILES: COc1ccc(-c2ccc3nc(-c4c(NC(=O)C5CCC(NC(C)(C)C)CC5)sc5c4CCNC5)sc3c2)cc1] | ESI-MS(M + H)⁺ = 575.2 |

135

| | |
|---|---|
| 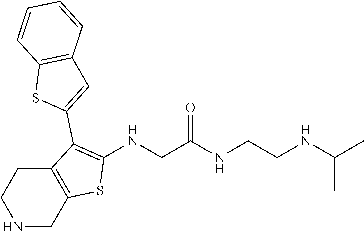 [SMILES: CC(C)NCCNC(=O)CNc1sc2c(c1-c1cc3ccccc3s1)CCNC2] | ESI-MS(M + H)⁺ = 430.2 |

136

| | |
|---|---|
| 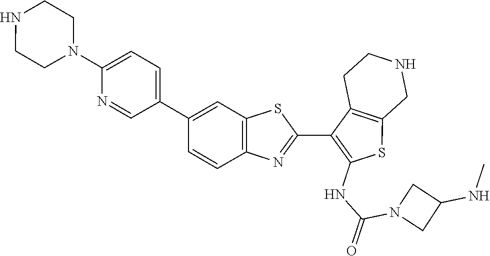 [SMILES: CNC1CN(C(=O)Nc2sc3c(c2-c2nc4ccc(-c5ccc(N6CCNCC6)nc5)cc4s2)CCNC3)C1] | ESI-MS(M + H)⁺ = 561.2 |

137

| | |
|---|---|
| 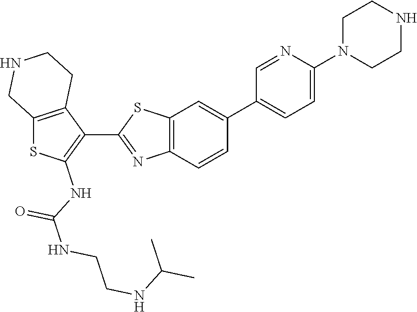 [SMILES: CC(C)NCCNC(=O)Nc1sc2c(c1-c1nc3ccc(-c4ccc(N5CCNCC5)nc4)cc3s1)CCNC2] | ESI-MS(M + H)⁺ = 577.3 |

138

| | |
|---|---|
| 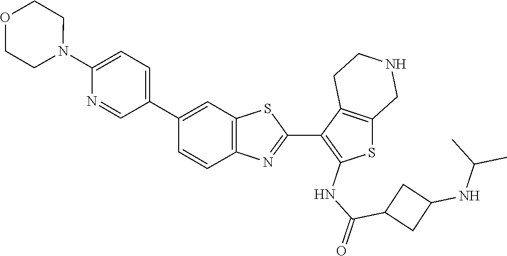 [SMILES: CC(C)NC1CC(C(=O)Nc2sc3c(c2-c2nc4ccc(-c5ccc(N6CCOCC6)nc5)cc4s2)CCNC3)C1] | ESI-MS(M + H)⁺ = 589.2 |

139

| | |
|---|---|
| 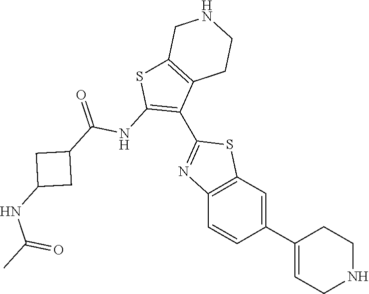 [SMILES: CC(=O)NC1CC(C(=O)Nc2sc3c(c2-c2nc4ccc(C5=CCNCC5)cc4s2)CCNC3)C1] | ESI-MS(M + H)⁺ = 508.2 |

140

| | |
|---|---|
| 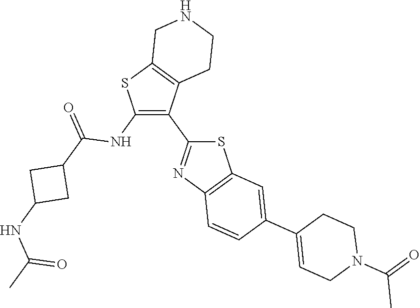 [SMILES: CC(=O)NC1CC(C(=O)Nc2sc3c(c2-c2nc4ccc(C5=CCN(C(C)=O)CC5)cc4s2)CCNC3)C1] | ESI-MS(M + H)⁺ = 550.2 |

141

| | |
|---|---|
| 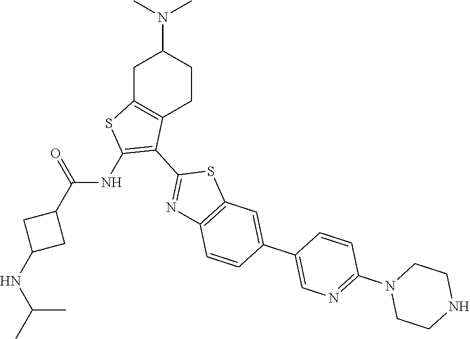 [SMILES: CC(C)NC1CC(C(=O)Nc2sc3c(c2-c2nc4ccc(-c5ccc(N6CCNCC6)nc5)cc4s2)CCC(N(C)C)C3)C1] | ESI-MS(M + H)⁺ = 630.3 |

142

| | |
|---|---|
| 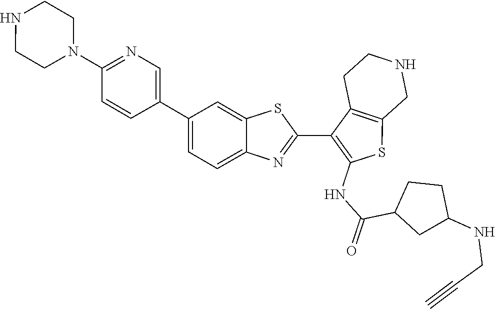 [SMILES: C#CCNC1CCC(C(=O)Nc2sc3c(c2-c2nc4ccc(-c5ccc(N6CCNCC6)nc5)cc4s2)CCNC3)C1] | ESI-MS(M + H)⁺ = 598.2 |
| 143 | |

### Biological Evaluation

### Test Example 1. Degradation Activity of the Compound of the Present Disclosure on c-Myc

(1) A549 cells in the logarithmic growth phase were digested with 0.25% trypsin and counted. The cells were inoculated into 24-well plates at 5000 cells per well, and the 24-well plates were placed in an incubator containing 5% CO₂ at 37° C. for 12 h of culture.

(2) Four concentration gradients for the compounds to be tested (with the maximum drug concentration of 1 μM and 4-fold dilution) were set. After the cells adhered to the wall, the compounds to be tested were serially diluted with DMSO, then diluted with 1640 complete medium to reach a concentration 1000 times the final concentration, and added to the corresponding wells.

(3) After the corresponding concentrations of the compounds to be tested were added, the plates were incubated in an incubator with 5% carbon dioxide at 37° C. for 24 h, and the degradation activity of the compound to be tested on c-myc was detected by western blot. The results are shown in Table 1.

TABLE 1
Degradation activity of the compound
of the present disclosure on c-myc

| Number of | Degradation |
|---|---|
| compound | rate % |
| 1 | ++ |
| 2 | ++ |
| 3 | ++ |
| 4 | ++ |
| 5 | ++ |
| 6 | ++ |
| 7 | ++ |
| 8 | ++ |
| 9 | ++ |
| 10 | ++ |
| 11 | ++ |
| 12 | ++ |
| 13 | ++ |
| 14 | ++ |
| 15 | ++ |
| 16 | ++ |
| 17 | ++ |
| 18 | ++ |
| 19 | ++ |
| 20 | ++ |
| 21 | ++ |
| 22 | ++ |
| 23 | ++ |
| 24 | ++ |
| 25 | ++ |
| 26 | ++ |
| 27 | ++ |
| 28 | ++ |
| 29 | ++ |
| 30 | ++ |
| 31 | ++ |
| 32 | ++ |
| 33 | ++ |
| 34 | ++ |
| 35 | ++ |
| 36 | ++ |
| 37 | ++ |
| 38 | ++ |
| 39 | ++ |
| 40 | ++ |
| 41 | ++ |
| 42 | ++ |
| 43 | ++ |
| 44 | ++ |
| 45 | ++ |
| 46 | ++ |
| 47 | ++ |
| 48 | ++ |
| 49 | ++ |
| 50 | ++ |
| 51 | ++ |
| 52 | ++ |
| 53 | ++ |
| 54 | ++ |
| 55 | ++ |
| 56 | ++ |
| 57 | ++ |
| 58 | ++ |
| 59 | ++ |
| 60 | ++ |
| 61 | ++ |
| 62 | ++ |
| 63 | ++ |
| 64 | ++ |
| 65 | ++ |
| 66 | ++ |
| 67 | ++ |
| 68 | ++ |
| 69 | ++ |
| 70 | ++ |
| 71 | ++ |
| 72 | ++ |
| 73 | ++ |
| 74 | ++ |
| 75 | ++ |
| 76 | ++ |
| 77 | ++ |
| 78 | ++ |
| 79 | ++ |
| 80 | ++ |
| 81 | ++ |
| 82 | ++ |
| 83 | ++ |
| 84 | ++ |
| 85 | ++ |
| 86 | ++ |
| 87 | ++ |
| 88 | ++ |
| 89 | ++ |
| 90 | ++ |
| 91 | ++ |
| 92 | ++ |
| 93 | ++ |
| 94 | ++ |
| 95 | ++ |
| 96 | ++ |
| 97 | ++ |
| 98 | ++ |
| 99 | ++ |
| 100 | ++ |
| 101 | ++ |
| 102 | ++ |
| 103 | ++ |
| 104 | ++ |
| 105 | ++ |
| 106 | ++ |
| 107 | ++ |
| 108 | ++ |
| 109 | ++ |
| 110 | ++ |
| 111 | ++ |
| 112 | ++ |
| 113 | ++ |
| 114 | ++ |
| 115 | ++ |
| 116 | ++ |
| 117 | ++ |
| 118 | ++ |
| 119 | ++ |
| 120 | ++ |
| 121 | ++ |
| 122 | ++ |
| 123 | ++ |
| 124 | ++ |
| 125 | ++ |
| 126 | ++ |
| 127 | ++ |
| 128 | ++ |
| 129 | ++ |
| 130 | ++ |
| 131 | ++ |
| 132 | ++ |
| 133 | ++ |
| 134 | ++ |
| 135 | ++ |
| 136 | ++ |
| 137 | ++ |
| 138 | ++ |
| 139 | ++ |
| 140 | ++ |
| 141 | ++ |
| 142 | ++ |
| 143 | ++ |

+: degradation rate < 50%;
++: degradation rate ≥ 50%
Conclusion: The compounds of the present disclosure have degradation activity on c-myc.

(1) A549 cells in the logarithmic growth phase were digested with 0.25% trypsin and counted. The cells were inoculated into 96-well plates at 2000 cells per well, and the 96-well plates were placed in an incubator containing 5% CO₂ at 37° C. for 12 h of culture.

(2) Six concentration gradients for the compounds to be tested (with the maximum drug concentration of 10 μM and 5-fold dilution) were set. After the cells adhered to the wall, the compounds to be tested were serially diluted with DMSO, then diluted with 1640 complete medium to reach a concentration 1000 times the final concentration, and added to the corresponding wells.

(3) After the corresponding concentrations of the compounds to be tested were added, the plates were incubated in an incubator with 5% carbon dioxide at 37° C., and then the cell viability rate at each drug concentration was determined by CCK8 or CTG method. In addition, the IC50 value was calculated. The results are shown in Table 2 below.

TABLE 2
Inhibitory activity IC₅₀ of the compound of the present disclosure on
proliferation of A549 cells

| Number of compound | Inhibition on proliferation IC₅₀ |
|---|---|
| 001 | 0.49 |
| 009 | 0.53 |
| 017 | 4.41 |
| 047 | 18.27 |
| 048 | 3.35 |
| 054 | 21.75 |
| 060 | 16.76 |
| 069 | 7.80 |
| 077 | 43.38 |
| 120 | 2.77 |
| 124 | 7.37 |
| 125 | 65.23 |
| 134 | 0.66 |
| Compound a | 74.02 |
| Compound b | 98.32 |

Compound a: 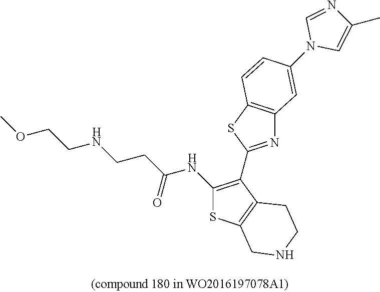
Compound b:

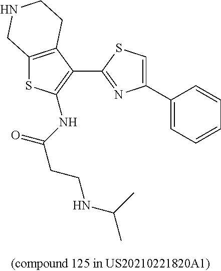 [SMILES: CC(C)NCCC(=O)Nc1sc2c(c1-c1nc(-c3ccccc3)cs1)CCNC2]

Conclusion: The compounds of the present disclosure have obvious inhibitory activity on proliferation of A549 cells.

### Test Example 3: Determination of the Inhibitory Activity of the Compound of the Present Disclosure on Proliferation of HL60 Tumor Cells

The effect of the compound of the present disclosure on the proliferation of tumor cells was determined using the following method. The HL60 cells used in the method were purchased from Nanjing Kebai Biotechnology Co., Ltd., and the kit was the Cell Counting Kit-8/CCK-8 kit of MCE Company (article number: HY—K0301, specification: 3000T).

### Experimental Steps:

(1) HL60 cells in logarithmic growth phase were prepared into a cell suspension. The cell suspension was added into a 96-well plate at 5000 cells/well. The 96-well cell culture plate was placed in a CO₂ incubator for 4 h of incubation of suspended cells or for 12 h of incubation of adherent cells.

(2) The compounds to be tested were diluted with IMDM complete culture medium to reach a concentration twice the testing concentration, and added to the corresponding wells.

(3) After the corresponding concentrations of the compounds to be tested were added, the plates were incubated in an incubator with 5% carbon dioxide at 37° C., and then the cell viability rate at each drug concentration was determined by CCK8 or CTG method. In addition, the IC50 value was calculated. The results are shown in Table 3 below.

TABLE 3
Inhibitory activity IC₅₀ of the compound of the
present disclosure on proliferation of HL60

| | Inhibition on proliferation |
|---|---|
| Number of compound | of HL60 IC₅₀ |
| Compound a | +++ |
| 009 | ++++ |
| 014 | ++++ |
| 017 | ++++ |
| 022 | ++++ |
| 047 | ++++ |
| 048 | ++++ |
| 069 | ++++ |
| 077 | ++++ |
| 119 | ++++ |
| 120 | ++++ |
| 121 | ++++ |

Note:
+: IC₅₀ > 10 μm;
++: 1500 nM ≤ IC₅₀ < 10 μM;
+++: 150 nM ≤ IC₅₀ < 1500 nM;
++++: IC₅₀ < 150 nM
Conclusion: The compounds of the present disclosure have excellent inhibitory activity on cell proliferation.

### Test Example 4: Determination of the Inhibitory Activity of the Compound of the Present Disclosure on Proliferation of HK-2 Cells

(1) HK-2 cells in the logarithmic growth phase were digested with 0.25% trypsin and counted. The cells were inoculated into 96-well plates at 2000 cells per well, and the 96-well plates were placed and cultured in an incubator containing 5% CO₂ at 37° C. for 12 h.

(2) Six concentration gradients for the compound to be tested (with the maximum drug concentration of 10 μM and 5-fold dilution) were set. After the cells adhered to the wall, the compounds to be tested were serially diluted with DMSO, then diluted with 1640 complete medium to reach a concentration 1000 times the final concentration, and added to the corresponding wells.

(3) After the corresponding concentrations of the compounds to be tested were added, the plates were incubated in an incubator with 5% carbon dioxide at 37° C., and then the cell viability rate at each drug concentration was determined by CCK8 or CTG method. In addition, the IC50 value was calculated. The results are shown in Table 4 below.

TABLE 4
Inhibitory activity IC₅₀ of the compound of the
present disclosure on proliferation of HK-2

| | Inhibition on |
|---|---|
| Number of | proliferation of HK-2 |
| compound | IC₅₀ |
| 001 | 759 |
| 047 | 1613 |
| 048 | 1164 |
| 060 | 1102 |
| 069 | 2436 |
| 077 | 1260 |
| 082 | 4511 |
| 086 | 7561 |
| 120 | 760 |
| 121 | 725 |
| 122 | 1043 |
| 123 | 937 |
| 124 | 3265 |
| 125 | 11100 |
| 126 | 1179 |
| 139 | 1985 |
| 140 | 3902 |
| 141 | 1457 |
| 142 | 1072 |
| Compound a | 520.5 |
| Compound b | 241.90 |

Conclusion: The compounds of the present disclosure have no obvious inhibitory activity on proliferation of normal cells.

1. A compound represented by formula (I) or a stereoisomer, a tautomer or a pharmaceutically acceptable salt thereof: 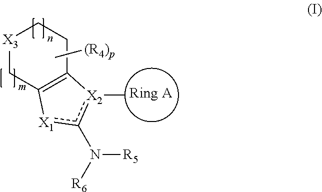
wherein:
X₁ is selected from the group consisting of N, NR₁, O and S;
X₂ is selected from the group consisting of C, CH and N;
X₃ is selected from the group consisting of C(O), C(═NR₃), CR₂R₂′, NR₃, O, S, S(O)₂ and S(═NH) O;
R₁ is selected from the group consisting of H, C₁-C₈ alkyl, C₁-C₈ haloalkyl, C₃-C₁₂ cycloalkyl, 3- to 12-membered heterocyclyl, C₆-C₁₀ aryl, 5- to 12-membered heteroaryl, amino, hydroxyl, nitro and cyano;
R₂ and R₂′ are each independently selected from the group consisting of H, deuterium, halogen, C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, amino, hydroxyl, nitro, cyano, C₁-C₈ alkylamino, C₁-C₈ haloalkylamino, C₁-C₈ alkyl-C(O)O—, and C₁-C₈ alkyl-C(O)NH—; or R₂ and R₂′ form a 3- to 12-membered ring together with the carbon atom to which they are jointly attached, and the 3- to 12-membered ring contains 0, 1 or 2 heteroatoms selected from the group consisting of N, O, S and P; the alkyl, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, nitro, cyano, —C(O)—(C₁-C₅)alkyl, —C(O)O—(C₁-C₈)alkyl, —C(O)NH—(C₁-C₈)alkyl and —NHC (O)—(C₁-C₈)alkyl, and R₂ and R₂′ are not both H;
R₃ and R₄ are each independently selected from the group consisting of H, deuterium, halogen, C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, amino, hydroxyl, nitro, cyano, carboxyl, C₁-C₈ alkylamino, C₁-C₈ haloalkylamino, —OR_{g}, —SR_{g}, —C₁-C₈ alkylene-R_{g}, —OC(O)R_{g}, —C(O)R_{g}, —C(O)OR_{g}, —C(O)N(Rₓ)R_{y}, —NRₓR_{y}, —N(CH₃)R_{g}, —N(Rₓ)C(O)R_{y}, —N(Rₓ)C(O)NRₓR_{y}, —N(Rₓ)C(O)OR_{g}, —N(Rₓ)S(O)NRₓR_{y}, —N(Rₓ)S(O)₂NRₓR_{y}, —N(Rₓ)S(O)₂R_{g}, —S(O)R_{g}, —S(O)₂R_{g}, —S(O)₂NRₓR_{y} and —P(O)RₓR_{y}; the alkyl, alkylene, alkenyl, alkynyl, cycloalkyl, heterocyclyl, alkoxy, aryl, heteroaryl, amino or hydroxyl is optionally further substituted by one or more Rₙ; or
two R₄ form a 3- to 8-membered ring together with one or more atoms to which they are attached (provided that the valence is satisfied), the 3- to 8-membered ring contains 0, 1 or 2 heteroatoms selected from the group consisting of N, O, S and P, and the 3- to 8-membered ring is optionally further substituted by one or more substituents selected from the group consisting of halogen, C₁-C₈alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano; or
R₃ and R₄ form a 3- to 8-membered heterocyclic ring together with the atom to which they are attached, and the 3- to 8-membered heterocyclic ring is optionally further substituted by one or more substituents selected from the group consisting of halogen, C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano; or
R₂ and R₄ form a 3- to 8-membered ring together with the atom to which they are attached, the 3- to 8-membered ring contains 0, 1 or 2 heteroatoms selected from the group consisting of N, O, S and P, and the 3- to 8-membered ring is optionally further substituted by one or more substituents selected from the group consisting of halogen, C₁-C₈ alkyl, C₁-C₈ haloalkyl, C₃-C₁₂ cycloalkyl, 3- to 12-membered heterocyclyl, C₁-C₈ alkoxy, halogen, amino, hydroxyl, oxo, nitro and cyano;
R₅ is selected from the group consisting of H, C₁-C₈ alkyl, C₃-C₁₂ cycloalkyl, 3- to 12-membered heterocyclyl and C₁-C₈ haloalkyl, and the alkyl, cycloalkyl, heterocyclyl or haloalkyl is optionally further substituted by one or more substituents selected from the group consisting of C₁-C₈ alkyl, C₃-C₁₂ cycloalkyl, 3- to 12-membered heterocyclyl, C₁-C₈ alkoxy, halogen, amino, hydroxyl, nitro and cyano;
R₆ is selected from the group consisting of —C(RₐR_{b})R_{c}, —C(O)OR_{c}, —C(O)NRₕR_{c}, —C(S)C(RₐR_{b})R_{c}, —C(S) N(Rₕ)C(RₐR_{b})R_{c}, C₆-C₁₀ aryl, 5- to 12-membered heteroaryl-NR_{d}Rₑ, 3- to 12-membered heterocyclyl, C₃-C₁₂ cycloalkyl, —C(O)—C(O)—C(RₐR_{b})R_{c}, —C(O)—C(O)—N(Rₕ)—C(RₐR_{b})R_{c}, 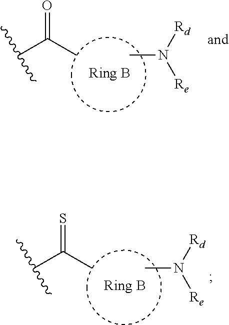
or
R₅ and R₆ form a 3- to 14-membered heterocyclic ring substituted by one or more substituents together with the atom to which they are attached, the substituent is selected from the group consisting of C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, oxo, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —C₁-C₈ alkylene-O—(C₁-C₈)alkyl and —NR_{d}Rₑ; or, when two substituents are attached to the same atom on the 3- to 14-membered heterocyclic ring, the substituents form a 3- to 12-membered ring together with the atom to which they are attached, and the 3- to 12-membered ring contains 0, 1 or 2 heteroatoms selected from the group consisting of N, O, S and P; the alkyl, alkylene, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈alkyl, C₃-C₁₂ cycloalkyl, 3- to 12-membered heterocyclyl, C₁-C₈ alkoxy, C₁-C₈ haloalkoxy, halogen, amino, hydroxyl, nitro and cyano;
Rₐ and R_{b} are each independently selected from the group consisting of H, halogen, C₁-C₈ alkyl, C₃-C₁₂ cycloalkyl, C₁-C₈ haloalkyl, 3- to 12-membered heterocyclyl, C₆-C₁₀ aryl, 5- to 12-membered heteroaryl, C₂-C₈ alkynyl, C₂-C₈ alkenyl, —OH, —NH₂, C₁-C₈ alkoxy, C₁-C₈ alkylamino and —CN, the alkyl, cycloalkyl, haloalkyl, heterocyclyl, aryl, heteroaryl, alkynyl or alkenyl is optionally further substituted by one or more Rₙ;
R_{c} is selected from —(CH₂)s-NR_{d}Rₑ, wherein one or more CH₂ are optionally replaced by one or more groups selected from the group consisting of —N(Rₕ′)—, —C(RₐR_{b})—, —O—, —S—, —C(O)—, C₃-C₁₂ cycloalkyl, 3- to 12-membered heterocyclyl, phenyl, 5- to 12-membered heteroaryl, —C(Rₐ)—C(R_{b})—, alkyne bond, —S(O)—, —S(O)₂— and —P(O)Rₕ′;
R_{d} is selected from the group consisting of H, —(CH₂)s-Rₙ, wherein one or more CH₂ are optionally replaced by one or more groups selected from the group consisting of —N(Rₕ′)—, —C(RₐR_{b})—, —O—, —S—, —C(O)—, C₃-C₁₂ cycloalkyl, 3- to 12-membered heterocyclyl, phenyl, 5- to 12-membered heteroaryl, —C(Rₐ)—C(R_{b})—, alkyne bond, —S(O)—, —S(O)₂— and —P(O)Rₕ′, the —CH₂—, cycloalkyl, heterocyclyl, phenyl or heteroaryl is optionally further substituted by one or more Rₙ;
R_{c} is selected from the group consisting of H, C₁-C₈alkyl, C₃-C₁₂ cycloalkyl, C₁-C₈ haloalkyl, 3- to 12-membered heterocyclyl, phenyl and 5- to 12-membered heteroaryl; the alkyl, cycloalkyl, heterocyclyl, phenyl or heteroaryl is optionally further substituted by one or more Rₘ;
Rₘ is selected from the group consisting of H, halogen, C₁-C₈ alkyl, C₃-C₁₂ cycloalkyl, C₁-C₈alkoxy, C₁-C₈alkylamino, amino, hydroxyl, cyano and nitro;
Rₕ and Rₕ′ are each independently selected from the group consisting of H, C₁-C₈ alkyl, C₁-C₈ haloalkyl, C₃-C₁₂ cycloalkyl, phenyl, 5- to 12-membered heteroaryl and 3- to 12-membered heterocyclyl; the alkyl, haloalkyl, cycloalkyl, phenyl, heteroaryl or heterocyclyl is optionally further substituted by one or more Rₙ;
Ring A is selected from the group consisting of 5- to 6-membered heterocyclic ring, 5- to 12-membered heteroaromatic ring, benzene ring, 9- to 10-membered bicyclic heterocyclyl and 9- to 10-membered fused ring; the heterocyclic ring, heteroaromatic ring, benzene ring or fused ring is optionally further substituted by one or more Rₙ; or, when two Rₙ are attached to the same atom, the two Rₙ form a 3- to 6-membered ring together with the atom to which they are attached; or, when two Rₙ are attached to adjacent atoms, the two Rₙ form a 3- to 12-membered ring together with the atom to which they are attached;
Ring B is selected from the group consisting of C₃-C₁₂ cycloalkyl, 3- to 12-membered heterocyclyl, C₆-C₁₀ aryl and 5- to 12-membered heteroaryl, the cycloalkyl, heterocyclyl, aryl or heteroaryl is optionally further substituted by one or more Rₙ′;
when Ring B is selected from benzene ring, Rₐ is selected from H;
Rₙ and Rₙ′ are each independently selected from the group consisting of H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heterocyclyloxy, amino sulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —OR_{g}, —SR_{g}, —C₁-C₈ alkylene-R_{g}, —OC(O)R_{g}, —C(O)R_{g}, —C(O)OR_{g}, —C(O)N(Rₓ)R_{y}, —NRₓR_{y}, —N(CH₃)R_{g}, —N(Rₓ)C(O)R_{y}, —N(Rₓ)C(O)NRₓR_{y}, —N(Rₓ)C(O)OR_{g}, —N(Rₓ)S(O)NRₓR_{y}, —N(Rₓ)S(O)₂NRₓR_{y}, —N(Rₓ)S(O)₂R_{g}, —S(O)R_{g}, —S(O)₂R_{g}, —S(O)₂NRₓR_{y} and —P(O)RₓR_{y}; or
two Rₙ form a 3- to 12-membered ring together with the atom to which they are jointly attached, and the 3- to 12-membered ring contains 0, 1 or 2 heteroatoms selected from the group consisting of N, O, S and P; the alkyl, alkylene, alkoxy, alkenyl, alkynyl, alkenyloxy, cycloalkyl, cycloalkoxy, heterocyclyl, heterocyclyloxy, aryl, heteroaryl, 3- to 12-membered ring, amino, hydroxyl or amide is optionally further substituted by one or more Rₒ; or
two Rₙ′ form a 3- to 12-membered ring together with the atom to which they are jointly attached, and the 3- to 12-membered ring contains 0, 1 or 2 heteroatoms selected from the group consisting of N, O, S and P; the alkyl, alkylene, alkoxy, alkenyl, alkynyl, alkenyloxy, cycloalkyl, cycloalkoxy, heterocyclyl, heterocyclyloxy, aryl, heteroaryl, 3- to 12-membered ring, amino, hydroxyl or amide is optionally further substituted by one or more Rₒ; or
when two Rₒ are attached to the same atom, the two Rₒ form a 3- to 6-membered ring together with the atom to which they are attached; or, when two Rₒ are attached to adjacent atoms, the two Rₒ form a 3- to 12-membered ring together with the atom to which they are attached;
R_{g}, Rₓ, R_{y} and Rₒ are each independently selected from the group consisting of H, deuterium, halogen, C₁-C₈ alkyl, C₁-C₈ alkoxy, C₂-C₈ alkenyl, C₂-C₈ alkynyl, C₂-C₈ alkenyloxy, C₃-C₁₂ cycloalkyl, C₃-C₈ cycloalkoxy, 3- to 12-membered heterocyclyl, 3- to 12-membered heterocyclyloxy, aminosulfonyl, C₆-C₁₀ aryl, 5- to 12-membered heteroaryl, cyano, amino, nitro, hydroxyl, oxo, carboxyl, amide, hydroxyalkyl, aminoalkyl, alkylcarbonyl, alkoxycarbonyl, C₁-C₈ alkylamino, C₁-C₈ haloalkylamino, —ORₛ, —SRₛ, —C₁-C₈ alkylene-Rₛ, —OC(O)Rₛ, —C(O)Rₛ, —C(O)ORₛ, —C(O)N(Rₛ)Rₜ, —NRₛRₜ, —N(CH₃)Rₛ, —N(Rₛ)C(O)Rₜ, —N(Rₛ)C(O)NRₛRₜ, —N(Rₛ)C(O)ORₜ, —N(Rₛ)S(O)NRₛRₜ, —N(Rₛ)S(O)₂NRₛRₜ, —N(Rₛ)S(O)₂Rₜ, —S(O)Rₛ, —S(O)₂Rₛ, —S(O)₂NRₛRₜ and —P(O)RₛRₜ, the alkyl, alkylene, cycloalkyl, heterocyclyl, aryl or heteroaryl is optionally further substituted by one or more Rᵣ; or
when two Rᵣ are attached to the same atom, the two Rᵣ form a 3- to 6-membered ring together with the atom to which they are attached, or when the two Rᵣ are attached to adjacent atoms, the two Rᵣ form a 3- to 12-membered ring together with the atom to which they are attached;
Rᵣ, Rₛ and Rₜ are each independently selected from the group consisting of H, deuterium, C₁-C₈ alkyl, C₂-C₈ alkenyl, C₂-C₈ alkynyl, halogen, cyano, amino, nitro, hydroxyl, oxo, C₁-C₈ alkoxy, C₁-C₈ haloalkyl, hydroxyalkyl, aminoalkyl, C₁-C₈ alkylamino, alkylcarbonyl, alkoxycarbonyl, halohydroxyalkyl, C₁-C₈ haloalkylamino, C₃-C₁₂ cycloalkyl, 3- to 12-membered heterocyclyl, carboxyl, amide, C₆-C₁₀ aryl and 5- to 12-membered heteroaryl;
- - - represents a chemical bond or is absent, and at most one chemical bond presents;
m is selected from the group consisting of 0, 1 and 2;
n is selected from the group consisting of 0, 1 and 2;
p is selected from the group consisting of 0, 1, 2, 3, 4, 5 and 6; and
s is selected from the group consisting of 0, 1, 2, 3, 4, 5, 6, 7, 8 and 9.

2. The compound according to claim 1 or the stereoisomer, the tautomer or the pharmaceutically acceptable salt thereof, which is a compound represented by formula (IIa) or formula (IIb) or a stereoisomer, a tautomer or a pharmaceutically acceptable salt thereof, 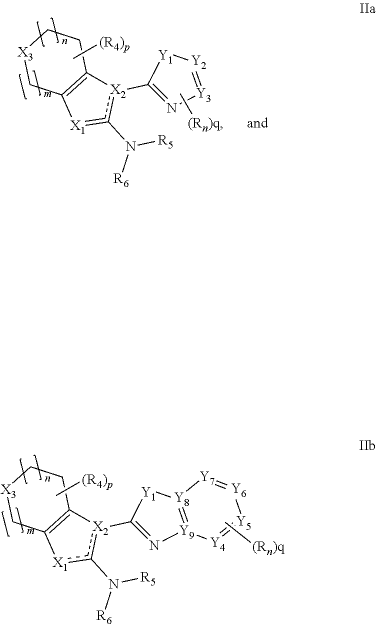
wherein:
Y₁ is selected from the group consisting of CH₂, NH, O and S;
Y₂ is selected from the group consisting of CH and N;
Y₃ is selected from the group consisting of CH and N;
Y₈ is selected from C;
Y₉ is selected from C;
Y₄, Y₅, Y₆ and Y₇ are each independently selected from the group consisting of CH and N;
q is selected from the group consisting of 0, 1, 2, 3, 4 and 5; and
X₁, X₂, X₃, R₄, R₅, R₆, Rₙ, m, n, p and q are as defined in claim 1.

3. The compound according to claim 2 or the stereoisomer, the tautomer or the pharmaceutically acceptable salt thereof, which is a compound represented by formula (IIIa) or formula (IIIb) or a stereoisomer, a tautomer or a pharmaceutically acceptable salt thereof: 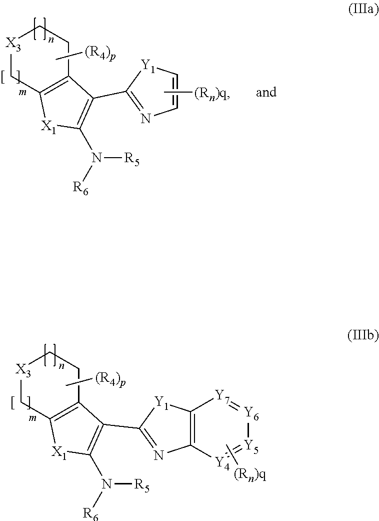
wherein:
X₁ is selected from the group consisting of NH, O and S;
Y₁ is selected from the group consisting of CH₂, NH, O and S;
Y₄, Y₅, Y₆ and Y₇ are each independently selected from the group consisting of CH and N; and
X₃, R₄, R₅, R₆, Rₙ, m, n, p and q are as defined in claim 2.

4. The compound according to claim 3 or the stereoisomer, the tautomer or the pharmaceutically acceptable salt thereof, which is a compound represented by formula (IVa) or formula (IVb) or a stereoisomer, a tautomer or a pharmaceutically acceptable salt thereof: 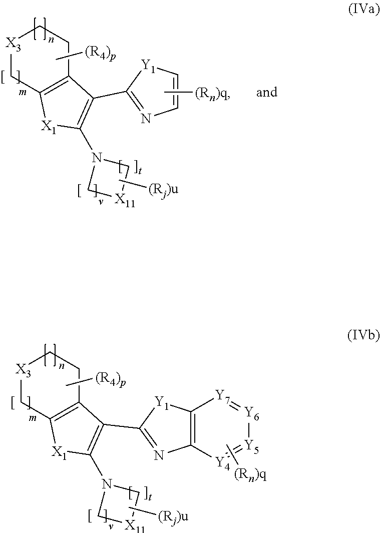
wherein:
X₁₁ is selected from the group consisting of CR₇R₇′, NR₇ and O;
R₇ and R₇′ are each independently selected from the group consisting of H, C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —(C₁-C₈)alkylene-O—(C₁-C₈)alkyl and —NR_{d}Rₑ; or, R₇ and R₇′ form a 3- to 12-membered ring together with the carbon atom to which they are jointly attached, and the 3- to 12-membered ring contains 0, 1 or 2 heteroatoms selected from the group consisting of N, O, S and P; the alkyl, alkylene, alkenyl, alkynyl, cycloalkyl, heterocyclyl, alkoxy, aryl, heteroaryl, 3- to 12-membered ring, amino or hydroxyl is optionally further substituted by one or more substituents selected from the group consisting of C₁-C₈ alkyl, C₃-C₁₂ cycloalkyl, 3- to 12-membered heterocyclyl, C₁-C₈ alkoxy, C₁-C₈ haloalkoxy, halogen, amino, hydroxyl, nitro and cyano;
Rⱼ is selected from the group consisting of C₁-C₈ alkyl, C₂-C₈ alkenyl, C₂-C₈ alkynyl, C₁-C₈ haloalkyl, C₃-C₁₂ cycloalkyl, 3- to 12-membered heterocyclyl, C₁-C₈ alkoxy, C₁-C₈ haloalkoxy, C₆-C₁₀ aryl, 5- to 12-membered heteroaryl, halogen, oxo, amino, hydroxyl, nitro, cyano, C₁-C₈ alkylamino, C₁-C₈ haloalkylamino, C₁-C₈ alkyl-C(O)O—, C₁-C₈ alkyl-C(O)NH—, —(C₁-C₈)alkylene-O—(C₁-C₈)alkyl and —NR_{d}Rₑ, or two Rⱼ form a 3- to 12-membered ring together with the atom to which they are jointly attached, and the 3- to 12-membered ring contains 0, 1 or 2 heteroatoms selected from the group consisting of N, O, S and P;
v is selected from the group consisting of 0, 1, 2, 3, 4, 5 and 6;
t is selected from the group consisting of 0, 1, 2, 3, 4, 5 and 6;
u is selected from the group consisting of 0, 1, 2, 3, 4, 5 and 6;
wherein, v and t are not both 0; and
X₁, X₃, Y₁, Y₄, Y₅, Y₆, Y₇, R₄, Rₙ, R_{d}, Rₑ, m, n, p and q are as defined in claim 3.

5. The compound according to claim 3 or the stereoisomer, the tautomer or the pharmaceutically acceptable salt thereof, which is a compound represented by formula (V) or a stereoisomer, a tautomer or a pharmaceutically acceptable salt thereof: 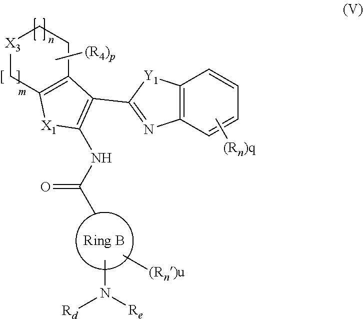
wherein:
Y₁ is selected from the group consisting of CH₂, NH, O and S;
X₁ is selected from the group consisting of NH, O and S;
Ring B is selected from the group consisting of C₃-C₁₂ cycloalkyl, 3- to 12-membered heterocyclyl, C₆-C₁₀ aryl and 5- to 12-membered heteroaryl;
u is selected from the group consisting of 0, 1, 2, 3, 4, 5 and 6; and
X₃, R₄, Rₐ, Rₑ, Rₙ, Rₙ′, m, n, p and q are as defined in claim 3.

6. The compound according to claim 3 or the stereoisomer, the tautomer or the pharmaceutically acceptable salt thereof, wherein:
Rₛ is selected from H;
R₆ is selected from the group consisting of —C(RₐR_{b})R_{c}, —C(O)OR_{c}, —C(O)NRₙR_{c}, —C(O)—C(RₐR_{b})R_{c} and —C(O)—C(O)—N(Rₕ)—C(RₐR_{b})R_{c};
Rₐ, R_{b}, R_{c} and Rₙ are as defined in claim 3.

7. The compound according to claim 6 or the stereoisomer, the tautomer or the pharmaceutically acceptable salt thereof, wherein:
R_{c} is selected from the group consisting of 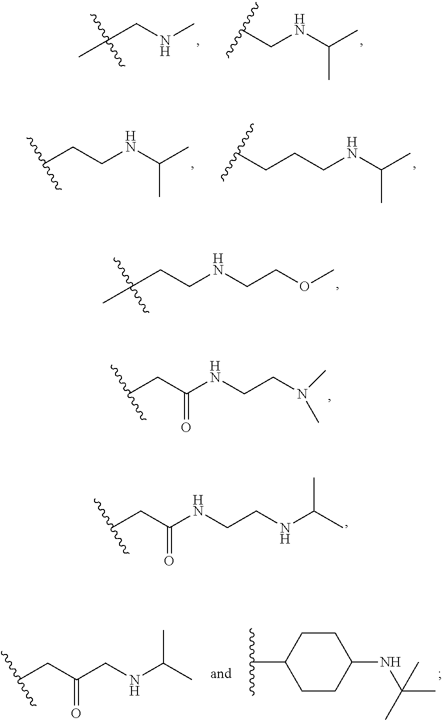
and
Rₐ, R_{b} and Rₕ are as defined in claim 6.

8. The compound according to claim 5 or the stereoisomer, the tautomer or the pharmaceutically acceptable salt thereof, which is a compound represented by formula (VIa) to formula (VId) or a stereoisomer, a tautomer or a pharmaceutically acceptable salt thereof: 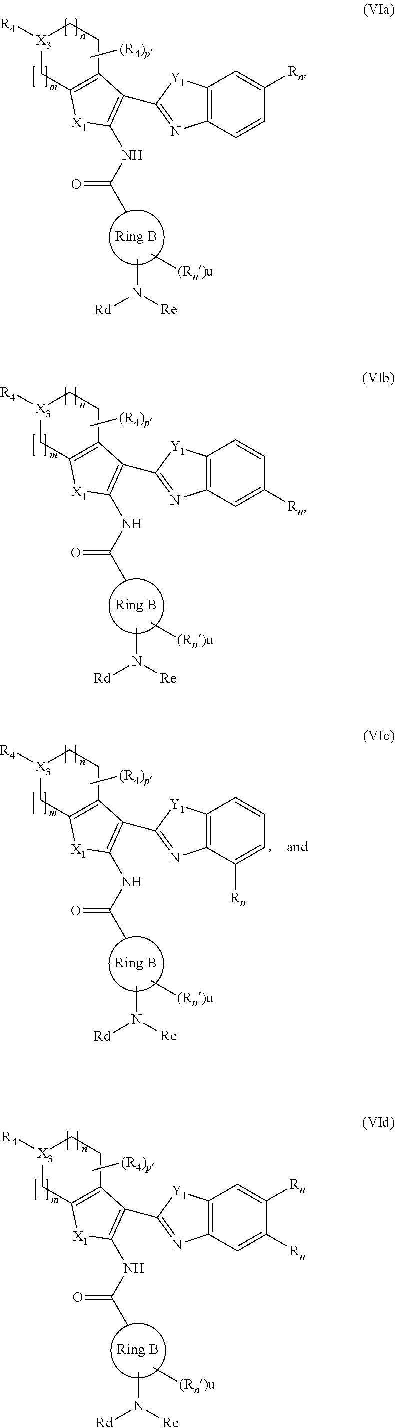
P′ is selected from the group consisting of 0, 1, 2, 3, 4 and 5; and
X₃, R₄, R_{d}, Rₑ, Rₙ, Rₙ′, m, n and q are as defined in claim 5.

9. The compound according to claim 1, or the stereoisomer, the tautomer or the pharmaceutically acceptable salt thereof, wherein Ring B is selected from the group consisting of: 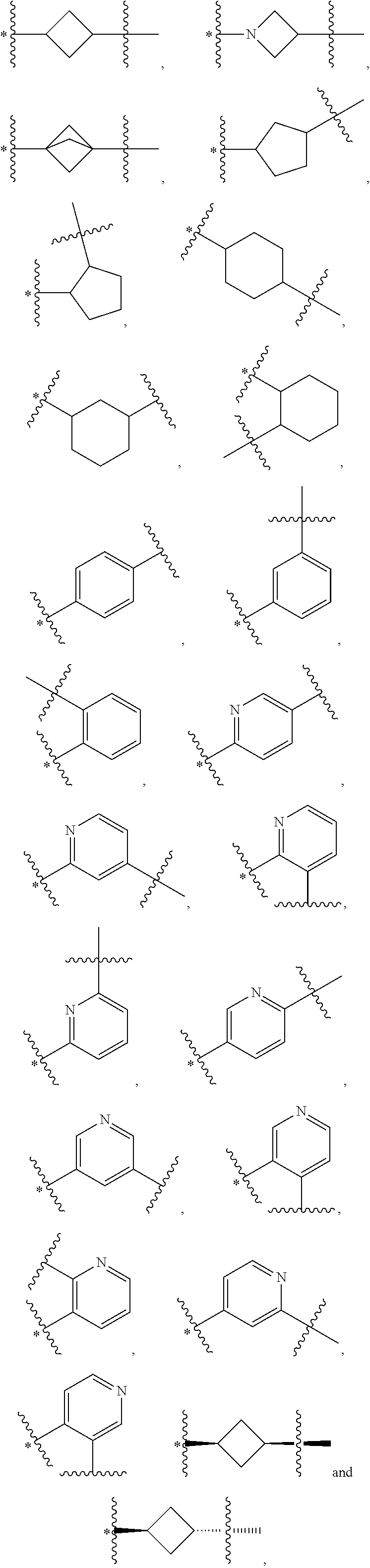
and
*end is attached to carbonyl or carbon-sulfur group.

10. The compound according to claim 1 or the stereoisomer, the tautomer or the pharmaceutically acceptable salt thereof, wherein R_{d} is selected from the group consisting of C₁-C₄ alkyl,

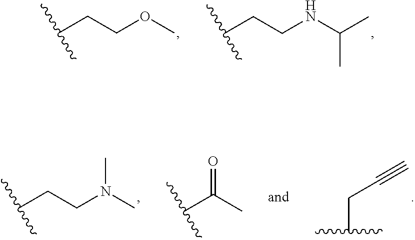

11. The compound according to claim 1 or the stereoisomer, the tautomer or the pharmaceutically acceptable salt thereof, wherein:
Rₙ is selected from the group consisting of H, methyl, halogen, trifluoromethyl, cyano, methoxy, 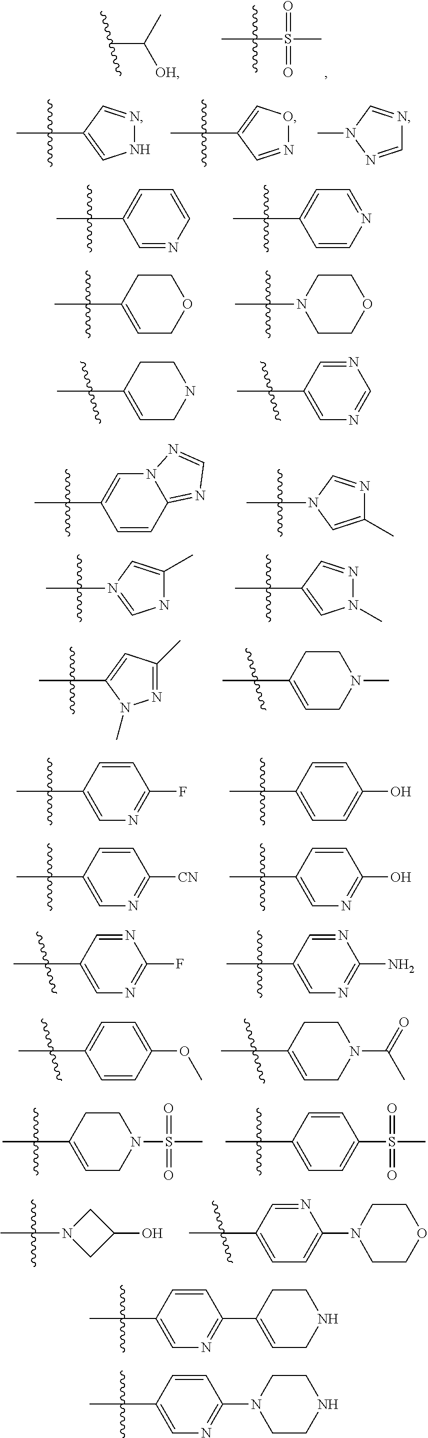 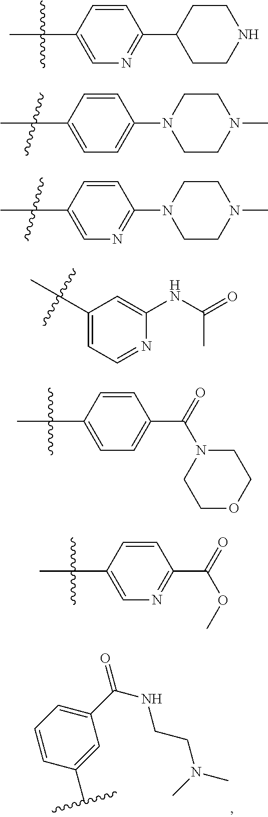
R₄ is selected from the group consisting of H, deuterium, halogen, C₁-C₃ alkyl, C₁-C₃ haloalkyl, C₃-C₆ cycloalkyl, 4- to 6-membered heterocyclyl and —C(O)R_{g}; and
R_{g} is selected from the group consisting of H and C₁-C₃ alkyl.

12. The compound according to claim 1 or the stereoisomer, the tautomer or the pharmaceutically acceptable salt thereof, wherein the compound is selected from the group consisting of:

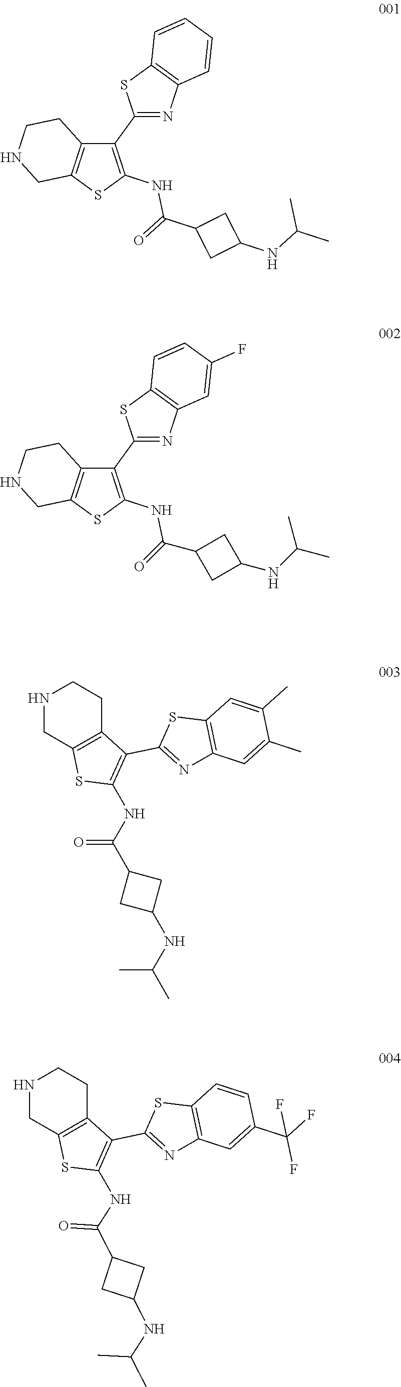 [SMILES: CC(C)NC1CC(C(=O)Nc2sc3c(c2-c2nc4ccccc4s2)CCNC3)C1 ; CC(C)NC1CC(C(=O)Nc2sc3c(c2-c2nc4cc(F)ccc4s2)CCNC3)C1 ; Cc1cc2nc(-c3c(NC(=O)C4CC(NC(C)C)C4)sc4c3CCNC4)sc2cc1C ; CC(C)NC1CC(C(=O)Nc2sc3c(c2-c2nc4cc(C(F)(F)F)ccc4s2)CCNC3)C1]

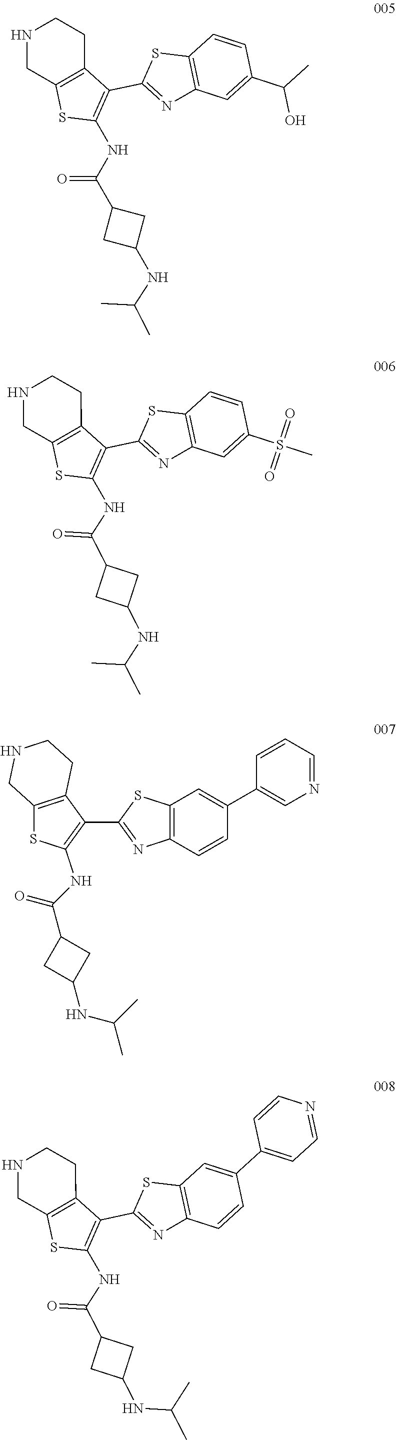 [SMILES: CC(C)NC1CC(C(=O)Nc2sc3c(c2-c2nc4cc(C(C)O)ccc4s2)CCNC3)C1 ; CC(C)NC1CC(C(=O)Nc2sc3c(c2-c2nc4cc(S(C)(=O)=O)ccc4s2)CCNC3)C1 ; CC(C)NC1CC(C(=O)Nc2sc3c(c2-c2nc4ccc(-c5cccnc5)cc4s2)CCNC3)C1 ; CC(C)NC1CC(C(=O)Nc2sc3c(c2-c2nc4ccc(-c5ccncc5)cc4s2)CCNC3)C1]

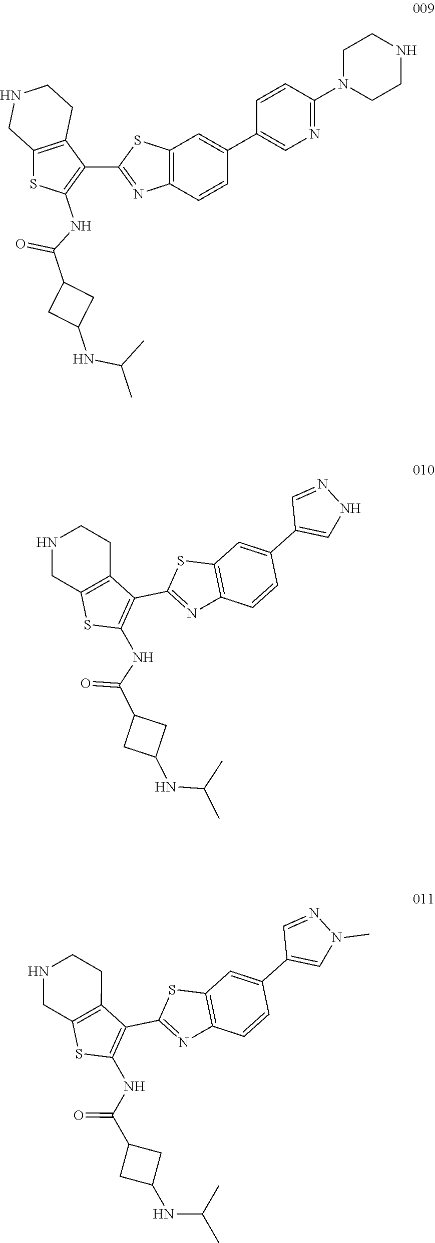 [SMILES: CC(C)NC1CC(C(=O)Nc2sc3c(c2-c2nc4ccc(-c5ccc(N6CCNCC6)nc5)cc4s2)CCNC3)C1 ; CC(C)NC1CC(C(=O)Nc2sc3c(c2-c2nc4ccc(-c5cn[nH]c5)cc4s2)CCNC3)C1 ; CC(C)NC1CC(C(=O)Nc2sc3c(c2-c2nc4ccc(-c5cnn(C)c5)cc4s2)CCNC3)C1]

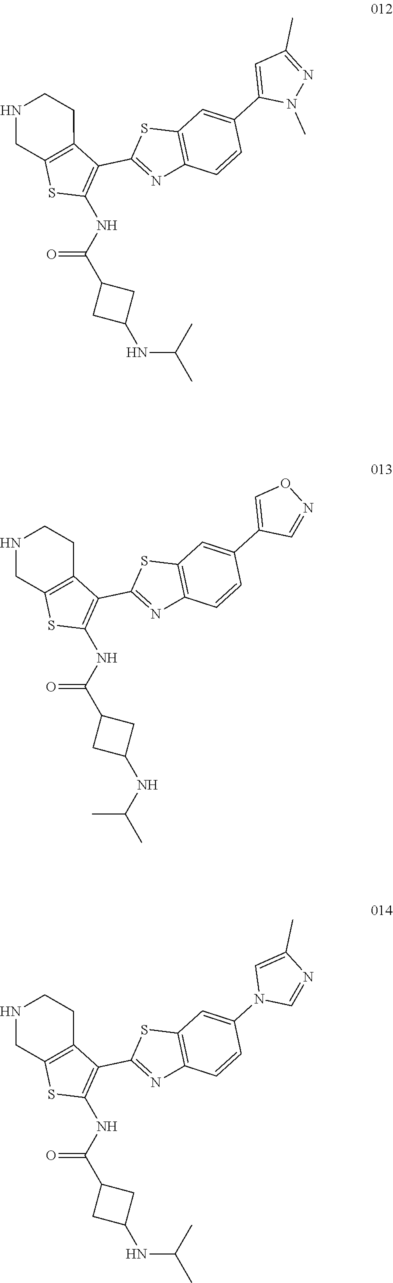 [SMILES: Cc1cc(-c2ccc3nc(-c4c(NC(=O)C5CC(NC(C)C)C5)sc5c4CCNC5)sc3c2)n(C)n1 ; CC(C)NC1CC(C(=O)Nc2sc3c(c2-c2nc4ccc(-c5cnoc5)cc4s2)CCNC3)C1 ; Cc1cn(-c2ccc3nc(-c4c(NC(=O)C5CC(NC(C)C)C5)sc5c4CCNC5)sc3c2)cn1]

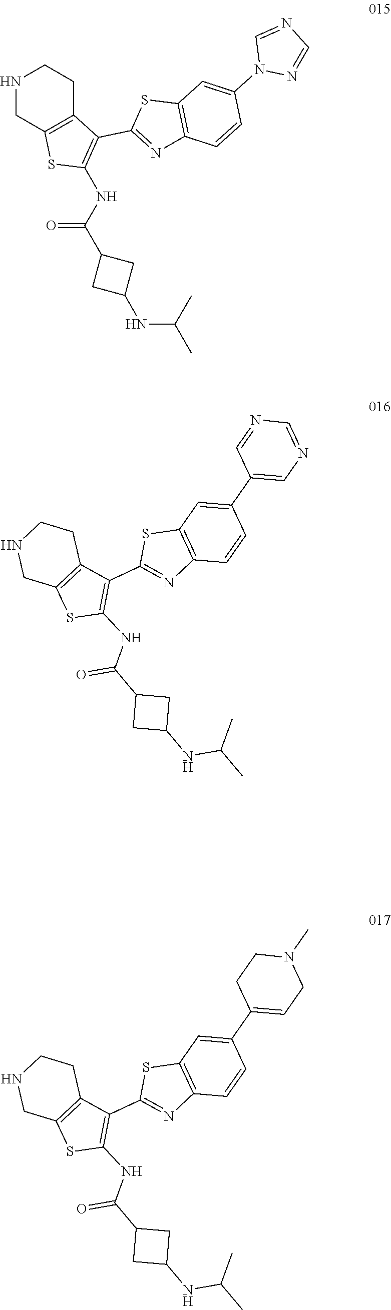 [SMILES: CC(C)NC1CC(C(=O)Nc2sc3c(c2-c2nc4ccc(-n5cncn5)cc4s2)CCNC3)C1 ; CC(C)NC1CC(C(=O)Nc2sc3c(c2-c2nc4ccc(-c5cncnc5)cc4s2)CCNC3)C1 ; CC(C)NC1CC(C(=O)Nc2sc3c(c2-c2nc4ccc(C5=CCN(C)CC5)cc4s2)CCNC3)C1]

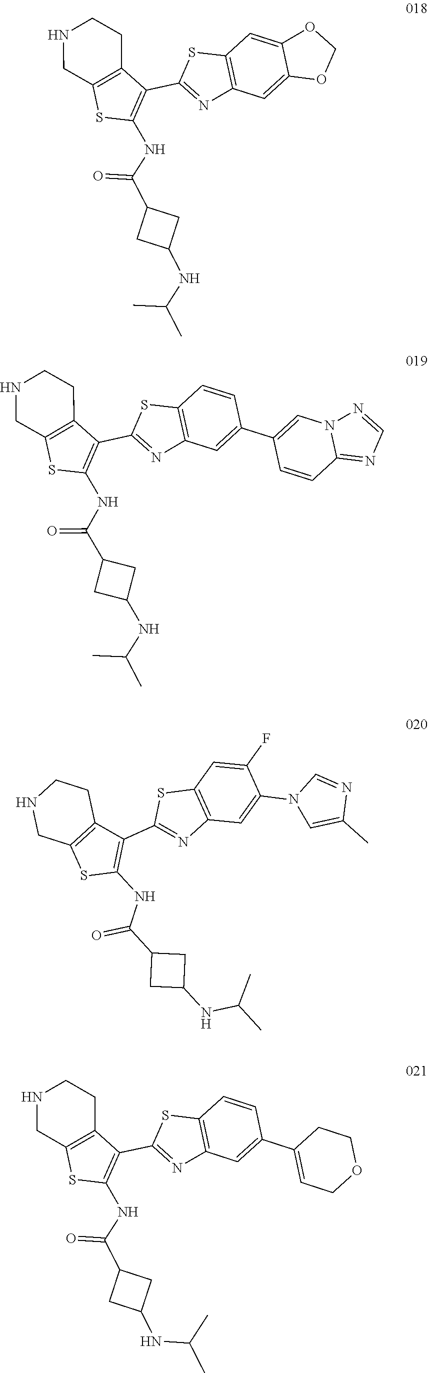 [SMILES: CC(C)NC1CC(C(=O)Nc2sc3c(c2-c2nc4cc5c(cc4s2)OCO5)CCNC3)C1 ; CC(C)NC1CC(C(=O)Nc2sc3c(c2-c2nc4cc(-c5ccc6ncnn6c5)ccc4s2)CCNC3)C1 ; Cc1cn(-c2cc3nc(-c4c(NC(=O)C5CC(NC(C)C)C5)sc5c4CCNC5)sc3cc2F)cn1 ; CC(C)NC1CC(C(=O)Nc2sc3c(c2-c2nc4cc(C5=CCOCC5)ccc4s2)CCNC3)C1]

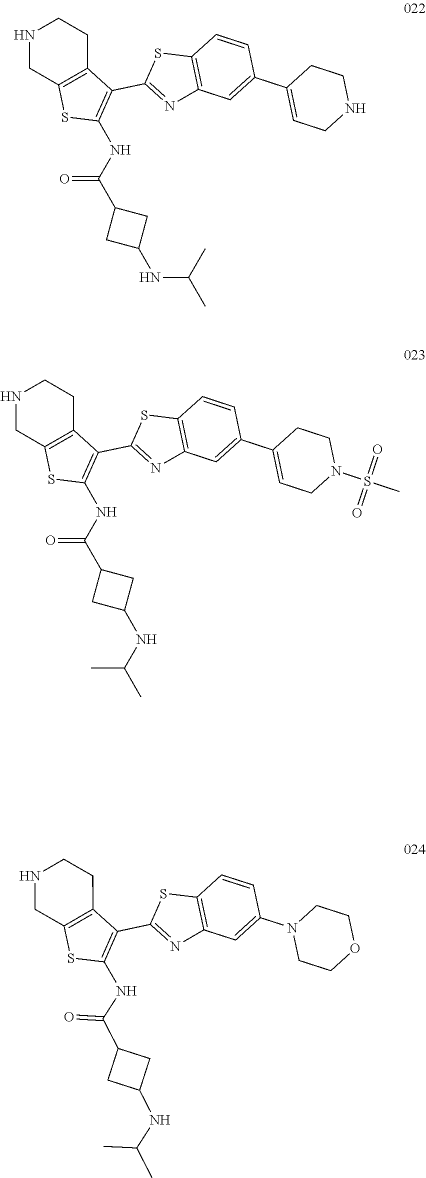 [SMILES: CC(C)NC1CC(C(=O)Nc2sc3c(c2-c2nc4cc(C5=CCNCC5)ccc4s2)CCNC3)C1 ; CC(C)NC1CC(C(=O)Nc2sc3c(c2-c2nc4cc(C5=CCN(S(C)(=O)=O)CC5)ccc4s2)CCNC3)C1 ; CC(C)NC1CC(C(=O)Nc2sc3c(c2-c2nc4cc(N5CCOCC5)ccc4s2)CCNC3)C1]

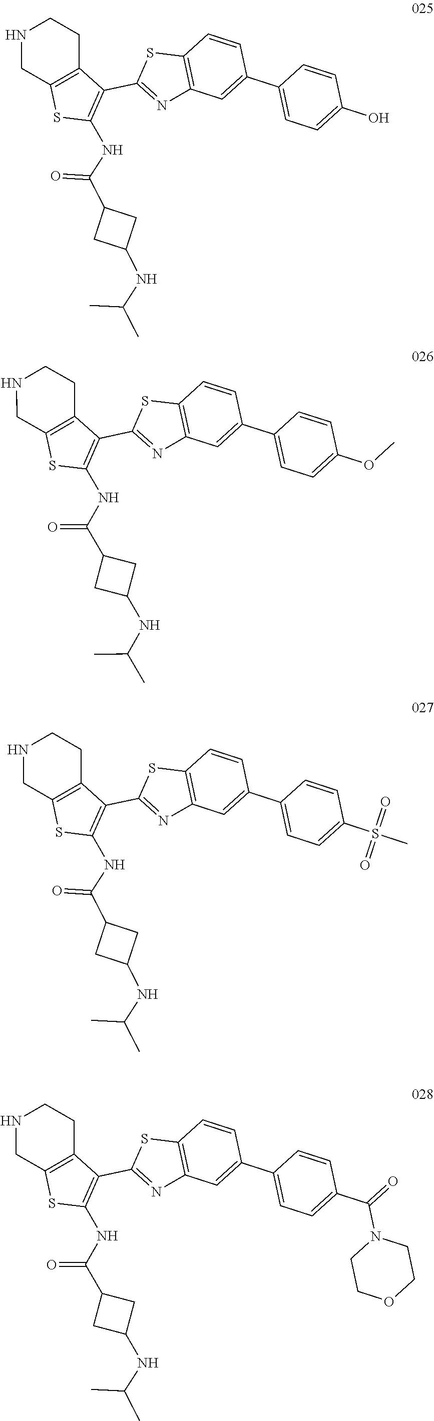 [SMILES: CC(C)NC1CC(C(=O)Nc2sc3c(c2-c2nc4cc(-c5ccc(O)cc5)ccc4s2)CCNC3)C1 ; COc1ccc(-c2ccc3sc(-c4c(NC(=O)C5CC(NC(C)C)C5)sc5c4CCNC5)nc3c2)cc1 ; CC(C)NC1CC(C(=O)Nc2sc3c(c2-c2nc4cc(-c5ccc(S(C)(=O)=O)cc5)ccc4s2)CCNC3)C1 ; CC(C)NC1CC(C(=O)Nc2sc3c(c2-c2nc4cc(-c5ccc(C(=O)N6CCOCC6)cc5)ccc4s2)CCNC3)C1]

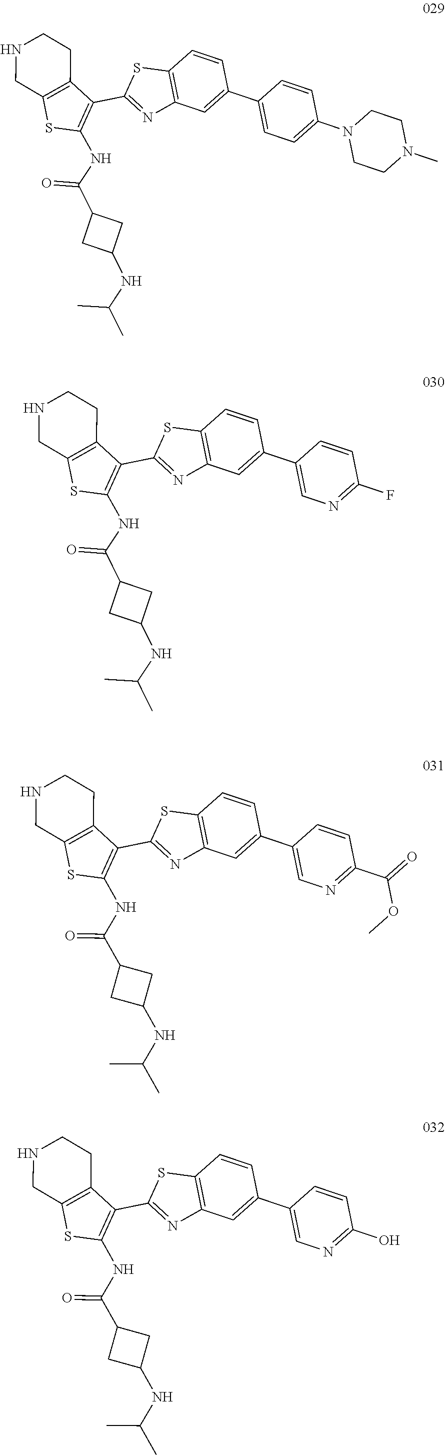 [SMILES: CC(C)NC1CC(C(=O)Nc2sc3c(c2-c2nc4cc(-c5ccc(N6CCN(C)CC6)cc5)ccc4s2)CCNC3)C1 ; CC(C)NC1CC(C(=O)Nc2sc3c(c2-c2nc4cc(-c5ccc(F)nc5)ccc4s2)CCNC3)C1 ; COC(=O)c1ccc(-c2ccc3sc(-c4c(NC(=O)C5CC(NC(C)C)C5)sc5c4CCNC5)nc3c2)cn1 ; CC(C)NC1CC(C(=O)Nc2sc3c(c2-c2nc4cc(-c5ccc(O)nc5)ccc4s2)CCNC3)C1]

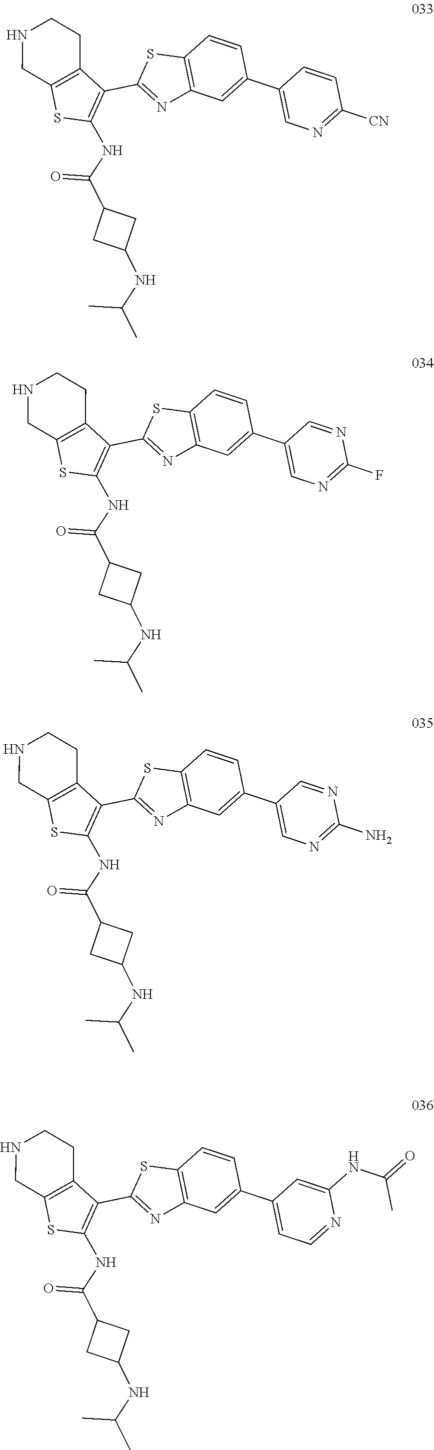 [SMILES: CC(C)NC1CC(C(=O)Nc2sc3c(c2-c2nc4cc(-c5ccc(C#N)nc5)ccc4s2)CCNC3)C1 ; CC(C)NC1CC(C(=O)Nc2sc3c(c2-c2nc4cc(-c5cnc(F)nc5)ccc4s2)CCNC3)C1 ; CC(C)NC1CC(C(=O)Nc2sc3c(c2-c2nc4cc(-c5cnc(N)nc5)ccc4s2)CCNC3)C1 ; CC(=O)Nc1cc(-c2ccc3sc(-c4c(NC(=O)C5CC(NC(C)C)C5)sc5c4CCNC5)nc3c2)ccn1]

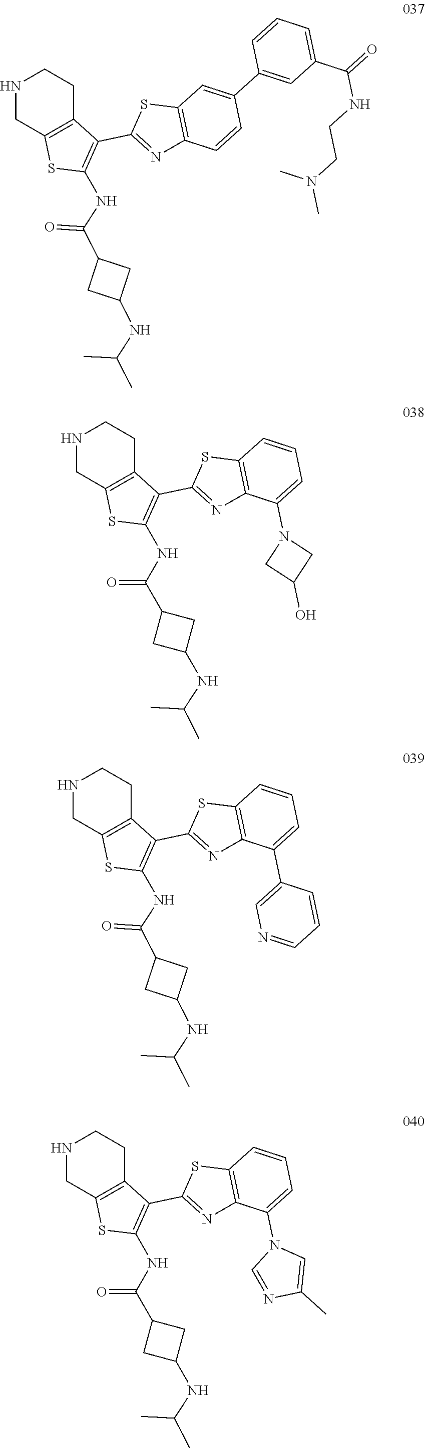

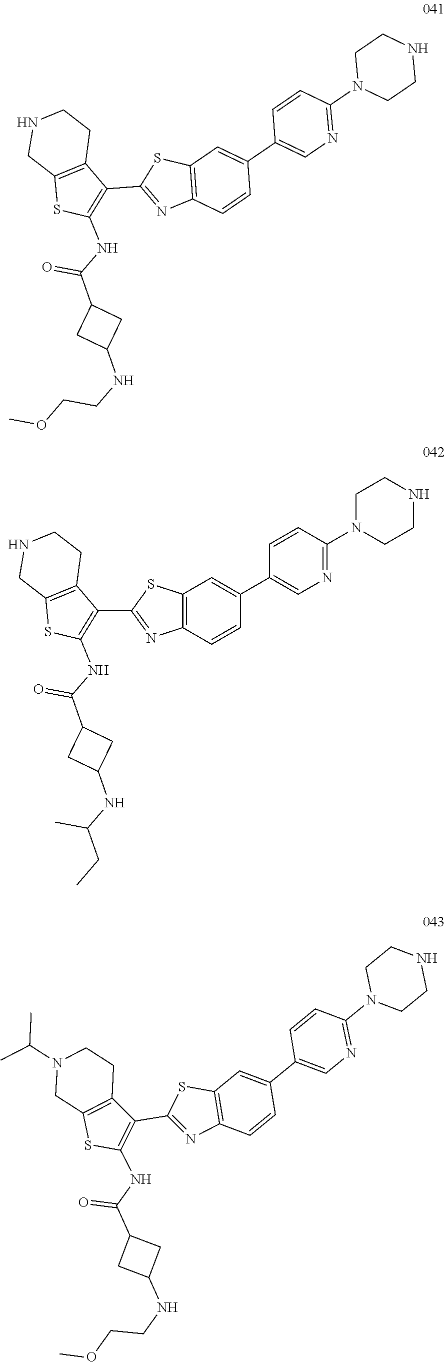 [SMILES: COCCNC1CC(C(=O)Nc2sc3c(c2-c2nc4ccc(-c5ccc(N6CCNCC6)nc5)cc4s2)CCNC3)C1 ; CCC(C)NC1CC(C(=O)Nc2sc3c(c2-c2nc4ccc(-c5ccc(N6CCNCC6)nc5)cc4s2)CCNC3)C1 ; COCCNC1CC(C(=O)Nc2sc3c(c2-c2nc4ccc(-c5ccc(N6CCNCC6)nc5)cc4s2)CCN(C(C)C)C3)C1]

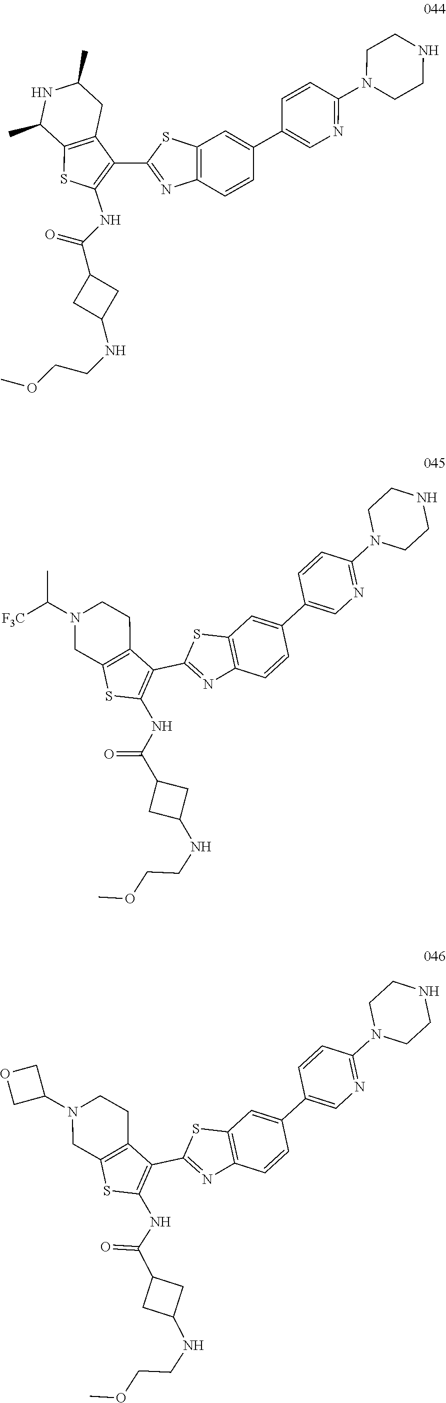 [SMILES: COCCNC1CC(C(=O)Nc2sc3c(c2-c2nc4ccc(-c5ccc(N6CCNCC6)nc5)cc4s2)C[C@H](C)N[C@@H]3C)C1 ; COCCNC1CC(C(=O)Nc2sc3c(c2-c2nc4ccc(-c5ccc(N6CCNCC6)nc5)cc4s2)CCN(C(C)C(F)(F)F)C3)C1 ; COCCNC1CC(C(=O)Nc2sc3c(c2-c2nc4ccc(-c5ccc(N6CCNCC6)nc5)cc4s2)CCN(C2COC2)C3)C1]

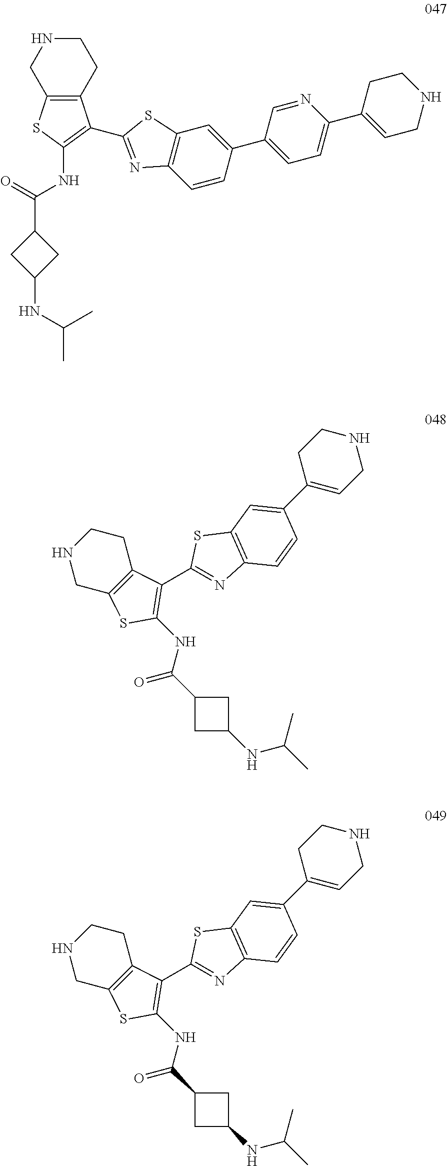 [SMILES: CC(C)NC1CC(C(=O)Nc2sc3c(c2-c2nc4ccc(-c5ccc(C6=CCNCC6)nc5)cc4s2)CCNC3)C1 ; CC(C)NC1CC(C(=O)Nc2sc3c(c2-c2nc4ccc(C5=CCNCC5)cc4s2)CCNC3)C1 ; CC(C)N[C@H]1C[C@@H](C(=O)Nc2sc3c(c2-c2nc4ccc(C5=CCNCC5)cc4s2)CCNC3)C1]

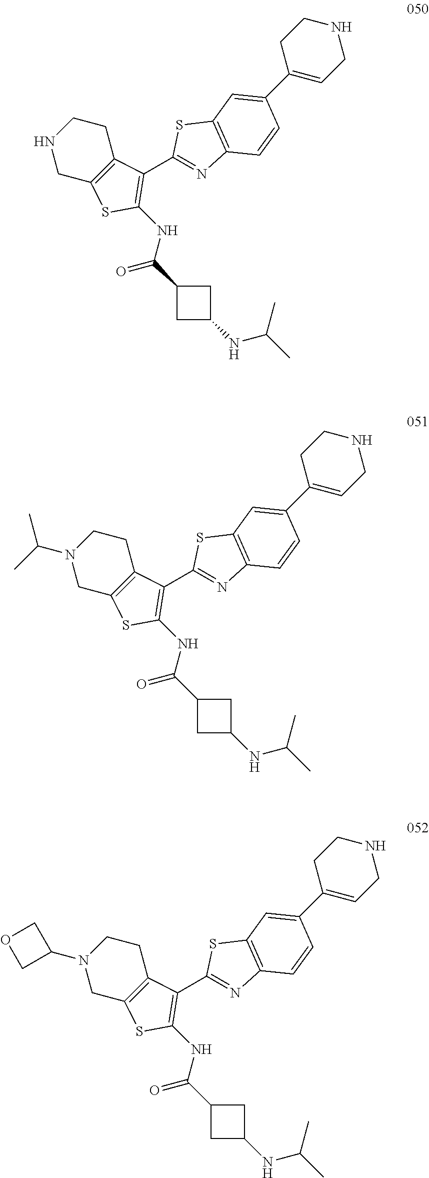 [SMILES: CC(C)N[C@H]1C[C@H](C(=O)Nc2sc3c(c2-c2nc4ccc(C5=CCNCC5)cc4s2)CCNC3)C1 ; CC(C)NC1CC(C(=O)Nc2sc3c(c2-c2nc4ccc(C5=CCNCC5)cc4s2)CCN(C(C)C)C3)C1 ; CC(C)NC1CC(C(=O)Nc2sc3c(c2-c2nc4ccc(C5=CCNCC5)cc4s2)CCN(C2COC2)C3)C1]

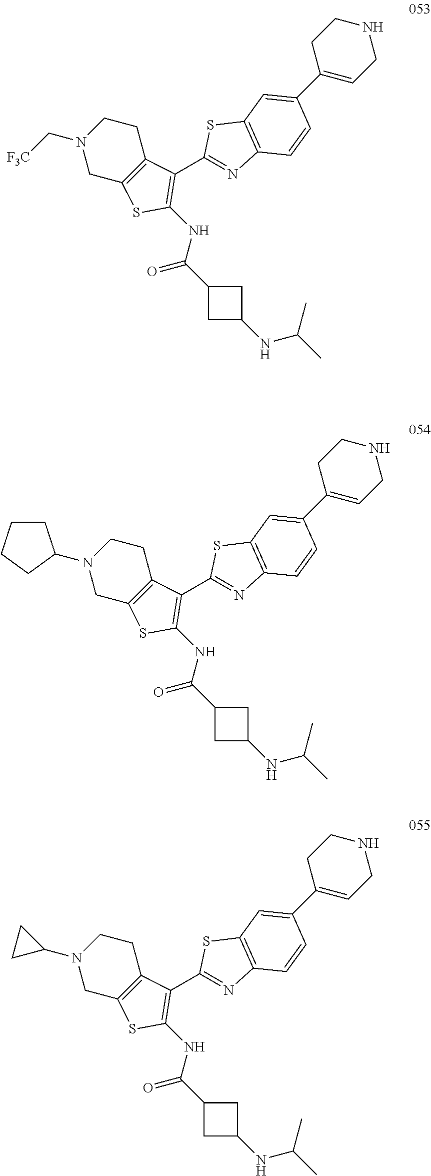 [SMILES: CC(C)NC1CC(C(=O)Nc2sc3c(c2-c2nc4ccc(C5=CCNCC5)cc4s2)CCN(CC(F)(F)F)C3)C1 ; CC(C)NC1CC(C(=O)Nc2sc3c(c2-c2nc4ccc(C5=CCNCC5)cc4s2)CCN(C2CCCC2)C3)C1 ; CC(C)NC1CC(C(=O)Nc2sc3c(c2-c2nc4ccc(C5=CCNCC5)cc4s2)CCN(C2CC2)C3)C1]

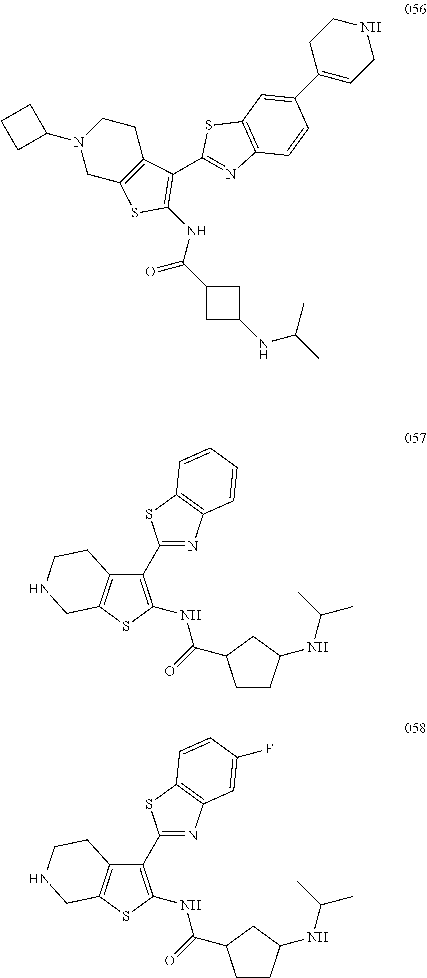 [SMILES: CC(C)NC1CC(C(=O)Nc2sc3c(c2-c2nc4ccc(C5=CCNCC5)cc4s2)CCN(C2CCC2)C3)C1 ; CC(C)NC1CCC(C(=O)Nc2sc3c(c2-c2nc4ccccc4s2)CCNC3)C1 ; CC(C)NC1CCC(C(=O)Nc2sc3c(c2-c2nc4cc(F)ccc4s2)CCNC3)C1]

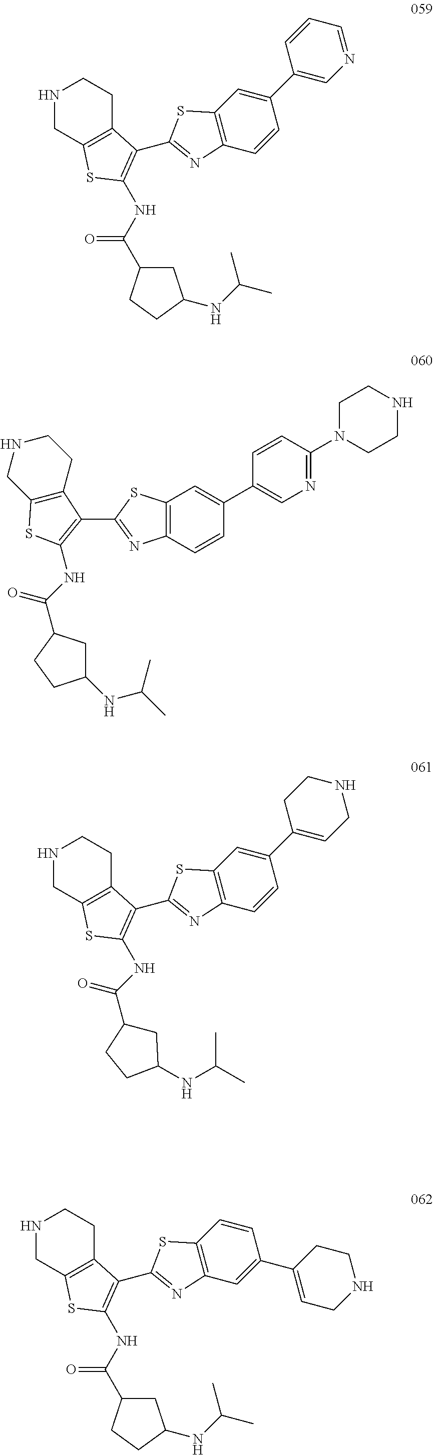 [SMILES: CC(C)NC1CCC(C(=O)Nc2sc3c(c2-c2nc4ccc(-c5cccnc5)cc4s2)CCNC3)C1 ; CC(C)NC1CCC(C(=O)Nc2sc3c(c2-c2nc4ccc(-c5ccc(N6CCNCC6)nc5)cc4s2)CCNC3)C1 ; CC(C)NC1CCC(C(=O)Nc2sc3c(c2-c2nc4ccc(C5=CCNCC5)cc4s2)CCNC3)C1 ; CC(C)NC1CCC(C(=O)Nc2sc3c(c2-c2nc4cc(C5=CCNCC5)ccc4s2)CCNC3)C1]

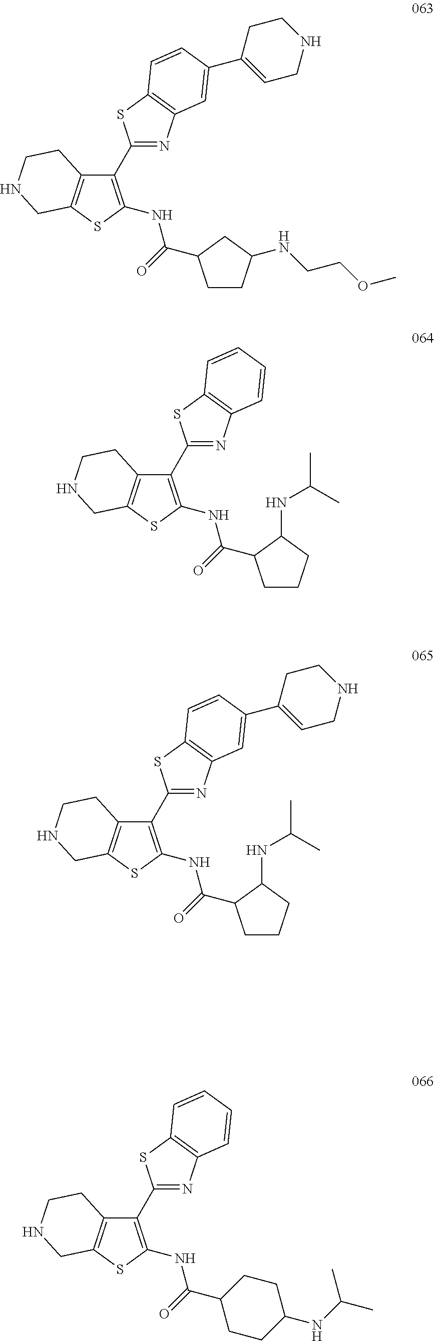 [SMILES: COCCNC1CCC(C(=O)Nc2sc3c(c2-c2nc4cc(C5=CCNCC5)ccc4s2)CCNC3)C1 ; CC(C)NC1CCCC1C(=O)Nc1sc2c(c1-c1nc3ccccc3s1)CCNC2 ; CC(C)NC1CCCC1C(=O)Nc1sc2c(c1-c1nc3cc(C4=CCNCC4)ccc3s1)CCNC2 ; CC(C)NC1CCC(C(=O)Nc2sc3c(c2-c2nc4ccccc4s2)CCNC3)CC1]

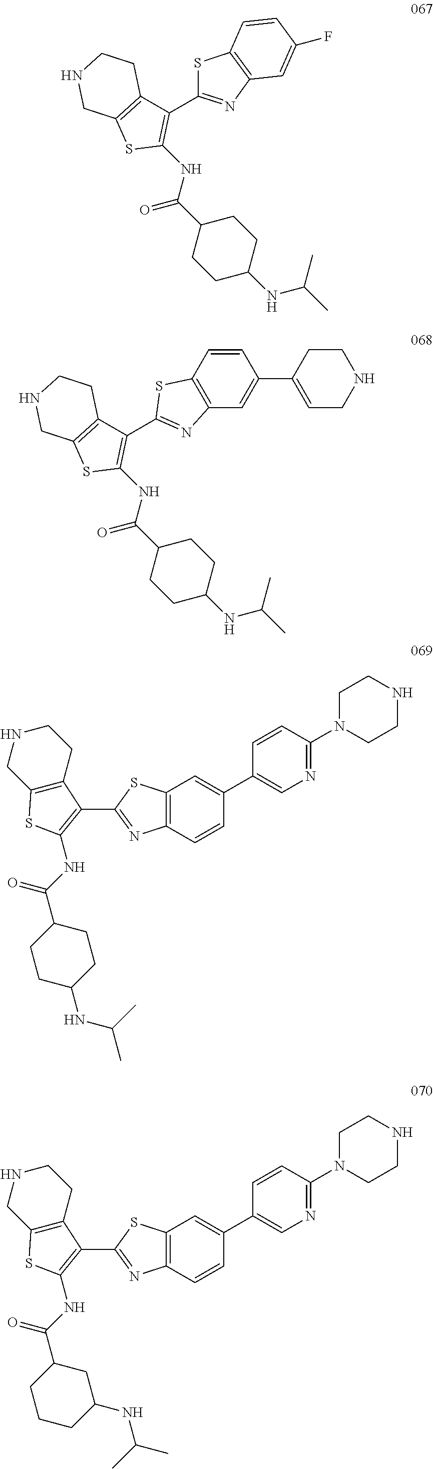

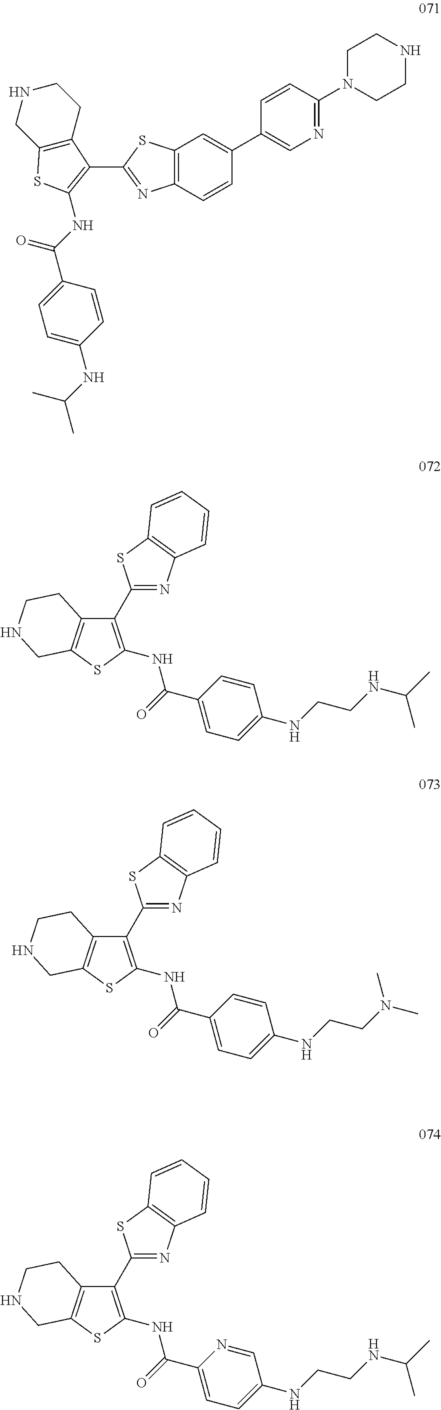 [SMILES: CC(C)Nc1ccc(C(=O)Nc2sc3c(c2-c2nc4ccc(-c5ccc(N6CCNCC6)nc5)cc4s2)CCNC3)cc1 ; CC(C)NCCNc1ccc(C(=O)Nc2sc3c(c2-c2nc4ccccc4s2)CCNC3)cc1 ; CN(C)CCNc1ccc(C(=O)Nc2sc3c(c2-c2nc4ccccc4s2)CCNC3)cc1 ; CC(C)NCCNc1ccc(C(=O)Nc2sc3c(c2-c2nc4ccccc4s2)CCNC3)nc1]

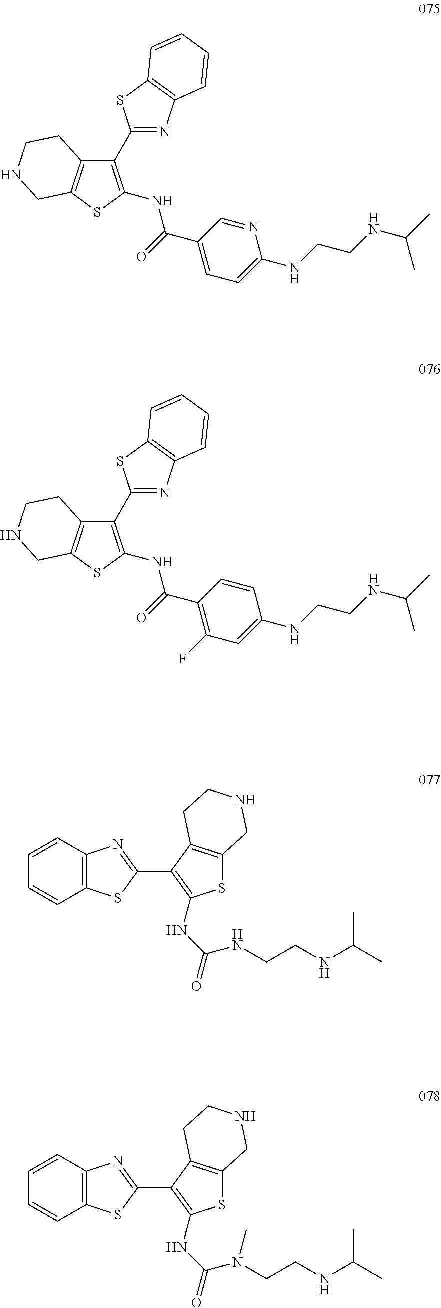 [SMILES: CC(C)NCCNc1ccc(C(=O)Nc2sc3c(c2-c2nc4ccccc4s2)CCNC3)cn1 ; CC(C)NCCNc1ccc(C(=O)Nc2sc3c(c2-c2nc4ccccc4s2)CCNC3)c(F)c1 ; CC(C)NCCNC(=O)Nc1sc2c(c1-c1nc3ccccc3s1)CCNC2 ; CC(C)NCCN(C)C(=O)Nc1sc2c(c1-c1nc3ccccc3s1)CCNC2]

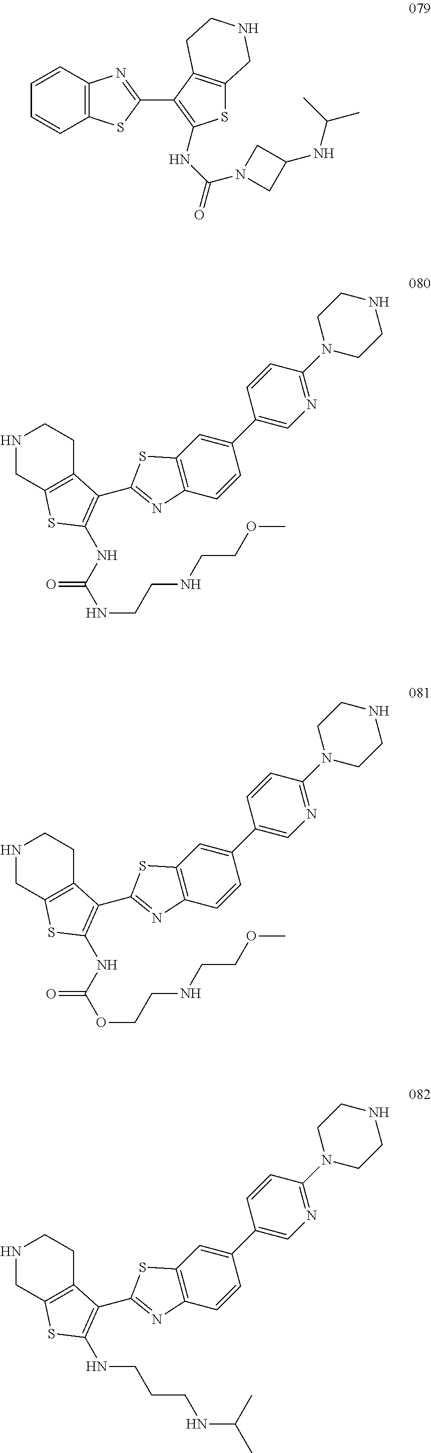 [SMILES: CC(C)NC1CN(C(=O)Nc2sc3c(c2-c2nc4ccccc4s2)CCNC3)C1 ; COCCNCCNC(=O)Nc1sc2c(c1-c1nc3ccc(-c4ccc(N5CCNCC5)nc4)cc3s1)CCNC2 ; COCCNCCOC(=O)Nc1sc2c(c1-c1nc3ccc(-c4ccc(N5CCNCC5)nc4)cc3s1)CCNC2 ; CC(C)NCCCNc1sc2c(c1-c1nc3ccc(-c4ccc(N5CCNCC5)nc4)cc3s1)CCNC2]

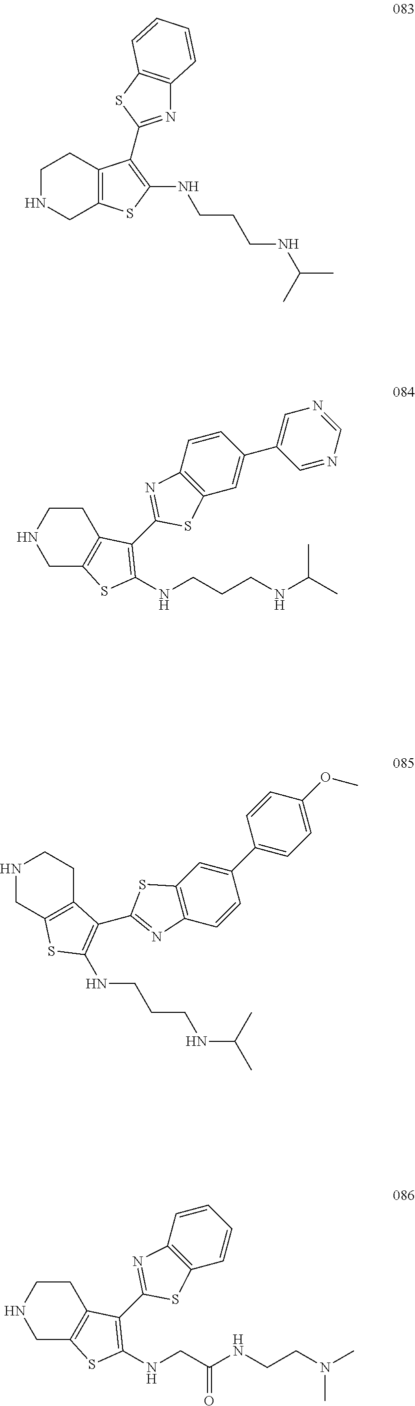 [SMILES: CC(C)NCCCNc1sc2c(c1-c1nc3ccccc3s1)CCNC2 ; CC(C)NCCCNc1sc2c(c1-c1nc3ccc(-c4cncnc4)cc3s1)CCNC2 ; COc1ccc(-c2ccc3nc(-c4c(NCCCNC(C)C)sc5c4CCNC5)sc3c2)cc1 ; CN(C)CCNC(=O)CNc1sc2c(c1-c1nc3ccccc3s1)CCNC2]

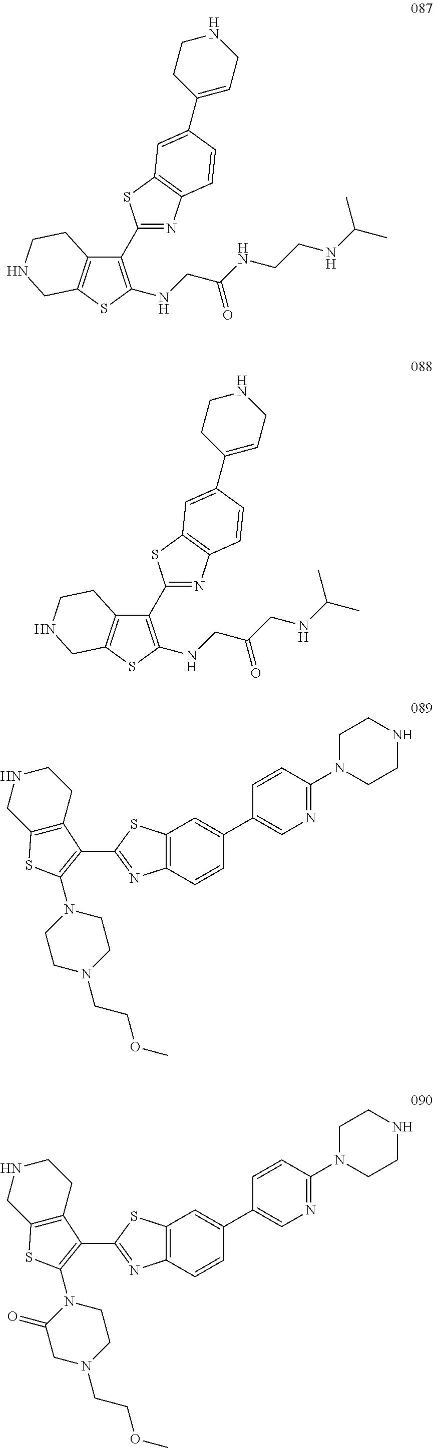 [SMILES: CC(C)NCCNC(=O)CNc1sc2c(c1-c1nc3ccc(C4=CCNCC4)cc3s1)CCNC2 ; CC(C)NCC(=O)CNc1sc2c(c1-c1nc3ccc(C4=CCNCC4)cc3s1)CCNC2 ; COCCN1CCN(c2sc3c(c2-c2nc4ccc(-c5ccc(N6CCNCC6)nc5)cc4s2)CCNC3)CC1 ; COCCN1CCN(c2sc3c(c2-c2nc4ccc(-c5ccc(N6CCNCC6)nc5)cc4s2)CCNC3)C(=O)C1]

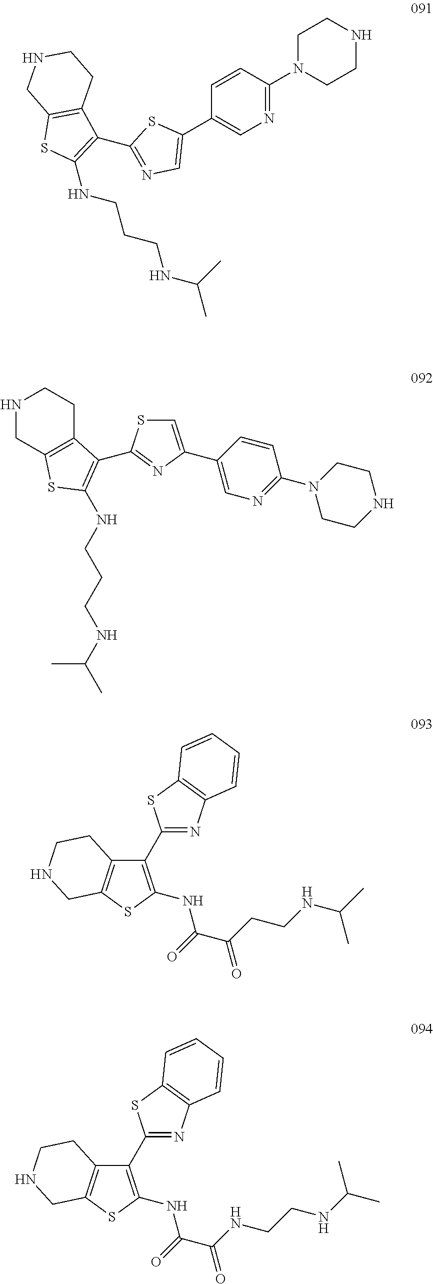 [SMILES: CC(C)NCCCNc1sc2c(c1-c1ncc(-c3ccc(N4CCNCC4)nc3)s1)CCNC2 ; CC(C)NCCCNc1sc2c(c1-c1nc(-c3ccc(N4CCNCC4)nc3)cs1)CCNC2 ; CC(C)NCCC(=O)C(=O)Nc1sc2c(c1-c1nc3ccccc3s1)CCNC2 ; CC(C)NCCNC(=O)C(=O)Nc1sc2c(c1-c1nc3ccccc3s1)CCNC2]

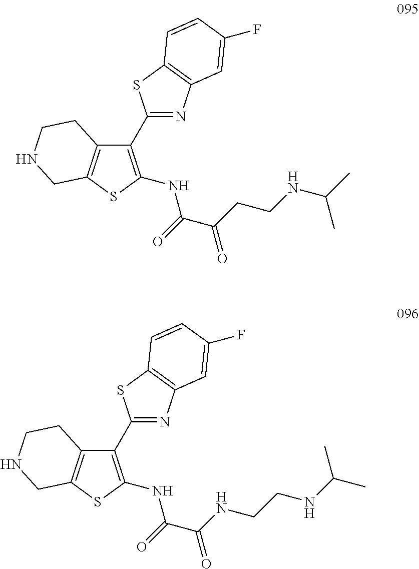 [SMILES: CC(C)NCCC(=O)C(=O)Nc1sc2c(c1-c1nc3cc(F)ccc3s1)CCNC2 ; CC(C)NCCNC(=O)C(=O)Nc1sc2c(c1-c1nc3cc(F)ccc3s1)CCNC2]

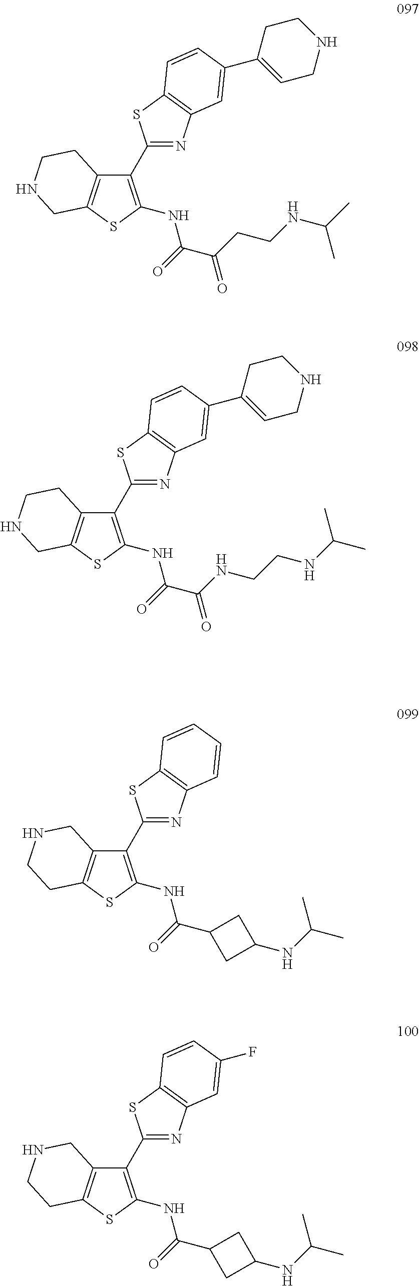 [SMILES: CC(C)NCCC(=O)C(=O)Nc1sc2c(c1-c1nc3cc(C4=CCNCC4)ccc3s1)CCNC2 ; CC(C)NCCNC(=O)C(=O)Nc1sc2c(c1-c1nc3cc(C4=CCNCC4)ccc3s1)CCNC2 ; CC(C)NC1CC(C(=O)Nc2sc3c(c2-c2nc4ccccc4s2)CNCC3)C1 ; CC(C)NC1CC(C(=O)Nc2sc3c(c2-c2nc4cc(F)ccc4s2)CNCC3)C1]

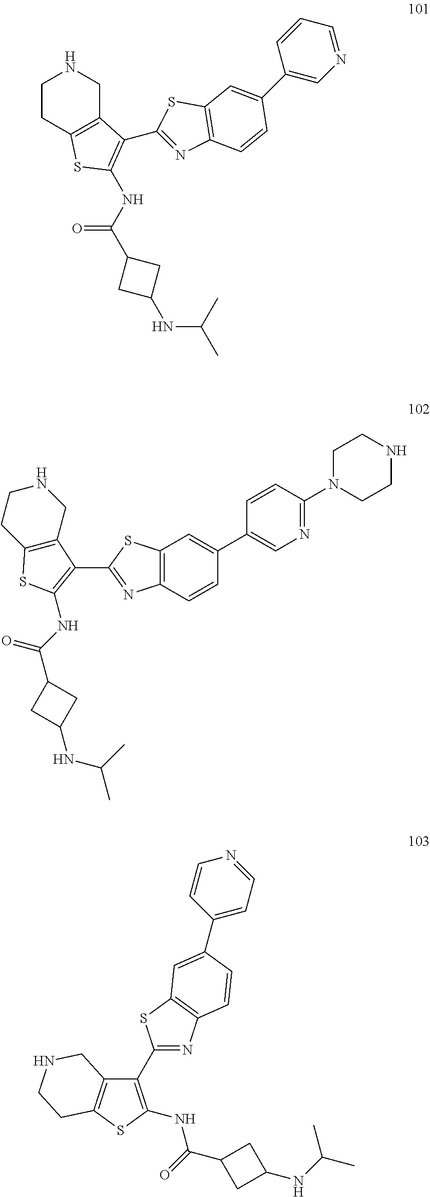 [SMILES: CC(C)NC1CC(C(=O)Nc2sc3c(c2-c2nc4ccc(-c5cccnc5)cc4s2)CNCC3)C1 ; CC(C)NC1CC(C(=O)Nc2sc3c(c2-c2nc4ccc(-c5ccc(N6CCNCC6)nc5)cc4s2)CNCC3)C1 ; CC(C)NC1CC(C(=O)Nc2sc3c(c2-c2nc4ccc(-c5ccncc5)cc4s2)CNCC3)C1]

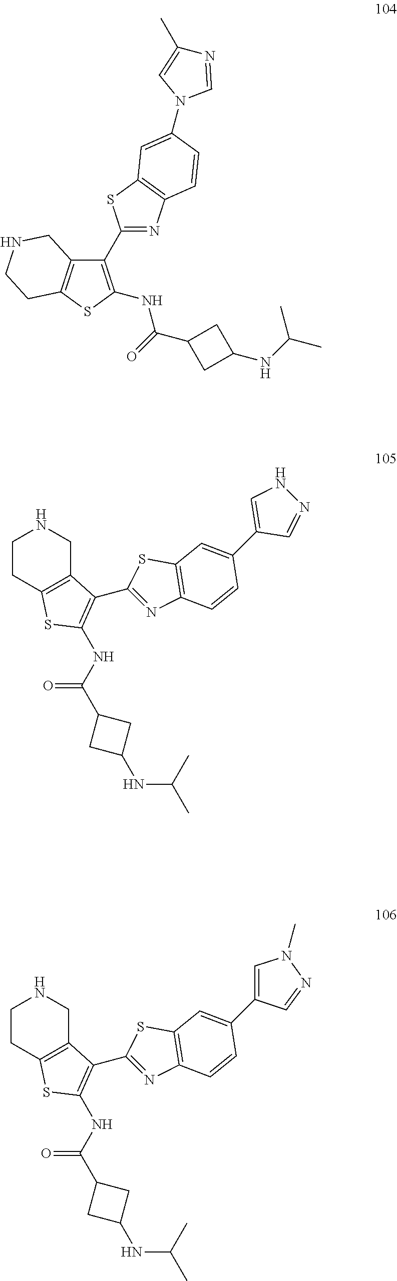 [SMILES: Cc1cn(-c2ccc3nc(-c4c(NC(=O)C5CC(NC(C)C)C5)sc5c4CNCC5)sc3c2)cn1 ; CC(C)NC1CC(C(=O)Nc2sc3c(c2-c2nc4ccc(-c5cn[nH]c5)cc4s2)CNCC3)C1 ; CC(C)NC1CC(C(=O)Nc2sc3c(c2-c2nc4ccc(-c5cnn(C)c5)cc4s2)CNCC3)C1]

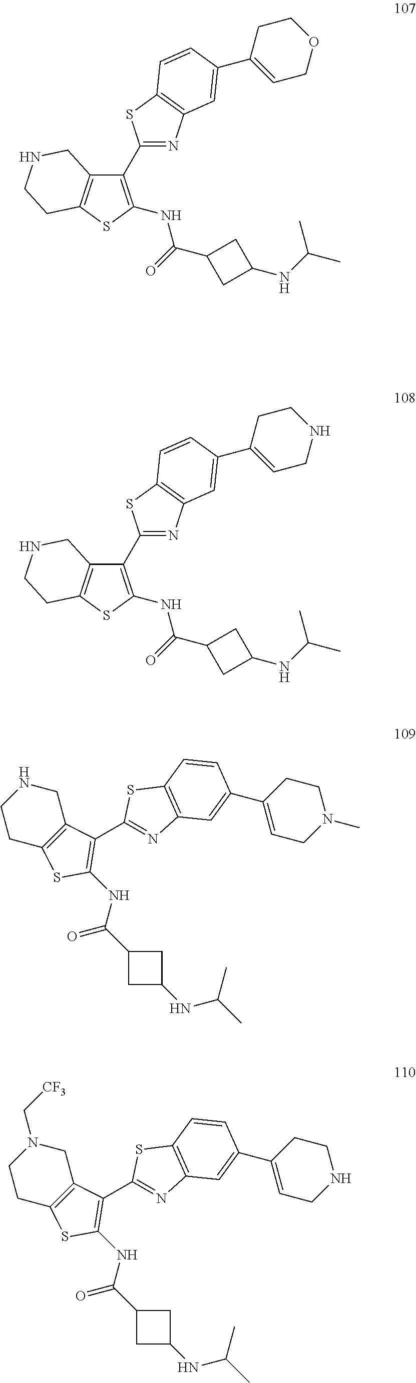 [SMILES: CC(C)NC1CC(C(=O)Nc2sc3c(c2-c2nc4cc(C5=CCOCC5)ccc4s2)CNCC3)C1 ; CC(C)NC1CC(C(=O)Nc2sc3c(c2-c2nc4cc(C5=CCNCC5)ccc4s2)CNCC3)C1 ; CC(C)NC1CC(C(=O)Nc2sc3c(c2-c2nc4cc(C5=CCN(C)CC5)ccc4s2)CNCC3)C1 ; CC(C)NC1CC(C(=O)Nc2sc3c(c2-c2nc4cc(C5=CCNCC5)ccc4s2)CN(CC(F)(F)F)CC3)C1]

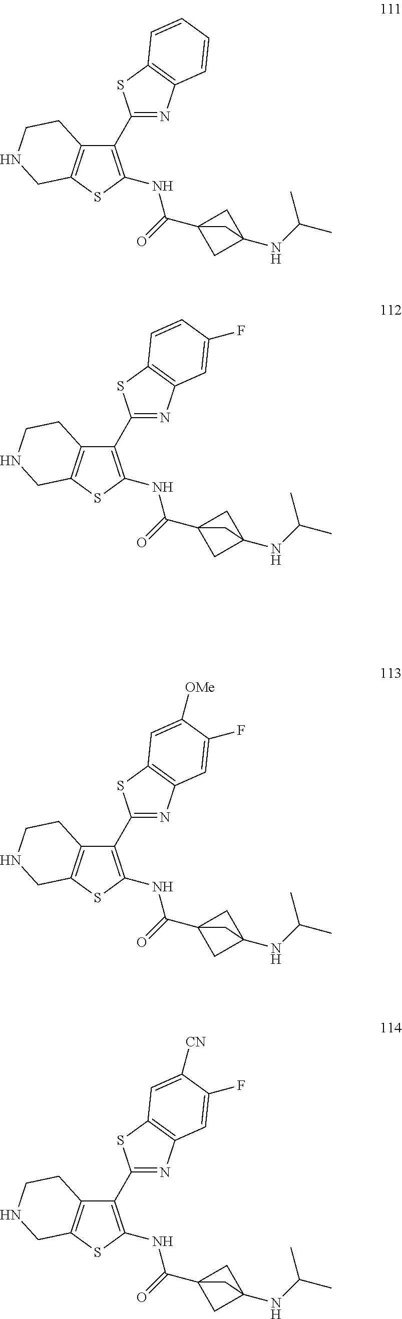 [SMILES: CC(C)NC12CC(C(=O)Nc3sc4c(c3-c3nc5ccccc5s3)CCNC4)(C1)C2 ; CC(C)NC12CC(C(=O)Nc3sc4c(c3-c3nc5cc(F)ccc5s3)CCNC4)(C1)C2 ; COc1cc2sc(-c3c(NC(=O)C45CC(NC(C)C)(C4)C5)sc4c3CCNC4)nc2cc1F ; CC(C)NC12CC(C(=O)Nc3sc4c(c3-c3nc5cc(F)c(C#N)cc5s3)CCNC4)(C1)C2]

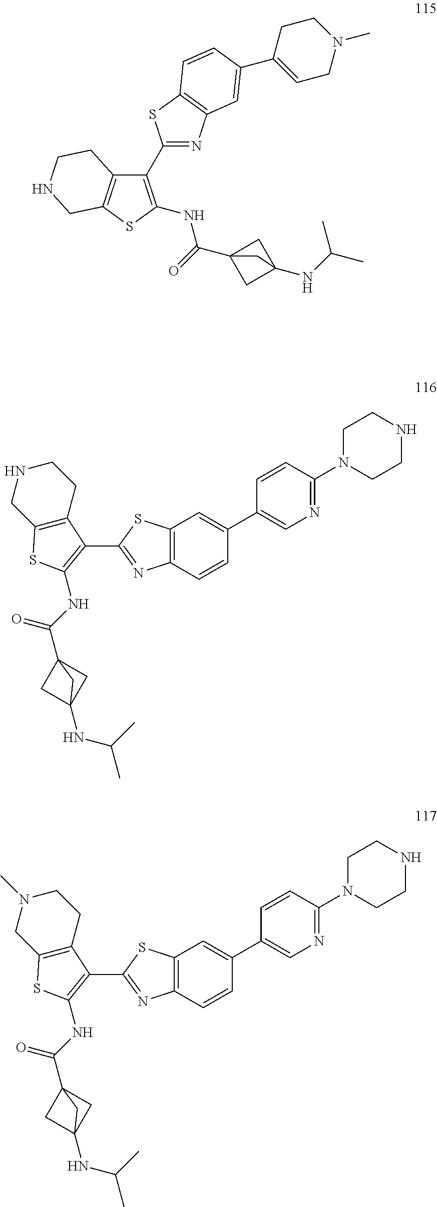

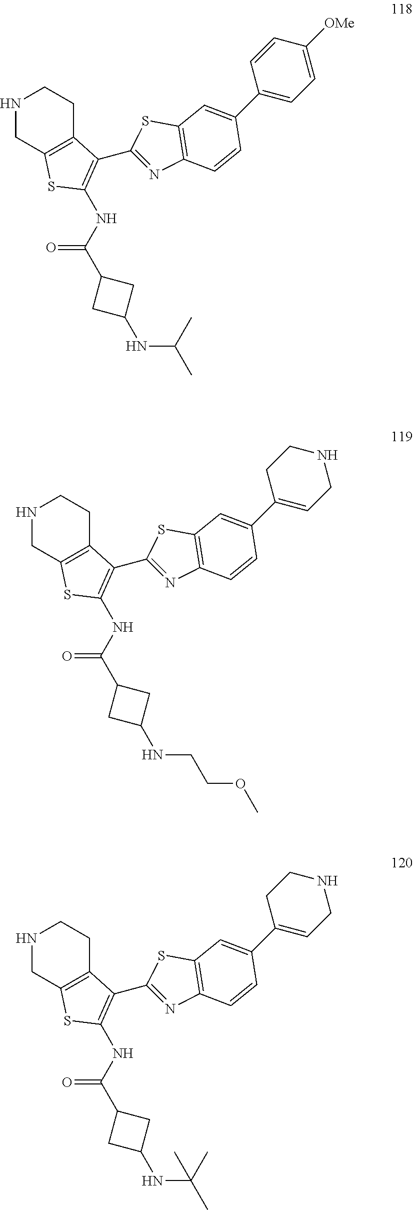 [SMILES: COc1ccc(-c2ccc3nc(-c4c(NC(=O)C5CC(NC(C)C)C5)sc5c4CCNC5)sc3c2)cc1 ; COCCNC1CC(C(=O)Nc2sc3c(c2-c2nc4ccc(C5=CCNCC5)cc4s2)CCNC3)C1 ; CC(C)(C)NC1CC(C(=O)Nc2sc3c(c2-c2nc4ccc(C5=CCNCC5)cc4s2)CCNC3)C1]

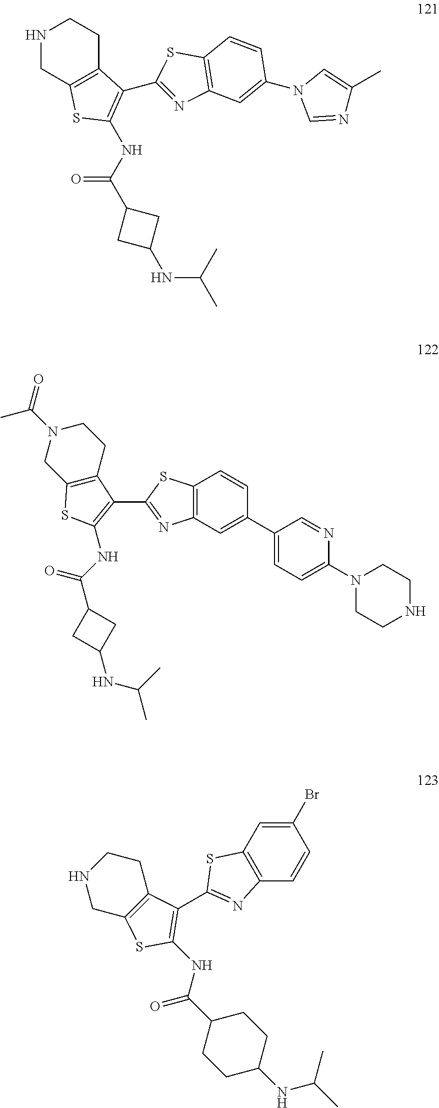

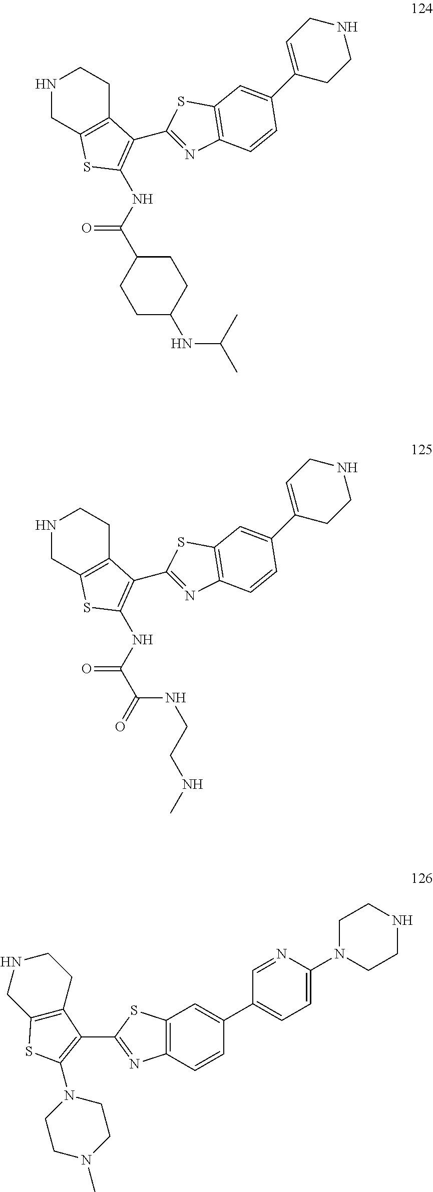 [SMILES: CC(C)NC1CCC(C(=O)Nc2sc3c(c2-c2nc4ccc(C5=CCNCC5)cc4s2)CCNC3)CC1 ; CNCCNC(=O)C(=O)Nc1sc2c(c1-c1nc3ccc(C4=CCNCC4)cc3s1)CCNC2 ; CN1CCN(c2sc3c(c2-c2nc4ccc(-c5ccc(N6CCNCC6)nc5)cc4s2)CCNC3)CC1]

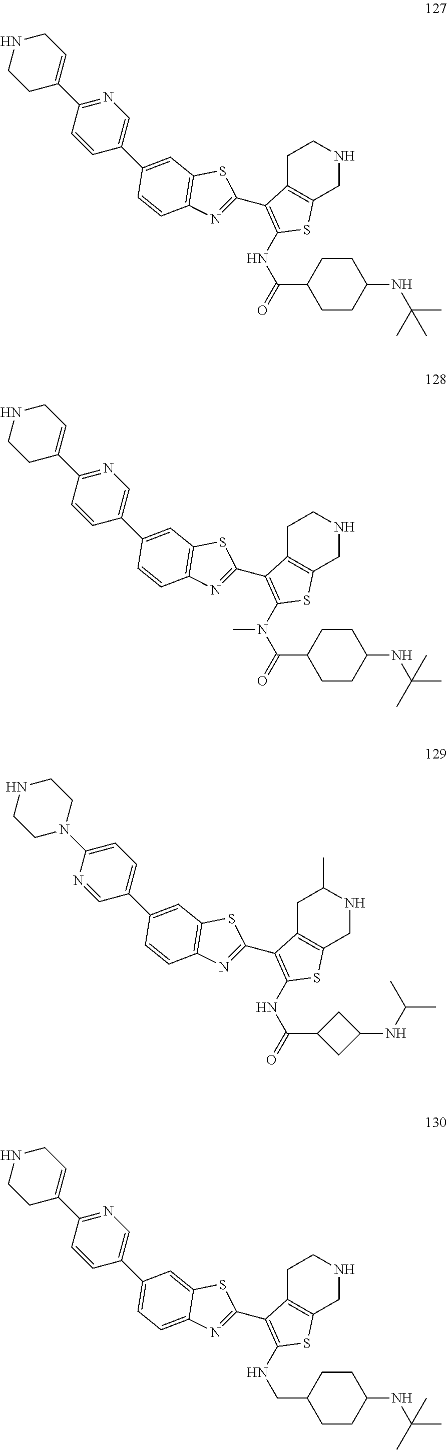 [SMILES: CC(C)(C)NC1CCC(C(=O)Nc2sc3c(c2-c2nc4ccc(-c5ccc(C6=CCNCC6)nc5)cc4s2)CCNC3)CC1 ; CN(C(=O)C1CCC(NC(C)(C)C)CC1)c1sc2c(c1-c1nc3ccc(-c4ccc(C5=CCNCC5)nc4)cc3s1)CCNC2 ; CC(C)NC1CC(C(=O)Nc2sc3c(c2-c2nc4ccc(-c5ccc(N6CCNCC6)nc5)cc4s2)CC(C)NC3)C1 ; CC(C)(C)NC1CCC(CNc2sc3c(c2-c2nc4ccc(-c5ccc(C6=CCNCC6)nc5)cc4s2)CCNC3)CC1]

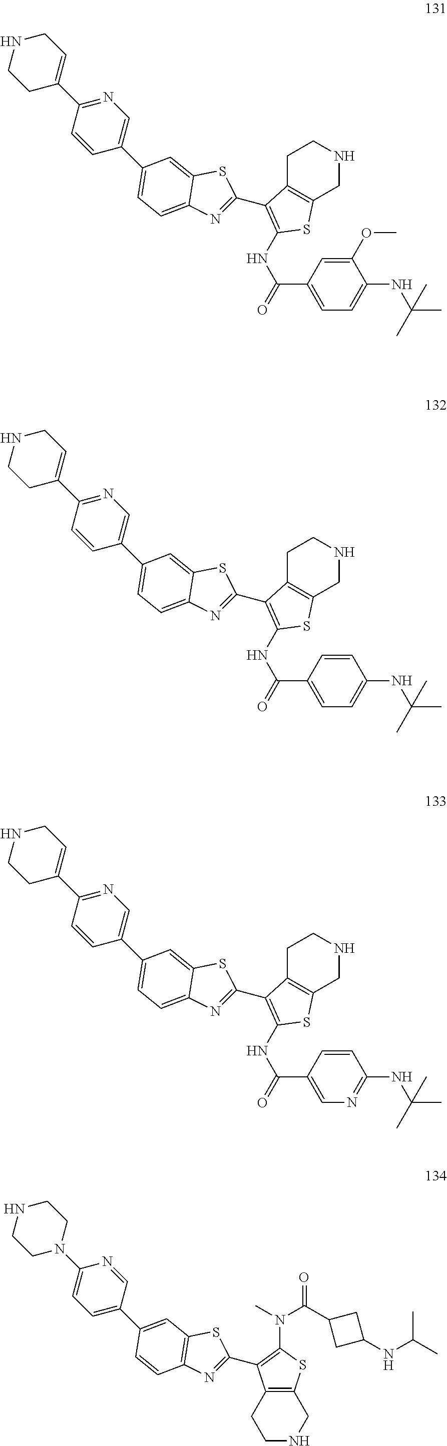 [SMILES: COc1cc(C(=O)Nc2sc3c(c2-c2nc4ccc(-c5ccc(C6=CCNCC6)nc5)cc4s2)CCNC3)ccc1NC(C)(C)C ; CC(C)(C)Nc1ccc(C(=O)Nc2sc3c(c2-c2nc4ccc(-c5ccc(C6=CCNCC6)nc5)cc4s2)CCNC3)cc1 ; CC(C)(C)Nc1ccc(C(=O)Nc2sc3c(c2-c2nc4ccc(-c5ccc(C6=CCNCC6)nc5)cc4s2)CCNC3)cn1 ; CC(C)NC1CC(C(=O)N(C)c2sc3c(c2-c2nc4ccc(-c5ccc(N6CCNCC6)nc5)cc4s2)CCNC3)C1]

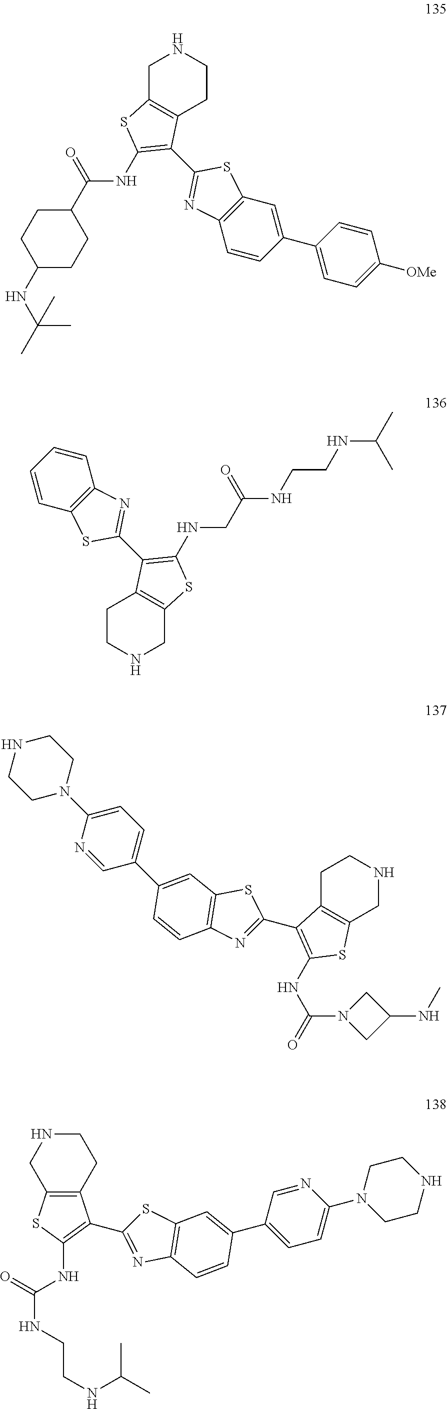 [SMILES: COc1ccc(-c2ccc3nc(-c4c(NC(=O)C5CCC(NC(C)(C)C)CC5)sc5c4CCNC5)sc3c2)cc1 ; CC(C)NCCNC(=O)CNc1sc2c(c1-c1nc3ccccc3s1)CCNC2 ; CNC1CN(C(=O)Nc2sc3c(c2-c2nc4ccc(-c5ccc(N6CCNCC6)nc5)cc4s2)CCNC3)C1 ; CC(C)NCCNC(=O)Nc1sc2c(c1-c1nc3ccc(-c4ccc(N5CCNCC5)nc4)cc3s1)CCNC2]

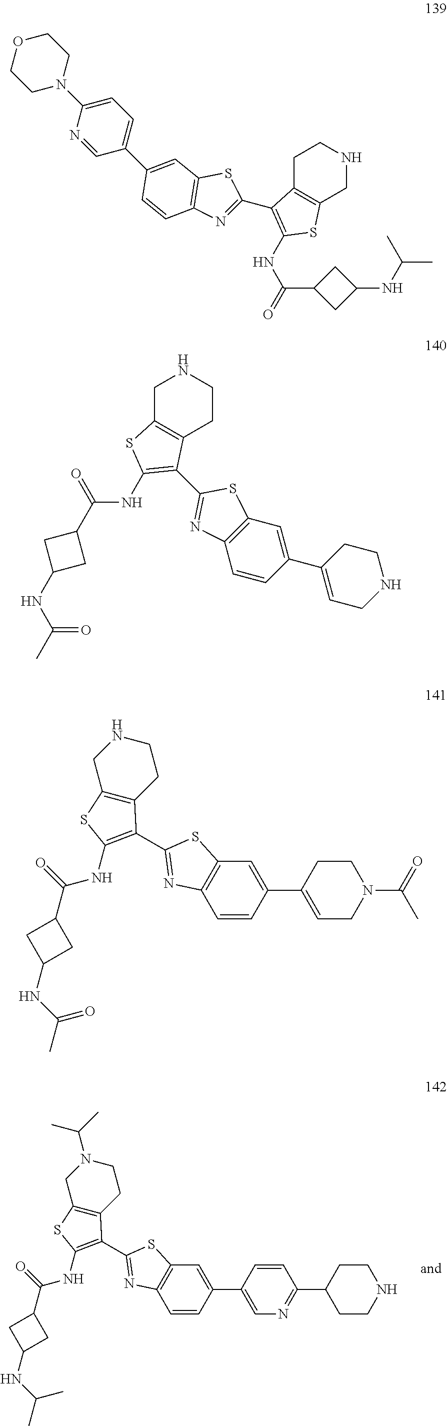 [SMILES: CC(C)NC1CC(C(=O)Nc2sc3c(c2-c2nc4ccc(-c5ccc(N6CCOCC6)nc5)cc4s2)CCNC3)C1 ; CC(=O)NC1CC(C(=O)Nc2sc3c(c2-c2nc4ccc(C5=CCNCC5)cc4s2)CCNC3)C1 ; CC(=O)NC1CC(C(=O)Nc2sc3c(c2-c2nc4ccc(C5=CCN(C(C)=O)CC5)cc4s2)CCNC3)C1 ; CC(C)NC1CC(C(=O)Nc2sc3c(c2-c2nc4ccc(-c5ccc(C6CCNCC6)nc5)cc4s2)CCN(C(C)C)C3)C1]

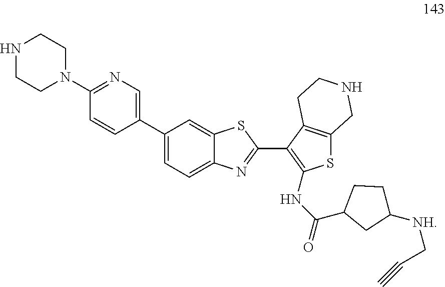 [SMILES: C#CCNC1CCC(C(=O)Nc2sc3c(c2-c2nc4ccc(-c5ccc(N6CCNCC6)nc5)cc4s2)CCNC3)C1]

13. A pharmaceutical composition, comprising an effective amount of the compound or the stereoisomer, the tautomer or the pharmaceutically acceptable salt thereof according to claim 1, and a pharmaceutically acceptable carrier, excipient or a combination thereof.

14. A method for treating a disease mediated by Myc in a subject in need thereof, comprising administering the compound or the stereoisomer, tautomer, or pharmaceutically acceptable salt thereof according to claim 1 to the subject in need thereof, wherein the Myc is preferably c-Myc.

15. The method according to claim 14, wherein the disease mediated by Myc is a solid tumor or a malignant disease of blood system; preferably, the disease mediated by Myc is selected from the group consisting of lymphoma, leukemia, osteosarcoma, liver cancer, kidney cancer, lung cancer, pancreatic cancer and breast cancer.

16. A method for treating lymphoma, leukemia, osteosarcoma, liver cancer, kidney cancer, lung cancer, pancreatic cancer or breast cancer in a subject in need thereof, comprising administering the compound or the stereoisomer, tautomer, or pharmaceutically acceptable salt thereof according to claim 1 to the subject in need thereof.
